(19) United States
(12) Patent Application Publication
(10) Pub. No.: US 2019/0201542 A1
(43) Pub. Date: Jul. 4, 2019

(54) ANTI-DLL3 DRUG CONJUGATES FOR TREATING TUMORS AT RISK OF NEUROENDOCRINE TRANSITION

(71) Applicant: ABBVIE STEMCENTRX LLC, NORTH CHICAGO, IL (US)

(72) Inventors: Laura SAUNDERS, SAN FRANCISCO, CA (US); Robert A. STULL, ALAMEDA, CA (US); Daniel R. HYDUKE, SAN FRANCISCO, CA (US); Enying YOU, SOUTH SAN FRANCISCO, CA (US)

(73) Assignee: ABBVIE STEMCENTRX LLC, NORTH CHICAGO, IL (US)

(21) Appl. No.: 16/302,975

(22) PCT Filed: May 19, 2017
(86) PCT No.: PCT/US17/33601

§ 371 Date: Nov. 19, 2018

Related U.S. Application Data
(60) Provisional application No. 62/339,776, filed on May 20, 2016.
(60) Provisional application No. 62/505,539, filed on May 12, 2017.

(51) Int. Cl. A61K 47/68; C07K 16/28; A61K 31/5517; A61P 35/00; A61K 47/65; A61K 47/54; G01N 33/574

(52) U.S. Cl. CPC A61K 47/6803; C07K 16/28; A61K 47/6849; A61K 31/5517; A61P 35/00; C07K 2317/56; A61K 47/542; A61K 47/545; G01N 33/57496; C07K 2317/24; C07K 2317/565; A61K 47/65

(57) ABSTRACT

Methods of treating tumors at risk for neuroendocrine transition using anti-delta-like ligand 3 (DLL3) antibody drug conjugates (ADCs) are provided.

### CROSS REFERENCED APPLICATIONS

This application claims the benefit of U.S. Provisional Application No. 62/339,776 filed on May 20, 2016 and U.S. Provisional Application No. 62/505,539 filed on May 12, 2017, each of which is incorporated herein by reference in its entirety.

### SEQUENCE LISTING

This application contains a sequence listing that has been submitted in ASCII format via EFS-Web and is hereby incorporated by reference in its entirety. Said ASCII copy, created on May 16, 2017, is named sc1606WOO1_Sequence_Listing and is 663 KB (679,869 bytes) in size.

### FIELD OF THE INVENTION

This application generally relates to methods of treating cancer and any recurrence, relapse, or metastasis thereof. In a broad aspect, the present invention relates to the use of delta-like ligand 3 (DLL3) antibody drug conjugates for the treatment of cancer.

### BACKGROUND OF THE INVENTION

Conventional treatments for cancer include chemotherapy, radiotherapy, surgery, immunotherapy, targeted therapeutics or combinations thereof. Unfortunately, certain cancers are non-responsive or minimally responsive to such treatments. For example, in some patients tumors exhibit gene mutations that render them non-responsive despite the general effectiveness of selected therapies. Moreover, depending on the type of cancer and what form it takes some available treatments, such as surgery, may not be viable alternatives. Limitations inherent in current standard of care therapeutics are particularly evident when attempting to treat patients who have undergone previous treatments and have subsequently relapsed. In such cases the failed therapeutic regimens and resulting patient deterioration may contribute to refractory and/or relapsed tumors, which often manifest themselves as a relatively aggressive disease that ultimately proves to be incurable. Given the highly aggressive nature of relapsed disease, the sooner the relapse is detected and treated the better the prognosis.

Although there have been great improvements in the diagnosis and treatment of cancer over the years, overall survival rates for many solid tumors have remained largely unchanged due to failure of existing therapies to prevent relapse, tumor recurrence and metastases. Thus, it remains a challenge to develop more targeted and potent therapies for cancer, especially for patients with recurrent cancer.

### SUMMARY OF THE INVENTION

In a broad aspect, the present invention provides methods of treating adenocarcinoma at risk of transitioning to a neuroendocrine phenotype and methods of reducing or inhibiting recurrence of an adenocarcinoma at risk of transitioning to a neuroendocrine phenotype. Other aspects of the invention provides isolated anti-DLL3 antibodies, isolated anti-ASCL1 antibodies, and corresponding antibody drug conjugates (ADCs).

In one embodiment, a method of treating an adenocarcinoma at risk of transitioning to a neuroendocrine phenotype in a subject is provided. Such a method comprises administering to the subject a therapeutically effective amount of an anti-DLL3 antibody drug conjugate, or a pharmaceutically acceptable salt thereof, wherein the antibody drug conjugate (ADC) comprises the formula M-[L-D]n wherein: M comprises an anti-DLL3 antibody; L comprises an optional linker; D comprises a cytotoxic agent; and n is an integer from 1 to 20. In certain embodiments the adenocarcinoma expresses relatively little or no detectable level of DLL3 prior to transition to a neuroendocrine phenotype. Significantly, in certain embodiments the adenocarcinoma expresses relatively little or no detectable level of DLL3 protein (e.g., it is DLL3^{-/low}) at the time of treatment with a DLL3 ADC though it may express marker proteins (e.g., ASCL1) indicating that it is at risk of transitioning to a tumor comprising a neuroendocrine phenotype.

In another embodiment, a method of reducing or inhibiting recurrence of an adenocarcinoma at risk of transitioning to a neuroendocrine phenotype in a subject is provided. Such a method comprises administering to the subject a therapeutically effective amount of an anti-DLL3 antibody drug conjugate, or a pharmaceutically acceptable salt thereof, wherein the antibody drug conjugate (ADC) comprises the formula M-[L-D]n wherein: M comprises an anti-DLL3 antibody; L comprises an optional linker; D comprises a cytotoxic agent; and n is an integer from 1 to 20. In selected embodiments the adenocarcinoma expresses relatively little or no detectable levels of DLL3 prior to transition to a neuroendocrine phenotype. Again, in certain embodiments the adenocarcinoma expresses relatively little or no detectable level of DLL3 protein (e.g., it is DLL3^{-/low}) at the time of treatment with a DLL3 ADC though it may express marker proteins (e.g., ASCL1) indicating that it is at risk of transitioning to a tumor comprising a neuroendocrine phenotype.

In some embodiments, the adenocarcinoma comprises ASCL1⁺ cells. In certain embodiments, the adenocarcinoma shows reduced expression of one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG), as compared to a control sample. In other embodiments, the adenocarcinoma shows increased expression of paternally expressed 10 (PEG10) as compared to a control sample.

In some aspects of the invention, the subject is or has undergone a targeted therapy or chemotherapy. In other aspects, the adenocarcinoma is recurrent, refractory, relapsed or resistant. In yet other aspects, the subject has previously undergone a debulking procedure.

As indicated, the present invention provides methods of treating adenocarcinoma and methods of preventing, reducing or inhibiting recurrence of adenocarcinoma at risk of transitioning to a neuroendocrine phenotype. In some aspects, the adenocarcinoma occurs in lung, prostate, genitourinary tract (including bladder), gastrointestinal tract, thyroid, or kidney.

In one aspect, the adenocarcinoma comprises prostate cancer. In certain embodiments, the prostate cancer comprises castration resistant prostate cancer (CPRC). In further embodiments, the adenocarcinoma is resistant to androgen deprivation therapy and in certain embodiments the CPRC is resistant to androgen deprivation therapy (AR-CPRC).

In another aspect, the adenocarcinoma comprises lung cancer. In certain embodiments, the lung cancer comprises non-small cell lung cancer. In a further embodiment, the adenocarcinoma is characterized as having an activating EGFR mutation. In yet another embodiment, the adenocarcinoma is resistant to EGFR inhibitor therapy.

The present invention comprises anti-DLL3 antibodies or ADCs comprising anti-DLL3 antibodies. In some aspects, the anti-DLL3 antibodies bind specifically to an epitope within the DSL domain of a DLL3 protein set forth as SEQ ID NO: 3 or 4. In certain aspects, the anti-DLL3 antibody specifically binds to an epitope comprising amino acids G203, R205 and P206 (SEQ ID NO: 4).

In one embodiment, the anti-DLL3 antibody comprises or competes for binding to human DLL3 protein with an antibody comprising a light chain variable region set forth as SEQ ID NO: 149 and a heavy chain variable region set forth as SEQ ID NO: 151. In another embodiment, the anti-DLL3 antibody comprises three complementarity determining regions of a light chain variable region set forth as SEQ ID NO: 149, and three complementarity determining regions of a heavy chain variable region set forth as SEQ ID NO: 151. In yet another embodiment, the anti-DLL3 antibody comprises residues 24-34 of SEQ ID NO: 149 for CDR-L1, residues 50-56 of SEQ ID NO: 149 for CDR-L2, residues 89-97 of SEQ ID NO: 149 for CDR-L3, residues 31-35 of SEQ ID NO: 151 for CDR-H1, residues 50-65 of SEQ ID NO: 151 for CDR-H2 and residues 95-102 of SEQ ID NO: 151 for CDR-H3, wherein the residues are numbered according to Kabat. In certain embodiments, the anti-DLL3 antibody comprises a light chain variable region comprising an amino acid sequence set forth as SEQ ID NO: 405 and a heavy chain variable region comprising an amino acid sequence set forth as SEQ ID NO: 407.

In some aspects, the anti-DLL3 antibody is selected from the group consisting of a monoclonal antibody, primatized antibody, multispecific antibody, bispecific antibody, monovalent antibody, multivalent antibody, anti-idiotypic antibody, diabody, Fab fragment, F(ab′)₂ fragment, Fv fragment, and ScFv fragment; or an immunoreactive fragment thereof. In certain aspects, the anti-DLL3 antibody is selected from the group consisting of a chimeric antibody, a CDR-grafted antibody, and a humanized antibody.

As indicated, certain embodiments of the invention comprise an antibody drug conjugate of the formula M-[L-D]n, wherein D comprises a cytotoxic agent. In some embodiments, the cytotoxic agent is a pyrrolobenzodiazepine (PBD), an auristatin, a maytansinoid, a calicheamicin, or a radioisotope. In certain embodiments, the cytotoxic agent is a pyrrolobenzodiazepine (PBD). In some aspects, the PBD is covalently linked to the anti-DLL3 antibody via a linker.

In certain aspects the invention comprises an ADC wherein the cytotoxic agent is a pyrrolobenzodiazepine (PBD) comprising formula AC:

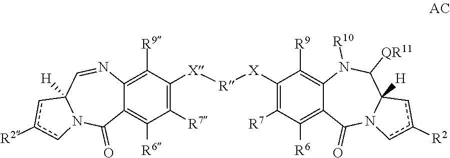

wherein: the dotted lines indicate the optional presence of a double bond, and wherein only one of the dotted lines in a given ring can be a double bond; R² is selected from H, OH, ═O, ═CH₂, CN, R, OR, ═CH—R^{D}, ═C(R^{D})₂, O SO₂ R, CO₂R, COR, and halo, where R^{D} is selected from R, CO₂R, COR, CHO, CO₂H, and halo; R⁶ and R⁹ are each independently selected from H, R, OH, OR, SH, SR, NH₂, NHR, NRR′, NO₂, Me₃Sn and halo; R⁷ is selected from H, R, OH, OR, SH, SR, NH₂, NHR, NRR′, NO₂, Me₃Sn and halo; R¹⁰ is the linker L connected to the anti-DLL3 antibody; Q is selected from O, S and NH; R¹¹ is either H, or R or, where Q is O, R¹¹ may be SO₃M, where M is a metal cation; R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring; X is selected from O, S, and N(H); R^{2″}, R^{6″}, R^{7″}, R^{9″}, and X″ are as defined according to R², R⁶, R⁷, R⁹, and X, respectively; and R″ is a C₃₋₁₂ alkylene group, which comprises a chain optionally interrupted by one or more heteroatoms, one or more rings, or both one or more heteroatoms and one or more rings, wherein the optional one or more rings are optionally substituted. In certain aspects, R² is R, wherein R is an optionally substituted C₁₋₁₂ alkyl; R⁶ and R⁹ are H; R⁷ is OR, and wherein R is a C₁ alkyl; Q is O, and wherein R¹¹ is H; and/or X and X″ are O.

In another aspect, the invention comprises an ADC wherein, following cleavage from any linker, the PBD comprises:

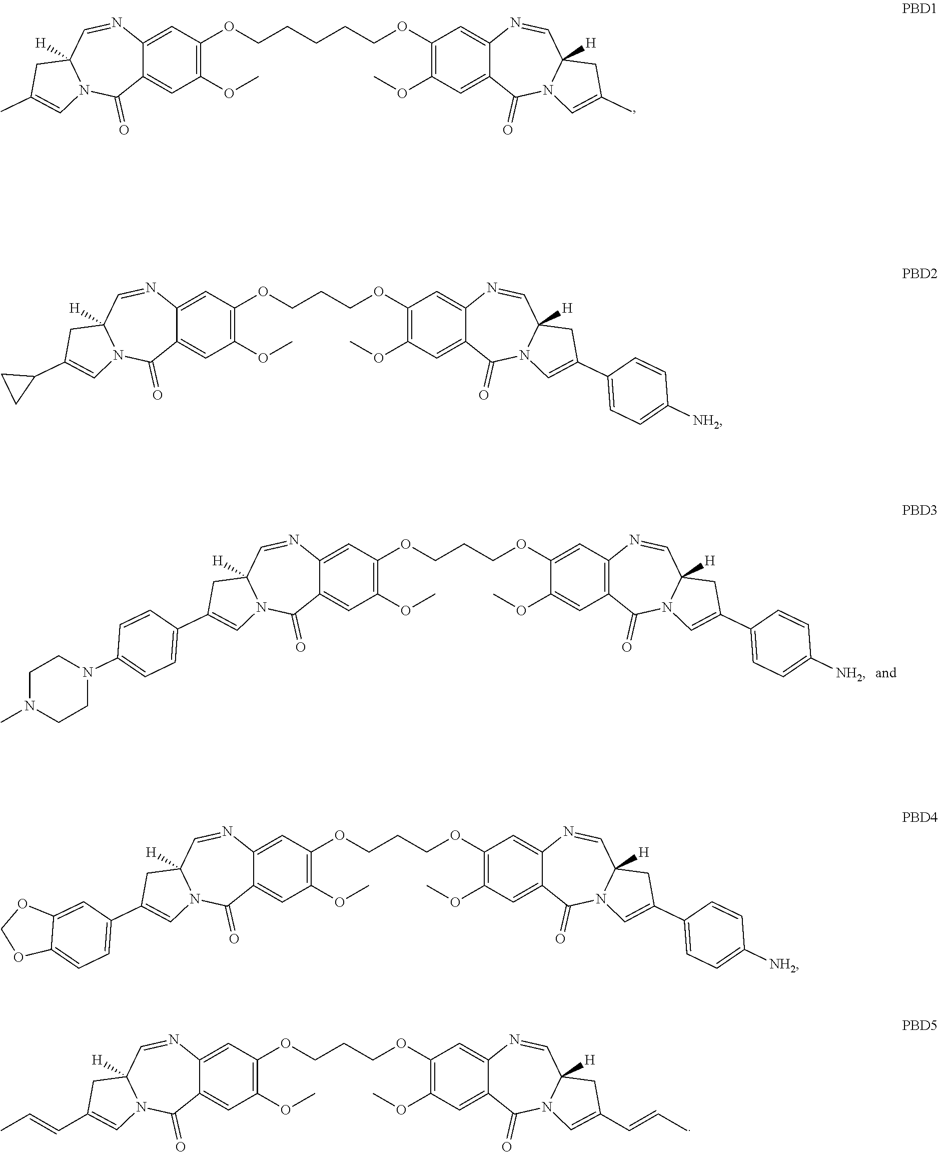

In other aspects of the invention, the ADC comprises a linker. In one aspect, the linker comprises a cleavable linker. In certain aspects, the cleavable linker comprises a dipeptide. In further aspects, the dipeptide is Phe-Lys, Val-Ala, Val-Lys, Ala-Lys, Val-Cit, Phe-Cit, Leu-Cit, Ile-Cit, Phe-Arg, or Trp-Cit. In yet a further aspect, the dipeptide is Val-Ala. In yet other aspects, the linker further comprises a maleimide group.

In certain aspects, the antibody drug conjugate comprises the structure:

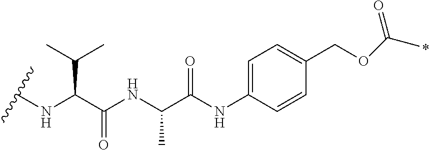 [SMILES: *N[C@H](C(=O)N[C@@H](C)C(=O)Nc1ccc(COC(C)=O)cc1)C(C)C]

wherein the asterisk indicates the point of attachment of the linker to the cytotoxic agent, and wherein the wavy line indicates the point of attachment to the remaining portion of the linker.

Another aspect of the invention provides methods of selecting a subject for treatment with an anti-DLL3 antibody drug conjugate (ADC). Such methods comprise: (a) contacting a tumor sample obtained from the subject with an ASCL1 antibody, wherein the ASCL1 antibody may comprise or compete for binding to a human ASCL1 protein with an antibody comprising a light chain variable region set forth as SEQ ID NO: 521 and a heavy chain variable region set forth as SEQ ID NO: 523; a light chain variable region set forth as SEQ ID NO: 525 and a heavy chain variable region set forth as SEQ ID NO: 527; a light chain variable region set forth as SEQ ID NO: 529 and a heavy chain variable region set forth as SEQ ID NO: 531; a light chain variable region set forth as SEQ ID NO: 533 and a heavy chain variable region set forth as SEQ ID NO: 535; a light chain variable region set forth as SEQ ID NO: 537 and a heavy chain variable region set forth as SEQ ID NO: 539; a light chain variable region set forth as SEQ ID NO: 541 and a heavy chain variable region set forth as SEQ ID NO: 543; a light chain variable region set forth as SEQ ID NO: 545 and a heavy chain variable region set forth as SEQ ID NO: 547; a light chain variable region set forth as SEQ ID NO: 549 and a heavy chain variable region set forth as SEQ ID NO: 551; a light chain variable region set forth as SEQ ID NO: 553 and a heavy chain variable region set forth as SEQ ID NO: 555; a light chain variable region set forth as SEQ ID NO: 557 and a heavy chain variable region set forth as SEQ ID NO: 559; a light chain variable region set forth as SEQ ID NO: 561 and a heavy chain variable region set forth as SEQ ID NO: 563; a light chain variable region set forth as SEQ ID NO: 565 and a heavy chain variable region set forth as SEQ ID NO: 567; a light chain variable region set forth as SEQ ID NO: 569 and a heavy chain variable region set forth as SEQ ID NO: 571; or a light chain variable region set forth as SEQ ID NO: 521 and a heavy chain variable region set forth as SEQ ID NO: 573; (b) detecting the ASCL1 antibody bound to the tumor sample; and (c) selecting a subject having an ASCL1⁺ tumor sample for treatment with an anti-DLL3 antibody drug conjugate (ADC). In certain embodiments the ASCL1 antibody will comprise or compete with the aforementioned antibodies. In the step of detecting the ASCL1 antibody, the antibody may be conjugated or otherwise associated with a detectable label, or this step may be performed using a secondary and/or tertiary antibody that specifically binds to the ASCL1 antibody to amplify the signal, as is well known in the art. In other selected embodiments the selected subject may express relatively little or no detectable levels of DLL3 at the time of selection.

In a further aspect, the invention comprises an antibody that binds to human ASCL1 comprising a light chain variable region and a heavy chain variable region, wherein the light chain variable region has three CDRs of a light chain variable region set forth as SEQ ID NO: 521, SEQ ID NO: 525, SEQ ID NO: 529, SEQ ID NO: 533, SEQ ID NO: 537, SEQ ID NO: 541, SEQ ID NO: 545, SEQ ID NO: 549, SEQ ID NO: 553, SEQ ID NO: 557, SEQ ID NO: 561, SEQ ID NO: 565 or SEQ ID NO: 569 and the heavy chain variable region has three CDRs of a heavy chain variable region set forth as SEQ ID NO: 523, SEQ ID NO: 527, SEQ ID NO: 531, SEQ ID NO: 535, SEQ ID NO: 539, SEQ ID NO: 543, SEQ ID NO: 547, SEQ ID NO: 551, SEQ ID NO: 555, SEQ ID NO: 559 and SEQ ID NO: 563, SEQ ID NO: 567, SEQ ID NO: 571 or SEQ ID NO: 573. As described in some detail below the CDRs may be defined according to Kabat, Chothia, MacCallum or AbM methodology.

It will be appreciated that certain aspects of the methods of selecting a subject for treatment with an anti-DLL3 ADC comprise the use of ASCL1 antibodies for immunohistochemistry. In one embodiment, detecting the ASCL1 antibody is performed using immunohistochemistry. In some embodiments, the tumor sample is chemically fixed. In certain embodiments, the tumor sample is chemically fixed using formalin. In other embodiments, the tumor sample is paraffin embedded.

In other aspects, the methods of selecting a subject for treatment with an anti-DLL3 ADC can further comprise any or all of the steps: contacting the tumor sample with one or more agents that detect one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG); detecting the one or more agents in the tumor sample; and observing a reduced expression of one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG), as compared to a control sample.

In still other aspects, the methods of selecting a subject for treatment with an anti-DLL3 ADC can further comprise the steps of: contacting the tumor sample with an agent that detects paternally expressed 10 (PEG10); detecting the agent in the tumor sample; and observing an increase in expression of paternally expressed 10 (PEG10) as compared to a control sample.

In yet a further aspect, each of the aforementioned methods of selecting a subject for treatment with an anti-DLL3 ADC can further comprise the step of administering an anti-DLL3 antibody drug conjugate to the subject.

In this regard certain aspects of the invention comprise a method of treating a subject suffering from a tumor at risk of transitioning to a neuroendocrine phenotype comprising the steps of (a) contacting a tumor sample obtained from the subject with an ASCL1 antibody; (b) detecting the ASCL1 antibody bound to the tumor sample; (c) selecting a subject having an ASCL1⁺ tumor phenotype; and (d) treating the subject selected in step (c) with an anti-DLL3 antibody drug conjugate (DLL3 ADC). In certain preferred aspects the tumor will comprise an adenocarcinoma. In further embodiments the subject may tested for DLL3 expression (e.g., using a DLL3 antibody). In such embodiments the subject may be treated with a DLL3 ADC even though the adenocarcinoma comprises an ASCL1⁺DLL3^{-/low} phenotype.

In some aspects of the aforementioned methods of selecting a subject for treatment with an anti-DLL3 ADC, the tumor is at risk of transitioning to a neuroendocrine phenotype. In certain aspects, the subject is and/or has undergone a targeted therapy or chemotherapy. In further aspects, the tumor is recurrent, refractory, relapsed or resistant. In yet other aspects, the subject has previously undergone a debulking procedure.

In certain aspects of the methods of selecting a subject for treatment with an anti-DLL3 ADC, the tumor occurs in lung, prostate, genitourinary tract, gastrointestinal tract, thyroid, or kidney.

In one aspect, the tumor comprises prostate cancer. In a certain aspect, the prostate cancer comprises castration resistant prostate cancer. In yet a further aspect, the prostate cancer is resistant to androgen deprivation therapy.

In another aspect, the tumor comprises lung cancer. In certain aspects, the lung cancer comprises small cell lung cancer. In a further aspect, the lung cancer comprises non-small cell lung cancer. In still another aspect, the tumor is characterized as having an activating EGFR mutation. In yet a further aspect, the tumor is resistant to EGFR inhibitor therapy.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIGS. 1A and 1B provide, in a tabular form, contiguous amino acid sequences (SEQ ID NOS: 21-407, odd numbers) of light and heavy chain variable regions of a number of murine and humanized exemplary DLL3 antibodies;

FIG. 2 depicts in schematic form the results of domain level mapping analysis of exemplary DLL3 antibodies;

FIG. 3A-3C provide, in tabular form, contiguous amino acid sequences of light (FIG. 3A) and heavy chain (FIG. 3B) variable regions, and the nucleotide sequences (FIG. 3C) of the light chain and heavy chain variable regions of exemplary murine anti-ASCL1 antibodies (SEQ ID NOS: 521-573).

FIG. 4 depicts DLL3 expression in androgen resistant castration resistant prostate cancer (AR-CRPC);

FIG. 5 depicts DLL3 expression in metastatic prostate cancer;

FIG. 6 depicts DLL3 expression in androgen resistant prostate cancer with neuroendocrine differentiation (NEPC);

FIG. 7 depicts DLL3 expression over time during mouse host castration as the tumor initially regresses but rapidly relapses as NEPC;

FIG. 8 depicts PEG10, DLL3, SPDEF, PTGER4 and ERG expression over time in LTL331 during mouse host castration as the tumor initially regresses but rapidly relapses as NEPC;

FIG. 9 depicts DLL3 expression in AR-CRPC;

FIG. 10 depicts EGFR mutated, TKI resistant lung adenocarcinoma tumors that exhibit elevated levels of DLL3 after small cell transformation and EMT;

FIG. 11 depicts DLL3 and CHGA expression on a common cancer array with multiple types of tumor; and

FIG. 12 provides, in tabular form, immunohistochemistry results for ASCL1 expression in various small cell lung cancer PDX samples with anti-ASCL1 antibody clone SC72.2.

### DETAILED DESCRIPTION OF THE INVENTION

The invention may be embodied in many different forms. Disclosed herein are non-limiting, illustrative embodiments of the invention that exemplify the principles thereof. Any section headings used herein are for organizational purposes only and are not to be construed as limiting the subject matter described. For the purposes of the instant disclosure all identifying sequence accession numbers may be found in the NCBI Reference Sequence (RefSeq) database and/or the NCBI GenBank® archival sequence database unless otherwise noted.

### I. Introduction

DLL3 expression has surprisingly been found to correlate with a number of tumor types and, as a determinant, may be exploited in the treatment of such tumors. Among such tumors, DLL3 expression in adenocarcinoma has been found to correlate with tumors that have become resistant to a targeted cancer therapy or chemotherapy and have transitioned to a neuroendocrine phenotype. The present invention provides methods and compositions for identifying tumors at risk for transitioning to a neuroendocrine phenotype, such that patients having the disclosed risk factors are identifiable as candidates for treatment with an anti-DLL3 antibody drug conjugate. For example, as detailed herein, several proteins are expressed in tumors during the transition to a neuroendocrine phenotype and prior to detectable DLL3 expression. Such marker proteins are indicative as to subjects that may respond to treatment with a DLL3 ADC even though the tumor expresses relatively little or no detectable level of DLL3 at the time of treatment. Also as disclosed herein, tumors treated previously with a targeted therapy are particularly vulnerable to undergoing a neuroendocrine transition and are susceptible to the methods of treatment or prophylaxis as disclosed herein. As such, identification of the disclosed risk factors and/or biomarkers as disclosed herein provides new therapeutic strategies for treating DLL3^{-/low} tumors with an anti-DLL3 antibody drug conjugate. It will be appreciated that such treatments may be used to prevent or retard the incidence of recurrent, refractory, relapsed or resistant tumors that would ultimately exhibit a neuroendocrine phenotype.

### II. Risk Factors for Neuroendocrine Transition

Numerous tumors, and particularly adenocarcinomas, can transition, transform, or differentiate to a neuroendocrine phenotype and take on neuroendocrine features. The mechanism of this transition is not well understood, but the neuroendocrine phenotype may arise from rare neuroendocrine cells present in the original tumor. Upon certain therapeutic treatments such cells may preferentially survive and expand as the cells of the original tumor are eliminated. Alternatively, the tumor may undergo a histological transformation to neuroendocrine cells. In any event such transitions may stimulated, induced or otherwise triggered by therapeutic intervention designed to treat the tumor. In other cases such neuroendocrine transitions may be spontaneous.

The present application provides risk factors for tumors that are capable of, or likely to, transition to a neuroendocrine phenotype. In one aspect of the invention, tumors at risk for neuroendocrine transition are identified by increased expression of particular markers (e.g., ASCL1) and/or reduced expression of selected markers, as disclosed herein. In another aspect of the invention, the present inventors have discovered that tumors previously treated with a targeted therapy, as defined herein, are also at risk of transitioning to a neuroendocrine phenotype. Such tumors at risk may be effectively treated using an anti-DLL3 antibody drug conjugate, notwithstanding that these tumors show negative or low expression of DLL3 (DLL3^{-/low}) at the time of treatment.

A. The Neuroendocrine Phenotype

True or canonical neuroendocrine tumors (NETs) arising from the dispersed endocrine system are relatively rare, with an incidence of 2-5 per 100,000 people, but highly aggressive. Neuroendocrine tumors occur in the kidney, genitourinary tract (bladder, prostate, ovary, cervix, and endometrium), gastrointestinal tract (colon, stomach), thyroid (medullary thyroid cancer), and lung (small cell lung carcinoma and large cell neuroendocrine carcinoma). These tumors may secrete several hormones including serotonin and/or chromogranin A that can cause debilitating symptoms known as carcinoid syndrome. Such tumors can be denoted by positive immunohistochemical markers such as neuron-specific enolase (NSE, also known as gamma enolase, gene symbol=ENO2), CD56 (or NCAM1), chromogranin A (CHGA), and synaptophysin (SYP) or by genes known to exhibit elevated expression such as ASCL1. Unfortunately traditional chemotherapies have not been particularly effective in treating NETs and liver metastasis is a common outcome.

Pseudo neuroendocrine tumors (pNETs) are tumors that genotypically or phenotypically mimic, resemble or exhibit common traits with canonical neuroendocrine tumors. Pseudo neuroendocrine tumors or tumors with neuroendocrine features are tumors that arise from cells of the diffuse neuroendocrine system or from cells in which a neuroendocrine differentiation cascade has been aberrantly reactivated during the oncogenic process. Such pNETs commonly share certain phenotypic or biochemical characteristics with traditionally defined neuroendocrine tumors, including the ability to produce subsets of biologically active amines, neurotransmitters, and peptide hormones. Histologically, such tumors (NETs and pNETs) share a common appearance often showing densely connected small cells with minimal cytoplasm of bland cytopathology and round to oval stippled nuclei. For the purposes of the instant invention, the factors identified herein as characterizing a risk of neuroendocrine transition are equally applicable to identifying a risk for a pseudo neuroendocrine transition.

B. Biomarkers for Identifying Tumors at Risk of Neuroendocrine Transition

As disclosed herein, changes in expression of various biomarkers can be used to identify tumors at risk of transitioning to a neuroendocrine phenotype. Representative biomarkers include (1) an increase in expression of one or more of Achaete-scute Homolog 1 (ASCL1), Paternally Expressed 10 (PEG10), or Serine/Arginine Repetitive Matrix 4 (SRRM4), and (2) a decrease or reduction of expression of one or more of Retinoblastoma 1 (RB1), Repressor Element-1 Silencing Transcription Factor (REST), SAM Pointed Domain-containing Ets Transcription Factor (SPDEF), Prostaglandin E2 Receptor 4 (PTGER4), or ETS-Related Gene (ERG). These relative changes in expression may be attributed to a change in the number of cells expressing a marker, i.e., more or fewer cells within the sample characterizable as having positive, low or negative expression. Alternatively or in addition, these relative changes may be the result of a change in the level of expression in any given cell or group of cells.

In selected aspects of the invention the expression of the selected determinant may be measured using flow cytometry comprising fluorescent antibody staining. When using such techniques tumor cells may be defined as exhibiting positive, low and negative marker levels based fluorescent signals. Cells with negative expression (i.e. “FMO⁻”) may be defined as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one control”, or “FMO control”, staining. Cells with expression greater than the 95th percentile of expression observed with an isotype control antibody using the FMO staining procedure described above may be defined as “positive” (i.e. “FMO⁺”). As defined herein there are various populations of cells broadly defined as “positive” including those that may be defined as FMO⁺. A cell is defined as FMO⁺ if the mean observed expression of the antigen is above the 95th percentile determined using FMO staining with an isotype control antibody as described above. The positive cells may be termed cells with low expression (i.e. “FMO-lo”) if the mean observed expression is above the 95th percentile determined by FMO staining and is within one standard deviation of the 95th percentile. Alternatively, the positive cells may be termed cells with high expression (i.e. “FMO-hi”) if the mean observed expression is above the 95th percentile determined by FMO staining and greater than one standard deviation above the 95th percentile. In other embodiments the 99th percentile may preferably be used as a demarcation point between negative and positive FMO staining. A sample that is FMO⁺ for a particular marker, for example, ASCL1⁺, has a detectable level of expression for the marker as compared to a control sample. A tumor that is positive for a particular marker can have detectable levels of the marker in one or more cells.

In certain aspects of the invention increased expression of a given marker (ASCL1⁺) is meant to encompass any significant increase in expression level of the marker in a sample (e.g., a tumor sample) as compared to expression level of the corresponding marker in a control sample (e.g., normal, non-tumorigenic tissue). For example, an increased or higher expression level for a given marker can be any statistically significant increase in the expression level of the marker of at least 5%, 10%, 15%, 20%, 25%, 30%, 40%, 50%, 60%, 70%, 80%, 85%, 90%, 95%, 96%, 97%, 98%, 99%, 100%, 200%, 400% or more as compared to a reference expression level in a control sample. In accordance with the instant disclosure tumor samples showing such increases in expression may be classified as “+” or “hi” when compared to appropriate controls. Alternatively, an increase in the expression level for a given marker can be any fold increase of at least 1.5-fold, 2-fold, 3-fold, 4-fold, 5-fold, 6-fold, 7-fold, 8-fold, 9-fold, 10-fold, 12-fold, 14-fold, 16-fold, 20-fold or more over the value for the expression level of the corresponding marker in a control sample. In accordance with the instant disclosure tumor samples showing such increases in expression level may be classified as “+” or “hi” when compared to appropriate controls. In either case increased expression of a particular marker as observed in a sample may be the result of an increase in the number of positive cells expressing the marker in the sample, and/or an increase in the level of marker expression in any given cell or group of cells within the sample.

Decreased, lower or reduced expression level, or a loss of expression for a given marker refers to any significant decrease in the expression level of the marker in a sample as compared to the expression level of the corresponding marker in a control sample. For example, a significant reduction in the expression level of a marker in a subject sample of at least 5%, 10%, 15%, 20%, 25%, 30%, 40%, 50%, 60%, 70%, 80%, 85%, 90%, 95%, 96%, 97%, 98%, 99% or more as compared to a reference expression level in a control sample. In accordance with the instant disclosure tumor samples showing such increases in expression level may be classified as “−” or “low” when compared to appropriate controls. Alternatively, a decrease in the expression level for a given marker can be any fold decrease of at least 1.5-fold, 2-fold, 3-fold, 4-fold, 5-fold, 6-fold, 7-fold, 8-fold, 9-fold, 10-fold, 12-fold, 14-fold, 16-fold, 20-fold or more as compared to the expression level of the corresponding marker in a control sample. In accordance with the instant disclosure tumor samples showing such increases in expression level may be classified as “−” or “low” when compared to appropriate controls. Decreased expression of a particular marker as observed in a sample may be the result of a decrease in the number of positive cells expressing the marker in the sample, and/or a decrease in the level of marker expression in any given cell or group of cells within the sample.

A control or control sample provides a reference point for measuring changes in expression of markers in the sample. The control may be a predetermined value based on a group of samples or it may be a single value based on an individual sample. The control may be a sample tested in parallel with the subject sample. In certain aspects a control sample may comprise, for example, any normal tissue sample or any tumor sample that has not undergone a transition to a neuroendocrine phenotype.

It will be appreciated that the expression level of a marker can be measured by expression of protein levels of any given marker or by nucleic acid expression levels of any given marker, for example, the expression level of the RNA that encodes the marker. Moreover, expression levels of more than one marker may be determined for a given sample thorough the use of different detection reagents (e.g., different antibodies) or through a combination of different methodologies. Singularly or in combination such expression measurements may be used to provide a descriptive cell or tumor phenotype (e.g., ASCL1⁺, DLL3^{-/low}).

Various assays to measure for expression of a marker are known in the art and are discussed in more detail below and in the Examples. Representative techniques for assessing protein levels include, for example, fluorescence detection assays, such as fluorescence microscopy or flow cytometry, immuno-assays, such as immunohistochemistry, or enzyme detection assays. In preferred aspects immunohistochemistry (IHC) techniques will be used to provide cell or tumor phenotypes in accordance with the teachings and markers herein. By way of example, antibodies that specifically recognize Achaete-scute Homolog 1 (ASCL1), Paternally Expressed 10 (PEG10), or Serine/Arginine Repetitive Matrix 4 (SRRM4), Retinoblastoma 1 (RB1), Repressor Element-1 Silencing Transcription Factor (REST), SAM Pointed Domain-containing Ets Transcription Factor (SPDEF), Prostaglandin E2 Receptor 4 (PTGER4), or ETS-Related Gene (ERG) are disclosed herein, commercially available or otherwise known in the art and may be used for IHC. Representative assays to measure RNA expression levels of these markers include, for example, RT-PCR, such as quantitative RT-PCR. Any art-recognized techniques for assessing protein or RNA expression levels may be used in accordance with the present invention to assess marker expression as described herein that indicates a risk of neuroendocrine transition.

In certain preferred embodiments the assays may comprise immunohistochemistry (IHC) assays or variants thereof (e.g., fluorescent, chromogenic, standard ABC, standard LSAB, etc.) or immunocytochemistry or variants thereof (e.g., direct, indirect, fluorescent, chromogenic, etc.).

In this regard certain aspects of the instant invention comprise the use of labeled ASCL1 for immunohistochemistry (IHC). More particularly IHC (e.g., ASCL1 IHC) may be used as a diagnostic tool to aid in the diagnosis of various proliferative diseases including tumors at risk of transitioning to a neuroendocrine phenotype and to monitor the potential response to treatments, e.g., DLL3 ADC therapy. Compatible diagnostic assays may be performed on tissues that have been chemically fixed (compatible techniques include, but are not limited to: formaldehyde, glutaraldehyde, osmium tetroxide, potassium dichromate, acetic acid, alcohols, zinc salts, mercuric chloride, chromium tetroxide and picric acid) and embedded (compatible methods include but are not limited to: glycol methacrylate, paraffin and resins) or preserved via freezing. Such assays can be used to guide treatment decisions and determine dosing regimens and timing.

As known in the art immunohistochemistry techniques may be used to derive an H-score as known in the art using the antibodies to the disclosed markers. Briefly tumor sections are viewed (preferably by brightfield microscopy) and marker (e.g., ASCL1) expression on sectioned tumor is noted to derive an H-score. The exemplary H-score may be obtained by the formula: 3×percentage of strongly staining nucleus+2×percentage of moderately staining nucleus+percentage of weakly staining nucleus, giving a range of 0 to 300.

Such H-scores may be used to indicate which patients may be amenable to treatment with a suitable composition (e.g., an anti-DLL3 ADC). H-scores of approximately 90, approximately 100, approximately 110, approximately 120, approximately 130, approximately 140, approximately 150, approximately 160, approximately 170, approximately 180, approximately 190 or approximately 200 or above on a 300 point scale may be used in selected embodiments to indicate which patients may respond favorably to the treatment methods of the instant invention (e.g., with a DLL3 ADC and/or chemotherapeutic agent). For example, in certain embodiments, a patient to be treated with an DLL3 ADC will have an ASCL1 H-score of at least 90 (i.e., the tumor is ASCL1⁺) on a 300 point scale. In other embodiments a patient to be treated with a DLL3 ADC as set forth in the instant invention will have an ASCL1 H-score of at least 120. In yet other embodiments a patient to be treated with the DLL3 ADCs of the instant invention will have an H-score of at least 180. For the purposes of the instant disclosure any tumor exhibiting an H-score of 90 or above on a 300 point scale will be considered ASCL1⁺ and subject to treatment with any known therapy useful for treating neuroendocrine tumors, including targeting of transcriptional targets of ASCL1, as described further below.

In other selected embodiments an H-score comprising a 200 point scale may also be used to select or diagnose patients that may respond to treatments as disclosed herein. In such 200 H-score scales an H-score of 120 is approximately equivalent to an H-score of 180 on a 300 H-score scale. In both cases (e.g., 120/200 or 180/300) such H-scores may be classified as positive (e.g., ASCL1⁺ in that they are both above an H-score of 90 on a 300 point scale and/or ≥10% of the constituent cells express ASCL1 as described below) and are suggestive of patients that may respond favorably to the treatment methods of the instant invention.

In other embodiments patient selection may be based on the measurement of percent of positively stained cells in a tumor sample. In this regard patients exhibiting a certain percentage of positively stained cells in an IHC sample when interrogated with a marker antibody, for example an anti-ASCL1 antibody, would be considered ASCL1⁺ and would be selected for treatment in accordance with the teachings herein. In such embodiments tumor samples exhibiting greater than 10%, greater than 20%, greater than 30%, greater than 40% or greater than 50% positive cell staining may be classified as positive for a marker (e.g., ASCL1+) when measured as percent positive cells. In other embodiments tumor samples exhibiting greater than 60%, greater than 70%, greater than 80%, greater than 90% or greater than 95% positive cell staining may be classified as marker positive when measured as percent positive. In certain preferred aspects the ASCL1⁺ tumor will express ASCL1 in ≥10%, ≥20%, ≥30%, ≥40%, or ≥50% of the constituent cells when measured as percent positive. In each of the forgoing embodiments patients suffering from ASCL1 percent positive tumors may be treated with DLL3 ADCs as set forth herein. While the ASCL1 marker was exemplified it will be appreciated that the other disclosed markers (e.g., PEG10 and SRRM4) are also predictive as to which patients will be candidates for the treatment regimens of the instant invention and are expressly included within its scope.

In still other embodiments patient selection may be predicated on the percent of marker positive cells staining with a certain intensity. By way of example, a tumor with >20% of the cells exhibiting 2+ ASCL1 intensity or greater will be ASCL1+ and a candidate for treatment with a DLL3 ADC. In other embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 1+ intensity or greater when stained with a marker antibody (e.g., an anti-ASCL1 antibody) and examined in accordance with standard IHC protocols as disclosed herein. In other certain embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 2+ intensity or greater when stained with a marker antibody and examined in accordance with standard IHC protocols as disclosed herein. In yet other selected embodiments a patient will be a candidate for treatment with a DLL3 ADC or other chemotherapeutic agent if ≥10%, ≥20%, ≥30%, ≥40% or ≥50% of the tumor cells exhibit 1+ intensity or greater when stained with a marker antibody and examined in accordance with standard IHC protocols as disclosed herein. In still other embodiments a patient will be a candidate for treatment with a DLL3 ADC or other chemotherapeutic agent if ≥10%, ≥20%, ≥30%, ≥40% or ≥50% of the tumor cells exhibit 2+ intensity or greater when stained with a marker antibody and examined in accordance with standard IHC protocols as disclosed herein.

Yet another embodiment comprises a method of treating a subject having a tumor comprising tumor cells wherein ≥10% of the tumor cells exhibit 1+ intensity or greater when stained with a marker antibody and examined in accordance with standard IHC protocols comprising the step of administering an anti-DLL3 ADC. With regard to each of the aforementioned embodiments it will be appreciated that the intensity of staining with a marker antibody may be readily determined using standard pathology techniques and methodology familiar to those of skill in the art.

In certain aspects the present invention provides a method of selecting a subject for treatment with an anti-DLL3 antibody drug conjugate by detecting ASCL1 expression in a tumor sample. Such a method can comprise (a) contacting an anti-ASCL1 antibody with a tumor sample obtained from the subject; (b) detecting the anti-ASCL1 antibody in the tumor sample; and (c) selecting a subject having an ASCL1⁺ tumor sample for treatment with an anti-DLL3 antibody drug conjugate (ADC). Concurrently or sequentially, antibodies that specifically bind to one or more of the additional markers disclosed herein (e.g., PEG10, SRRM4, RB1, REST, SAM, SPDEF, PTGER4, and ERG) may be used to further assess or characterized the risk of neuroendocrine transition. For example, the combination of particular markers may be quantified as presenting different levels of risk to guide selection of appropriate treatments, including anti-DLL3 ADC therapy. In certain embodiments the tumor sample will express relatively low levels of DLL3.

As discussed above certain marker levels and DLL3 expression will be decreased or reduced as compared to a reference expression level in a control sample. More specifically, tumors at risk of transitioning to a neuroendocrine phenotype may express lower levels of one markers selected from the group consisting of Retinoblastoma 1 (RB1), Repressor Element-1 Silencing Transcription Factor (REST), SAM Pointed Domain-containing Ets Transcription Factor (SPDEF), Prostaglandin E2 Receptor 4 (PTGER4), and ETS-Related Gene (ERG). In addition, such tumors may express relatively low levels of DLL3 protein and may be classified as ASCL1⁺, DLL3^{-/low} wherein DLL3⁻ is indicative of non-detectable or barely detectable levels of expression and DLL3^{low} is indicative of relatively depressed levels of DLL3 found in certain tumors (e.g., adenocarcinoma). In this regard DLL3^{low} will be held to mean any tumor comprising a DLL3 expression level that is reduced by 5%, 10%, 15%, 20%, 25%, 30%, 40%, 50%, 60%, 70%, 80%, 85%, 90%, 95%, 96%, 97%, 98%, 99% or more as compared to a reference expression level in a control sample (e.g., a DLL3+ or hi tumor). In certain embodiments DLL3 expression will be reduced by 50%, 60%, 70%, 80%, 85%, 90%, 95%, 96%, 97%, 98%, 99% or more as compared to a reference expression level in a control sample. In still other embodiments DLL3 expression will be reduced by 80%, 85%, 90%, 95%, 96%, 97%, 98%, 99% or more as compared to a reference expression level in a control sample while in other embodiments DLL3 expression will be reduced by 90%, 95%, 96%, 97%, 98%, 99% or more as compared to a reference expression level in a control sample. In selected embodiments DLL3 expression will be reduced by at least 90%, by at least 95%, by at least 97% or by at least 99% when compared to a sample obtained from a DLL3⁺ tumor.

In other embodiments the tumor sample may compared to control tumor samples known not to express DLL3 (negative control). When such comparisons are made the tumor sample obtained from the subject may be classified as DLL3⁻ if it exhibits substantially the same level of DLL3 as the negative control.

In yet other embodiments DLL3^{-/low} tumors may readily be identified by trained pathologists using IHC in view of the instant disclosure. More specifically tumor samples may be obtained, preferably fixed and stained with anti-DLL3 antibodies as disclosed herein and read using art-recognized techniques. In certain embodiments the expression of DLL3 may be visually determined by the pathologist using appropriate positive and negative controls. In other embodiments the scoring could be based on a derived H-score may comprise the measurement of percent of positively stained cells in a tumor sample. With respect to the latter a tumor may be found to be DLL3^{-/low} if less than about 20%, 15%, 10%, 8%, 5%, 4%, 3%, 2% or 1% of the cells stain positive using standard IHC techniques. In other embodiments a tumor may be found to be DLL3^{-/low} if less than about 0.8%, 0.6%, 0.4%, 0.2% or 0.1% of the cells stain positive when interrogated with a DLL3 antibody as described herein. Thus, in a preferred aspect the invention will comprise treatment of a patient suffering from a tumor wherein the percentage of cells in the DLL3^{-/low} tumor that stain positive when interrogated with a DLL3 antibody is less than about 10%. In another preferred aspect the invention will comprise treatment of a patient suffering from a tumor wherein the percentage of cells in the DLL3^{-/low} tumor that stain positive when interrogated with a DLL3 antibody is less than about 5%. And in another preferred aspect the invention will comprise treatment of a patient suffering from a tumor wherein the percentage of cells in the DLL3^{-/low} tumor that stain positive when interrogated with a DLL3 antibody is less than about 1%.

In other embodiments the skilled artisan may make a qualitative judgement as to what constitutes a DLL3^{-/low} tumor upon review of the slides based on factors such as relative intensity, staining patterns, sample origin and preparation, antibody and reporter employed, etc. As previously alluded to any determination as to the level of DLL3 expression is made in the context of appropriate positive and negative controls and is relatively accurate. Accordingly such determinations are indicative as to which patients are susceptible to treatment with DLL3 ADCs as described herein.

More generally, based on the teachings herein one skilled in the art could readily determine which tumors comprise DLL3^{-/low} phenotypes using a number of compatible techniques and, as potential neuroendocrine tumors, are candidates for treatment with an anti-DLL3 ADC using the disclosed methods.

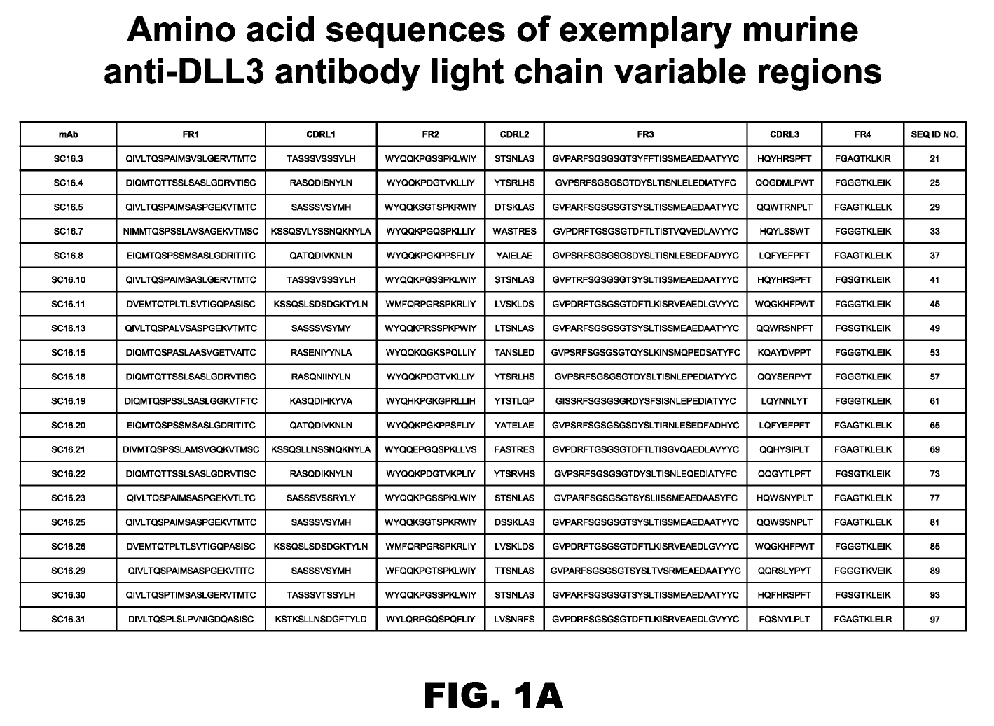

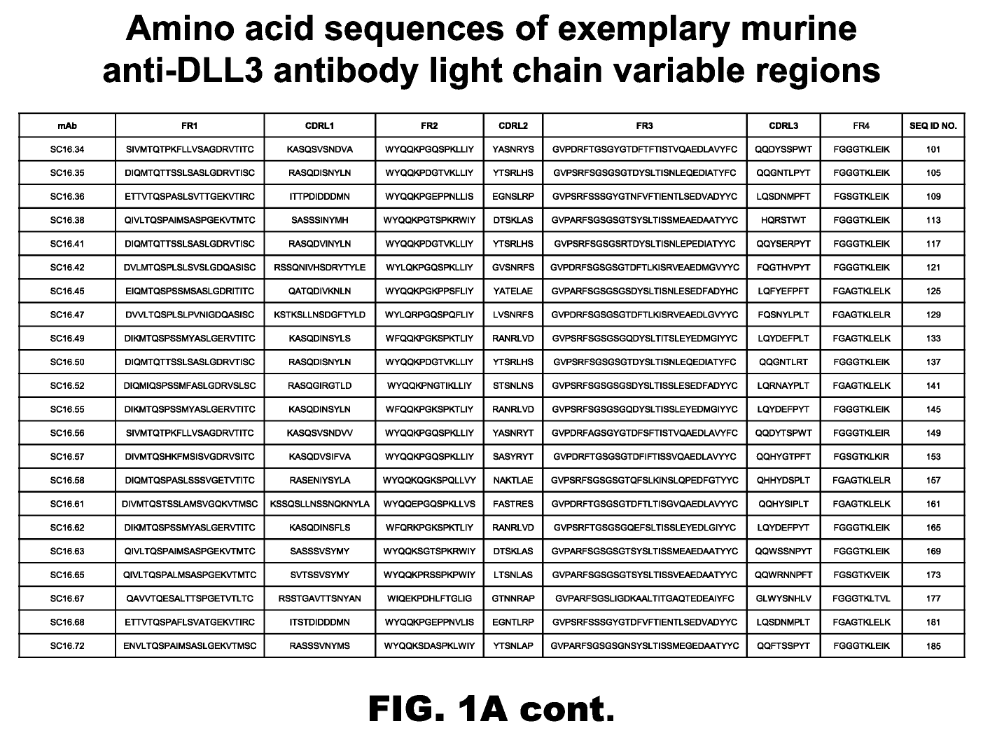

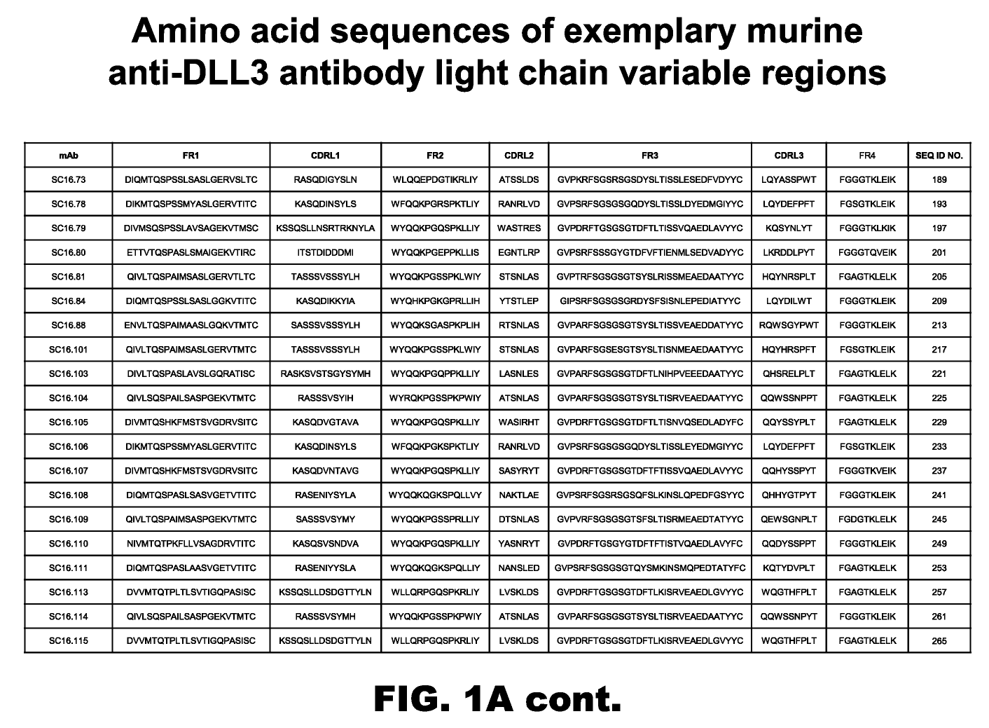

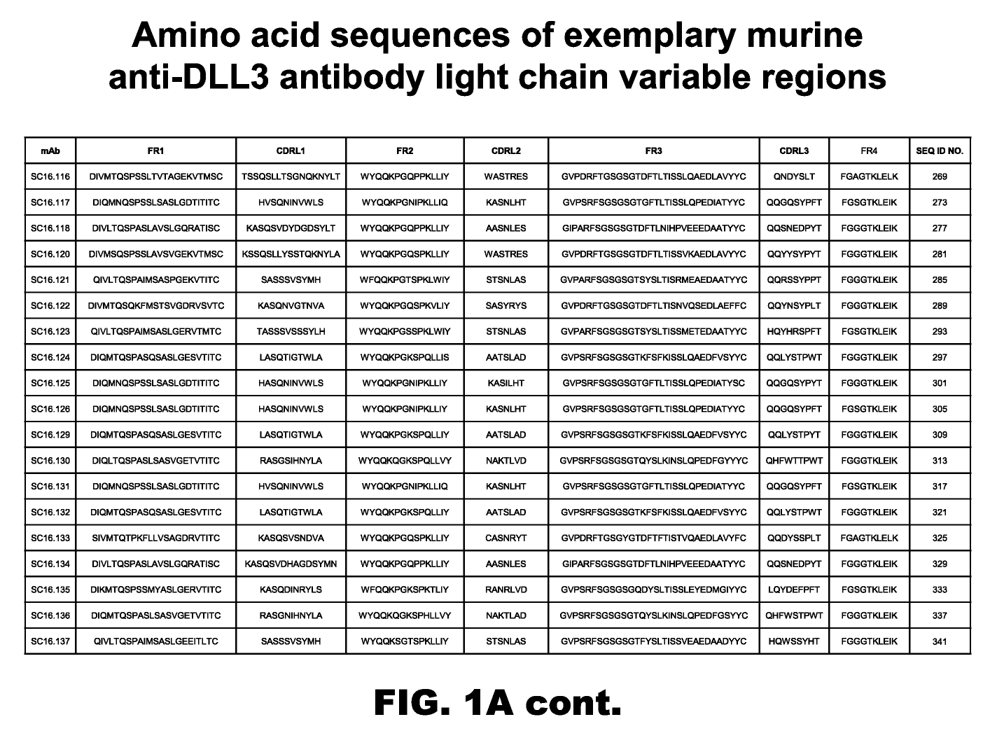

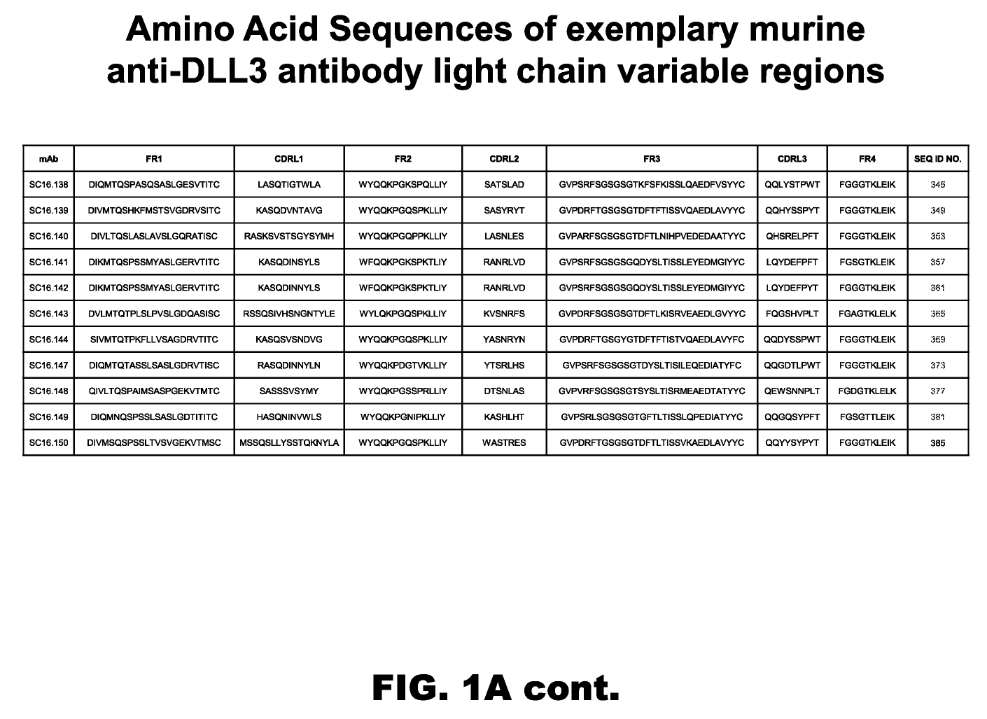

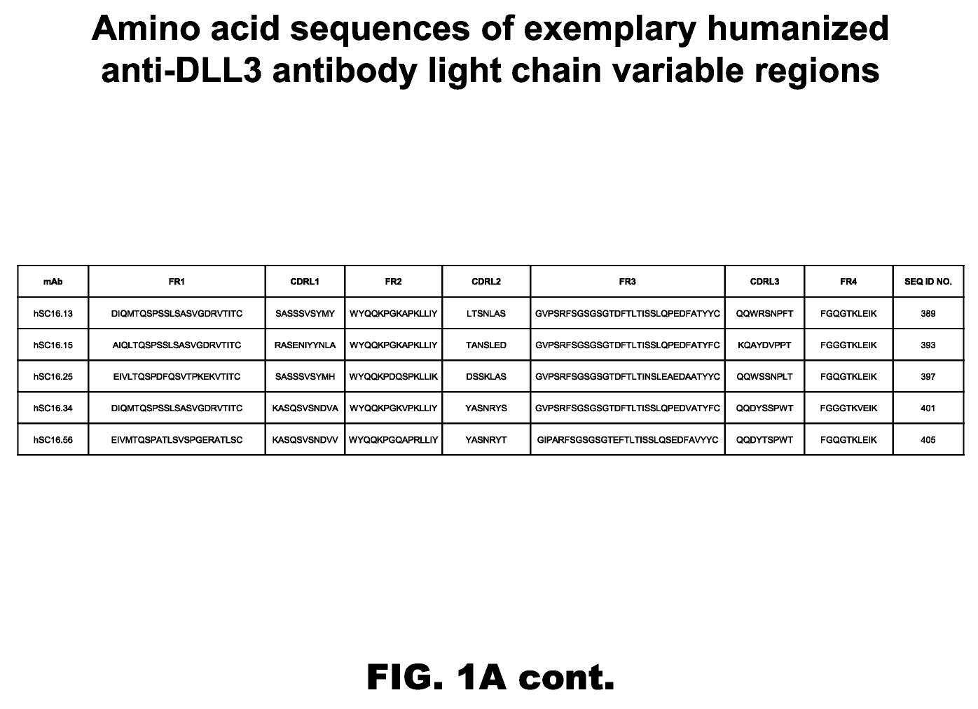

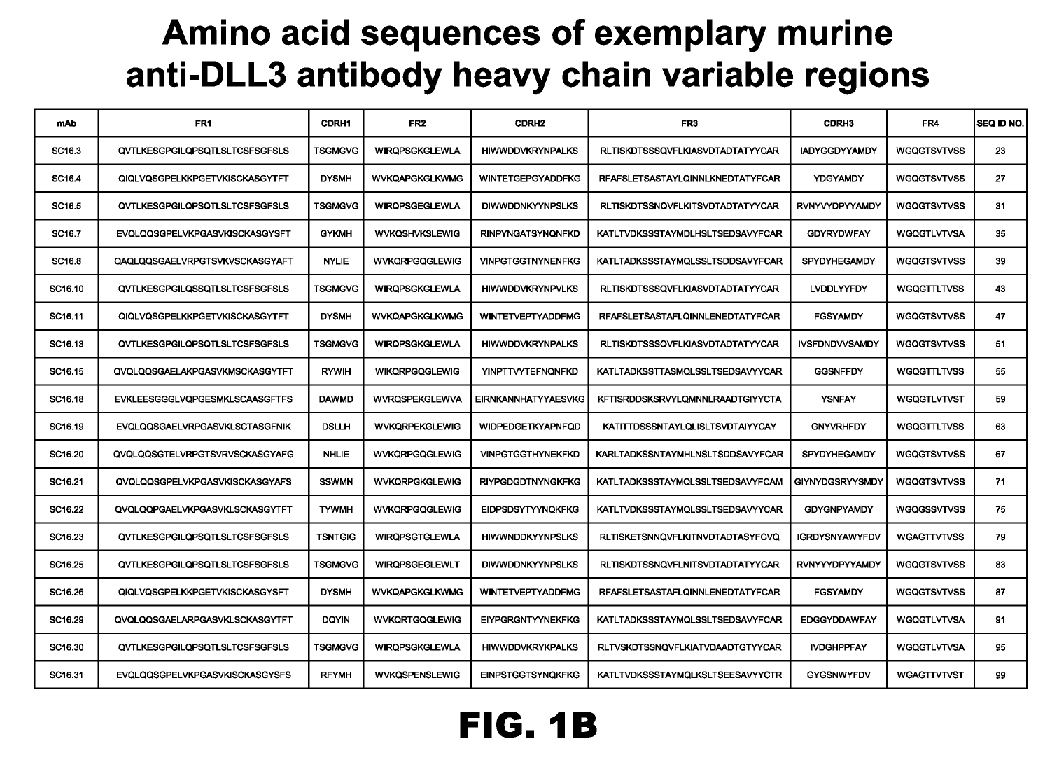

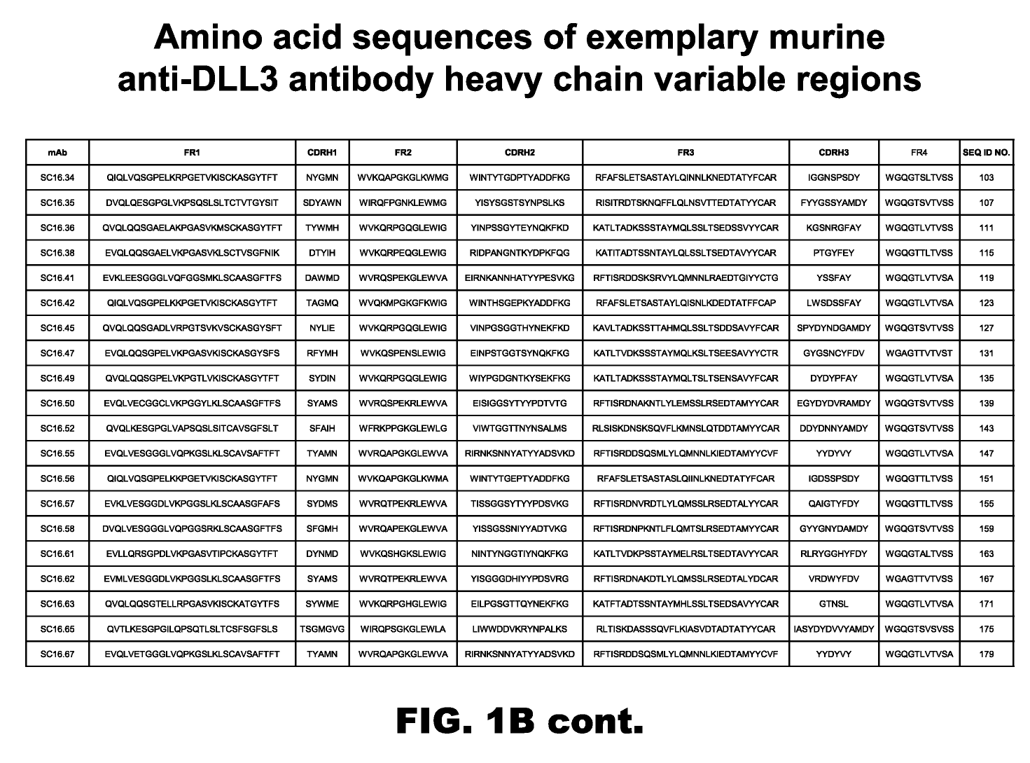

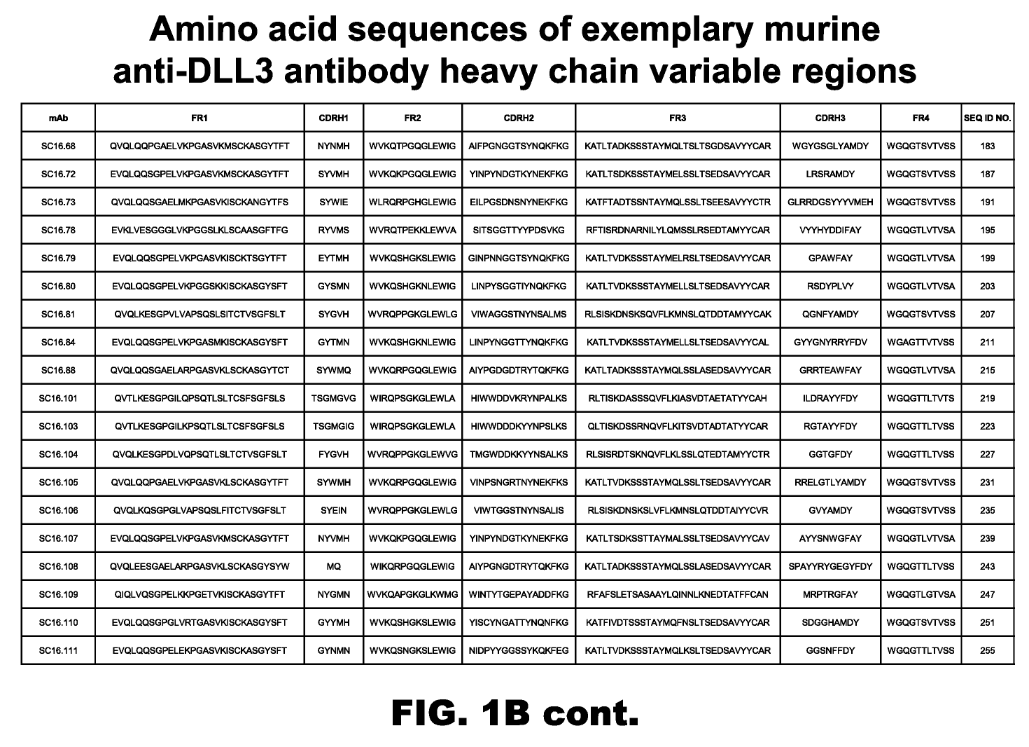

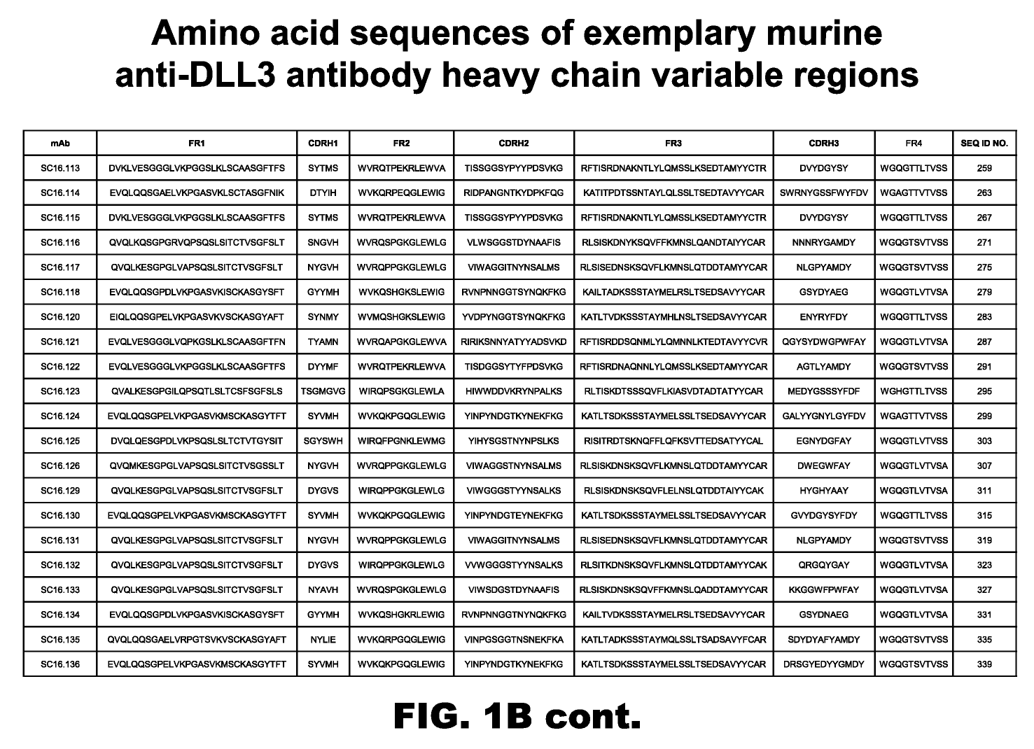

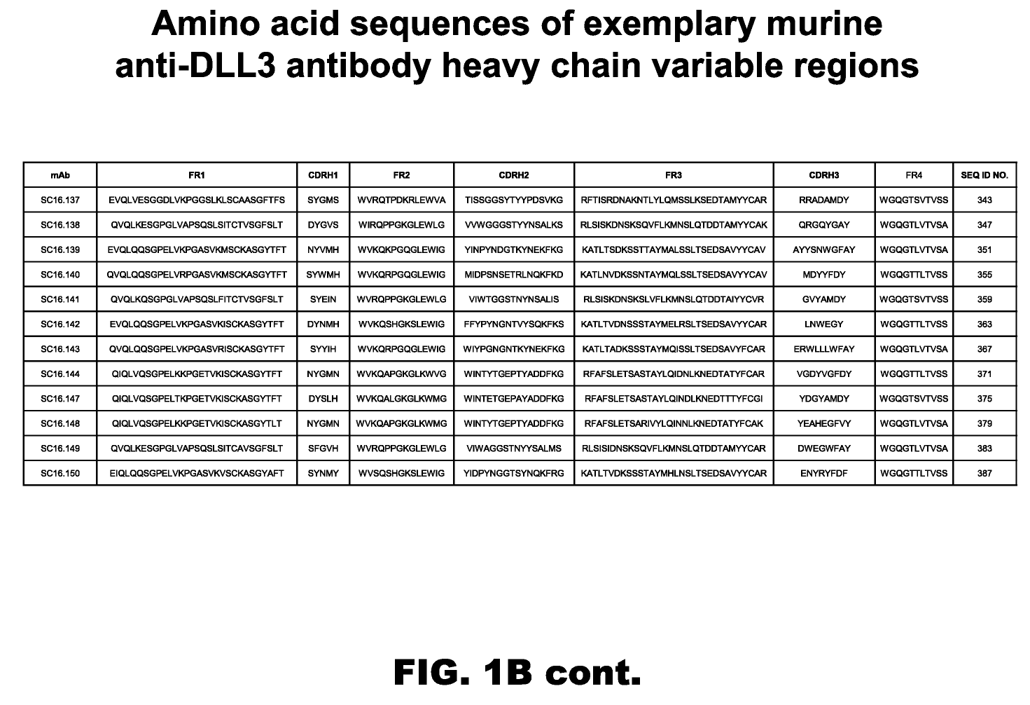

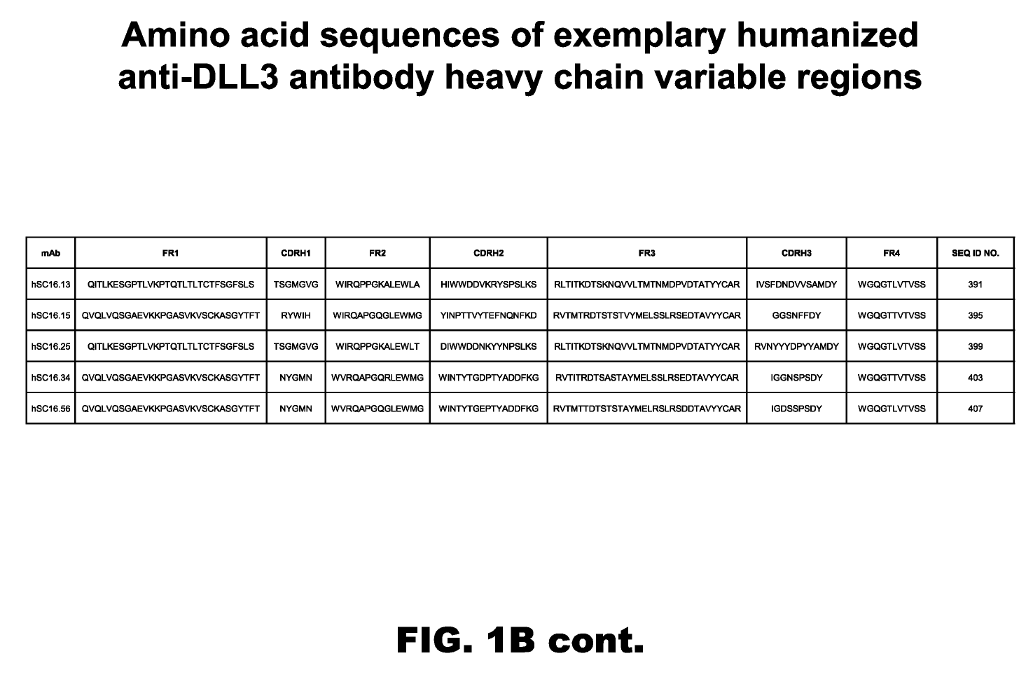

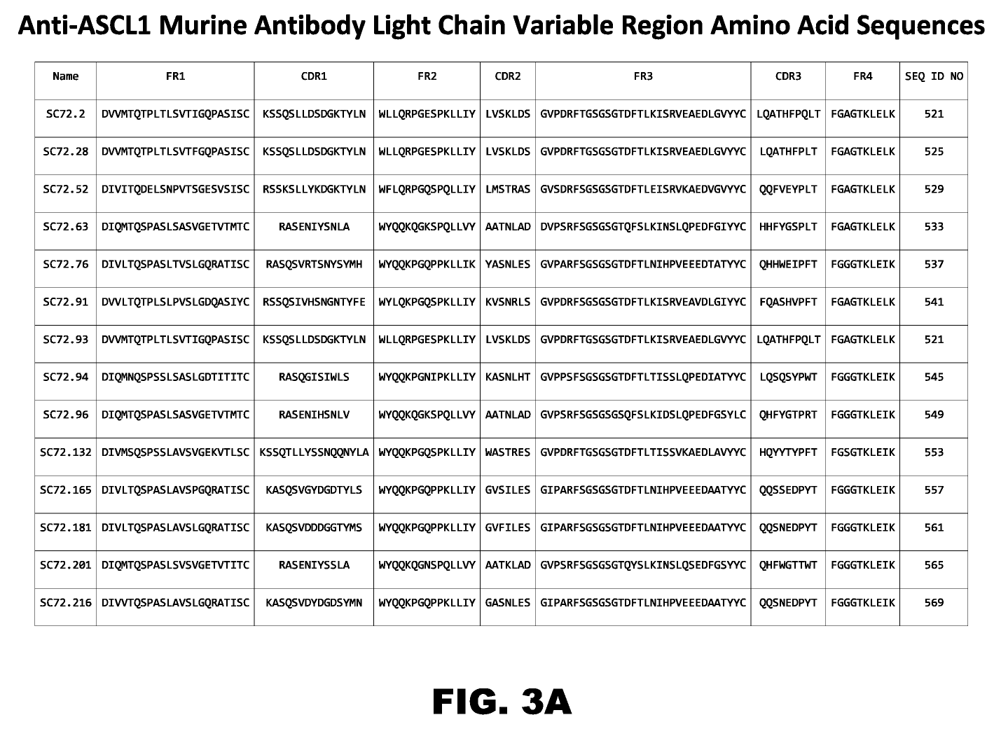

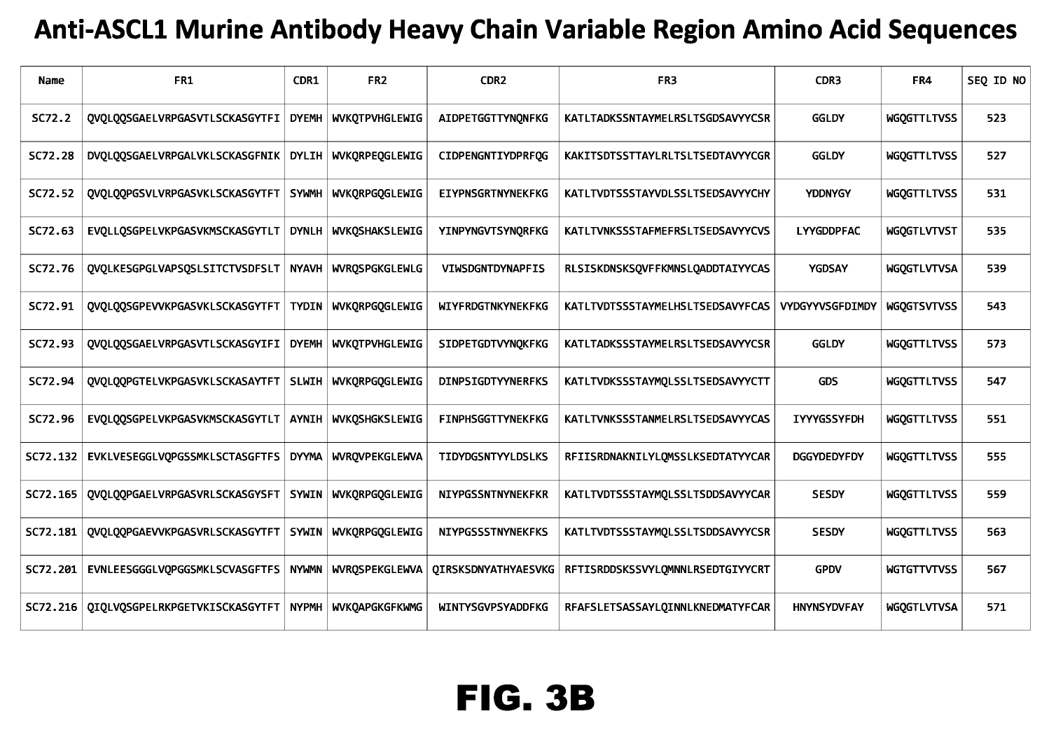

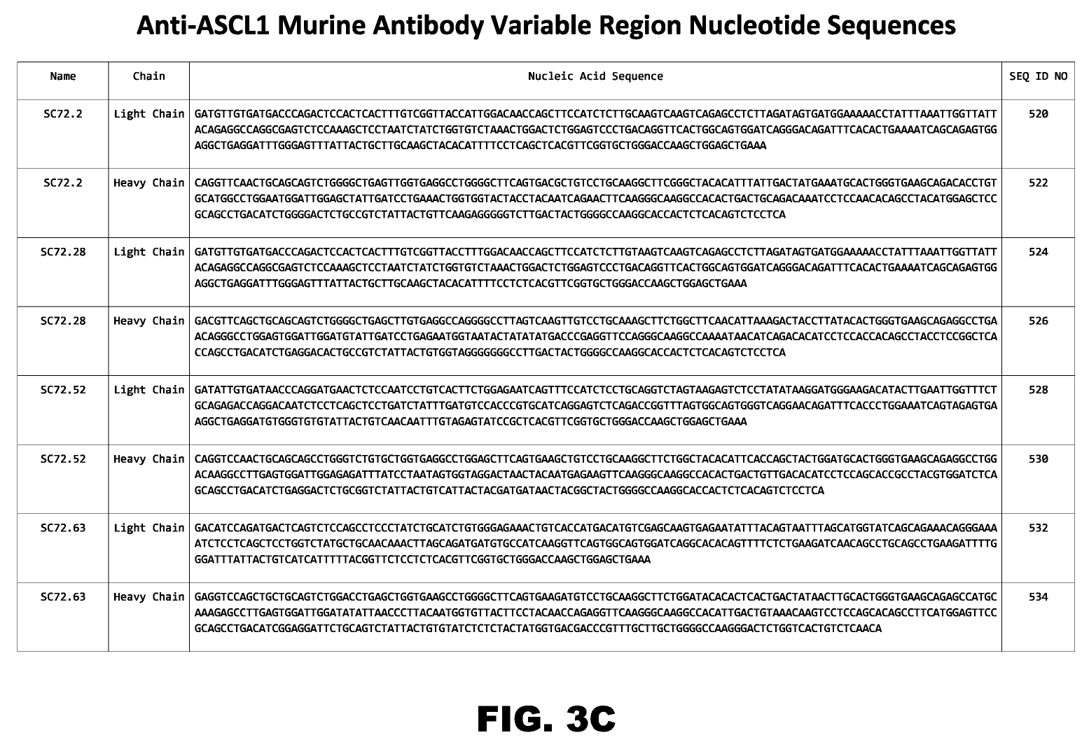

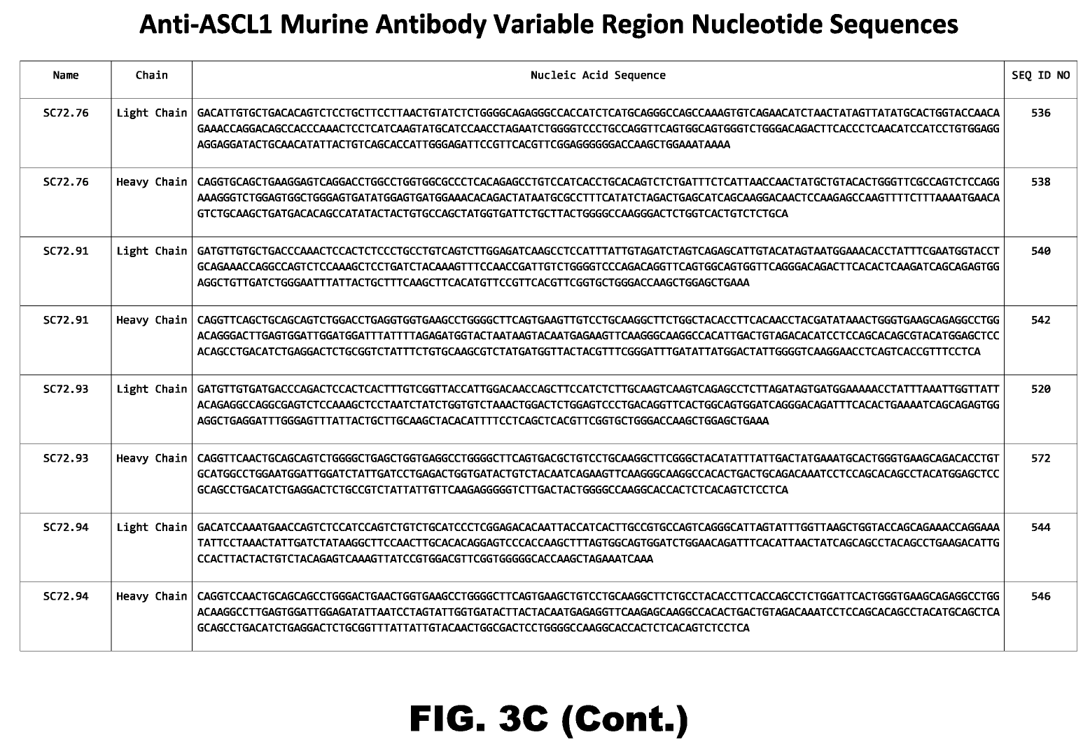

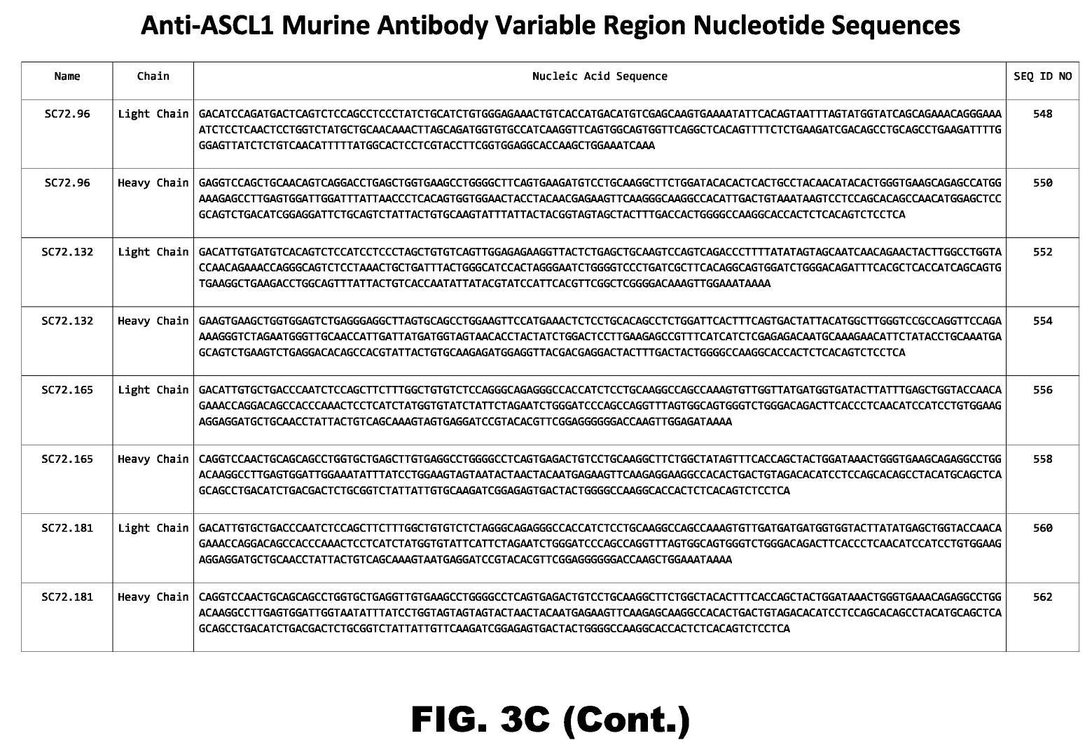

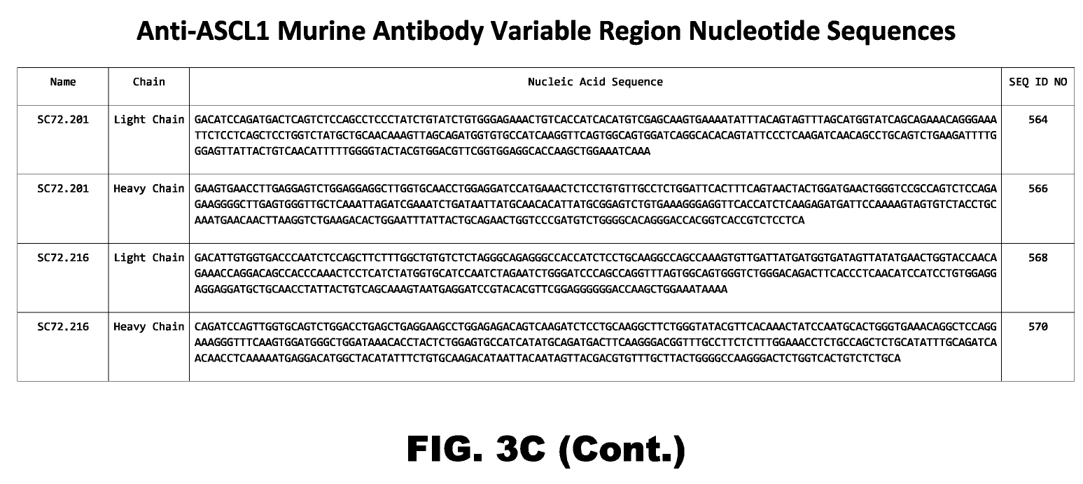

Moreover, representative antibodies for use in such methods include novel anti-DLL3 and anti-ASCL1 antibodies produced as described in the Examples provided herein. FIGS. 1A and 1B and FIGS. 3A and 3B provide, respectively, annotated sequences of numerous anti-DLL3 and anti-ASCL1 binding domains. More particularly the amino acid sequences of the light chain variable regions and heavy chain variable regions of the ASCL1 antibodies are depicted in FIGS. 3A and 3B, respectively, and are represented by SEQ ID NOS: 521-573, odd numbers. Similarly representative anti-DLL3 antibodies compatible with the disclosed diagnostic and therapeutic methods are set forth in FIGS. 1A and 1B (SEQ ID NOS: 21-407, odd numbers).

C. Targeted Therapies Associated with Risk of Neuroendocrine Transition

Targeted cancer therapy, as used herein, is a therapy that targets specific types of cancer, targets specific molecules that are associated with tumors, and/or targets specific molecules needed for tumor development, tumor survival, tumor growth, and/or metastases. Targeted cancer therapies offer the benefit of matching a specific therapeutic agent to the underlying molecular alterations of a tumor, affording an improvement in the selective inhibitory effect on tumor cells while minimizing toxic side effects to normal cells and tissues. This strategy includes targeting driver oncogene mutations or altered gene expression pathways in tumors. As one example, androgen deprivation therapy, which removes androgens essential for prostate cancer growth, is an effective treatment for many patients with prostate adenocarcinoma. As another example, tumor-specific aberrations in the epidermal growth factor receptor (EGFR) protein or the anaplastic lymphoma kinase (ALK) locus, which occur in some lung adenocarcinomas can be targeted by EGFR tyrosine kinase inhibitors (EGFR-TKIs) like erlotinib and gefitinib, or by targeted ALK inhibitors like crizotinib and ceritinib respectively (Roviello, PMID: 26082421).

Notwithstanding the initial clinical response often observed using targeted therapies, numerous patients ultimately develop resistance to the agents via a variety of mechanisms. In addition, the present inventors have discovered that tumors treated with a targeted therapy present a higher incidence of transition to a neuroendocrine phenotype as compared with tumors having been treated with a non-targeted therapy, i.e., therapies effective for many or all tumor types, such as chemotherapy and radiation.

While anti-DLL3 antibody drug conjugates may be used to treat any adenocarcinoma at risk of transitioning to a neuroendocrine phenotype, in particular aspects of the invention, the adenocarcinoma is prostate cancer, lung cancer, or bladder cancer given the targeted therapies currently in use for treating these cancer types.

C.1. Lung Cancer

As described above, patients with lung adenocarcinoma having an activating EGFR mutation can be treated with targeted therapy using EGFR inhibitors (e.g. EGFR-TKIs). An activating epidermal growth factor receptor (EGFR) mutation is any mutation that results in activation of the epidermal growth factor receptor. For example, an activating EGFR mutation can comprise deletion of the EGFR exon 19, or a L858R point mutation in exon 21 of EGFR.

Cancer cells having activating EGFR mutations and are treated with an EGFR inhibitor can acquire resistance to EGFR inhibitor therapy. Tumor cells that are resistant to EGFR inhibitor therapy are at risk of transitioning to a neuroendocrine phenotype. Several mechanisms of acquired resistance are known and include, for example, a substitution of methionine for threonine at position 790 (T790M), small cell transformation, MET amplification, epithelial-mesenchymal transition, and PIK3CA mutation.

Resistance to EGFR-TKIs is observed after a median time of 10 months. The primary mechanism of acquired resistance is the emergence of a dominant second-site EGFR T790M mutation, seen in 50-65% of patients. This gatekeeper mutation, which may represent a minor allele present in the tumor before EGFR-TKI therapy, hinders the ability of the drugs to bind the active conformation of the EGFR protein. Deep sequencing technologies have shown that pre-existing EGFR T790M clones are detected in up to 68% of patients before treatment with an EGFR-TKI, and that treatment with an EGFR-TKI allows for the emergence of the EGFR T790M tumor that is resistant to those inhibitors (Watanabe M. et al., 2015). Second generation EGFR-TKIs (e.g., dacomitinib, afatinib and neratinib) have a higher affinity for EGFR, but have shown limited activity in patients with acquired resistance. Alternatively, anti-EGFR monoclonal antibodies like cetuximab do show some clinical benefit after acquired resistance to EGFR-TKIs, but combining these antibodies with second-generation EGFR-TKIs have adverse side effects. Third-generation EGFR-TKIs (e.g., rociletinib and AZD9291), designed to selectively target both the initial activating and the dominant acquired T790M resistance mutations, have shown clinical activity in early trials, although amplification of other oncogenic signaling molecules may ultimately give rise to rociletinib-resistance in patients (Haringsma et al., 2015). As is the case for the EGFR-TKIs, patients taking targeted ALK inhibitors like crizotinib do show a clinical benefit over standard chemotherapy, but inevitably relapse. Acquired resistance to crizotinib is seen in the clinic due to additional second-site ALK mutations or due to ALK gene amplification (Roviello G. et al., 2015). Again, both of these mechanisms of crizotinib-resistance may represent the emergence of tumor cell subclones with altered target properties in the context of selection pressure due to the targeted therapeutic.

An alternative mechanism of acquired resistance to targeted therapies does not involve acquisition of or selection for secondary mutations or gene amplifications in the therapeutic target; instead, the tumor appears to undergo a histological transformation. For example, in about 10-15% of EGFR-mutant lung adenocarcinomas, while these tumors maintain the original EGFR mutation, they no longer respond to EGFR-TKIs and instead display a histological transformation to resemble small cell lung cancer (SCLC) (Niederst M. J. et al., 2015; Sequist L. V. et al., 2011). Strikingly, acquired resistance to third-generation EGFR-TKIs due to SCLC transformation was seen in 2 of 12 patients (17%) biopsied after relapse (Piotrowska Z. et al., 2015). Separately, two additional cases of EGFR mutated lung adenocarcinoma transitioning to SCLC following treatment with a third generation EGFR-TKI have been reported (Ham J. S. et al., 2015). Cell lines generated from SCLC-transformed EGFR-mutant lung adenocarcinomas are resistant to gefitinib as well as to the third-generation EGFR-TKI WZ4002 (Niederst M. J. et al., 2015). The lack of response to the EGFR-TKIs reflects a reduction or silencing of EGFR expression, since the EGFR mutation status in these tumors is unchanged relative to the original tumor (Niederst M. J. et al., 2015). The transition to a SCLC phenotype is accompanied by acquisition of genetic changes which include expression of neuroendocrine markers, acquisition of ASCL1 expression, and loss or reduction of RB1 (Niederst M. J. et al., 2015).

Transformation of ALK rearranged lung adenocarcinoma into SCLC as a resistance mechanism has also been reported in six patients in response to ALK inhibitor targeted therapies crizotinib and alectinib (Levacq D. et al., 2016; Fujita S. et al., 2016; Caumont C. et al., 2016; Cha Y. J. et al. 2016; Takegawa, N. et al., 2016; Miyamoto S. et al., 2016). These transformed tumors retain ALK translocations, and gain additional mutations typically seen in SCLC, including loss and mutation of RB1, TP53 and PTEN. Sometimes the tumors retain a mix of both adenocarcinoma and SCLC, with chemotherapeutic and target agents influencing clonal detection.

EGFR-mutant lung adenocarcinomas have also been observed to undergo histological transformation into large cell neuroendocrine carcinomas (LCNEC) during treatment with EGFR-TKIs (Kogo M. et al., 2015). In one instance, the original tumor expressed EGFR and RB1 protein as detected by IHC, but following transformation to LCNEC, expression of both EGFR and RB1 protein was lost, while the EGFR mutation was retained (Kogo M. et al., 2015). Likewise, acquired resistance to the ALK inhibitor crizotinib has been reported to occur through transition to LCNEC (Omachi N. et al., 2014; Caumont C. et al., 2016). Again, the original ALK fusion was maintained in the LCNEC cells, while no additional ALK mutations that could confer resistance were detected.

It is probable that as is observed in the acquisition of secondary mutations that confer targeted therapy resistance, emergence of SCLC-transformed non-small cell lung tumors represents a selection for tumor cell subclones from an originally heterogenous tumor. Despite being classified as non-small cell lung cancer, about 10-20% of non-small cell lung cancers exhibit some neuroendocrine properties (Berendsen H. H. et al., 1989), and a subset of lung adenocarcinoma (30/171; 17%) express ASCL1 and other neuroendocrine genes, and have poor prognoses (Fujiwara T. et al., 2011). That targeted therapies can mediate changes in tumor phenotype due to tumor heterogeneity is exemplified by the case of a patient diagnosed with lung adenocarcinoma with an EGFR L858R mutation, who was treated with erlotinib, and whose tumor underwent transformation into SCLC. The SCLC tumor retained the original EGFR mutation with a similar allele frequency, while gaining common SCLC mutations including an activating mutation in PIK3CA and loss or reduction of both TP53 and RB1. Treatment of the transformed SCLC tumor with cisplatin/etoposide, standard of care for SCLC, led to the reemergence of lung adenocarcinoma with an L858R EGFR mutation that was again sensitive to erlotinib. At time of autopsy, this patient harbored two SCLC transformed tumors in the lung and liver, as well as lung lesion with adenocarcinoma histology. All three sites harbored the original EGFR mutation, with the adenocarcinoma lesion harboring a T790M resistance mutation, whereas the SCLC lesions did not (Niederst M. J. et al., 2015). Together, these data are consistent with rare neuroendocrine cells present in the original tumor, which in the context of selection pressure due to the targeted therapeutic, become the bulk of the tumor provided additional mutations are present or acquired (e.g., loss or reduction of RB1).

As demonstrated in the Examples herein, DLL3 expression is increased in lung adenocarcinoma that is resistant to EGFR inhibitor therapy and has transitioned to a neuroendocrine phenotype. The present invention provides that anti-DLL3 antibody drug conjugates may be used as an effective therapeutic treatment strategy for patients with lung adenocarcinoma identified as at risk for the transitioning to a neuroendocrine phenotype, including those patients having received EGFR inhibitor therapy.

Such tumors are typically recurrent, refractory, relapsed or resistant. In particular aspects of the invention, the lung adenocarcinoma tumor comprises non-small cell lung cancer previously treated with EGFR inhibitor therapy.

C.2. Prostate Cancer

Histological transformation as a resistance mechanism is not confined to lung adenocarcinoma; tumor transformation in response to targeted therapy is also observed in prostate adenocarcinoma. Early stage prostate adenocarcinoma is treated with radiation, while locally advanced and metastatic prostate adenocarcinoma is treated with medical or surgical castration to target androgen receptor (AR)-driven tumor growth (Parimi V. et al., 2014). However, most patients eventually relapse and become resistant to androgen deprivation therapy, progressing to a lethal stage of the disease termed castration resistant prostate cancer (CPRC). CPRC is estimated to kill 27,540 men in the US in 2015 (American Cancer Society) and the 5-year overall survival for CRPC is 12.6%, making it the most deadly and aggressive subtype of prostate cancer. Similar to what is observed for lung adenocarcinoma, the mechanisms underlying the development of targeted-therapy resistance in CRPC vary, but can be grouped into (1) restored AR signaling via acquisition of secondary mutations or AR amplification, (2) bypass of direct signaling mediated by the AR protein through acquisition of other oncogenic drivers in the AR signaling pathway, or (3) complete AR independence (Watson et al., 2015). Notably, complete AR independence in CRPC can manifest as histologically transformed tumors with small cell neuroendocrine features (e.g., expression of classic neuroendocrine markers like CHGA, ENO2, NCAM1, SYP, etc). ASCL1, the main neuroendocrine transcription factor that drives SCLC, is upregulated by androgen deprivation and is associated with onset of the neuroendocrine phenotype in CRPC (Rapa I. et al., 2013). Loss or reduction of RB1 and concurrent overexpression of AURKA and MYCN, are enriched in CRPC (Robinson D. et al., 2015; Beltran H. et al., 2011).

Transformation of CRPC into a tumor with a small cell neuroendocrine phenotype results in disease with distinct clinical features: rapid progression with large tumor masses, high frequency of visceral disease, frequent bone metastasis, and disproportionately low PSA levels for the large disease burden, poor response to hormone therapies, and short response to chemotherapeutics (median survival 1 year) (Aparicio A. et al., 2011). About 10-20% of autopsied patients who die from CRPC have small cell neuroendocrine differentiation (Turbat-Herrera E. A. et al., 1988; Tanaka M. et al., 2001). The degree of neuroendocrine differentiation increases with disease progression and in response to androgen deprivation therapy (Parimi V. et al., 2014). With the recent advent of total androgen blockade therapies, there could be a significant increase in CRPC with neuroendocrine features (Zhang X., 2015). A direct link between androgen deprivation therapy and development of CRPC with neuroendocrine differentiation is further supported by studies comparing intermittent androgen deprivation versus continuous androgen deprivation therapy, wherein it was observed that intermittent androgen deprivation delayed or prevented neuroendocrine differentiation (Sciarra A., 2003).

The factors that determine which prostate tumors will undergo a neuroendocrine transition are not well understood. Some data suggests that almost all prostate adenocarcinoma tumors are heterogeneous with sites of focal neuroendocrine differentiation (Cindolo L. et al., 2007; Nelson C. E. et al., 2007). Neuroendocrine cells are indistinguishable from typical adenocarcinoma cells by routine hematoxylin and eosin staining, but can be distinguished with typical neuroendocrine immunohistochemistry (CHGA, SYP, NCAM1, etc.; Priemer D. S. et al., 2016). The number of neuroendocrine cells is associated with neoplastic progression rate, and clusters of neuroendocrine cells as opposed to individual isolated neuroendocrine cells, are associated with poor prognosis (Cindolo L. et al., 2007). Rare neuroendocrine cells might contribute to prostate adenocarcinoma through production of neuropeptides and growth factors that support the rapidly proliferating cells and promote angiogenesis. Additional genetic mutations like loss or reduction of RB1 or amplification of MYC might further contribute to a full transition to a small cell neuroendocrine phenotype. One early marker that has been shown to be expressed at the onset of neuroendocrine transition is PEG10 (Akamatsu S. et al., 2015). Another gene significantly upregulated is PTGER4 (Terada N. et al., 2010), while SRRM4 expression and loss or reduction of REST activity through altered splicing has also been correlated to the onset of neuroendrocrine phenotype in CRPC (Zhang X. et al., 2015). REST is a known repressor of neuronal gene expression in non-neuronal tissues, so its loss or reduction might precede upregulation of neuroendocrine genes.

In general, CRPC that are morphologically NE transformed (CRPC-NE) have a similar mutational landscape to those that retain an adenocarcinoma morphology (CRPC-Ad). CRPC-NE tumors can be distinguished from CRPC-Ad tumors in having lower AR protein expression despite fewer AR mutations, loss or reduction of RB1, mutation or loss or reduction of TP53, and loss or reduction of CYLD, hypermethylation and loss or reduction of SPDEF expression and higher EZH2 expression (Beltran H. et al., 2016). Evaluation of multiple biopsy samples from patients before and after development of CRPC-NE, and from patients who have two types of resistant tumors development, both CRPC-Ad with T790M EGFR mutations, and CRPC-NE, argue for a common adenocarcinoma precursor that gives rise to multiple clones due to the similar molecular genetics pointing towards divergent clonal evolution of metastatic CRPC (Beltran H. et al., 2016). There are strong epigenetic differences between CRPC-Ad and CRPC-NE tumors in the same patient, mainly with aberrant DNA methylation seen in CRPC-NE (Beltran H. et al., 2016), suggesting that transition to a NE state involves epigenetic dysregulation.

In addition to the association of androgen deprivation therapy and neuroendocrine differentiation, it has been shown that radiation therapy can also induce prostate cancer neuroendocrine differentiation, and patients treated with radiation therapy show elevated serum chromogranin A levels, indicating an increase in neuroendocrine cells in the tumor (Hu C. D. et al., 2015). This notion supports the idea that rare neuroendocrine cells are likely present in the original heterogeneous prostate adenocarcinoma, and upon therapies that can effectively eradicate the bulk of the tumor, rare neuroendocrine cells are resistant to these therapies and transform the nature of the tumor.

It has also been proposed that a large cell neuroendocrine phenotype is a transitional stage between conventional adenocarcinoma and small cell neuroendocrine carcinoma, but often tumor heterogeneity is seen and all three phenotypes are present in the same tumor (Aparicio A. et al., 2011; Epstein J. L. et al., 2014). About half of all prostate adenocarcinomas express ERG or another ETS gene family member, and ERG suppresses genes involved in neuroendocrine differentiation (Mounir Z. et al., 2015). Androgen deprivation therapy downregulates ERG and leads to a rapid increase in neuroendocrine cells in the tumor which are resistant to androgen deprivation therapy (Mounir Z. et al., 2015). Similarly, gene amplification of AR plays a role in CRPC development and possibly the differentiation into a neuroendocrine phenotype (Priemer D. S. et al., 2016).

Since CRPC rapidly leads to lethality, patients at risk transitioning to a neuroendocrine phenotype could benefit from treatment before full blown metastatic disease is detected. Novel strategies are needed to treat the neuroendocrine differentiated cells that arise following treatment with targeted therapies that do not eradicate the rare neuroendocrine cells that are present in tumors. These treatment strategies could involve adjunctive therapies that are combined with androgen deprivation to prevent and block CRPC development. Alternatively, salvage therapy after development of CRPC could be administered, but since disease progression is so rapid, preventing rather than treating metastatic disease is preferred.

As demonstrated in the Examples herein, DLL3 expression is increased in prostate adenocarcinoma that is resistant to androgen deprivation therapy and has transitioned to a neuroendocrine phenotype. The present invention provides that anti-DLL3 antibody drug conjugates may be used as an effective therapeutic treatment strategy for patients with prostate adenocarcinoma identified as at risk for the transitioning to a neuroendocrine phenotype, including those patients castration resistant prostate cancer, such as patients having received androgen deprivation therapy. These tumors at risk are often recurrent, refractory, relapsed or resistant.

C.3. Bladder Cancer

As described in the Examples provided herein, DLL3 positive cells are present in bladder adenocarcinoma samples. Small cell neuroendocrine tumors do arise in the bladder and are very aggressive, but perhaps due to the lack of targeted therapies, bladder TCC treated with standard chemotherapy has not been reported to transform into small cell neuroendocrine tumors. Concurrent adenocarcinoma with a neuroendocrine carcinoma has been described (Jiang Y 2014), so bladder carcinoma may be at risk for transitioning to a neuroendocrine phenotype due to tumor heterogeneity.

The present invention provides that anti-DLL3 antibody drug conjugates may be used as an effective therapeutic treatment strategy for patients with bladder adenocarcinoma identified as at risk for the transitioning to a neuroendocrine phenotype, including recurrent, refractory, relapsed or resistant bladder tumors.

C.4. Additional Adenocarcinomas

De novo small cell or neuroendocrine tumors arise at very low frequency in additional tissues including adrenal gland, breast, gall bladder, ovary, cervix, endometrium, kidney, pancreas, colon, stomach, and thyroid. Adenocarcinomas arise in cells with glandular structures from the lung, colon, pancreas, prostate, breast, esophagus, stomach, kidney, cervix, endometrium, and thyroid. For the majority of these adenocarcinomas, therapies targeted the specific oncogenic pathways that drive tumorigenesis have not yet been developed. Resistance to targeted therapies could arise through small cell transformation of these adenocarcinomas.

### III. DLL3 Physiology

It has been found that DLL3 phenotypic determinants are clinically associated with various proliferative disorders, including neoplasia exhibiting neuroendocrine features, and that DLL3 protein and variants or isoforms thereof provide useful tumor markers which may be exploited in the treatment of related diseases. In this regard the present invention provides a number of antibody drug conjugates comprising an anti-DLL3 antibody targeting agent and a payload (e.g., a payload comprising a PBD warhead). As discussed in more detail below, the disclosed anti-DLL3 ADCs are particularly effective at eliminating tumorigenic cells and therefore useful for the treatment and prophylaxis of certain proliferative disorders or the progression or recurrence thereof.

Moreover, it has been found that DLL3 markers or determinants such as cell surface DLL3 protein are therapeutically associated with cancer stem cells (also known as tumor perpetuating cells) and may be effectively exploited to eliminate or silence the same. The ability to selectively reduce or eliminate cancer stem cells through the use of anti-DLL3 conjugates as disclosed herein is surprising in that such cells are known to generally be resistant to many conventional treatments. That is, the effectiveness of traditional, as well as more recent targeted treatment methods, is often limited by the existence and/or emergence of resistant cancer stem cells that are capable of perpetuating tumor growth even in face of these diverse treatment methods. Further, determinants associated with cancer stem cells often make poor therapeutic targets due to low or inconsistent expression, failure to remain associated with the tumorigenic cell or failure to present at the cell surface. In sharp contrast to the teachings of the prior art, the instantly disclosed ADCs and methods effectively overcome this inherent resistance and to specifically eliminate, deplete, silence or promote the differentiation of such cancer stem cells thereby negating their ability to sustain or re-induce the underlying tumor growth.

Thus DLL3 conjugates such as those disclosed herein may advantageously be used in the treatment and/or prevention of selected proliferative (e.g., neoplastic) disorders or progression or recurrence thereof. It will be appreciated that, while preferred embodiments of the invention will be discussed extensively below, particularly in terms of particular domains, regions or epitopes or in the context of cancer stem cells or tumors comprising neuroendocrine features and their interactions with the disclosed antibody drug conjugates, those skilled in the art will appreciate that the scope of the instant invention is not limited by such exemplary embodiments. Rather, the most expansive embodiments of the present invention and the appended claims are broadly and expressly directed to the disclosed anti-DLL3 conjugates and their use in the treatment and/or prevention of a variety of DLL3 associated or mediated disorders, including neoplastic or cell proliferative disorders, regardless of any particular mechanism of action or specifically targeted tumor, cellular or molecular component.

The Notch signaling pathway, first identified in C. elegans and Drosophila and subsequently shown to be evolutionarily conserved from invertebrates to vertebrates, participates in a series of fundamental biological processes including normal embryonic development, adult tissue homeostasis, and stem cell maintenance (D'Souza et al., 2010; Liu et al., 2010). Notch signaling is critical for a variety of cell types during specification, patterning and morphogenesis. Frequently, this occurs through the mechanism of lateral inhibition, in which cells expressing Notch ligand(s) adopt a default cell fate, yet suppress this fate in adjacent cells via stimulation of Notch signaling (Sternberg, 1988; Cabrera 1990). This binary cell fate choice mediated by Notch signaling is found to play a role in numerous tissues, including the developing nervous system (de la Pompa et al., 1997), the hematopoietic and immune systems (Bigas and Espinosoa, 2012; Hoyne et al, 2011; Nagase et al., 2011), the gut (Fre et al., 2005; Fre et al., 2009), the endocrine pancreas (Apelqvist et al., 1999; Jensen et al., 2000), the pituitary (Raetzman et al., 2004), and the diffuse neuroendocrine system (Ito et al., 2000; Schonhoff et al, 2004). A generalized mechanism for implementing this binary switch appears conserved despite the wide range of developmental systems in which Notch plays a role—in cells where the default cell fate choice is determined by transcriptional regulators known as basic helix-loop-helix (bHLH) proteins, Notch signaling leads to activation of a class of Notch responsive genes, which in turn suppress the activity of the bHLH proteins (Ball, 2004). These binary decisions take place in the wider context of developmental and signaling cues that permit Notch signaling to effect proliferation or inhibit it, and to trigger self-renewal or inhibit it.

In Drosophila, Notch signaling is mediated primarily by one Notch receptor gene and two ligand genes, known as Serrate and Delta (Wharton et al, 1985; Rebay et al., 1991). In humans, there are four known Notch receptors and five DSL (Delta-Serrate LAG2) ligands—two homologs of Serrate, known as Jagged1 and Jagged 2, and three homologs of Delta, termed delta-like ligands or DLL1, DLL3 and DLL4. In general, Notch receptors on the surface of the signal-receiving cell are activated by interactions with ligands expressed on the surface of an opposing, signal-sending cell (termed a trans-interaction). These trans-interactions lead to a sequence of protease mediated cleavages of the Notch receptor. In consequence, the Notch receptor intracellular domain is free to translocate from the membrane to the nucleus, where it partners with the CSL family of transcription factors (RBPJ in humans) and converts them from transcriptional repressors into activators of Notch responsive genes.

Of the human Notch ligands, DLL3 is different in that it seems incapable of activating the Notch receptor via trans-interactions (Ladi et al., 2005). Notch ligands may also interact with Notch receptors in cis (on the same cell) leading to inhibition of the Notch signal, although the exact mechanisms of cis-inhibition remain unclear and may vary depending upon the ligand (for instance, see Klein et al., 1997; Ladi et al., 2005; Glittenberg et al., 2006). Two hypothesized modes of inhibition include modulating Notch signaling at the cell surface by preventing trans-interactions, or by reducing the amount of Notch receptor on the surface of the cell by perturbing the processing of the receptor or by physically causing retention of the receptor in the endoplasmic reticulum or Golgi (Sakamoto et al., 2002; Dunwoodie, 2009). It is clear, however, that stochastic differences in expression of Notch receptors and ligands on neighboring cells can be amplified through both transcriptional and non-transcriptional processes, and subtle balances of cis- and trans-interactions can result in a fine tuning of the Notch mediated delineation of divergent cell fates in neighboring tissues (Sprinzak et al., 2010).

DLL3 (also known as Delta-like 3 or SCDO1) is a member of the Delta-like family of Notch DSL ligands. Representative DLL3 protein orthologs include, but are not limited to, human (GenBank Accession Nos. NP_058637 and NP_982353), chimpanzee (GenBank Accession No. XP_003316395), mouse (GenBank Accession No. NP_031892), and rat (GenBank Accession No. NP_446118). In humans, the DLL3 gene consists of 8 exons spanning 9.5 kBp located on chromosome 19q13. Alternate splicing within the last exon gives rise to two processed transcripts, one of 2389 bases (GenBank Accession No. NM_016941) and one of 2052 bases (GenBank Accession No. NM_203486). The former transcript encodes a 618 amino acid protein (GenBank Accession No. NP_058637; SEQ ID NO: 1), whereas the latter encodes a 587 amino acid protein (GenBank Accession No. NP_982353; SEQ ID NO: 2). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The biological relevance of the isoforms is unclear, although both isoforms can be detected in tumor cells. In general, DSL ligands are composed of a series of structural domains: a unique N-terminal domain, followed by a conserved DSL domain, multiple tandem epidermal growth factor (EGF)-like repeats, a transmembrane domain, and a cytoplasmic domain not highly conserved across ligands but one which contains multiple lysine residues that are potential sites for ubiquitination by unique E3 ubiquitin ligases. The DSL domain is a degenerate EGF-domain that is necessary but not sufficient for interactions with Notch receptors (Shimizu et al., 1999). Additionally, the first two EGF-like repeats of most DSL ligands contain a smaller protein sequence motif known as a DOS domain that co-operatively interacts with the DSL domain when activating Notch signaling.

The extracellular region of the DLL3 protein comprises six EGF-like domains, a single DSL domain and an N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3 (SEQ ID NOS: 1 and 2). Each of the EGF-like domains, the DSL domain and the N-terminal domain comprise part of the DLL3 protein as defined by a distinct amino acid sequence. Note that, for the purposes of the instant disclosure the respective EGF-like domains may be termed EGF1 to EGF6 with EGF1 being closest to the N-terminal portion of the protein. In regard to the structural composition of the protein one significant aspect of the instant invention is that the disclosed DLL3 modulators may be generated, fabricated, engineered or selected so as to react with a selected domain, motif or epitope. In certain cases such site specific modulators may provide enhanced reactivity and/or efficacy depending on their primary mode of action. In particularly preferred embodiments the anti-DLL3 ADCs will bind to the DSL domain and, in even more preferred embodiments, will bind to an epitope comprising G203, R205, P206 (SEQ ID NO: 4) within the DSL domain.

Note that, as used herein the terms “mature protein” or “mature polypeptide” as used herein refers to the form(s) of the protein produced by expression in a mammalian cell. It is generally hypothesized that once export of a growing protein chain across the rough endoplasmic reticulum has been initiated, proteins secreted by mammalian cells have a signal peptide (SP) sequence which is cleaved from the complete polypeptide to produce a “mature” form of the protein. In both isoforms of DLL3 the mature protein comprises a signal peptide of 26 amino acids that may be clipped prior to cell surface expression. Thus, in mature proteins the N-terminal domain will extend from position 27 in the protein until the beginning of the DSL domain. Of course, if the protein is not processed in this manner the N-terminal domain would be held to extend to position one of SEQ ID NOS: 3 & 4.

Of the various Delta-like ligands, DLL3 is the most divergent from the others in the family, since it contains a degenerate DSL domain, no DOS motifs, and an intracellular domain which lacks lysine residues. The degenerate DSL and lack of DOS motifs are consistent with the inability of DLL3 to trigger Notch signaling in trans (between cells), suggesting that DLL3, unlike DLL1 or DLL4, acts only as an inhibitor of Notch signaling (Ladi et al., 2005). Studies have shown that DLL3 may be resident primarily in the cis-Golgi (Geffers et al., 2007), which would be consistent with a hypothesized ability to retain Notch receptor intracellularly, or to interfere with processing of Notch receptors, preventing export to the cell surface and instead retargeting it to the lysosome (Chapman et al., 2011). Some DLL3 protein may appear at the cell surface, however, when the protein is artificially overexpressed in model systems (Ladi et al., 2005), but it is not obvious that this would be the case in normal biological contexts nor in tumors in which the DLL3 mRNA transcript is elevated; somewhat surprisingly, the protein levels detected in tumor types indicate significant DLL3 protein is escaping to the cell surface of various tumors.

Defects in the DLL3 gene have been linked to spondylocostal dysostosis in humans, a severe congenital birth defect resulting in abnormal vertebrae formation and rib abnormalities (Dunwoodie, 2009). This is linked to alterations in Notch signaling, known to play a crucial role in determining the polarity and patterning of somites, the embryonic precursors to the vertebrae that require a finely regulated oscillating interplay between Notch, Wnt, and FGF signaling pathways for proper development (Kageyama et al., 2007; Goldbeter and Pourquie, 2008). Although DLL1 and DLL3 are typically expressed in similar locations within the developing mouse embryo, experiments with transgenic mice have demonstrated that DLL3 does not compensate for DLL1 (Geffers et al., 2007). DLL1 knock-out mice are embryonic lethal, but DLL3 mutant mice do survive yet show a phenotype similar to that found in humans with spondylocostal dysostosis (Kusumi et al., 1998; Shinkai et al., 2004). These results data are consistent with a subtle interplay of Notch trans- and cis-interactions crucial for normal development.

Further, as discussed above Notch signaling plays a role in the development and maintenance of neuroendocrine cells and tumors exhibiting neuroendocrine features. In this regard Notch signaling is involved in a wide range of cell fate decisions in normal endocrine organs and in the diffuse neuroendocrine system. For instance, in the pancreas, Notch signaling is required to suppress the development of a default endocrine phenotype mediated by the bHLH transcription factor NGN3 (Habener et al, 2005). Similar Notch mediated suppression of endocrine cell fates occurs in enteroendocrine cells (Schonhoff et al., 2004), thyroid parafollicular cells (Cook et al., 2010), in specifying the relative ratios of neuroendocrine cell types in the pituitary (Dutta et al., 2011), and is likely involved in decisions of cells within the lungs to adopt a neuroendocrine or non-neuroendocrine phenotype (Chen et al., 1997; Ito et al., 2000; Sriuranpong et al., 2002). Hence it is clear that in many tissues, suppression of Notch signaling is linked to neuroendocrine phenotypes.

Inappropriate reactivation of developmental signaling pathways or disregulation of normal signaling pathways are commonly observed in tumors, and in the case of Notch signaling, have been associated with numerous tumor types (Koch and Radtke, 2010; Harris et al., 2012). The Notch pathway has been studied as an oncogene in lymphomas, colorectal, pancreatic, and some types of non-small cell lung cancer (see Zarenczan and Chen, 2010 and references therein). In contrast, Notch is reported to act as a tumor suppressor in tumors with neuroendocrine features (see Zarenczan and Chen, 2010 supra). Tumors with neuroendocrine features arise infrequently in a wide range of primary sites, and while their exhaustive classification remains problematic (Yao et al., 2008; Klimstra et al., 2010; Köppel, 2011), they may be classified into four major types: low grade benign carcinoids, low-grade well-differentiated neuroendocrine tumors with malignant behavior, tumors with mixed neuroendocrine and epithelial features, and high-grade poorly differentiated neuroendocrine carcinomas. Of these classifications, the poorly differentiated neuroendocrine carcinomas, which include small cell lung cancer (SCLC) and subsets of non-small cell lung cancer (NSCLC), are cancer types with dismal prognoses. It has been postulated that SCLC is bronchogenic in origin, arising in part from pulmonary neuroendocrine cells (Galluzzo and Bocchetta, 2011). Whatever the specific cellular source of origin for each of these tumors possessing a neuroendocrine phenotype, it may be expected that suppression of Notch signaling, either by direct lesions in the Notch pathway genes themselves, or by activation of other genes that suppress Notch signaling, may lead to the acquisition of the neuroendocrine phenotype of these tumors. By extension, the genes that lead to the perturbation of the Notch pathway may afford therapeutic targets for the treatment of tumors with neuroendocrine phenotypes, particularly for indications that currently have poor clinical outcomes.

ASCL1 is one such gene that appears to interact with Notch signaling pathway via DLL3. ASCL1 (achaete-scute homolog 1) is a member of the basic helix-loop-helix family of transcription factors and controls transcription by binding to DNA consensus sequences termed E-boxes (5′-CANNTG-3′).

Representative ASCL1 protein orthologs include, but are not limited to, human (GenBank Accession No. NP_004307), chimpanzee (GenBank Accession No. XP_009424458), cynomolgus monkey (GenBank Accession No. XP_005572101), rat (GenBank Accession No. NP_032579) and mouse (GenBank Accession No. NP_032579). In humans, the ASCL1 gene consists of 2 exons spanning approximately 3 kBp at chromosome 12q23.2. Transcription of the human ASCL1 locus yields a 2490 nucleotide transcript (GenBank Accession No. NM_004316) that encodes a 236 amino acid protein (GenBank Accession No. NP_004307). No alternative spliced transcripts or variant proteins have been reported.

It is clear that many neuroendocrine tumors show a poorly differentiated (i.e. partially complete) endocrine phenotype; for instance, marked elevation or expression of various endocrine proteins and polypeptides (e.g. chromogranin A, CHGA; calcitonin, CALCA; propiomelanocorin, POMC; somatostatin, SST), proteins associated with secretory vesicles (e.g., synaptophysin, SYP), and genes involved in the biochemical pathways responsible for the synthesis of bioactive amines (e.g., dopa decarboxylase, DDC). Perhaps not surprisingly, these tumors frequently over-express ASCL1 (also known as mASH1 in mice, or hASH1 in humans), a transcription factor known to play a role in orchestrating gene cascades leading to neural and neuroendocrine phenotypes. Although the specific molecular details of the cascade remain ill-defined, it is increasingly clear that for certain cell types, particularly thyroid parafollicular cells (Kameda et al., 2007), chromaffin cells of the adrenal medulla (Huber et al., 2002) and cells found in the diffuse neuroendocrine system of the lung (Chen et al., 1997; Ito et al., 2000; Sriuranpong et al., 2002), ASCL1 is part of a finely tuned developmental regulatory loop in which cell fate choices are mediated by the balance of ASCL1-mediated and Notch-mediated gene expression cascades. For instance, ASCL1 was found in to be expressed in normal mouse pulmonary neuroendocrine cells, while the Notch signaling effector HES1, was expressed in pulmonary non-neuroendocrine cells (Ito et al., 2000). That these two cascades are in a fine balance with potential cross-regulation is increasingly appreciated. The Notch effector HES1 has been shown to downregulate ASCL1 expression (Chen et al., 1997; Sriuranpong et al., 2002). These results clearly demonstrate that Notch signaling can suppress neuroendocrine differentiation. However, demonstration that ASCL1 binding to the DLL3 promoter activates DLL3 expression (Henke et al., 2009) and the observation that DLL3 attenuates Notch signaling (Ladi et al., 2005) closes the genetic circuit for cell fate choices between neuroendocrine and non-neuroendocrine phenotypes.

Given that Notch signaling appears to have evolved to amplify subtle differences between neighboring cells to permit sharply bounded tissue domains with divergent differentiation paths (e.g., “lateral inhibition,” as described above), these data together suggest that a finely tuned developmental regulatory loop has become reactivated and disregulated in cancers with neuroendocrine phenotypes. While it is not obvious that DLL3 would provide a suitable cell surface target for the development of antibody therapeutics given its normal residence within interior membranous compartments of the cell (Geffers et al., 2007) and its presumed interactions with Notch therein, it is possible that the resultant elevation of DLL3 expression in neuroendocrine tumors may offer a unique therapeutic target for tumors with the neuroendocrine phenotype (e.g., NETs and pNETs). It is commonly observed that vast overexpression of proteins in laboratory systems may cause mislocalization of the overexpressed protein within the cell. Therefore it is a reasonable hypothesis, yet not obvious without experimental verification, that overexpression of DLL3 in tumors may lead to some cell surface expression of the protein, and thereby present a target for the development of antibody therapeutics.

### IV. Antibodies

A. Antibody Structure

Antibodies and variants and derivatives thereof, including accepted nomenclature and numbering systems, have been extensively described, for example, in Abbas et al. (2010), Cellular and Molecular Immunology (6^{th} Ed.), W.B. Saunders Company; or Murphey et al. (2011), Janeway's Immunobiology (8^{th} Ed.), Garland Science.

An “antibody” or “intact antibody” typically refers to a Y-shaped tetrameric protein comprising two heavy (H) and two light (L) polypeptide chains held together by covalent disulfide bonds and non-covalent interactions. Each light chain is composed of one variable domain (VL) and one constant domain (CL). Each heavy chain comprises one variable domain (VH) and a constant region, which in the case of IgG, IgA, and IgD antibodies, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (from about 10 to about 60 amino acids in various IgG subclasses). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues.

As used herein the term “antibody” includes polyclonal antibodies, multiclonal antibodies, monoclonal antibodies, chimeric antibodies, humanized and primatized antibodies, CDR grafted antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof, immunospecific antibody fragments such as Fd, Fab, F(ab′)₂, F(ab′) fragments, single-chain fragments (e.g. ScFv and ScFvFc); and derivatives thereof including Fc fusions and other modifications, and any other immunoreactive molecule so long as it exhibits preferential association or binding with a determinant. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter α, δ, ε, γ, and μ, respectively. Light chains of the antibodies from any vertebrate species can be assigned to one of two clearly distinct types, called kappa (κ) and lambda (λ), based on the amino acid sequences of their constant domains.

The variable domains of antibodies show considerable variation in amino acid composition from one antibody to another and are primarily responsible for antigen recognition and binding. Variable regions of each light/heavy chain pair form the antibody binding site such that an intact IgG antibody has two binding sites (i.e. it is bivalent). VH and VL domains comprise three regions of extreme variability, which are termed hypervariable regions, or more commonly, complementarity-determining regions (CDRs), framed and separated by four less variable regions known as framework regions (FRs). The non-covalent association between the VH and the VL region forms the Fv fragment (for “fragment variable”) which contains one of the two antigen-binding sites of the antibody. ScFv fragments (for single chain fragment variable), which can be obtained by genetic engineering, associates in a single polypeptide chain, the VH and the VL region of an antibody, separated by a peptide linker.

As used herein, the assignment of amino acids to each domain, framework region and CDR may be in accordance with one of the schemes provided by Kabat et al. (1991) Sequences of Proteins of Immunological Interest (5^{th} Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co or AbM (Oxford Molecular/MSI Pharmacopia) unless otherwise noted. As is well known in the art variable region residue numbering is typically as set forth in Chothia or Kabat. Amino acid residues which comprise CDRs as defined by Kabat, Chothia, MacCallum (also known as Contact) and AbM as obtained from the Abysis website database (infra.) are set out below. Note that MacCallum uses the Chothia numbering system.

TABLE 1

| | Kabat | Chothia | MacCallum | AbM |
|---|---|---|---|---|
| VH CDR1 | 31-35 | 26-32 | 30-35 | 26-35 |
| VH CDR2 | 50-65 | 52-56 | 47-58 | 50-58 |
| VH CDR3 | 95-102 | 95-102 | 93-101 | 95-102 |
| VL CDR1 | 24-34 | 24-34 | 30-36 | 24-34 |
| VL CDR2 | 50-56 | 50-56 | 46-55 | 50-56 |
| VL CDR3 | 89-97 | 89-97 | 89-96 | 89-97 |

Variable regions and CDRs in an antibody sequence can be identified according to general rules that have been developed in the art (as set out above, such as, for example, the Kabat nomenclature system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J., 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671-D674 (2005).

Preferably the sequences are analyzed using the Abysis database, which integrates sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat et al.

For heavy chain constant region amino acid positions discussed in the invention, numbering is according to the Eu index first described in Edelman et al. 1969, Proc. Natl. Acad. Sci. USA 63(1): 78-85 describing the amino acid sequence of the myeloma protein Eu, which reportedly was the first human IgG1 sequenced. The Eu index of Edelman is also set forth in Kabat et al., 1991 (supra.). Thus, the terms “Eu index as set forth in Kabat” or “Eu index of Kabat” or “Eu index” or “Eu numbering” in the context of the heavy chain refers to the residue numbering system based on the human IgG1 Eu antibody of Edelman et al. as set forth in Kabat et al., 1991 (supra.) The numbering system used for the light chain constant region amino acid sequence is similarly set forth in Kabat et al., (supra.). An exemplary kappa light chain constant region amino acid sequence compatible with the present invention is set forth immediately below:

(SEQ ID NO: 5)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Similarly, an exemplary IgG1 heavy chain constant region amino acid sequence compatible with the present invention is set forth immediately below:

(SEQ ID NO: 6)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG.

The disclosed constant region sequences, or variations or derivatives thereof, may be operably associated with the disclosed heavy and light chain variable regions using standard molecular biology techniques to provide full-length antibodies that may be used as such or incorporated in the ADCs of the invention.

There are two types of disulfide bridges or bonds in immunoglobulin molecules: interchain and intrachain disulfide bonds. As is well known in the art the location and number of interchain disulfide bonds vary according to the immunoglobulin class and species. While the invention is not limited to any particular class or subclass of antibody, the IgG1 immunoglobulin shall be used throughout the instant disclosure for illustrative purposes. In wild-type IgG1 molecules there are twelve intrachain disulfide bonds (four on each heavy chain and two on each light chain) and four interchain disulfide bonds. Intrachain disulfide bonds are generally somewhat protected and relatively less susceptible to reduction than interchain bonds. Conversely, interchain disulfide bonds are located on the surface of the immunoglobulin, are accessible to solvent and are usually relatively easy to reduce. Two interchain disulfide bonds exist between the heavy chains and one from each heavy chain to its respective light chain. It has been demonstrated that interchain disulfide bonds are not essential for chain association. The IgG1 hinge region contain the cysteines in the heavy chain that form the interchain disulfide bonds, which provide structural support along with the flexibility that facilitates Fab movement. The heavy/heavy IgG1 interchain disulfide bonds are located at residues C226 and C229 (Eu numbering) while the IgG1 interchain disulfide bond between the light and heavy chain of IgG1 (heavy/light) are formed between C214 of the kappa or lambda light chain and C220 in the upper hinge region of the heavy chain.

B. Antibody Generation and Production

Antibodies of the invention can be produced using a variety of methods known in the art.

1. Generation of Polyclonal Antibodies in Host Animals

The production of polyclonal antibodies in various host animals is well known in the art (see for example, Harlow and Lane (Eds.) (1988) Antibodies: A Laboratory Manual, CSH Press; and Harlow et al. (1989) Antibodies, NY, Cold Spring Harbor Press). In order to generate polyclonal antibodies, an immunocompetent animal (e.g., mouse, rat, rabbit, goat, non-human primate, etc.) is immunized with an antigenic protein or cells or preparations comprising an antigenic protein. After a period of time, polyclonal antibody-containing serum is obtained by bleeding or sacrificing the animal. The serum may be used in the form obtained from the animal or the antibodies may be partially or fully purified to provide immunoglobulin fractions or isolated antibody preparations.

In this regard antibodies of the invention may be generated from any DLL3 or ASCL1 determinant that induces an immune response in an immunocompetent animal. As used herein “determinant” or “target” means any detectable trait, property, marker or factor that is identifiably associated with, or specifically found in or on a particular cell, cell population or tissue. Determinants or targets may be morphological, functional or biochemical in nature and are preferably phenotypic. In preferred embodiments a determinant is a protein that is differentially expressed (over- or under-expressed) by specific cell types or by cells under certain conditions (e.g., during specific points of the cell cycle or cells in a particular niche). For the purposes of the instant invention a determinant preferably is differentially expressed on aberrant cancer cells and may comprise a DLL3 protein, or any of its splice variants, isoforms, homologs or family members, or specific domains, regions or epitopes thereof. An “antigen”, “immunogenic determinant”, “antigenic determinant” or “immunogen” means any DLL3 or ASCL1 protein or any fragment, region or domain thereof that can stimulate an immune response when introduced into an immunocompetent animal and is recognized by the antibodies produced by the immune response. The presence or absence of the determinants contemplated herein may be used to identify a cell, cell subpopulation or tissue (e.g., tumors, tumorigenic cells or CSCs).

Any form of antigen, or cells or preparations containing the antigen, can be used to generate an antibody that is specific for the DLL3 determinant. As set forth herein the term “antigen” is used in a broad sense and may comprise any immunogenic fragment or determinant of the selected target including a single epitope, multiple epitopes, single or multiple domains or the entire extracellular domain (ECD) or protein. The antigen may be an isolated full-length protein, a cell surface protein (e.g., immunizing with cells expressing at least a portion of the antigen on their surface), or a soluble protein (e.g., immunizing with only the ECD portion of the protein) or protein construct (e.g., Fc-antigen). The antigen may be produced in a genetically modified cell. Any of the aforementioned antigens may be used alone or in combination with one or more immunogenicity enhancing adjuvants known in the art. DNA encoding the antigen may be genomic or non-genomic (e.g., cDNA) and may encode at least a portion of the ECD, sufficient to elicit an immunogenic response. Any vectors may be employed to transform the cells in which the antigen is expressed, including but not limited to adenoviral vectors, lentiviral vectors, plasmids, and non-viral vectors, such as cationic lipids.

2. Monoclonal Antibodies

In selected embodiments, the invention contemplates use of monoclonal antibodies. As known in the art, the term “monoclonal antibody” or “mAb” refers to an antibody obtained from a population of substantially homogeneous antibodies, i.e., the individual antibodies comprising the population are identical except for possible mutations (e.g., naturally occurring mutations), that may be present in minor amounts.

Monoclonal antibodies can be prepared using a wide variety of techniques known in the art including hybridoma techniques, recombinant techniques, phage display technologies, transgenic animals (e.g., a XenoMouse®) or some combination thereof. For example, monoclonal antibodies can be produced using hybridoma and biochemical and genetic engineering techniques such as described in more detail in An, Zhigiang (ed.) Therapeutic Monoclonal Antibodies. From Bench to Clinic, John Wiley and Sons, 1^{st} ed. 2009; Shire et. al. (eds.) Current Trends in Monoclonal Antibody Development and Manufacturing, Springer Science+Business Media LLC, 1^{st} ed. 2010; Harlow et al., Antibodies: A Laboratory Manual, Cold Spring Harbor Laboratory Press, 2nd ed. 1988; Hammerling, et al., in: Monoclonal Antibodies and T-Cell Hybridomas 563-681 (Elsevier, N.Y., 1981). Following production of multiple monoclonal antibodies that bind specifically to a determinant, particularly effective antibodies may be selected through various screening processes, based on, for example, its affinity for the determinant or rate of internalization. Antibodies produced as described herein may be used as “source” antibodies and further modified to, for example, improve affinity for the target, improve its production in cell culture, reduce immunogenicity in vivo, create multispecific constructs, etc. A more detailed description of monoclonal antibody production and screening is set out below and in the appended Examples.

3. Human Antibodies

In another embodiment, the antibodies may comprise fully human antibodies. The term “human antibody” refers to an antibody which possesses an amino acid sequence that corresponds to that of an antibody produced by a human and/or has been made using any of the techniques for making human antibodies described below.

Human antibodies can be produced using various techniques known in the art. One technique is phage display in which a library of (preferably human) antibodies is synthesized on phages, the library is screened with the antigen of interest or an antibody-binding portion thereof, and the phage that binds the antigen is isolated, from which one may obtain the immunoreactive fragments. Methods for preparing and screening such libraries are well known in the art and kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; and Barbas et al., Proc. Natl. Acad. Sci. USA 88:7978-7982 (1991)).

In one embodiment, recombinant human antibodies may be isolated by screening a recombinant combinatorial antibody library prepared as above. In one embodiment, the library is a scFv phage display library, generated using human VL and VH cDNAs prepared from mRNA isolated from B-cells.

The antibodies produced by naive libraries (either natural or synthetic) can be of moderate affinity (Kₐ of about 10⁶ to 10⁷ M⁻¹), but affinity maturation can also be mimicked in vitro by constructing and reselecting from secondary libraries as described in the art. For example, mutation can be introduced at random in vitro by using error-prone polymerase (reported in Leung et al., Technique, 1: 11-15 (1989)). Additionally, affinity maturation can be performed by randomly mutating one or more CDRs, e.g. using PCR with primers carrying random sequence spanning the CDR of interest, in selected individual Fv clones and screening for higher-affinity clones. WO 9607754 described a method for inducing mutagenesis in a CDR of an immunoglobulin light chain to create a library of light chain genes. Another effective approach is to recombine the VH or VL domains selected by phage display with repertoires of naturally occurring V domain variants obtained from unimmunized donors and to screen for higher affinity in several rounds of chain reshuffling as described in Marks et al., Biotechnol., 10: 779-783 (1992). This technique allows the production of antibodies and antibody fragments with a dissociation constant K_{D} (k_{off}/kₒₙ) of about 10⁻⁹ M or less.

In other embodiments, similar procedures may be employed using libraries comprising eukaryotic cells (e.g., yeast) that express binding pairs on their surface. See, for example, U.S. Pat. No. 7,700,302 and U.S. Ser. No. 12/404,059. In one embodiment, the human antibody is selected from a phage library, where that phage library expresses human antibodies (Vaughan et al. Nature Biotechnology 14:309-314 (1996): Sheets et al. Proc. Natl. Acad. Sci. USA 95:6157-6162 (1998). In other embodiments, human binding pairs may be isolated from combinatorial antibody libraries generated in eukaryotic cells such as yeast. See e.g., U.S. Pat. No. 7,700,302. Such techniques advantageously allow for the screening of large numbers of candidate modulators and provide for relatively easy manipulation of candidate sequences (e.g., by affinity maturation or recombinant shuffling).

Human antibodies can also be made by introducing human immunoglobulin loci into transgenic animals, e.g., mice in which the endogenous immunoglobulin genes have been partially or completely inactivated and human immunoglobulin genes have been introduced. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and 6,075,181 and 6,150,584 regarding XenoMouse® technology; and Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual suffering from a neoplastic disorder or may have been immunized in vitro). See, e.g., Cole et al., Monoclonal Antibodies and Cancer Therapy, Alan R. Liss, p. 77 (1985); Boerner et al., J Immunol, 147 (1):86-95 (1991); and U.S. Pat. No. 5,750,373.

Whatever the source it will be appreciated that the human antibody sequence may be fabricated using art-known molecular engineering techniques and introduced into expression systems and host cells as described herein. Such non-natural recombinantly produced human antibodies (and subject compositions) are entirely compatible with the teachings of this disclosure and are expressly held to be within the scope of the instant invention. In certain select aspects ADCs of the invention will comprise a recombinantly produced human antibody acting as a cell binding agent.

4. Derived Antibodies

Once source antibodies have been generated, selected and isolated as described above they may be further altered to provide anti-DLL3 or anti-ASCL1 antibodies having improved pharmaceutical characteristics. Preferably the source antibodies are modified or altered using known molecular engineering techniques to provide derived antibodies having the desired therapeutic properties.

4.1. Chimeric and Humanized Antibodies

Selected embodiments of the invention comprise murine monoclonal antibodies that immunospecifically bind to DLL3 or immunospecifically bind to ASCL1 and which can be considered “source” antibodies. In selected embodiments, antibodies of the invention can be derived from such “source” antibodies through optional modification of the constant region and/or the epitope-binding amino acid sequences of the source antibody. In certain embodiments an antibody is “derived” from a source antibody if selected amino acids in the source antibody are altered through deletion, mutation, substitution, integration or combination. In another embodiment, a “derived” antibody is one in which fragments of the source antibody (e.g., one or more CDRs or the entire heavy and light chain variable regions) are combined with or incorporated into an acceptor antibody sequence to provide the derivative antibody (e.g. chimeric or humanized antibodies). These “derived” antibodies can be generated using standard molecular biological techniques as described below, such as, for example, to improve affinity for the determinant; to improve antibody stability; to improve production and yield in cell culture; to reduce immunogenicity in vivo; to reduce toxicity; to facilitate conjugation of an active moiety; or to create a multispecific antibody. Such antibodies may also be derived from source antibodies through modification of the mature molecule (e.g., glycosylation patterns or pegylation) by chemical means or post-translational modification.

In one embodiment, the antibodies of the invention comprise chimeric antibodies that are derived from protein segments from at least two different species or class of antibodies that have been covalently joined. The term “chimeric” antibody is directed to constructs in which a portion of the heavy and/or light chain is identical or homologous to corresponding sequences in antibodies from a particular species or belonging to a particular antibody class or subclass, while the remainder of the chain(s) is identical or homologous to corresponding sequences in antibodies from another species or belonging to another antibody class or subclass, as well as fragments of such antibodies (U.S. Pat. No. 4,816,567; Morrison et al., 1984, PMID: 6436822). In some embodiments chimeric antibodies of the instant invention may comprise all or most of the selected murine heavy and light chain variable regions operably linked to human light and heavy chain constant regions. In other selected embodiments, anti-DLL3 or anti-ASCL1 antibodies may be “derived” from the mouse antibodies disclosed herein.

In other embodiments, chimeric antibodies of the invention are “CDR-grafted” antibodies, where the CDRs (as defined using Kabat, Chothia, McCallum, etc.) are derived from a particular species or belonging to a particular antibody class or subclass, while the remainder of the antibody is largely derived from an antibody from another species or belonging to another antibody class or subclass. For use in humans, one or more selected rodent CDRs (e.g., mouse CDRs) may be grafted into a human acceptor antibody, replacing one or more of the naturally occurring CDRs of the human antibody. These constructs generally have the advantages of providing full strength human antibody functions, e.g., complement dependent cytotoxicity (CDC) and antibody-dependent cell-mediated cytotoxicity (ADCC) while reducing unwanted immune responses to the antibody by the subject. In one embodiment the CDR grafted antibodies will comprise one or more CDRs obtained from a mouse incorporated in a human framework sequence.

Similar to the CDR-grafted antibody is a “humanized” antibody. As used herein, a “humanized” antibody is a human antibody (acceptor antibody) comprising one or more amino acid sequences (e.g. CDR sequences) derived from one or more non-human antibodies (donor or source antibody). In certain embodiments, “back mutations” can be introduced into the humanized antibody, in which residues in one or more FRs of the variable region of the recipient human antibody are replaced by corresponding residues from the non-human species donor antibody. Such back mutations may to help maintain the appropriate three-dimensional configuration of the grafted CDR(s) and thereby improve affinity and antibody stability. Antibodies from various donor species may be used including, without limitation, mouse, rat, rabbit, or non-human primate. Furthermore, humanized antibodies may comprise new residues that are not found in the recipient antibody or in the donor antibody to, for example, further refine antibody performance. CDR grafted and humanized antibodies compatible with the instant invention comprising murine components from source antibodies and human components from acceptor antibodies are provided as set forth in the Examples below.

Various art-recognized techniques can be used to determine which human sequences to use as acceptor antibodies to provide humanized constructs in accordance with the instant invention. Compilations of compatible human germline sequences and methods of determining their suitability as acceptor sequences are disclosed, for example, in Dubel and Reichert (Eds.) (2014) Handbook of Therapeutic Antibodies, 2^{nd} Edition, Wiley-Blackwell GmbH; Tomlinson, I. A. et al. (1992) J. Mol. Biol. 227:776-798; Cook, G. P. et al. (1995) Immunol. Today 16: 237-242; Chothia, D. et al. (1992) J Mol. Biol. 227:799-817; and Tomlinson et al. (1995) EMBO J 14:4628-4638). The V-BASE directory (VBASE2—Retter et a., Nucleic Acid Res. 33; 671-674, 2005) which provides a comprehensive directory of human immunoglobulin variable region sequences (compiled by Tomlinson, I. A. et al. MRC Centre for Protein Engineering, Cambridge, UK) may also be used to identify compatible acceptor sequences. Additionally, consensus human framework sequences described, for example, in U.S. Pat. No. 6,300,064 may also prove to be compatible acceptor sequences are can be used in accordance with the instant teachings. In general, human framework acceptor sequences are selected based on homology with the murine source framework sequences along with an analysis of the CDR canonical structures of the source and acceptor antibodies. The derived sequences of the heavy and light chain variable regions of the derived antibody may then be synthesized using art recognized techniques.

By way of example CDR grafted and humanized antibodies, and associated methods, are described in U.S. Pat. Nos. 6,180,370 and 5,693,762. For further details, see, e.g., Jones et al., 1986, (PMID: 3713831); and U.S. Pat. Nos. 6,982,321 and 7,087,409.

The sequence identity or homology of the CDR grafted or humanized antibody variable region to the human acceptor variable region may be determined as discussed herein and, when measured as such, will preferably share at least 60% or 65% sequence identity, more preferably at least 70%, 75%, 80%, 85%, or 90% sequence identity, even more preferably at least 93%, 95%, 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution.

It will be appreciated that the annotated CDRs and framework sequences as provided in the appended FIGS. 1A, 1B, 3A, and 3B are defined as per Kabat et al. using a proprietary Abysis database. However, as discussed herein one skilled in the art could readily identify CDRs in accordance with definitions provided by Chothia et al., ABM or MacCallum et al as well as Kabat et al. As such, anti-DLL3 or anti-ASCL1 humanized antibodies comprising one or more CDRs derived according to any of the aforementioned systems are explicitly held to be within the scope of the instant invention.

4.2. Site-Specific Antibodies

The antibodies of the instant invention may be engineered to facilitate conjugation to a cytotoxin or other anti-cancer agent (as discussed in more detail below). It is advantageous for the antibody drug conjugate (ADC) preparation to comprise a homogenous population of ADC molecules in terms of the position of the cytotoxin on the antibody and the drug to antibody ratio (DAR). Based on the instant disclosure one skilled in the art could readily fabricate site-specific engineered constructs as described herein. As used herein a “site-specific antibody” or “site-specific construct” means an antibody, or immunoreactive fragment thereof, wherein at least one amino acid in either the heavy or light chain is deleted, altered or substituted (preferably with another amino acid) to provide at least one free cysteine. Similarly, a “site-specific conjugate” shall be held to mean an ADC comprising a site-specific antibody and at least one cytotoxin or other compound (e.g., a reporter molecule) conjugated to the unpaired or free cysteine(s). In certain embodiments the unpaired cysteine residue will comprise an unpaired intrachain cysteine residue. In other embodiments the free cysteine residue will comprise an unpaired interchain cysteine residue. In still other embodiments the free cysteine may be engineered into the amino acid sequence of the antibody (e.g., in the CH3 domain). In any event the site-specific antibody can be of various isotypes, for example, IgG, IgE, IgA or IgD; and within those classes the antibody can be of various subclasses, for example, IgG1, IgG2, IgG3 or IgG4. For IgG constructs the light chain of the antibody can comprise either a kappa or lambda isotype each incorporating a C214 that, in selected embodiments, may be unpaired due to a lack of a C220 residue in the IgG1 heavy chain.

Thus, as used herein, the terms “free cysteine” or “unpaired cysteine” may be used interchangeably unless otherwise dictated by context and shall mean any cysteine (or thiol containing) constituent (e.g., a cysteine residue) of an antibody, whether naturally present or specifically incorporated in a selected residue position using molecular engineering techniques, that is not part of a naturally occurring (or “native”) disulfide bond under physiological conditions. In certain selected embodiments the free cysteine may comprise a naturally occurring cysteine whose native interchain or intrachain disulfide bridge partner has been substituted, eliminated or otherwise altered to disrupt the naturally occurring disulfide bridge under physiological conditions thereby rendering the unpaired cysteine suitable for site-specific conjugation. In other preferred embodiments the free or unpaired cysteine will comprise a cysteine residue that is selectively placed at a predetermined site within the antibody heavy or light chain amino acid sequences. It will be appreciated that, prior to conjugation, free or unpaired cysteines may be present as a thiol (reduced cysteine), as a capped cysteine (oxidized) or as part of a non-native intra- or intermolecular disulfide bond (oxidized) with another cysteine or thiol group on the same or different molecule depending on the oxidation state of the system. As discussed in more detail below, mild reduction of the appropriately engineered antibody construct will provide thiols available for site-specific conjugation. Accordingly, in particularly preferred embodiments the free or unpaired cysteines (whether naturally occurring or incorporated) will be subject to selective reduction and subsequent conjugation to provide homogenous DAR compositions.

It will be appreciated that the favorable properties exhibited by the disclosed engineered conjugate preparations is predicated, at least in part, on the ability to specifically direct the conjugation and largely limit the fabricated conjugates in terms of conjugation position and the absolute DAR value of the composition. Unlike most conventional ADC preparations the present invention need not rely entirely on partial or total reduction of the antibody to provide random conjugation sites and relatively uncontrolled generation of DAR species. Rather, in certain aspects the present invention preferably provides one or more predetermined unpaired (or free) cysteine sites by engineering the targeting antibody to disrupt one or more of the naturally occurring (i.e., “native”) interchain or intrachain disulfide bridges or to introduce a cysteine residue at any position. To this end it will be appreciated that, in selected embodiments, a cysteine residue may be incorporated anywhere along the antibody (or immunoreactive fragment thereof) heavy or light chain or appended thereto using standard molecular engineering techniques. In other preferred embodiments disruption of native disulfide bonds may be effected in combination with the introduction of a non-native cysteine (which will then comprise the free cysteine) that may then be used as a conjugation site.

In certain embodiments the engineered antibody comprises at least one amino acid deletion or substitution of an intrachain or interchain cysteine residue. As used herein “interchain cysteine residue” means a cysteine residue that is involved in a native disulfide bond either between the light and heavy chain of an antibody or between the two heavy chains of an antibody while an “intrachain cysteine residue” is one naturally paired with another cysteine in the same heavy or light chain. In one embodiment the deleted or substituted interchain cysteine residue is involved in the formation of a disulfide bond between the light and heavy chain. In another embodiment the deleted or substituted cysteine residue is involved in a disulfide bond between the two heavy chains. In a typical embodiment, due to the complementary structure of an antibody, in which the light chain is paired with the VH and CH1 domains of the heavy chain and wherein the CH2 and CH3 domains of one heavy chain are paired with the CH2 and CH3 domains of the complementary heavy chain, a mutation or deletion of a single cysteine in either the light chain or in the heavy chain would result in two unpaired cysteine residues in the engineered antibody.

In some embodiments an interchain cysteine residue is deleted. In other embodiments an interchain cysteine is substituted for another amino acid (e.g., a naturally occurring amino acid). For example, the amino acid substitution can result in the replacement of an interchain cysteine with a neutral (e.g. serine, threonine or glycine) or hydrophilic (e.g. methionine, alanine, valine, leucine or isoleucine) residue. In selected embodiments an interchain cysteine is replaced with a serine.

In some embodiments contemplated by the invention the deleted or substituted cysteine residue is on the light chain (either kappa or lambda) thereby leaving a free cysteine on the heavy chain. In other embodiments the deleted or substituted cysteine residue is on the heavy chain leaving the free cysteine on the light chain constant region. Upon assembly it will be appreciated that deletion or substitution of a single cysteine in either the light or heavy chain of an intact antibody results in a site-specific antibody having two unpaired cysteine residues.

In one embodiment the cysteine at position 214 (C214) of the IgG light chain (kappa or lambda) is deleted or substituted. In another embodiment the cysteine at position 220 (C220) on the IgG heavy chain is deleted or substituted. In further embodiments the cysteine at position 226 or position 229 on the heavy chain is deleted or substituted. In one embodiment C220 on the heavy chain is substituted with serine (C220S) to provide the desired free cysteine in the light chain. In another embodiment C214 in the light chain is substituted with serine (C214S) to provide the desired free cysteine in the heavy chain. Such site-specific constructs are described in more detail in the Examples below. A summary of compatible site-specific constructs is shown in Table 2 immediately below where numbering is generally according to the Eu index as set forth in Kabat, WT stands for “wild-type” or native constant region sequences without alterations and delta (Δ) designates the deletion of an amino acid residue (e.g., C214Δ indicates that the cysteine residue at position 214 has been deleted).

TABLE 2

| Designation | Antibody Component | Alteration |
|---|---|---|
| ss1 | Heavy Chain | C220S |
| | Light Chain | WT |
| ss2 | Heavy Chain | C220Δ |
| | Light Chain | WT |
| ss3 | Heavy Chain | WT |
| | Light Chain | C214Δ |
| ss4 | Heavy Chain | WT |
| | Light Chain | C214S |

With regard to the introduction or addition of a cysteine residue or residues to provide a free cysteine (as opposed to disrupting a native disulfide bond) compatible position(s) on the antibody or antibody fragment may readily be discerned by one skilled in the art. Accordingly, in selected embodiments the cysteine(s) may be introduced in the CH1 domain, the CH2 domain or the CH3 domain or any combination thereof depending on the desired DAR, the antibody construct, the selected payload and the antibody target. In other preferred embodiments the cysteines may be introduced into a kappa or lambda CL domain and, in particularly preferred embodiments, in the c-terminal region of the CL domain. In each case other amino acid residues proximal to the site of cysteine insertion may be altered, removed or substituted to facilitate molecular stability, conjugation efficiency or provide a protective environment for the payload once it is attached. In particular embodiments, the substituted residues occur at any accessible sites of the antibody. By substituting such surface residues with cysteine, reactive thiol groups are thereby positioned at readily accessible sites on the antibody and may be selectively reduced as described further herein. In particular embodiments, the substituted residues occur at accessible sites of the antibody. By substituting those residues with cysteine, reactive thiol groups are thereby positioned at accessible sites of the antibody and may be used to selectively conjugate the antibody. In certain embodiments, any one or more of the following residues may be substituted with cysteine: V205 (Kabat numbering) of the light chain; A118 (Eu numbering) of the heavy chain; and S400 (Eu numbering) of the heavy chain Fc region. Additional substitution positions and methods of fabricating compatible site-specific antibodies are set forth in U.S. Pat. No. 7,521,541 which is incorporated herein in its entirety.

The strategy for generating antibody drug conjugates with defined sites and stoichiometries of drug loading, as disclosed herein, is broadly applicable to all anti-DLL3 or anti-ASCL1antibodies as it primarily involves engineering of the conserved constant domains of the antibody. As the amino acid sequences and native disulfide bridges of each class and subclass of antibody are well documented, one skilled in the art could readily fabricate engineered constructs of various antibodies without undue experimentation and, accordingly, such constructs are expressly contemplated as being within the scope of the instant invention.

The strategy for generating antibody-drug conjugates with defined sites and stoichiometries of drug loading, as disclosed herein, is broadly applicable to all anti-DLL3 or anti-ASCL1 antibodies as it primarily involves engineering of the conserved constant domains of the antibody. As the amino acid sequences and native disulfide bridges of each class and subclass of antibody are well documented, one skilled in the art could readily fabricate engineered constructs of various DLL3 or ASCL1 antibodies without undue experimentation and, accordingly, such constructs are expressly contemplated as being within the scope of the instant invention.

4.3 Constant Region Modifications and Altered Glycosylation

Selected embodiments of the present invention may also comprise substitutions or modifications of the constant region (i.e. the Fc region), including without limitation, amino acid residue substitutions, mutations and/or modifications, which result in a compound with characteristics including, but not limited to: altered pharmacokinetics, increased serum half-life, increase binding affinity, reduced immunogenicity, increased production, altered Fc ligand binding to an Fc receptor (FcR), enhanced or reduced ADCC or CDC, altered glycosylation and/or disulfide bonds and modified binding specificity.

Compounds with improved Fc effector functions can be generated, for example, through changes in amino acid residues involved in the interaction between the Fc domain and an Fc receptor (e.g., FcγRI, FcγRIIA and B, FcγRIII and FcRn), which may lead to increased cytotoxicity and/or altered pharmacokinetics, such as increased serum half-life (see, for example, Ravetch and Kinet, Annu. Rev. Immunol 9:457-92 (1991); Capel et al., Immunomethods 4:25-34 (1994); and de Haas et al., J. Lab. Clin. Med. 126:330-41 (1995).

In selected embodiments, antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S.P.N. 2003/0190311). With regard to such embodiments, Fc variants may provide half-lives in a mammal, preferably a human, of greater than 5 days, greater than 10 days, greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-life results in a higher serum titer which thus reduces the frequency of the administration of the antibodies and/or reduces the concentration of the antibodies to be administered. Binding to human FcRn in vivo and serum half-life of human FcRn high affinity binding polypeptides can be assayed, e.g., in transgenic mice or transfected human cell lines expressing human FcRn, or in primates to which the polypeptides with a variant Fc region are administered. WO 2000/42072 describes antibody variants with improved or diminished binding to FcRns. See also, e.g., Shields et al. J. Biol. Chem. 9(2):6591-6604 (2001). Surprisingly, certain ADCs of the instant invention exhibit protracted terminal half-lives (e.g., on the order of two weeks) without any antibody constant region modifications other than those used to provide optional site-specific conjugates.

In other embodiments, Fc alterations may lead to enhanced or reduced ADCC or CDC activity. As in known in the art, CDC refers to the lysing of a target cell in the presence of complement, and ADCC refers to a form of cytotoxicity in which secreted Ig bound onto FcRs present on certain cytotoxic cells (e.g., Natural Killer cells, neutrophils, and macrophages) enables these cytotoxic effector cells to bind specifically to an antigen-bearing target cell and subsequently kill the target cell with cytotoxins. In the context of the instant invention antibody variants are provided with “altered” FcR binding affinity, which is either enhanced or diminished binding as compared to a parent or unmodified antibody or to an antibody comprising a native sequence FcR. Such variants which display decreased binding may possess little or no appreciable binding, e.g., 0-20% binding to the FcR compared to a native sequence, e.g. as determined by techniques well known in the art. In other embodiments the variant will exhibit enhanced binding as compared to the native immunoglobulin Fc domain. It will be appreciated that these types of Fc variants may advantageously be used to enhance the effective anti-neoplastic properties of the disclosed antibodies. In yet other embodiments, such alterations lead to increased binding affinity, reduced immunogenicity, increased production, altered glycosylation and/or disulfide bonds (e.g., for conjugation sites), modified binding specificity, increased phagocytosis; and/or down regulation of cell surface receptors (e.g. B cell receptor; BCR), etc.

Still other embodiments comprise one or more engineered glycoforms, e.g., a site-specific antibody comprising an altered glycosylation pattern or altered carbohydrate composition that is covalently attached to the protein (e.g., in the Fc domain). See, for example, Shields, R. L. et al. (2002) J. Biol. Chem. 277:26733-26740. Engineered glycoforms may be useful for a variety of purposes, including but not limited to enhancing or reducing effector function, increasing the affinity of the antibody for a target or facilitating production of the antibody. In certain embodiments where reduced effector function is desired, the molecule may be engineered to express an aglycosylated form. Substitutions that may result in elimination of one or more variable region framework glycosylation sites to thereby eliminate glycosylation at that site are well known (see e.g. U.S. Pat. Nos. 5,714,350 and 6,350,861). Conversely, enhanced effector functions or improved binding may be imparted to the Fc containing molecule by engineering in one or more additional glycosylation sites.

Other embodiments include an Fc variant that has an altered glycosylation composition, such as a hypofucosylated antibody having reduced amounts of fucosyl residues or an antibody having increased bisecting GlcNAc structures. Such altered glycosylation patterns have been demonstrated to increase the ADCC ability of antibodies. Engineered glycoforms may be generated by any method known to one skilled in the art, for example by using engineered or variant expression strains, by co-expression with one or more enzymes (for example N-acetylglucosaminyltransferase III (GnTIII)), by expressing a molecule comprising an Fc region in various organisms or cell lines from various organisms or by modifying carbohydrate(s) after the molecule comprising Fc region has been expressed (see, for example, WO 2012/117002).

4.4 Fragments

Regardless of which form of antibody (e.g. chimeric, humanized, etc.) is selected to practice the invention it will be appreciated that immunoreactive fragments, either by themselves or as part of an antibody drug conjugate, of the same may be used in accordance with the teachings herein. An “antibody fragment” comprises at least a portion of an intact antibody. As used herein, the term “fragment” of an antibody molecule includes antigen-binding fragments of antibodies, and the term “antigen-binding fragment” refers to a polypeptide fragment of an immunoglobulin or antibody that immunospecifically binds or reacts with a selected antigen or immunogenic determinant thereof or competes with the intact antibody from which the fragments were derived for specific antigen binding.

Exemplary site-specific fragments include: variable light chain fragments (VL), an variable heavy chain fragments (VH), scFv, F(ab′)2 fragment, Fab fragment, Fd fragment, Fv fragment, single domain antibody fragments, diabodies, linear antibodies, single-chain antibody molecules and multispecific antibodies formed from antibody fragments. In addition, an active site-specific fragment comprises a portion of the antibody that retains its ability to interact with the antigen/substrates or receptors and modify them in a manner similar to that of an intact antibody (though maybe with somewhat less efficiency). Such antibody fragments may further be engineered to comprise one or more free cysteines as described herein.

In other embodiments, an antibody fragment is one that comprises the Fc region and that retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence comprising at least one free cysteine capable of conferring in vivo stability to the fragment.

As would be well recognized by those skilled in the art, fragments can be obtained by molecular engineering or via chemical or enzymatic treatment (such as papain or pepsin) of an intact or complete antibody or antibody chain or by recombinant means. See, e.g., Fundamental Immunology, W. E. Paul, ed., Raven Press, N.Y. (1999), for a more detailed description of antibody fragments.

4.5 Multivalent Constructs

In other embodiments, the antibodies and conjugates of the invention may be monovalent or multivalent (e.g., bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen). See, for example, U.S.P.N. 2009/0130105.

In one embodiment, the antibodies are bispecific antibodies in which the two chains have different specificities, as described in Millstein et al., 1983, Nature, 305:537-539. Other embodiments include antibodies with additional specificities such as trispecific antibodies. Other more sophisticated compatible multispecific constructs and methods of their fabrication are set forth in U.S.P.N. 2009/0155255, as well as WO 94/04690; Suresh et al., 1986, Methods in Enzymology, 121:210; and WO96/27011.

Multivalent antibodies may immunospecifically bind to different epitopes of the desired target molecule or may immunospecifically bind to both the target molecule as well as a heterologous epitope, such as a heterologous polypeptide or solid support material. While selected embodiments may only bind two antigens (i.e. bispecific antibodies), antibodies with additional specificities such as trispecific antibodies are also encompassed by the instant invention. Bispecific antibodies also include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

5. Recombinant Production of Antibodies

Antibodies and fragments thereof may be produced or modified using genetic material obtained from antibody producing cells and recombinant technology (see, for example; Dubel and Reichert (Eds.) (2014) Handbook of Therapeutic Antibodies, 2^{nd} Edition, Wiley-Blackwell GmbH; Sambrook and Russell (Eds.) (2000) Molecular Cloning. A Laboratory Manual (3^{rd} Ed.), NY, Cold Spring Harbor Laboratory Press; Ausubel et al. (2002) Short Protocols in Molecular Biology: A Compendium of Methods from Current Protocols in Molecular Biology, Wiley, John & Sons, Inc.; and U.S. Pat. No. 7,709,611).

Another aspect of the invention pertains to nucleic acid molecules that encode the antibodies of the invention. The nucleic acids may be present in whole cells, in a cell lysate, or in a partially purified or substantially pure form. A nucleic acid is “isolated” or rendered substantially pure when separated from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the invention can be, for example, DNA (e.g. genomic DNA, cDNA), RNA and artificial variants thereof (e.g., peptide nucleic acids), whether single-stranded or double-stranded or RNA, RNA and may or may not contain introns. In selected embodiments the nucleic acid is a cDNA molecule.

Nucleic acids of the invention can be obtained using standard molecular biology techniques. For antibodies expressed by hybridomas (e.g., hybridomas prepared as described in the Examples below), cDNAs encoding the light and heavy chains of the antibody can be obtained by standard PCR amplification or cDNA cloning techniques. For antibodies obtained from an immunoglobulin gene library (e.g., using phage display techniques), nucleic acid encoding the antibody can be recovered from the library.

DNA fragments encoding VH and VL segments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein or protein fragment, such as an antibody constant region or a flexible linker. The term “operatively linked”, as used in this context, means that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3 in the case of IgG1). The sequences of human heavy chain constant region genes are known in the art (see e.g., Kabat, et al. (1991) (supra)) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. An exemplary IgG1 constant region is set forth in SEQ ID NO: 2. For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, et al. (1991) (supra)) and DNA fragments encompassing these regions can be obtained by standard PCR amplification. The light chain constant region can be a kappa or lambda constant region, but most preferably is a kappa constant region. An exemplary compatible kappa light chain constant region is set forth in SEQ ID NO: 5.

Contemplated herein are certain polypeptides (e.g. antigens or antibodies) that exhibit “sequence identity”, sequence similarity” or “sequence homology” to the polypeptides of the invention. For example, a derived humanized antibody VH or VL domain may exhibit a sequence similarity with the source (e.g., murine) or acceptor (e.g., human) VH or VL domain. A “homologous” polypeptide may exhibit 65%, 70%, 75%, 80%, 85%, or 90% sequence identity. In other embodiments a “homologous” polypeptides may exhibit 93%, 95% or 98% sequence identity. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Residue positions which are not identical may differ by conservative amino acid substitutions or by non-conservative amino acid substitutions. A “conservative amino acid substitution” is one in which an amino acid residue is substituted by another amino acid residue having a side chain with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. In cases where there is a substitution with a non-conservative amino acid, in embodiments the polypeptide exhibiting sequence identity will retain the desired function or activity of the polypeptide of the invention (e.g., antibody.)

Also contemplated herein are nucleic acids that that exhibit “sequence identity”, sequence similarity” or “sequence homology” to the nucleic acids of the invention. A “homologous sequence” means a sequence of nucleic acid molecules exhibiting at least about 65%, 70%, 75%, 80%, 85%, or 90% sequence identity. In other embodiments, a “homologous sequence” of nucleic acids may exhibit 93%, 95% or 98% sequence identity to the reference nucleic acid.

The instant invention also provides vectors comprising such nucleic acids described above, which may be operably linked to a promoter (see, e.g., WO 86/05807; WO 89/01036; and U.S. Pat. No. 5,122,464); and other transcriptional regulatory and processing control elements of the eukaryotic secretory pathway. The invention also provides host cells harboring those vectors and host-expression systems.

As used herein, the term “host-expression system” includes any type of cellular system that can be engineered to generate either the nucleic acids or the polypeptides and antibodies of the invention. Such host-expression systems include, but are not limited to microorganisms (e.g., E. coli or B. subtilis) transformed or transfected with recombinant bacteriophage DNA or plasmid DNA; yeast (e.g., Saccharomyces) transfected with recombinant yeast expression vectors; or mammalian cells (e.g., COS, CHO—S, HEK293T, 3T3 cells) harboring recombinant expression constructs containing promoters derived from the genome of mammalian cells or viruses (e.g., the adenovirus late promoter). The host cell may be co-transfected with two expression vectors, for example, the first vector encoding a heavy chain derived polypeptide and the second vector encoding a light chain derived polypeptide.

Methods of transforming mammalian cells are well known in the art. See, for example, U.S. Pat. Nos. 4,399,216, 4,912,040, 4,740,461, and 4,959,455. The host cell may also be engineered to allow the production of an antigen binding molecule with various characteristics (e.g. modified glycoforms or proteins having GnTIII activity).

For long-term, high-yield production of recombinant proteins stable expression is preferred. Accordingly, cell lines that stably express the selected antibody may be engineered using standard art recognized techniques and form part of the invention. Rather than using expression vectors that contain viral origins of replication, host cells can be transformed with DNA controlled by appropriate expression control elements (e.g., promoter or enhancer sequences, transcription terminators, polyadenylation sites, etc.), and a selectable marker. Any of the selection systems well known in the art may be used, including the glutamine synthetase gene expression system (the GS system) which provides an efficient approach for enhancing expression under selected conditions. The GS system is discussed in whole or part in connection with EP 0 216 846, EP 0 256 055, EP 0 323 997 and EP 0 338 841 and U.S. Pat. Nos. 5,591,639 and 5,879,936. Another compatible expression system for the development of stable cell lines is the Freedom™ CHO—S Kit (Life Technologies).

Once an antibody of the invention has been produced by recombinant expression or any other of the disclosed techniques, it may be purified or isolated by methods known in the art in that it is identified and separated and/or recovered from its natural environment and separated from contaminants that would interfere with diagnostic or therapeutic uses for the antibody or related ADC. Isolated antibodies include antibodies in situ within recombinant cells.

These isolated preparations may be purified using various art-recognized techniques, such as, for example, ion exchange and size exclusion chromatography, dialysis, diafiltration, and affinity chromatography, particularly Protein A or Protein G affinity chromatography. Compatible methods are discussed more fully in the Examples below.

6. Post-Production Selection

No matter how obtained, antibody-producing cells (e.g., hybridomas, yeast colonies, etc.) may be selected, cloned and further screened for desirable characteristics including, for example, robust growth, high antibody production and desirable antibody characteristics such as high affinity for the antigen of interest. Hybridomas can be expanded in vitro in cell culture or in vivo in syngeneic immunocompromised animals. Methods of selecting, cloning and expanding hybridomas and/or colonies are well known to those of ordinary skill in the art. Once the desired antibodies are identified the relevant genetic material may be isolated, manipulated and expressed using common, art-recognized molecular biology and biochemical techniques.

The antibodies produced by naïve libraries (either natural or synthetic) may be of moderate affinity (Kₐ of about 10⁶ to 10⁷ M¹). To enhance affinity, affinity maturation may be mimicked in vitro by constructing antibody libraries (e.g., by introducing random mutations in vitro by using error-prone polymerase) and reselecting antibodies with high affinity for the antigen from those secondary libraries (e.g. by using phage or yeast display). WO 9607754 describes a method for inducing mutagenesis in a CDR of an immunoglobulin light chain to create a library of light chain genes.

Various techniques can be used to select antibodies, including but not limited to, phage or yeast display in which a library of human combinatorial antibodies or scFv fragments is synthesized on phages or yeast, the library is screened with the antigen of interest or an antibody-binding portion thereof, and the phage or yeast that binds the antigen is isolated, from which one may obtain the antibodies or immunoreactive fragments (Vaughan et al., 1996, PMID: 9630891; Sheets et al., 1998, PMID: 9600934; Boder et al., 1997, PMID: 9181578; Pepper et al., 2008, PMID: 18336206). Kits for generating phage or yeast display libraries are commercially available. There also are other methods and reagents that can be used in generating and screening antibody display libraries (see U.S. Pat. No. 5,223,409; WO 92/18619, WO 91/17271, WO 92/20791, WO 92/15679, WO 93/01288, WO 92/01047, WO 92/09690; and Barbas et al., 1991, PMID: 1896445). Such techniques advantageously allow for the screening of large numbers of candidate antibodies and provide for relatively easy manipulation of sequences (e.g., by recombinant shuffling).

### V. Characteristics of Antibodies

In certain embodiments, antibody-producing cells (e.g., hybridomas or yeast colonies) may be selected, cloned and further screened for favorable properties including, for example, robust growth, high antibody production and, as discussed in more detail below, desirable site-specific antibody characteristics. In other cases characteristics of the antibody may be imparted by selecting a particular antigen (e.g., a specific DLL3 or ASCL1 isoform) or immunoreactive fragment of the target antigen for inoculation of the animal. In still other embodiments the selected antibodies may be engineered as described above to enhance or refine immunochemical characteristics such as affinity or pharmacokinetics.

A. Neutralizing Antibodies

In certain embodiments, the conjugates will comprise “neutralizing” antibodies or derivatives or fragments thereof. That is, the present invention may comprise antibody molecules that bind specific domains, motifs or epitopes and are capable of blocking, reducing or inhibiting the biological activity of DLL3 or ASCL1. More generally the term “neutralizing antibody” refers to an antibody that binds to or interacts with a target molecule or ligand and prevents binding or association of the target molecule to a binding partner such as a receptor or substrate, thereby interrupting a biological response that otherwise would result from the interaction of the molecules.

It will be appreciated that competitive binding assays known in the art may be used to assess the binding and specificity of an antibody or immunologically functional fragment or derivative thereof. With regard to the instant invention an antibody or fragment will be held to inhibit or reduce binding of DLL3 or ASCL1 to a binding partner or substrate when an excess of antibody reduces the quantity of binding partner bound to DLL3 or ASCL1 by at least about 20%, 30%, 40%, 50%, 60%, 70%, 80%, 85%, 90%, 95%, 97%, 99% or more as measured, for example, by Notch receptor activity or in an in vitro competitive binding assay. In the case of antibodies to DLL3 for example, a neutralizing antibody or antagonist will preferably alter Notch receptor activity by at least about 20%, 30%, 40%, 50%, 60%, 70%, 80%, 85%, 90%, 95%, 97%, 99% or more. It will be appreciated that this modified activity may be measured directly using art-recognized techniques or may be measured by the impact the altered activity has downstream (e.g., oncogenesis, cell survival or activation or suppression of Notch responsive genes). Preferably, the ability of an antibody to neutralize DLL3 activity is assessed by inhibition of DLL3 binding to a Notch receptor or by assessing its ability to relieve DLL3 mediated repression of Notch signaling.

B. Internalizing Antibodies

In certain embodiments the antibodies may comprise internalizing antibodies such that the antibody will bind to a determinant and will be internalized (along with any conjugated pharmaceutically active moiety) into a selected target cell including tumorigenic cells. The number of antibody molecules internalized may be sufficient to kill an antigen-expressing cell, especially an antigen-expressing tumorigenic cell. Depending on the potency of the antibody or, in some instances, antibody drug conjugate, the uptake of a single antibody molecule into the cell may be sufficient to kill the target cell to which the antibody binds. With regard to the instant invention there is evidence that a substantial portion of expressed DLL3 protein remains associated with the tumorigenic cell surface, thereby allowing for localization and internalization of the disclosed antibodies or ADCs. In selected embodiments such antibodies will be associated with, or conjugated to, one or more drugs that kill the cell upon internalization. In some embodiments the ADCs of the instant invention will comprise an internalizing site-specific ADC.

As used herein, an antibody that “internalizes” is one that is taken up (along with any conjugated cytotoxin) by a target cell upon binding to an associated determinant. The number of such ADCs internalized will preferably be sufficient to kill the determinant-expressing cell, especially a determinant-expressing cancer stem cell. Depending on the potency of the cytotoxin or ADC as a whole, in some instances the uptake of a few antibody molecules into the cell is sufficient to kill the target cell to which the antibody binds. For example, certain drugs such as PBDs or calicheamicin are so potent that the internalization of a few molecules of the toxin conjugated to the antibody is sufficient to kill the target cell. Whether an antibody internalizes upon binding to a mammalian cell can be determined by various art-recognized assays (e.g., saporin assays such as Mab-Zap and Fab-Zap; Advanced Targeting Systems). Methods of detecting whether an antibody internalizes into a cell are also described in U.S. Pat. No. 7,619,068.

C. Depleting Antibodies

In other embodiments the antibodies of the invention are depleting antibodies. The term “depleting” antibody refers to an antibody that preferably binds to an antigen on or near the cell surface and induces, promotes or causes the death of the cell (e.g., by CDC, ADCC or introduction of a cytotoxic agent). In embodiments, the selected depleting antibodies will be conjugated to a cytotoxin.

Preferably a depleting antibody will be able to kill at least 20%, 30%, 40%, 50%, 60%, 70%, 80%, 85%, 90%, 95%, 97%, or 99% of DLL3-expressing cells or ASCL1-expressing cells in a defined cell population. In some embodiments the cell population may comprise enriched, sectioned, purified or isolated tumorigenic cells, including cancer stem cells. In other embodiments the cell population may comprise whole tumor samples or heterogeneous tumor extracts that comprise cancer stem cells. Standard biochemical techniques may be used to monitor and quantify the depletion of tumorigenic cells in accordance with the teachings herein.

D. Binding Affinity

Disclosed herein are antibodies that have a high binding affinity for a specific determinant e.g. DLL3 or ASCL1. The term “K_{D}” refers to the dissociation constant or apparent affinity of a particular antibody-antigen interaction. An antibody of the invention can immunospecifically bind its target antigen when the dissociation constant K_{D} (k_{off}/kₒₙ) is ≤10⁻⁷M. The antibody specifically binds antigen with high affinity when the K_{D} is ≤5×10⁻⁹ M, and with very high affinity when the K_{D} is ≤5×10⁻¹⁰ M. In one embodiment of the invention, the antibody has a K_{D} of ≤10⁻⁹ M and an off-rate of about 1×10⁻⁴/sec. In one embodiment of the invention, the off-rate is ≤1×10⁻⁵/sec. In other embodiments of the invention, the antibodies will bind to a determinant with a K_{D} of between about 10⁻⁷ M and 10⁻¹⁰ M, and in yet another embodiment it will bind with a K_{D}≤2×10⁻¹⁰ M. Still other selected embodiments of the invention comprise antibodies that have a K_{D} (k_{off}/kₒₙ) of less than 10⁻⁶ M, less than 5×10⁻⁶ M, less than 10⁻⁷ M, less than 5×10⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M or less than 5×10⁻¹⁵ M.

In certain embodiments, an antibody of the invention that immunospecifically binds to a determinant e.g. DLL3 or ASCL1 may have an association rate constant or kₒₙ (or kₐ) rate (antibody+antigen (Ag)^{k}ₒₙ←antibody-Ag) of at least 10⁵ M⁻¹s⁻¹, at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an antibody of the invention that immunospecifically binds to a determinant e.g. DLL3 or ASCL1 may have a disassociation rate constant or k_{off} (or k_{d}) rate (antibody+antigen (Ag)^{k}_{off}←antibody-Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹ less than 10⁻⁷ s⁻¹, less than 5×10⁻⁷ s⁻¹, less than 10⁻⁸ s⁻¹, less than 5×10⁸ s⁻¹, less than 10⁻⁹ s⁻¹, less than 5×10⁻⁹ s⁻¹ or less than 10⁻¹⁰ s⁻¹.

Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry. s

E. Binning and Epitope Mapping

Antibodies disclosed herein may be characterized in terms of the discrete epitope with which they associate. An “epitope” is the portion(s) of a determinant to which the antibody or immunoreactive fragment specifically binds. Immunospecific binding can be confirmed and defined based on binding affinity, as described above, or by the preferential recognition by the antibody of its target antigen in a complex mixture of proteins and/or macromolecules (e.g. in competition assays). A “linear epitope”, is formed by contiguous amino acids in the antigen that allow for immunospecific binding of the antibody. The ability to preferentially bind linear epitopes is typically maintained even when the antigen is denatured. Conversely, a “conformational epitope”, usually comprises non-contiguous amino acids in the antigen's amino acid sequence but, in the context of the antigen's secondary, tertiary or quaternary structure, are sufficiently proximate to be bound concomitantly by a single antibody. When antigens with conformational epitopes are denatured, the antibody will typically no longer recognize the antigen. An epitope (contiguous or non-contiguous) typically includes at least 3, and more usually, at least 5 or 8-10 or 12-20 amino acids in a unique spatial conformation.

It is also possible to characterize the antibodies of the invention in terms of the group or “bin” to which they belong. “Binning” refers to the use of competitive antibody binding assays to identify pairs of antibodies that are incapable of binding an immunogenic determinant simultaneously, thereby identifying antibodies that “compete” for binding. Competing antibodies may be determined by an assay in which the antibody or immunologically functional fragment being tested prevents or inhibits specific binding of a reference antibody to a common antigen. Typically, such an assay involves the use of purified antigen (e.g., DLL3, ASCL1, or a domain or fragment thereof) bound to a solid surface or cells, an unlabeled test antibody and a labeled reference antibody. Competitive inhibition is measured by determining the amount of label bound to the solid surface or cells in the presence of the test antibody. Additional details regarding methods for determining competitive binding are provided in the Examples herein. Usually, when a competing antibody is present in excess, it will inhibit specific binding of a reference antibody to a common antigen by at least 30%, 40%, 45%, 50%, 55%, 60%, 65%, 70% or 75%. In some instance, binding is inhibited by at least 80%, 85%, 90%, 95%, or 97% or more. Conversely, when the reference antibody is bound it will preferably inhibit binding of a subsequently added test antibody (i.e., a DLL3 antibody or ASCL1 antibody) by at least 30%, 40%, 45%, 50%, 55%, 60%, 65%, 70% or 75%. In some instance, binding of the test antibody is inhibited by at least 80%, 85%, 90%, 95%, or 97% or more.

Generally binning or competitive binding may be determined using various art-recognized techniques, such as, for example, immunoassays such as western blots, radioimmunoassays, enzyme linked immunosorbent assay (ELISA), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such immunoassays are routine and well known in the art (see, Ausubel et al, eds, (1994) Current Protocols in Molecular Biology, Vol. 1, John Wiley & Sons, Inc., New York). Additionally, cross-blocking assays may be used (see, for example, WO 2003/48731; and Harlow et al. (1988) Antibodies, A Laboratory Manual, Cold Spring Harbor Laboratory, Ed Harlow and David Lane).

Other technologies used to determine competitive inhibition (and hence “bins”), include: surface plasmon resonance using, for example, the BIAcore™ 2000 system (GE Healthcare); bio-layer interferometry using, for example, a ForteBio® Octet RED (ForteBio); or flow cytometry bead arrays using, for example, a FACSCanto II (BD Biosciences) or a multiplex LUMINEX™ detection assay (Luminex).

Luminex is a bead-based immunoassay platform that enables large scale multiplexed antibody pairing. The assay compares the simultaneous binding patterns of antibody pairs to the target antigen. One antibody of the pair (capture mAb) is bound to Luminex beads, wherein each capture mAb is bound to a bead of a different color. The other antibody (detector mAb) is bound to a fluorescent signal (e.g. phycoerythrin (PE)). The assay analyzes the simultaneous binding (pairing) of antibodies to an antigen and groups together antibodies with similar pairing profiles. Similar profiles of a detector mAb and a capture mAb indicates that the two antibodies bind to the same or closely related epitopes. In one embodiment, pairing profiles can be determined using Pearson correlation coefficients to identify the antibodies which most closely correlate to any particular antibody on the panel of antibodies that are tested. In embodiments a test/detector mAb will be determined to be in the same bin as a reference/capture mAb if the Pearson's correlation coefficient of the antibody pair is at least 0.9. In other embodiments the Pearson's correlation coefficient is at least 0.8, 0.85, 0.87 or 0.89. In further embodiments, the Pearson's correlation coefficient is at least 0.91, 0.92, 0.93, 0.94, 0.95, 0.96, 0.97, 0.98, 0.99 or 1. Other methods of analyzing the data obtained from the Luminex assay are described in U.S. Pat. No. 8,568,992. The ability of Luminex to analyze 100 different types of beads (or more) simultaneously provides almost unlimited antigen and/or antibody surfaces, resulting in improved throughput and resolution in antibody epitope profiling over a biosensor assay (Miller, et al., 2011, PMID: 21223970).

Similarly binning techniques comprising surface plasmon resonance are compatible with the instant invention. As used herein “surface plasmon resonance,” refers to an optical phenomenon that allows for the analysis of real-time specific interactions by detection of alterations in protein concentrations within a biosensor matrix. Using commercially available equipment such as the BIAcore™ 2000 system it may readily be determined if selected antibodies compete with each other for binding to a defined antigen.

In other embodiments, a technique that can be used to determine whether a test antibody “competes” for binding with a reference antibody is “bio-layer interferometry”, an optical analytical technique that analyzes the interference pattern of white light reflected from two surfaces: a layer of immobilized protein on a biosensor tip, and an internal reference layer. Any change in the number of molecules bound to the biosensor tip causes a shift in the interference pattern that can be measured in real-time. Such biolayer interferometry assays may be conducted using a ForteBio® Octet RED machine as follows. A reference antibody (Ab1) is captured onto an anti-mouse capture chip, a high concentration of non-binding antibody is then used to block the chip and a baseline is collected. Monomeric, recombinant target protein is then captured by the specific antibody (Ab1) and the tip is dipped into a well with either the same antibody (Ab1) as a control or into a well with a different test antibody (Ab2). If no further binding occurs, as determined by comparing binding levels with the control Ab1, then Ab1 and Ab2 are determined to be “competing” antibodies. If additional binding is observed with Ab2, then Ab1 and Ab2 are determined not to compete with each other. This process can be expanded to screen large libraries of unique antibodies using a full row of antibodies in a 96-well plate representing unique bins. In embodiments a test antibody will compete with a reference antibody if the reference antibody inhibits specific binding of the test antibody to a common antigen by at least 40%, 45%, 50%, 55%, 60%, 65%, 70% or 75%. In other embodiments, binding is inhibited by at least 80%, 85%, 90%, 95%, or 97% or more.

Once a bin, encompassing a group of competing antibodies, has been defined further characterization can be carried out to determine the specific domain or epitope on the antigen to which that group of antibodies binds. Domain-level epitope mapping may be performed using a modification of the protocol described by Cochran et al., 2004, PMID: 15099763. Fine epitope mapping is the process of determining the specific amino acids on the antigen that comprise the epitope of a determinant to which the antibody binds.

In certain embodiments fine epitope mapping can be performed using phage or yeast display. Other compatible epitope mapping techniques include alanine scanning mutants, peptide blots (Reineke, 2004, PMID: 14970513), or peptide cleavage analysis. In addition, methods such as epitope excision, epitope extraction and chemical modification of antigens can be employed (Tomer, 2000, PMID: 10752610) using enzymes such as proteolytic enzymes (e.g., trypsin, endoproteinase Glu-C, endoproteinase Asp-N, chymotrypsin, etc.); chemical agents such as succinimidyl esters and their derivatives, primary amine-containing compounds, hydrazines and carbohydrazines, free amino acids, etc. In another embodiment Modification-Assisted Profiling, also known as Antigen Structure-based Antibody Profiling (ASAP) can be used to categorize large numbers of monoclonal antibodies directed against the same antigen according to the similarities of the binding profile of each antibody to chemically or enzymatically modified antigen surfaces (U.S.P.N. 2004/0101920).

Once a desired epitope on an antigen is determined, it is possible to generate additional antibodies to that epitope, e.g., by immunizing with a peptide comprising the selected epitope using techniques described herein.

### VI. Antibody Conjugates

In some embodiments the antibodies of the invention may be conjugated with pharmaceutically active or diagnostic moieties to form an “antibody drug conjugate” (ADC) or “antibody conjugate”. The term “conjugate” is used broadly and means the covalent or non-covalent association of any pharmaceutically active or diagnostic moiety with an antibody of the instant invention regardless of the method of association. In certain embodiments the association is effected through a lysine or cysteine residue of the antibody. In some embodiments the pharmaceutically active or diagnostic moieties may be conjugated to the antibody via one or more site-specific free cysteine(s). The disclosed ADCs may be used for therapeutic and diagnostic purposes.

It will be appreciated that the ADCs of the instant invention may be used to selectively deliver predetermined warheads to the target location (e.g., tumorigenic cells and/or cells expressing DLL3). As set forth herein the terms “drug” or “warhead” may be used interchangeably and will mean any biologically active (e.g., a pharmaceutically active compound or therapeutic moiety) or detectable molecule or compound that has a physiological effect or reporter function when introduced into a subject. For the avoidance of doubt such warheads include the anti-cancer agents or cytotoxins as described below. A “payload” may comprise a drug or warhead in combination with an optional linker compound (e.g., a therapeutic payload) that preferably provides a relatively stable pharmaceutical complex until the ADC reaches the target. By way of example the warhead or drug on the conjugate may comprise peptides, proteins or prodrugs which are metabolized to an active agent in vivo, polymers, nucleic acid molecules, small molecules, binding agents, mimetic agents, synthetic drugs, inorganic molecules, organic molecules and radioisotopes. In certain embodiments the drug or warhead will be covalently conjugated to the antibody through a linker. In other embodiments (e.g., a radioisotope) the drug or warhead will be directly conjugated to, or incorporated in, the antibody.

In preferred embodiments the disclosed ADCs will direct the bound payload (e.g., drug linker) to the target site in a relatively unreactive, non-toxic state before releasing and activating the warhead (e.g., PBDS 1-5 as disclosed herein). This targeted release of the warhead is preferably achieved through stable conjugation of the payloads (e.g., via one or more cysteines or lysines on the antibody) and relatively homogeneous composition of the ADC preparations which minimize over-conjugated toxic ADC species. Coupled with drug linkers that are designed to largely release the warhead upon delivery to the tumor site, the conjugates of the instant invention can substantially reduce undesirable non-specific toxicity. This advantageously provides for relatively high levels of the active cytotoxin at the tumor site while minimizing exposure of non-targeted cells and tissue thereby providing an enhanced therapeutic index.

It will be appreciated that, while some embodiments of the invention comprise payloads incorporating therapeutic moieties (e.g., cytotoxins), other payloads incorporating diagnostic agents and biocompatible modifiers may benefit from the targeted delivery provided by the disclosed conjugates. Accordingly, any disclosure directed to exemplary therapeutic payloads is also applicable to payloads comprising diagnostic agents or biocompatible modifiers as discussed herein unless otherwise dictated by context. The selected payload may be covalently or non-covalently linked to the antibody and exhibit various stoichiometric molar ratios depending, at least in part, on the method used to effect the conjugation.

Conjugates of the instant invention may be generally represented by the formula:

Ab-[L-D]n or a pharmaceutically acceptable salt thereof wherein:
a) Ab comprises an anti-DLL3 antibody;
b) L comprises an optional linker;
c) D comprises a drug; and
d) n is an integer from about 1 to about 20.

Those of skill in the art will appreciate that conjugates according to the aforementioned formula may be fabricated using a number of different linkers and drugs and that conjugation methodology will vary depending on the selection of components. As such, any drug or drug linker compound that associates with a reactive residue (e.g., cysteine or lysine) of the disclosed antibodies are compatible with the teachings herein. Similarly, any reaction conditions that allow for conjugation (including site-specific conjugation) of the selected drug to an antibody are within the scope of the present invention. Notwithstanding the foregoing, some preferred embodiments of the instant invention comprise selective conjugation of the drug or drug linker to free cysteines using stabilization agents in combination with mild reducing agents as described herein. Such reaction conditions tend to provide more homogeneous preparations with less non-specific conjugation and contaminants and correspondingly less toxicity.

A. Payloads and Warheads

1. Therapeutic Agents

As discussed the antibodies of the invention may be conjugated, linked or fused to or otherwise associated with any pharmaceutically active compound comprising a therapeutic moiety or a drug such as an anti-cancer agent including, but not limited to, cytotoxic agents (or cytotoxins), cytostatic agents, anti-angiogenic agents, debulking agents, chemotherapeutic agents, radiotherapeutic agents, targeted anti-cancer agents, biological response modifiers, cancer vaccines, cytokines, hormone therapies, anti-metastatic agents and immunotherapeutic agents.

Exemplary anti-cancer agents or cytotoxins (including homologs and derivatives thereof) comprise 1-dehydrotestosterone, anthramycins, actinomycin D, bleomycin, calicheamicins (including n-acetyl calicheamicin), colchicin, cyclophosphamide, cytochalasin B, dactinomycin (formerly actinomycin), dihydroxy anthracin, dione, duocarmycin, emetine, epirubicin, ethidium bromide, etoposide, glucocorticoids, gramicidin D, lidocaine, maytansinoids such as DM-1 and DM-4 (Immunogen), benzodiazepine derivatives (Immunogen), mithramycin, mitomycin, mitoxantrone, paclitaxel, procaine, propranolol, puromycin, tenoposide, tetracaine and pharmaceutically acceptable salts or solvates, acids or derivatives of any of the above.

Additional compatible cytotoxins comprise dolastatins and auristatins, including monomethyl auristatin E (MMAE) and monomethyl auristatin F (MMAF) (Seattle Genetics), amanitins such as alpha-amanitin, beta-amanitin, gamma-amanitin or epsilon-amanitin (Heidelberg Pharma), DNA minor groove binding agents such as duocarmycin derivatives (Syntarga), alkylating agents such as modified or dimeric pyrrolobenzodiazepines (PBD), mechlorethamine, thioepa, chlorambucil, melphalan, carmustine (BCNU), lomustine (CCNU), cyclothosphamide, busulfan, dibromomannitol, streptozotocin, mitomycin C and cisdichlorodiamine platinum (II) (DDP) cisplatin, splicing inhibitors such as meayamycin analogs or derivatives (e.g., FR901464 as set forth in U.S. Pat. No. 7,825,267), tubular binding agents such as epothilone analogs and tubulysins, paclitaxel and DNA damaging agents such as calicheamicins and esperamicins, antimetabolites such as methotrexate, 6-mercaptopurine, 6-thioguanine, cytarabine, and 5-fluorouracil decarbazine, anti-mitotic agents such as vinblastine and vincristine and anthracyclines such as daunorubicin (formerly daunomycin) and doxorubicin and pharmaceutically acceptable salts or solvates, acids or derivatives of any of the above.

In selected embodiments the antibodies of the instant invention may be associated with anti-CD3 binding molecules to recruit cytotoxic T-cells and have them target tumorigenic cells (BiTE technology; see e.g., Fuhrmann et. al. (2010) Annual Meeting of AACR Abstract No. 5625).

In further embodiments ADCs of the invention may comprise cytotoxins comprising therapeutic radioisotopes conjugated using appropriate linkers. Exemplary radioisotopes that may be compatible with such embodiments include, but are not limited to, iodine (¹³¹I, ¹²⁵I, ¹²³I, ¹²¹I,), carbon (¹⁴C), copper (⁶²Cu, ⁶⁴Cu, ⁶⁷Cu), sulfur (³⁵S), radium (²²³R), tritium (³H), indium (¹¹⁵In, ¹¹³In, ¹¹²In, ¹¹¹In,), bismuth (²¹²Bi, ²¹³Bi), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, ¹¹⁷Sn, ⁷⁶Br, ²¹¹At and ²²⁵Ac. Other radionuclides are also available as diagnostic and therapeutic agents, especially those in the energy range of 60 to 4,000 keV.

In other selected embodiments the ADCs of the instant invention will be conjugated to a cytotoxic benzodiazepine derivative warhead. Compatible benzodiazepine derivatives (and optional linkers) that may be conjugated to the disclosed antibodies are described, for example, in U.S. Pat. No. 8,426,402 and PCT filings WO2012/128868 and WO2014/031566. As with PBDs, compatible benzodiazepine derivatives are believed to bind in the minor grove of DNA and inhibit nucleic acid synthesis. Such compounds reportedly have potent antitumor properties and, as such, are particularly suitable for use in the ADCs of the instant invention.

In some embodiments, the ADCs of the invention may comprise PBDs, and pharmaceutically acceptable salts or solvates, acids or derivatives thereof, as warheads. PBDs are alkylating agents that exert antitumor activity by covalently binding to DNA in the minor groove and inhibiting nucleic acid synthesis. PBDs have been shown to have potent antitumor properties while exhibiting minimal bone marrow depression. PBDs compatible with the invention may be linked to an antibody using several types of linkers (e.g., a peptidyl linker comprising a maleimido moiety with a free sulfhydryl), and in certain embodiments are dimeric in form (i.e., PBD dimers). Compatible PBDs (and optional linkers) that may be conjugated to the disclosed antibodies are described, for example, in U.S. Pat. Nos. 6,362,331, 7,049,311, 7,189,710, 7,429,658, 7,407,951, 7,741,319, 7,557,099, 8,034,808, 8,163,736, 2011/0256157 and PCT filings WO2011/130613, WO2011/128650, WO2011/130616, WO2014/057073 and WO2014/057074. Examples of PBD compounds compatible with the instant invention are discussed in more detail immediately below.

With regard to the instant invention PBDs have been shown to have potent antitumor properties while exhibiting minimal bone marrow depression. PBDs compatible with the present invention may be linked to the DLL3 targeting agent using any one of several types of linker (e.g., a peptidyl linker comprising a maleimido moiety with a free sulflydryl) and, in certain embodiments are dimeric in form (i.e., PBD dimers). PBDs are of the general structure:

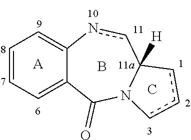

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as cytotoxic agents. As alluded to above, in order to increase their potency PBDs are often used in a dimeric form which may be conjugated to anti-DLL3 antibodies as described herein.

In particularly preferred embodiments compatible PBDs that may be conjugated to the disclosed modulators are described, in U.S.P.N. 2011/0256157. In this disclosure, PBD dimers, i.e. those comprising two PBD moieties may be preferred. Thus, preferred conjugates of the present invention are those having the formula (AB) or (AC):

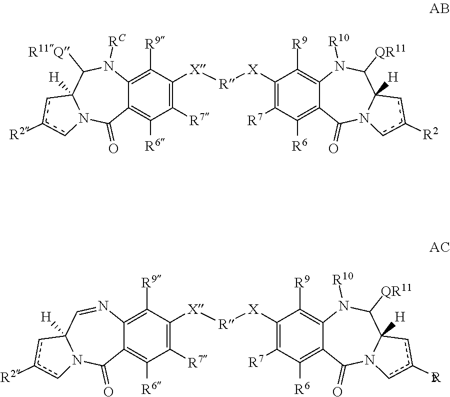

wherein:

the dotted lines indicate the optional presence of a double bond between C1 and C2 or C2 and C3;

R² is independently selected from H, OH, ═O, ═CH₂, CN, R, OR, ═CH—R^{D}, ═C(R^{D})₂, O—SO₂—R, CO₂R and COR, and optionally further selected from halo or dihalo;

where R^{D} is independently selected from R, CO₂R, COR, CHO, CO₂H, and halo;

R⁶ and R⁹ are independently selected from H, R, OH, OR, SH, SR, NH₂, NHR, NRR′, NO₂, Me₃Sn and halo;

R⁷ is independently selected from H, R, OH, OR, SH, SR, NH₂, NHR, NRR′, NO₂, Me₃Sn and halo;

R¹⁰ is a linker connected to a DLL3 antibody or fragment or derivative thereof, as described herein;

Q is independently selected from O, S and NH;

R¹¹ is either H, or R or, where Q is O, R¹¹ may be SO₃M, where M is a metal cation;

X is selected from O, S, or N(H) and in selected embodiments comprises O;

R″ is a C₃₋₁₂ alkylene group, which chain may be interrupted by one or more heteroatoms (e.g., O, S, N(H), NMe and/or aromatic rings, e.g. benzene or pyridine, which rings are optionally substituted);

R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring; and

wherein R^{2″}, R^{6″}, R^{7″}, R^{9″}, X″, Q″ and R^{11″} (where present) are as defined according to R², R⁶, R⁷, R⁹, X, Q and R¹¹ respectively, and R^{C} is a capping group.

Selected embodiments comprising the aforementioned structures are described in more detail immediately below.

### Double Bond

In one embodiment, there is no double bond present between C1 and C2, and C2 and C3.

In one embodiment, the dotted lines indicate the optional presence of a double bond between C2 and C3, as shown below:

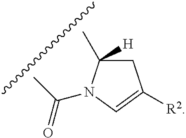

In one embodiment, a double bond is present between C2 and C3 when R² is C₅₋₂₀ aryl or C₁₋₁₂ alkyl. In a preferred embodiment R² comprises a methyl group.

In one embodiment, the dotted lines indicate the optional presence of a double bond between C1 and C2, as shown below:

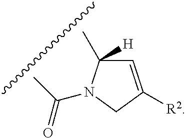

In one embodiment, a double bond is present between C1 and C2 when R² is C₅₋₂₀ aryl or C₁₋₁₂ alkyl. In a preferred embodiment R² comprises a methyl group.

### R²

In one embodiment, R² is independently selected from H, OH, ═O, ═CH₂, CN, R, OR, ═CH—R^{D}, ═C(R^{D})₂, O—SO₂—R, CO₂R and COR, and optionally further selected from halo or dihalo.

In one embodiment, R² is independently selected from H, OH, ═O, ═CH₂, CN, R, OR, ═CH—R^{D}, ═C(R^{D})₂, O—SO₂—R, CO₂R and COR.

In one embodiment, R² is independently selected from H, ═O, ═CH₂, R, ═CH—R^{D}, and ═C(R^{D})₂.

In one embodiment, R² is independently H.

In one embodiment R² is independently R wherein R comprises CH₃.

In one embodiment, R² is independently ═O.

In one embodiment, R² is independently ═CH₂.

In one embodiment, R² is independently ═CH—R^{D}. Within the PBD compound, the group ═CH—R^{D} may have either configuration shown below:

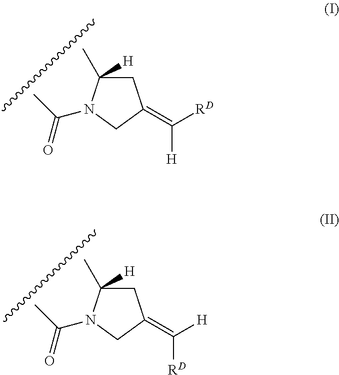

In one embodiment, the configuration is configuration (I).

In one embodiment, R² is independently ═C(R^{D})₂.

In one embodiment, R² is independently ═CF₂.

In one embodiment, R² is independently R.

In one embodiment, R² is independently optionally substituted C₅₋₂₀ aryl.

In one embodiment, R² is independently optionally substituted C₁₋₁₂ alkyl.

In one embodiment, R² is independently optionally substituted C₅₋₂₀ aryl.

In one embodiment, R² is independently optionally substituted C₅₋₇ aryl.

In one embodiment, R² is independently optionally substituted C₈₋₁₀ aryl.

In one embodiment, R² is independently optionally substituted phenyl.

In one embodiment. R² is independently optionally substituted napthyl.

In one embodiment, R² is independently optionally substituted pyridyl.

In one embodiment, R² is independently optionally substituted quinolinyl or isoquinolinyl.

In one embodiment, R² bears one to three substituent groups, with 1 and 2 being more preferred, and singly substituted groups being most preferred. The substituents may be any position.

Where R² is a C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

In one embodiment, R² is selected from:

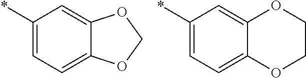

where the asterisk indicates the point of attachment.

Where R² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

In one embodiment, where R² is optionally substituted, the substituents are selected from those substituents given in the substituent section below.

Where R is optionally substituted, the substituents are preferably selected from:

Halo, Hydroxyl, Ether, Formyl, Acyl, Carboxy, Ester, Acyloxy, Amino, Amido, Acylamido, Aminocarbonyloxy, Ureido, Nitro, Cyano and Thioether.

In one embodiment, where R or R² is optionally substituted, the substituents are selected from the group consisting of R, OR, SR, NRR′, NO₂, halo, CO₂R, COR, CONH₂, CONHR, and CONRR′.

Where R² is C₁₋₁₂ alkyl, the optional substituent may additionally include C₃₋₂₀ heterocyclyl and C₅₋₂₀ aryl groups.

Where R² is C₃₋₂₀ heterocyclyl, the optional substituent may additionally include C₁₋₁₂ alkyl and C₅₋₂₀ aryl groups.

Where R² is C₅₋₂₀ aryl groups, the optional substituent may additionally include C₃₋₂₀ heterocyclyl and C₁₋₁₂ alkyl groups.

It is understood that the term “alkyl” encompasses the sub-classes alkenyl and alkynyl as well as cycloalkyl. Thus, where R² is optionally substituted C₁₋₁₂ alkyl, it is understood that the alkyl group optionally contains one or more carbon-carbon double or triple bonds, which may form part of a conjugated system. In one embodiment, the optionally substituted C₁₋₁₂ alkyl group contains at least one carbon-carbon double or triple bond, and this bond is conjugated with a double bond present between C1 and C2, or C2 and C3. In one embodiment, the C₁₋₁₂ alkyl group is a group selected from saturated C₁₋₁₂ alkyl, C₂₋₁₂ alkenyl, C₂₋₁₂ alkynyl and C₃₋₁₂ cycloalkyl.

If a substituent on R² is halo, it is preferably F or C1, more preferably C1.

If a substituent on R² is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy).

If a substituent on R² is C₁₋₇ alkyl, it may preferably be a C₁₋₄ alkyl group (e.g. methyl, ethyl, propyl, butyl).

If a substituent on R² is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If a substituent on R² is bis-oxy-C₁₋₃ alkylene, this is preferably bis-oxy-methylene or bis-oxy-ethylene.

Particularly preferred substituents for R² include methoxy, ethoxy, fluoro, chloro, cyano, bis-oxy-methylene, methyl-piperazinyl, morpholino and methyl-thienyl.

Particularly preferred substituted R² groups include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthienyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl.

In one embodiment, R² is halo or dihalo. In one embodiment, R² is —F or —F₂, which substituents are illustrated below as (III) and (IV) respectively:

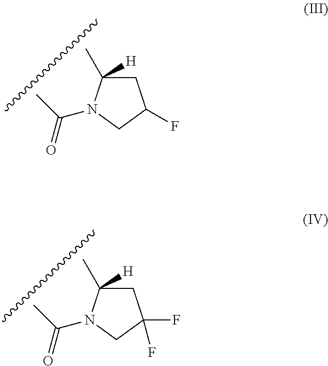

### R^{D}

In one embodiment, R^{D} is independently selected from R, CO₂R, COR, CHO, CO₂H, and halo.

In one embodiment, R^{D} is independently R.

In one embodiment, R^{D} is independently halo.

### R⁶

In one embodiment, R⁶ is independently selected from H, R, OH, OR, SH, SR, NH₂, NHR, NRR′, NO₂, Me₃Sn— and Halo.

In one embodiment, R⁶ is independently selected from H, OH, OR, SH, NH₂, NO₂ and Halo.

In one embodiment, R⁶ is independently selected from H and Halo.

In one embodiment, R⁶ is independently H.

In one embodiment, R⁶ and R⁷ together form a group —O—(CH₂)ₚ—O—, where p is 1 or 2.

### R⁷

R⁷ is independently selected from H, R, OH, OR, SH, SR, NH₂, NHR, NRR′, NO₂, Me₃Sn and halo.

In one embodiment, R⁷ is independently OR.

In one embodiment, R⁷ is independently OR^{7A}, where R^{7A} is independently optionally substituted C₁₋₆ alkyl.

In one embodiment, R^{7A} is independently optionally substituted saturated C₁₋₆ alkyl.

In one embodiment, R^{7A} is independently optionally substituted C₂₋₄ alkenyl.

In one embodiment, R^{7A} is independently Me.

In one embodiment, R^{7A} is independently CH₂Ph.

In one embodiment, R^{7A} is independently allyl.

In one embodiment, the compound is a dimer where the R⁷ groups of each monomer form together a dimer bridge having the formula X—R″—X linking the monomers. R⁹

In one embodiment, R⁹ is independently selected from H, R, OH, OR, SH, SR, NH₂, NHR, NRR′, NO₂, Me₃Sn— and Halo.

In one embodiment, R⁹ is independently H.

In one embodiment, R⁹ is independently R or OR.

### R¹⁰

Preferably compatible linkers such as those described herein attach the DLL3 antibody to the PBD drug moiety through covalent bond(s) at the R¹⁰ position (i.e., N10).

### Q

In certain embodiments Q is independently selected from O, S and NH.

In one embodiment, Q is independently O.

In one embodiment, Q is independently S.

In one embodiment, Q is independently NH.

### R¹¹

In selected embodiments R¹¹ is either H, or R or, where Q is O, may be SO₃M where M is a metal cation. The cation may be Na⁺.

In certain embodiments R¹¹ is H.

In certain embodiments R¹¹ is R.

In certain embodiments, where Q is O, R¹¹ may be SO₃M where M is a metal cation. The cation may be Na⁺.

In certain embodiments where Q is O, R¹¹ is H.

In certain embodiments where Q is O, R¹¹ is R.

### X

In one embodiment, X is selected from O, S, or N(H).

Preferably, X is O.

### R″

R″ is a C₃₋₁₂ alkylene group, which chain may be interrupted by one or more heteroatoms, e.g. O, S, N(H), NMe and/or aromatic rings, e.g. benzene or pyridine, which rings are optionally substituted.

In one embodiment, R″ is a C₃₋₁₂ alkylene group, which chain may be interrupted by one or more heteroatoms and/or aromatic rings, e.g. benzene or pyridine.

In one embodiment, the alkylene group is optionally interrupted by one or more heteroatoms selected from O, S, and NMe and/or aromatic rings, which rings are optionally substituted.

In one embodiment, the aromatic ring is a C₅₋₂₀ arylene group, where arylene pertains to a divalent moiety obtained by removing two hydrogen atoms from two aromatic ring atoms of an aromatic compound, which moiety has from 5 to 20 ring atoms.

In one embodiment, R″ is a C₃₋₁₂ alkylene group, which chain may be interrupted by one or more heteroatoms, e.g. O, S, N(H), NMe and/or aromatic rings, e.g. benzene or pyridine, which rings are optionally substituted by NH₂.

In one embodiment, R″ is a C₃₋₁₂ alkylene group.

In one embodiment, R″ is selected from a C₃, C₅, C₇, C₉ and a C₁₁ alkylene group.

In one embodiment, R″ is selected from a C₃, C₅ and a C₇ alkylene group.

In one embodiment, R″ is selected from a C₃ and a C₅ alkylene group.

In one embodiment, R″ is a C₃ alkylene group.

In one embodiment, R″ is a C₅ alkylene group.

The alkylene groups listed above may be optionally interrupted by one or more heteroatoms and/or aromatic rings, e.g. benzene or pyridine, which rings are optionally substituted.

The alkylene groups listed above may be optionally interrupted by one or more heteroatoms and/or aromatic rings, e.g. benzene or pyridine.

The alkylene groups listed above may be unsubstituted linear aliphatic alkylene groups.

### R and R′

In one embodiment, R is independently selected from optionally substituted C₁₋₁₂ alkyl, C₃₋₂₀ heterocyclyl and C₅₋₂₀ aryl groups.

In one embodiment, R is independently optionally substituted C₁₋₁₂ alkyl.

In one embodiment, R is independently optionally substituted C₃₋₂₀ heterocyclyl.

In one embodiment, R is independently optionally substituted C₅₋₂₀ aryl.

Described above in relation to R² are various embodiments relating to preferred alkyl and aryl groups and the identity and number of optional substituents. The preferences set out for R² as it applies to R are applicable, where appropriate, to all other groups R, for examples where R⁶, R⁷, R⁸ or R⁹ is R.

The preferences for R apply also to R′.

In some embodiments of the invention there is provided a compound having a substituent group —NRR′. In one embodiment, R and R′ together with the nitrogen atom to which they are attached form an optionally substituted 4-, 5-, 6- or 7-membered heterocyclic ring. The ring may contain a further heteroatom, for example N, O or S.

In one embodiment, the heterocyclic ring is itself substituted with a group R. Where a further N heteroatom is present, the substituent may be on the N heteroatom.

In addition to the aforementioned PBDs certain exemplary dimeric PBDs have been shown to be particularly active and may be used in conjunction with the instant invention. To this end antibody drug conjugates (i.e., ADCs 1-6 as disclosed herein) of the instant invention may comprise a PBD compound set forth immediately below as PBD 1-5. Note that PBDs 1-5 below comprise the cytotoxic warhead released following separation of a linker such as those described in more detail herein. The synthesis of each of PBD 1-5 as a component of drug linker compounds is presented in great detail in WO 2014/130879 which is hereby incorporated by reference as to such synthesis. In view of WO 2014/130879 cytotoxic compounds that may comprise selected warheads of the ADCs of the present invention could readily be generated and employed as set forth herein. Accordingly, selected PBD compounds that may be released from the disclosed ADCs upon separation from a linker are set forth immediately below:

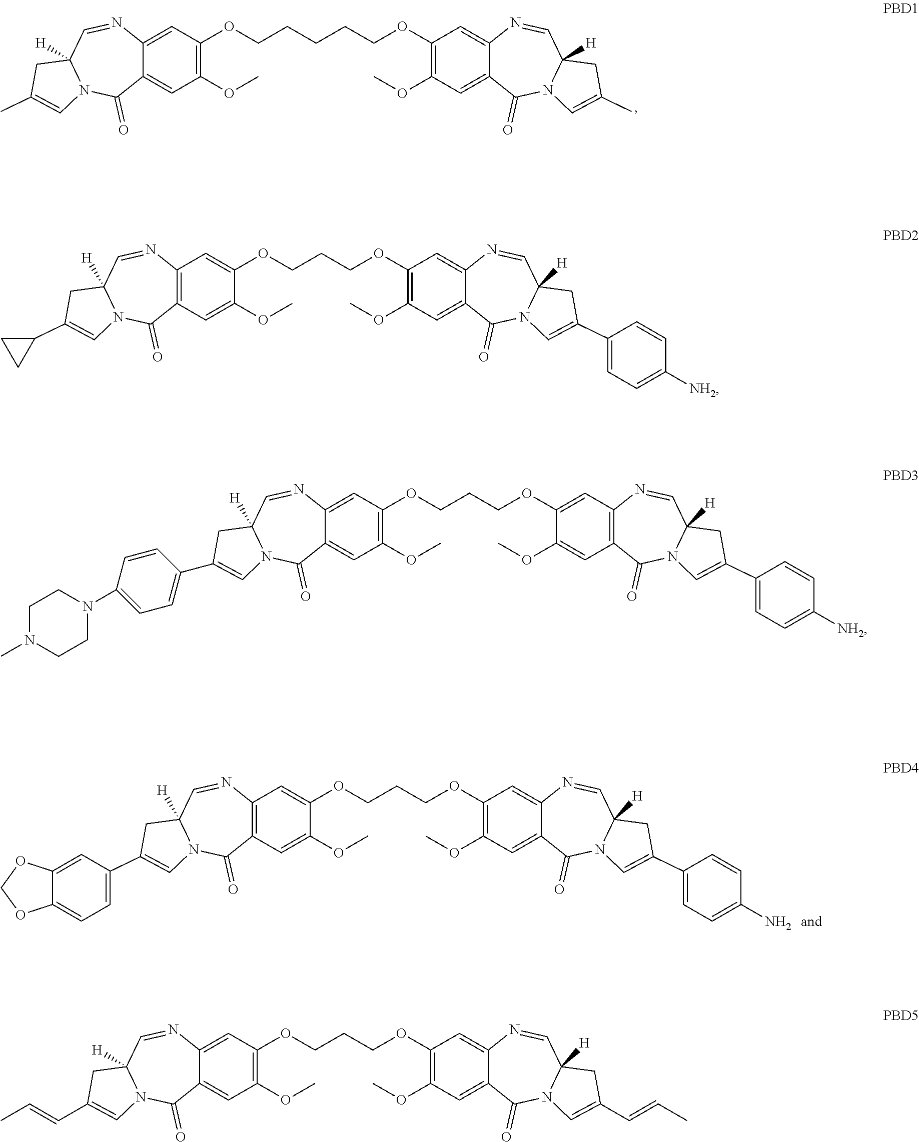

It will be appreciated that each of the aforementioned dimeric PBD warheads will preferably be released upon internalization by the target cell and destruction of the linker. As described in more detail below, certain linkers will comprise cleavable linkers which may incorporate a self-immolation moiety that allows release of the active PBD warhead without retention of any part of the linker. Upon release the PBD warhead will then bind and cross-link with the target cell's DNA. Such binding reportedly blocks division of the target cancer cell without distorting its DNA helix, thus potentially avoiding the common phenomenon of emergent drug resistance. In other preferred embodiments the warhead may be attached to the DLL3 targeting moiety through a cleavable linker that does not comprise a self-immolating moiety.

Delivery and release of such compounds at the tumor site(s) may prove clinically effective in treating or managing proliferative disorders in accordance with the instant disclosure. With regard to the compounds it will be appreciated that each of the disclosed PBDs have two sp² centers in each C-ring, which may allow for stronger binding in the minor groove of DNA (and hence greater toxicity), than for compounds with only one sp² center in each C-ring. Thus, when used in DLL3 ADCs as set forth herein the disclosed PBDs may prove to be particularly effective for the treatment of proliferative disorders.

The foregoing provides exemplary PBD compounds that are compatible with the instant invention and is in no way meant to be limiting as to other PBDs that may be successfully incorporated in anti-DLL3 conjugates according to the teachings herein. Rather, any PBD that may be conjugated to an antibody as described herein and set forth in the Examples below is compatible with the disclosed conjugates and expressly within the metes and bounds of the invention.

In addition to the aforementioned agents the antibodies of the present invention may also be conjugated to biological response modifiers. In certain embodiments the biological response modifier will comprise interleukin 2, interferons, or various types of colony-stimulating factors (e.g., CSF, GM-CSF, G-CSF).

More generally, the associated drug moiety can be a polypeptide possessing a desired biological activity. Such proteins may include, for example, a toxin such as abrin, ricin A, Onconase (or another cytotoxic RNase), pseudomonas exotoxin, cholera toxin, diphtheria toxin; an apoptotic agent such as tumor necrosis factor e.g. TNF-α or TNF-β, α-interferon, β-interferon, nerve growth factor, platelet derived growth factor, tissue plasminogen activator, AIM I (WO 97/33899), AIM II (WO 97/34911), Fas Ligand (Takahashi et al., 1994, PMID: 7826947), and VEGI (WO 99/23105), a thrombotic agent, an anti-angiogenic agent, e.g., angiostatin or endostatin, a lymphokine, for example, interleukin-1 (IL-1), interleukin-2 (IL-2), interleukin-6 (IL-6), granulocyte macrophage colony stimulating factor (GM-CSF), and granulocyte colony stimulating factor (G-CSF), or a growth factor e.g., growth hormone (GH).

2. Diagnostic or Detection Agents

In other embodiments, the anti-ASCL1 or anti-DLL3 antibodies of the invention, or fragments or derivatives thereof, are conjugated to a diagnostic or detectable agent, marker or reporter which may be, for example, a biological molecule (e.g., a peptide or nucleotide), a small molecule, fluorophore, or radioisotope. Labeled antibodies can be useful for monitoring the development or progression of a hyperproliferative disorder or as part of a clinical testing procedure to determine the efficacy of a particular therapy including the disclosed antibodies (i.e. theragnostics) or to determine a future course of treatment. Such markers or reporters may also be useful in purifying the selected antibody, for use in antibody analytics (e.g., epitope binding or antibody binning), separating or isolating tumorigenic cells or in preclinical procedures or toxicology studies.

Such diagnosis, analysis and/or detection can be accomplished by coupling the antibody to detectable substances including, but not limited to, various enzymes comprising for example horseradish peroxidase, alkaline phosphatase, beta-galactosidase, or acetylcholinesterase; prosthetic groups, such as but not limited to streptavidinlbiotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (¹³¹I, ¹²⁵I, ¹²³I, ¹²¹I,), carbon (¹⁴C), sulfur (³⁵S), tritium (³H), indium (¹¹⁵In, ¹¹³In, ¹¹²In, ¹¹¹In,), and technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh ⁹⁷Ru, ⁶⁸Ge, ⁵⁷Co, ⁶⁵Zn, ⁸⁵Sr, ³²P, ⁸⁹Zr, ¹⁵³Gd, ¹⁶⁹Yb, ⁵¹Cr ⁵⁴Mn, ⁷⁵Se, ¹¹³Sn, and ¹¹⁷Tin; positron emitting metals using various positron emission tomographies, non-radioactive paramagnetic metal ions, and molecules that are radiolabeled or conjugated to specific radioisotopes. In such embodiments appropriate detection methodology is well known in the art and readily available from numerous commercial sources.

In other embodiments the antibodies or fragments thereof can be fused or conjugated to marker sequences or compounds, such as a peptide or fluorophore to facilitate purification or diagnostic or analytic procedures such as immunohistochemistry, bio-layer interferometry, surface plasmon resonance, flow cytometry, competitive ELISA, FACs, etc. In some embodiments, the marker comprises a histidine tag such as that provided by the pQE vector (Qiagen), among others, many of which are commercially available. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag (U.S. Pat. No. 4,703,004).

3. Biocompatible Modifiers

In selected embodiments the antibodies of the invention may be conjugated with biocompatible modifiers that may be used to adjust, alter, improve or moderate antibody characteristics as desired. For example, antibodies or fusion constructs with increased in vivo half-lives can be generated by attaching relatively high molecular weight polymer molecules such as commercially available polyethylene glycol (PEG) or similar biocompatible polymers. Those skilled in the art will appreciate that PEG may be obtained in many different molecular weights and molecular configurations that can be selected to impart specific properties to the antibody (e.g. the half-life may be tailored). PEG can be attached to antibodies or antibody fragments or derivatives with or without a multifunctional linker either through conjugation of the PEG to the N- or C-terminus of said antibodies or antibody fragments or via epsilon-amino groups present on lysine residues. Linear or branched polymer derivatization that results in minimal loss of biological activity may be used. The degree of conjugation can be closely monitored by SDS-PAGE and mass spectrometry to ensure optimal conjugation of PEG molecules to antibody molecules. Unreacted PEG can be separated from antibody-PEG conjugates by, e.g., size exclusion or ion-exchange chromatography. In a similar manner, the disclosed antibodies can be conjugated to albumin in order to make the antibody or antibody fragment more stable in vivo or have a longer half-life in vivo. The techniques are well known in the art, see e.g., WO 93/15199, WO 93/15200, and WO 01/77137; and EP 0 413, 622. Other biocompatible conjugates are evident to those of ordinary skill and may readily be identified in accordance with the teachings herein.

B. Linker Compounds

As indicated above payloads compatible with the instant invention comprise one or more warheads and, optionally, a linker associating the warheads with the antibody targeting agent. Numerous linker compounds can be used to conjugate the antibodies of the invention to the relevant warhead. The linkers merely need to covalently bind with the reactive residue on the antibody (preferably a cysteine or lysine) and the selected drug compound. Accordingly, any linker that reacts with the selected antibody residue and may be used to provide the relatively stable conjugates (site-specific or otherwise) of the instant invention is compatible with the teachings herein.

Compatible linkers can advantageously bind to reduced cysteines and lysines, which are nucleophilic. Conjugation reactions involving reduced cysteines and lysines include, but are not limited to, thiol-maleimide, thiol-halogeno (acyl halide), thiol-ene, thiol-yne, thiol-vinylsulfone, thiol-bisulfone, thiol-thiosulfonate, thiol-pyridyl disulfide and thiol-parafluoro reactions. As further discussed herein, thiol-maleimide bioconjugation is one of the most widely used approaches due to its fast reaction rates and mild conjugation conditions. One issue with this approach is the possibility of the retro-Michael reaction and loss or transfer of the maleimido-linked payload from the antibody to other proteins in the plasma, such as, for example, human serum albumin. However, in some embodiments the use of selective reduction and site-specific antibodies as set forth herein in the Examples below may be used to stabilize the conjugate and reduce this undesired transfer. Thiol-acyl halide reactions provide bioconjugates that cannot undergo retro-Michael reaction and therefore are more stable. However, the thiol-halide reactions in general have slower reaction rates compared to maleimide-based conjugations and are thus not as efficient in providing undesired drug to antibody ratios. Thiol-pyridyl disulfide reaction is another popular bioconjugation route. The pyridyl disulfide undergoes fast exchange with free thiol resulting in the mixed disulfide and release of pyridine-2-thione. Mixed disulfides can be cleaved in the reductive cell environment releasing the payload. Other approaches gaining more attention in bioconjugation are thiol-vinylsulfone and thiol-bisulfone reactions, each of which are compatible with the teachings herein and expressly included within the scope of the invention.

In selected embodiments compatible linkers will confer stability on the ADCs in the extracellular environment, prevent aggregation of the ADC molecules and keep the ADC freely soluble in aqueous media and in a monomeric state. Before transport or delivery into a cell, the ADC is preferably stable and remains intact, i.e. the antibody remains linked to the drug moiety. While the linkers are stable outside the target cell they may be designed to be cleaved or degraded at some efficacious rate inside the cell. Accordingly an effective linker will: (i) maintain the specific binding properties of the antibody; (ii) allow intracellular delivery of the conjugate or drug moiety; (iii) remain stable and intact, i.e. not cleaved or degraded, until the conjugate has been delivered or transported to its targeted site; and (iv) maintain a cytotoxic, cell-killing effect or a cytostatic effect of the drug moiety (including, in some cases, any bystander effects). The stability of the ADC may be measured by standard analytical techniques such as HPLC/UPLC, mass spectroscopy, HPLC, and the separation/analysis techniques LC/MS and LC/MS/MS. As set forth above covalent attachment of the antibody and the drug moiety requires the linker to have two reactive functional groups, i.e. bivalency in a reactive sense. Bivalent linker reagents that are useful to attach two or more functional or biologically active moieties, such as MMAE and antibodies are known, and methods have been described to provide resulting conjugates compatible with the teachings herein.

Linkers compatible with the present invention may broadly be classified as cleavable and non-cleavable linkers. Cleavable linkers, which may include acid-labile linkers (e.g., oximes and hydrozones), protease cleavable linkers and disulfide linkers, are internalized into the target cell and are cleaved in the endosomal-lysosomal pathway inside the cell. Release and activation of the cytotoxin relies on endosome/lysosome acidic compartments that facilitate cleavage of acid-labile chemical linkages such as hydrazone or oxime. If a lysosomal-specific protease cleavage site is engineered into the linker the cytotoxins will be released in proximity to their intracellular targets. Alternatively, linkers containing mixed disulfides provide an approach by which cytotoxic payloads are released intracellularly as they are selectively cleaved in the reducing environment of the cell, but not in the oxygen-rich environment in the bloodstream. By way of contrast, compatible non-cleavable linkers containing amide linked polyethylene glycol or alkyl spacers liberate toxic payloads during lysosomal degradation of the ADC within the target cell. In some respects the selection of linker will depend on the particular drug used in the conjugate, the particular indication and the antibody target.

Accordingly, certain embodiments of the invention comprise a linker that is cleavable by a cleaving agent that is present in the intracellular environment (e.g., within a lysosome or endosome or caveolae). The linker can be, for example, a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, each of which is known to hydrolyze dipeptide drug derivatives resulting in the release of active drug inside target cells. Exemplary peptidyl linkers that are cleavable by the thiol-dependent protease cathepsin-B are peptides comprising Phe-Leu since cathepsin-B has been found to be highly expressed in cancerous tissue. Other examples of such linkers are described, for example, in U.S. Pat. No. 6,214,345. In specific embodiments, the peptidyl linker cleavable by an intracellular protease is a Val-Cit linker, a Val-Ala linker or a Phe-Lys linker. One advantage of using intracellular proteolytic release of the therapeutic agent is that the agent is typically attenuated when conjugated and the serum stabilities of the conjugates are relatively high.

In other embodiments, the cleavable linker is pH-sensitive. Typically, the pH-sensitive linker will be hydrolyzable under acidic conditions. For example, an acid-labile linker that is hydrolyzable in the lysosome (e.g., a hydrazone, oxime, semicarbazone, thiosemicarbazone, cis-aconitic amide, orthoester, acetal, ketal, or the like) can be used (See, e.g., U.S. Pat. Nos. 5,122,368; 5,824,805; 5,622,929). Such linkers are relatively stable under neutral pH conditions, such as those in the blood, but are unstable (e.g., cleavable) at below pH 5.5 or 5.0 which is the approximate pH of the lysosome.

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio)toluene). In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In certain aspects of the invention the selected linker will comprise a compound of the formula:

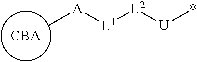

wherein the asterisk indicates the point of attachment to the drug, CBA (i.e. cell binding agent) comprises the anti-DLL3 antibody, L¹ comprises a linker unit and optionally a cleavable linker unit, A is a connecting group (optionally comprising a spacer) connecting L¹ to a reactive residue on the antibody, L² is preferably a covalent bond and U, which may or may not be present, can comprise all or part of a self-immolative unit that facilitates a clean separation of the linker from the warhead at the tumor site.

In some embodiments (such as those set forth in U.S.P.N. 2011/0256157) compatible linkers may comprise:

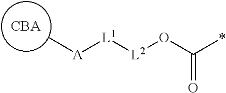

where the asterisk indicates the point of attachment to the drug, CBA (i.e. cell binding agent) comprises an anti-DLL3 antibody, L¹ comprises a linker and optionally a cleavable linker, A is a connecting group (optionally comprising a spacer) connecting L¹ to a reactive residue on the antibody and L² is a covalent bond or together with —OC(═O)— forms a self-immolative moiety.

It will be appreciated that the nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidizing conditions may also find use in the present invention.

In certain embodiments L¹ may comprise a contiguous sequence of amino acids. The amino acid sequence may be the target substrate for enzymatic cleavage, thereby allowing release of the drug.

In one embodiment, L¹ is cleavable by the action of an enzyme. In one embodiment, the enzyme is an esterase or a peptidase.

In another embodiment L¹ is as a Cathepsin labile linker.

In one embodiment, L¹ comprises a dipeptide. The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a Cathepsin labile linker, the dipeptide may be the site of action for Cathepsin-mediated cleavage.

Additionally, for those amino acids groups having carboxyl or amino side chain functionality, for example Glu and Lys respectively, CO and NH may represent that side chain functionality.

In one embodiment, the group —X₁—X₂— in dipeptide, —NH—X₁—X₂—CO—, is selected from: -Phe-Lys-, -Val-Ala-, -Val-Lys-, -Ala-Lys-, -Val-Cit-, -Phe-Cit-, -Leu-Cit-, -Ile-Cit-, -Phe-Arg- and -Trp-Cit- where Cit is citrulline.

Preferably, the group —X₁—X₂— in dipeptide, —NH—X₁—X₂—CO—, is selected from: -Phe-Lys-, -Val-Ala-, -Val-Lys-, -Ala-Lys-, and -Val-Cit-.

Most preferably, the group —X₁—X₂— in dipeptide, —NH—X₁—X₂—CO—, is -Phe-Lys- or -Val-Ala- or Val-Cit. In certain selected embodiments the dipeptide will comprise -Val-Ala-.

In one embodiment, L² is present and together with —C(═O)O— forms a self-immolative linker. In one embodiment, L² is a substrate for enzymatic activity, thereby allowing release of the warhead.

In one embodiment, where L¹ is cleavable by the action of an enzyme and L² is present, the enzyme cleaves the bond between L¹ and L².

L¹ and L², where present, may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

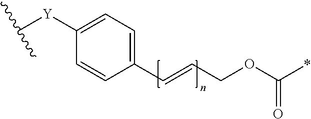

where the asterisk indicates the point of attachment to the drug or cytotoxic agent position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents. In one embodiment, the phenylene group is optionally substituted with halo, NO₂, alkyl or hydroxyalkyl.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

In other embodiments the linker may include a self-immolative linker and the dipeptide together form the group —NH-Val-Cit-CO—NH-PABC-. In other selected embodiments the linker may comprise the group —NH-Val-Ala-CO—NH-PABC-, which is illustrated below:

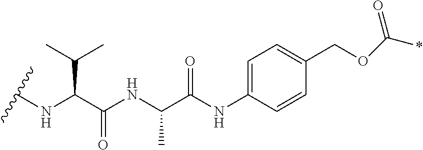 [SMILES: *N[C@H](C(=O)N[C@@H](C)C(=O)Nc1ccc(COC(*)=O)cc1)C(C)C]

where the asterisk indicates the point of attachment to the selected cytotoxic moiety, and the wavy line indicates the point of attachment to the remaining portion of the linker (e.g., the spacer-antibody binding segments) which may be conjugated to the antibody. Upon enzymatic cleavage of the dipeptide, the self-immolative linker will allow for clean release of the protected compound (i.e., the cytotoxin) when a remote site is activated, proceeding along the lines shown below:

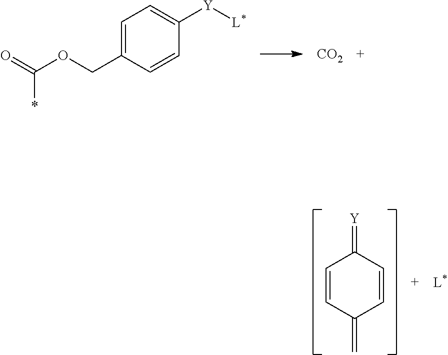

where the asterisk indicates the point of attachment to the selected cytotoxic moiety and where L* is the activated form of the remaining portion of the linker comprising the now cleaved peptidyl unit. The clean release of the warhead ensures it will maintain the desired toxic activity.

In one embodiment, A is a covalent bond. Thus, L¹ and the antibody are directly connected. For example, where L¹ comprises a contiguous amino acid sequence, the N-terminus of the sequence may connect directly to the antibody residue.

In another embodiment, A is a spacer group. Thus, ^{L} and the antibody are indirectly connected.

In certain embodiments L¹ and A may be connected by a bond selected from: —C(═O)NH—, —C(═O)O—, —NHC(═O)—, —OC(═O)—, —OC(═O)O—, —NHC(═O)O—, —OC(═O)NH—, and —NHC(═O)NH—.

As will be discussed in more detail below the drug linkers of the instant invention will preferably be linked to reactive thiol nucleophiles on cysteines, including free cysteines. To this end the cysteines of the antibodies may be made reactive for conjugation with linker reagents by treatment with various reducing agent such as DTT or TCEP or mild reducing agents as set forth herein. In other embodiments the drug linkers of the instant invention will preferably be linked to a lysine.

Preferably, the linker contains an electrophilic functional group for reaction with a nucleophilic functional group on the antibody. Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) maleimide groups (ii) activated disulfides, (iii) active esters such as NHS (N-hydroxysuccinimide) esters, HOBt (N-hydroxybenzotriazole) esters, haloformates, and acid halides; (iv) alkyl and benzyl halides such as haloacetamides; and (v) aldehydes, ketones and carboxyl groups.

Exemplary functional groups compatible with the invention are illustrated immediately below:

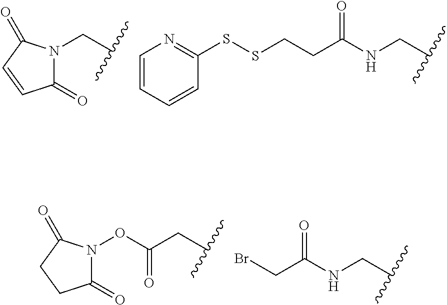

In some embodiments the connection between a cysteine (including a free cysteine of a site-specific antibody) and the drug-linker moiety is through a thiol residue and a terminal maleimide group of present on the linker. In such embodiments, the connection between the antibody and the drug-linker may be:

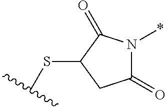 [SMILES: *SC1CC(=O)N(*)C1=O]

where the asterisk indicates the point of attachment to the remaining portion of drug-linker and the wavy line indicates the point of attachment to the remaining portion of the antibody. In this embodiment, the S atom is preferably derived from a site-specific free cysteine.

With regard to other compatible linkers the binding moiety may comprise a terminal iodoacetamide that may be reacted with activated residues on the antibody to provide the desired conjugate. In any event one skilled in the art could readily conjugate each of the disclosed drug-linker compounds with a compatible anti-DLL3 antibody (including site-specific antibodies) in view of the instant disclosure.

In accordance with the instant disclosure the invention provides methods of making compatible antibody drug conjugates comprising conjugating an anti-DLL3 antibody with a drug-linker compound selected from the group consisting of:

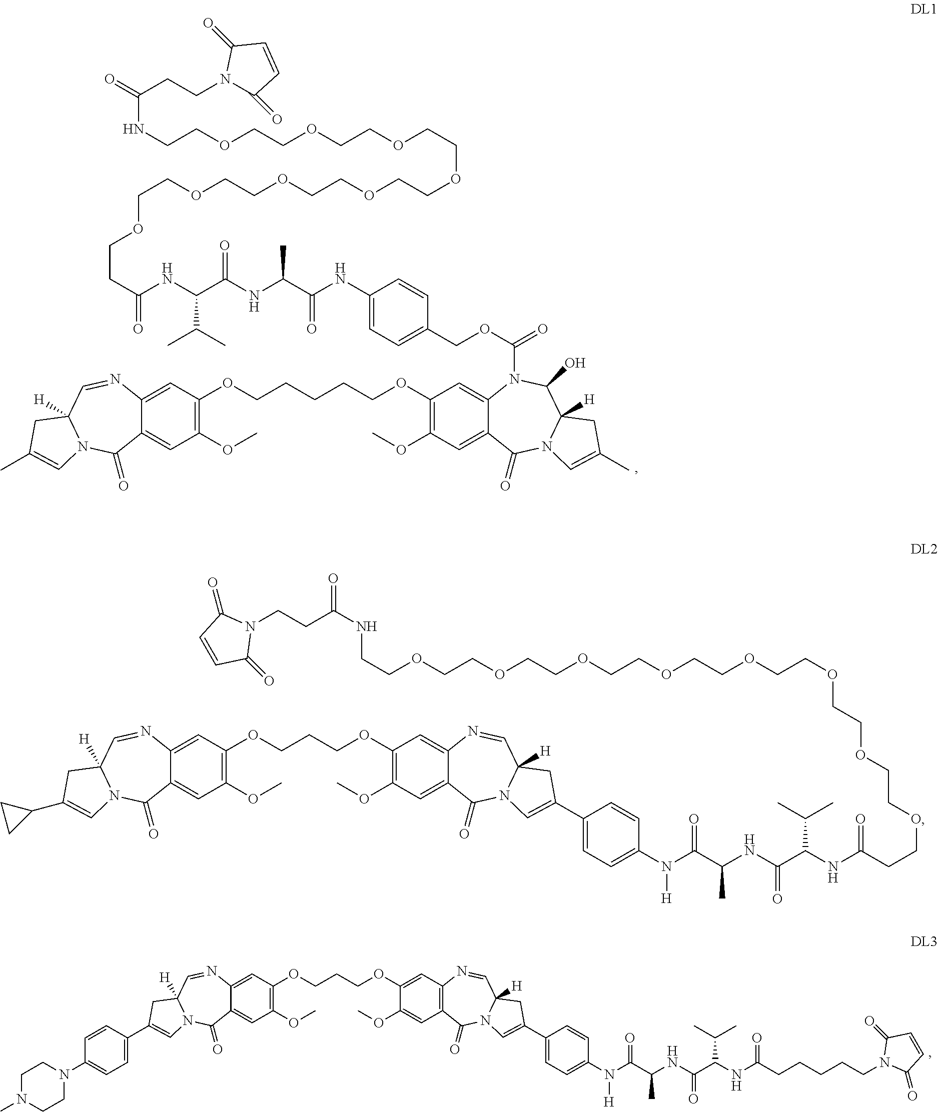

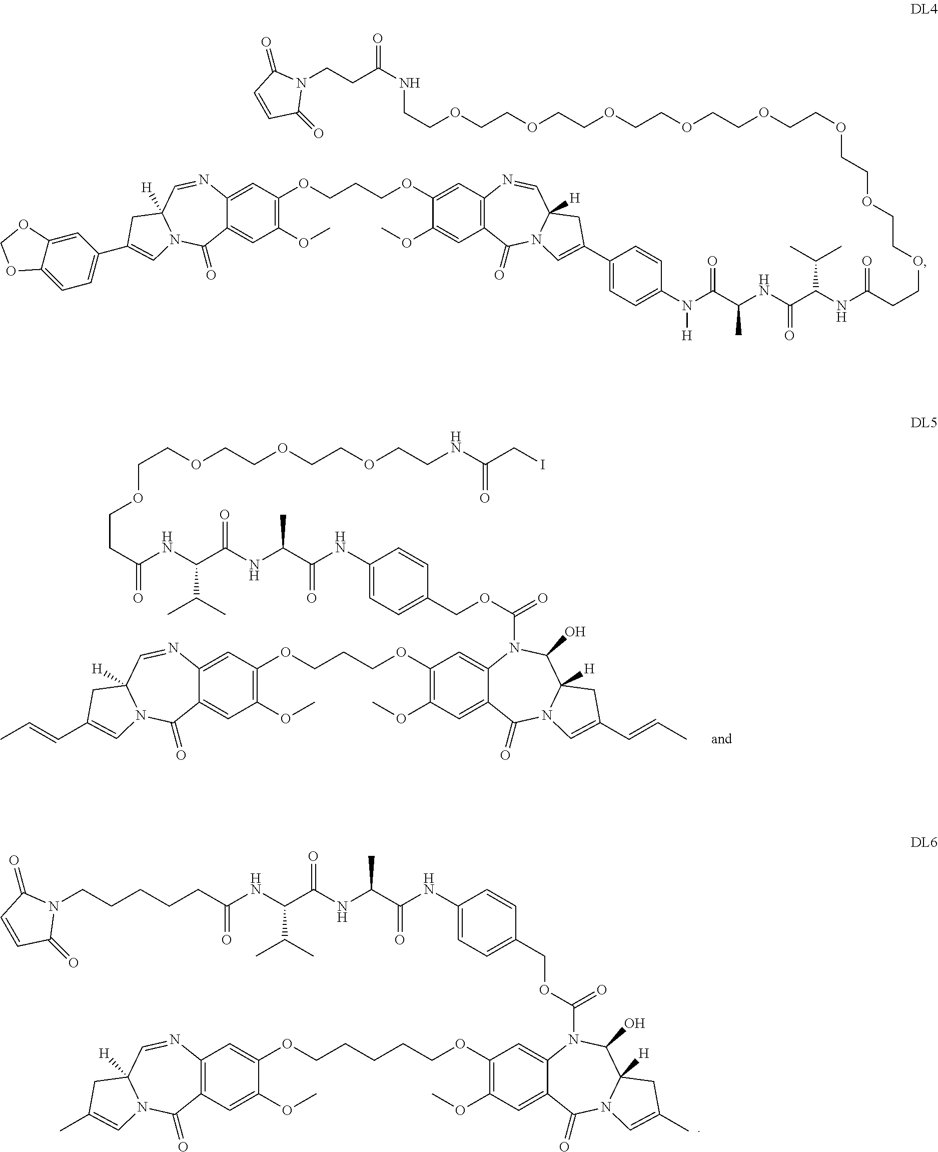

For the purposes of then instant application DL will be used as an abbreviation for “drug linker” (or linker-drug “L-D” in the formula Ab-[L-D]n) and will comprise drug linkers 1-6 (i.e., DL1, DL2, DL3, DL4 DL5, and DL6) as set forth above. Note that DL1 and DL6 comprise the same warhead and same dipeptide subunit but differ in the connecting group spacer. Accordingly, upon cleavage of the linker both DL1 and DL6 will release PBD 1.

It will be appreciated that the linker appended terminal maleimido moiety (DL1-DL4 and DL6) or iodoacetamide moiety (DL5) may be conjugated to free sulfhydryl(s) on the selected DLL3 antibody using art-recognized techniques as disclosed herein. Synthetic routes for the aforementioned compounds are set forth in WO2014/130879 which is incorporated herein by reference explicitly for the synthesis of the aforementioned DL compounds while specific methods of conjugating such PBDs linker combinations are set forth in the Examples below.

Thus, in selected aspects the present invention relates to DLL3 antibodies conjugated to the disclosed DL moieties to provide DLL3 immunoconjugates substantially set forth in ADCs 1-6 immediately below. Accordingly, in certain aspects the invention is directed to an ADC of the formula Ab-[L-D]n comprising a structure selected from the group consisting of:

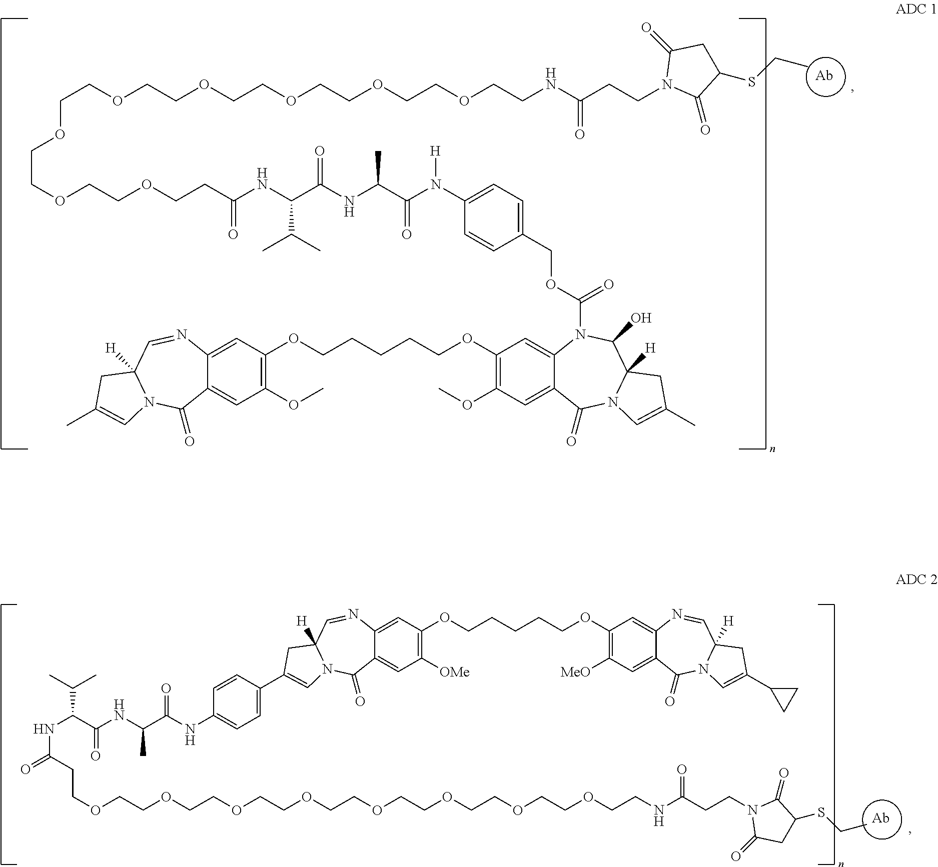

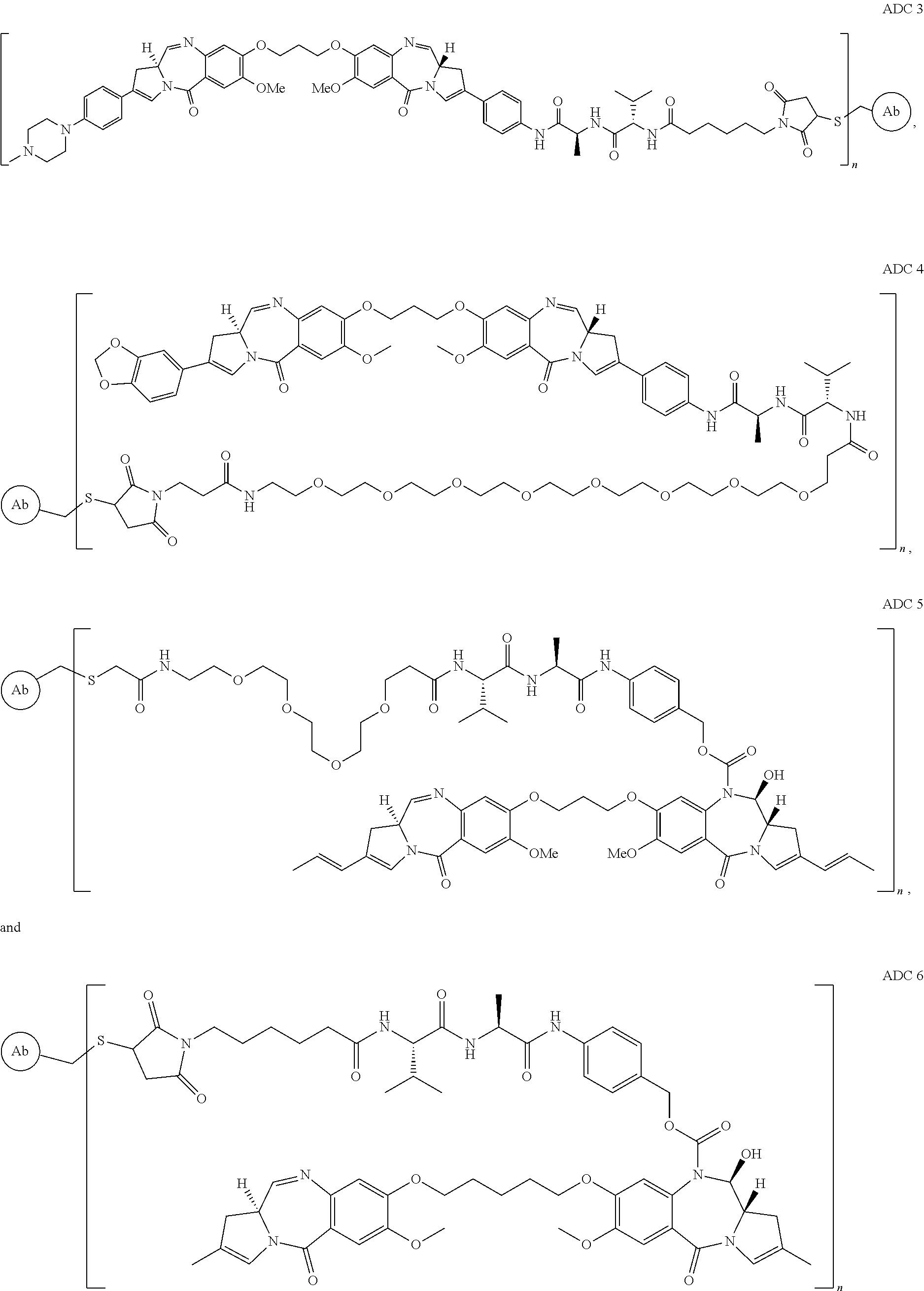

wherein Ab comprises an anti-DLL3 antibody or immunoreactive fragment thereof and n is an integer from 1 to 20. In certain embodiments n will comprise an integer from 1 to 8 and in selected embodiments n will comprise 2 or 4.

Those of skill in the art will appreciate that the aforementioned ADC structures are defined by the formula Ab-[L-D]n and more than one drug linker molecule as depicted therein may be covalently conjugated to the DLL3 antibody (e.g., n may be an integer from about 1 to about 20). More particularly, as discussed in more detail below it will be appreciated that more than one payload may be conjugated to each antibody and that the schematic representations above must be construed as such. By way of example ADC1 as set forth above may comprise a DLL3 antibody conjugated to 1, 2, 3, 4, 5, 6, 7 or 8 or more payloads and that compositions of such ADCs will generally comprise a mixture of drug loaded species.

In certain aspects the DLL3 PBD ADCs of the invention will comprise an anti-DLL3 antibody as set forth in the appended Examples or an immunoreactive fragment thereof. In a particular embodiment ADC3 will comprise hSC16.56 (e.g., hSC16.56 PBD1). In such embodiments the ADC will preferably comprise 2 payloads. In other preferred embodiments the DLL3 ADC will comprise ADC1 wherein n is 2.

C. Conjugation

It will be appreciated that a number of well-known reactions may be used to attach the drug moiety and/or linker to the selected antibody. For example, various reactions exploiting sulfhydryl groups of cysteines may be employed to conjugate the desired moiety. Some embodiments will comprise conjugation of antibodies comprising one or more free cysteines as discussed in detail below. In other embodiments ADCs of the instant invention may be generated through conjugation of drugs to solvent-exposed amino groups of lysine residues present in the selected antibody. Still other embodiments comprise activation of N-terminal threonine and serine residues which may then be used to attach the disclosed payloads to the antibody. The selected conjugation methodology will preferably be tailored to optimize the number of drugs attached to the antibody and provide a relatively high therapeutic index.

Various methods are known in the art for conjugating a therapeutic compound to a cysteine residue and will be apparent to the skilled artisan. Under basic conditions the cysteine residues will be deprotonated to generate a thiolate nucleophile which may be reacted with soft electrophiles such as maleimides and iodoacetamides. Generally reagents for such conjugations may react directly with a cysteine thiol to form the conjugated protein or with a linker-drug to form a linker-drug intermediate. In the case of a linker, several routes, employing organic chemistry reactions, conditions, and reagents are known to those skilled in the art, including: (1) reaction of a cysteine group of the protein of the invention with a linker reagent, to form a protein-linker intermediate, via a covalent bond, followed by reaction with an activated compound; and (2) reaction of a nucleophilic group of a compound with a linker reagent, to form a drug linker intermediate, via a covalent bond, followed by reaction with a cysteine group of a protein of the invention. As will be apparent to the skilled artisan from the foregoing, bifunctional (or bivalent) linkers are useful in the present invention. For example, the bifunctional linker may comprise a thiol modification group for covalent linkage to the cysteine residue(s) and at least one attachment moiety (e.g., a second thiol modification moiety) for covalent or non-covalent linkage to the compound.

Prior to conjugation, antibodies may be made reactive for conjugation with linker reagents by treatment with a reducing agent such as dithiothreitol (DTT) or (tris(2-carboxyethyl)phosphine (TCEP). In other embodiments additional nucleophilic groups can be introduced into antibodies through the reaction of lysines with reagents, including but not limited to, 2-iminothiolane (Traut's reagent), SATA, SATP or SAT(PEG)4, resulting in conversion of an amine into a thiol.

With regard to such conjugations cysteine thiol or lysine amino groups are nucleophilic and capable of reacting to form covalent bonds with electrophilic groups on linker reagents or compound-linker intermediates or drugs including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a compound or linker include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Conjugation reagents commonly include maleimide, haloacetyl, iodoacetamide succinimidyl ester, isothiocyanate, sulfonyl chloride, 2,6-dichlorotriazinyl, pentafluorophenyl ester, and phosphoramidite, although other functional groups can also be used. In certain embodiments methods include, for example, the use of maleimides, iodoacetimides or haloacetyl/alkyl halides, aziridne, acryloyl derivatives to react with the thiol of a cysteine to produce a thioether that is reactive with a compound. Disulphide exchange of a free thiol with an activated piridyldisulphide is also useful for producing a conjugate (e.g., use of 5-thio-2-nitrobenzoic (TNB) acid). Preferably, a maleimide is used.

As indicated above, lysine may also be used as a reactive residue to effect conjugation as set forth herein. The nucleophilic lysine residue is commonly targeted through amine-reactive succinimidylesters. To obtain an optimal number of deprotonated lysine residues, the pH of the aqueous solution must be below the pKa of the lysine ammonium group, which is around 10.5, so the typical pH of the reaction is about 8 and 9. The common reagent for the coupling reaction is NHS-ester which reacts with nucleophilic lysine through a lysine acylation mechanism. Other compatible reagents that undergo similar reactions comprise isocyanates and isothiocyanates which also may be used in conjunction with the teachings herein to provide ADCs. Once the lysines have been activated, many of the aforementioned linking groups may be used to covalently bind the warhead to the antibody.

Methods are also known in the art for conjugating a compound to a threonine or serine residue (preferably a N-terminal residue). For example methods have been described in which carbonyl precursors are derived from the 1,2-aminoalcohols of serine or threonine, which can be selectively and rapidly converted to aldehyde form by periodate oxidation. Reaction of the aldehyde with a 1,2-aminothiol of cysteine in a compound to be attached to a protein of the invention forms a stable thiazolidine product. This method is particularly useful for labeling proteins at N-terminal serine or threonine residues.

In some embodiments reactive thiol groups may be introduced into the selected antibody (or fragment thereof) by introducing one, two, three, four, or more free cysteine residues (e.g., preparing antibodies comprising one or more free non-native cysteine amino acid residues).

Such site-specific antibodies or engineered antibodies allow for conjugate preparations that exhibit enhanced stability and substantial homogeneity due, at least in part, to the provision of engineered free cysteine site(s) and/or the novel conjugation procedures set forth herein. Unlike conventional conjugation methodology that fully or partially reduces each of the intrachain or interchain antibody disulfide bonds to provide conjugation sites (and is fully compatible with the instant invention), the present invention additionally provides for the selective reduction of certain prepared free cysteine sites and attachment of the drug linker to the same.

In this regard it will be appreciated that the conjugation specificity promoted by the engineered sites and the selective reduction allows for a high percentage of site directed conjugation at the desired positions. Significantly some of these conjugation sites, such as those present in the terminal region of the light chain constant region, are typically difficult to conjugate effectively as they tend to cross-react with other free cysteines. However, through molecular engineering and selective reduction of the resulting free cysteines, efficient conjugation rates may be obtained which considerably reduces unwanted high-DAR contaminants and non-specific toxicity. More generally the engineered constructs and disclosed novel conjugation methods comprising selective reduction provide ADC preparations having improved pharmacokinetics and/or pharmacodynamics and, potentially, an improved therapeutic index.

In certain embodiments site-specific constructs present free cysteine(s) which, when reduced, comprise thiol groups that are nucleophilic and capable of reacting to form covalent bonds with electrophilic groups on linker moieties such as those disclosed above. As discussed above antibodies of the instant invention may have reducible unpaired interchain or intrachain cysteines or introduced non-native cysteines, i.e. cysteines providing such nucleophilic groups. Thus, in certain embodiments the reaction of free sulfhydryl groups of the reduced free cysteines and the terminal maleimido or haloacetamide groups of the disclosed drug linkers will provide the desired conjugation. In such cases free cysteines of the antibodies may be made reactive for conjugation with linker reagents by treatment with a reducing agent such as dithiothreitol (DTT) or (tris (2-carboxyethyl)phosphine (TCEP). Each free cysteine will thus present, theoretically, a reactive thiol nucleophile. While such reagents are particularly compatible with the instant invention it will be appreciated that conjugation of site-specific antibodies may be effected using various reactions, conditions and reagents generally known to those skilled in the art.

In addition it has been found that the free cysteines of engineered antibodies may be selectively reduced to provide enhanced site-directed conjugation and a reduction in unwanted, potentially toxic contaminants. More specifically “stabilizing agents” such as arginine have been found to modulate intra- and inter-molecular interactions in proteins and may be used, in conjunction with selected reducing agents (preferably relatively mild), to selectively reduce the free cysteines and to facilitate site-specific conjugation as set forth herein. As used herein the terms “selective reduction” or “selectively reducing” may be used interchangeably and shall mean the reduction of free cysteine(s) without substantially disrupting native disulfide bonds present in the engineered antibody. In selected embodiments this selective reduction may be effected by the use of certain reducing agents or certain reducing agent concentrations. In other embodiments selective reduction of an engineered construct will comprise the use of stabilization agents in combination with reducing agents (including mild reducing agents). It will be appreciated that the term “selective conjugation” shall mean the conjugation of an engineered antibody that has been selectively reduced in the presence of a cytotoxin as described herein. In this respect the use of such stabilizing agents (e.g., arginine) in combination with selected reducing agents can markedly improve the efficiency of site-specific conjugation as determined by extent of conjugation on the heavy and light antibody chains and DAR distribution of the preparation. Compatible antibody constructs and selective conjugation techniques and reagents are extensively disclosed in WO2015/031698 which is incorporated herein specifically as to such methodology and constructs.

While not wishing to be bound by any particular theory, such stabilizing agents may act to modulate the electrostatic microenvironment and/or modulate conformational changes at the desired conjugation site, thereby allowing relatively mild reducing agents (which do not materially reduce intact native disulfide bonds) to facilitate conjugation at the desired free cysteine site(s). Such agents (e.g., certain amino acids) are known to form salt bridges (via hydrogen bonding and electrostatic interactions) and can modulate protein-protein interactions in such a way as to impart a stabilizing effect that may cause favorable conformational changes and/or reduce unfavorable protein-protein interactions. Moreover, such agents may act to inhibit the formation of undesired intramolecular (and intermolecular) cysteine-cysteine bonds after reduction thus facilitating the desired conjugation reaction wherein the engineered site-specific cysteine is bound to the drug (preferably via a linker). Since selective reduction conditions do not provide for the significant reduction of intact native disulfide bonds, the subsequent conjugation reaction is naturally driven to the relatively few reactive thiols on the free cysteines (e.g., preferably 2 free thiols per antibody). As previously alluded to, such techniques may be used to considerably reduce levels of non-specific conjugation and corresponding unwanted DAR species in conjugate preparations fabricated in accordance with the instant disclosure.

In selected embodiments stabilizing agents compatible with the present invention will generally comprise compounds with at least one moiety having a basic pKa. In certain embodiments the moiety will comprise a primary amine while in other embodiments the amine moiety will comprise a secondary amine. In still other embodiments the amine moiety will comprise a tertiary amine or a guanidinium group. In other selected embodiments the amine moiety will comprise an amino acid while in other compatible embodiments the amine moiety will comprise an amino acid side chain. In yet other embodiments the amine moiety will comprise a proteinogenic amino acid. In still other embodiments the amine moiety comprises a non-proteinogenic amino acid. In some embodiments, compatible stabilizing agents may comprise arginine, lysine, proline and cysteine. In certain preferred embodiments the stabilizing agent will comprise arginine. In addition compatible stabilizing agents may include guanidine and nitrogen containing heterocycles with basic pKa.

In certain embodiments compatible stabilizing agents comprise compounds with at least one amine moiety having a pKa of greater than about 7.5, in other embodiments the subject amine moiety will have a pKa of greater than about 8.0, in yet other embodiments the amine moiety will have a pKa greater than about 8.5 and in still other embodiments the stabilizing agent will comprise an amine moiety having a pKa of greater than about 9.0. Other embodiments will comprise stabilizing agents where the amine moiety will have a pKa of greater than about 9.5 while certain other embodiments will comprise stabilizing agents exhibiting at least one amine moiety having a pKa of greater than about 10.0. In still other embodiments the stabilizing agent will comprise a compound having the amine moiety with a pKa of greater than about 10.5, in other embodiments the stabilizing agent will comprise a compound having a amine moiety with a pKa greater than about 11.0, while in still other embodiments the stabilizing agent will comprise a amine moiety with a pKa greater than about 11.5. In yet other embodiments the stabilizing agent will comprise a compound having an amine moiety with a pKa greater than about 12.0, while in still other embodiments the stabilizing agent will comprise an amine moiety with a pKa greater than about 12.5. Those of skill in the art will understand that relevant pKa's may readily be calculated or determined using standard techniques and used to determine the applicability of using a selected compound as a stabilizing agent.

The disclosed stabilizing agents are shown to be particularly effective at targeting conjugation to free site-specific cysteines when combined with certain reducing agents. For the purposes of the instant invention, compatible reducing agents may include any compound that produces a reduced free site-specific cysteine for conjugation without significantly disrupting the native disulfide bonds of the engineered antibody. Under such conditions, preferably provided by the combination of selected stabilizing and reducing agents, the activated drug linker is largely limited to binding to the desired free site-specific cysteine site(s). Relatively mild reducing agents or reducing agents used at relatively low concentrations to provide mild conditions are particularly preferred. As used herein the terms “mild reducing agent” or “mild reducing conditions” shall be held to mean any agent or state brought about by a reducing agent (optionally in the presence of stabilizing agents) that provides thiols at the free cysteine site(s) without substantially disrupting native disulfide bonds present in the engineered antibody. That is, mild reducing agents or conditions (preferably in combination with a stabilizing agent) are able to effectively reduce free cysteine(s) (provide a thiol) without significantly disrupting the protein's native disulfide bonds. The desired reducing conditions may be provided by a number of sulfhydryl-based compounds that establish the appropriate environment for selective conjugation. In embodiments mild reducing agents may comprise compounds having one or more free thiols while in some embodiments mild reducing agents will comprise compounds having a single free thiol. Non-limiting examples of reducing agents compatible with the selective reduction techniques of the instant invention comprise glutathione, n-acetyl cysteine, cysteine, 2-aminoethane-1-thiol and 2-hydroxyethane-1-thiol.

It will be appreciated that selective reduction process set forth above is particularly effective at targeted conjugation to the free cysteine. In this respect the extent of conjugation to the desired target site (defined here as “conjugation efficiency”) in site-specific antibodies may be determined by various art-accepted techniques. The efficiency of the site-specific conjugation of a drug to an antibody may be determined by assessing the percentage of conjugation on the target conjugation site(s) (e.g. free cysteines on the c-terminus of each light chain) relative to all other conjugated sites. In certain embodiments, the method herein provides for efficiently conjugating a drug to an antibody comprising free cysteines. In some embodiments, the conjugation efficiency is at least 5%, at least 10%, at least 15%, at least 20%, at least 25%, at least 30%, at least 35%, at least 40%, at least 45%, at least 50%, at least 55%, at least 60%, at least 70%, at least 75%, at least 80%, at least 85%, at least 90%, at least 95%, at least 98% or more as measured by the percentage of target conjugation relative to all other conjugation sites.

It will further be appreciated that engineered antibodies capable of conjugation may contain free cysteine residues that comprise sulfhydryl groups that are blocked or capped as the antibody is produced or stored. Such caps include small molecules, proteins, peptides, ions and other materials that interact with the sulfhydryl group and prevent or inhibit conjugate formation. In some cases the unconjugated engineered antibody may comprise free cysteines that bind other free cysteines on the same or different antibodies. As discussed herein such cross-reactivity may lead to various contaminants during the fabrication procedure. In some embodiments, the engineered antibodies may require uncapping prior to a conjugation reaction. In specific embodiments, antibodies herein are uncapped and display a free sulfhydryl group capable of conjugation. In specific embodiments, antibodies herein are subjected to an uncapping reaction that does not disturb or rearrange the naturally occurring disulfide bonds. It will be appreciated that in most cases the uncapping reactions will occur during the normal reduction reactions (reduction or selective reduction).

D. DAR Distribution and Purification

In selected embodiments conjugation and purification methodology compatible with the present invention advantageously provides the ability to generate relatively homogeneous ADC preparations comprising a narrow DAR distribution. In this regard the disclosed constructs (e.g., site-specific constructs) and/or selective conjugation provides for homogeneity of the ADC species within a sample in terms of the stoichiometric ratio between the drug and the engineered antibody and with respect to the toxin location. As briefly discussed above the term “drug to antibody ratio” or “DAR” refers to the molar ratio of drug to antibody in an ADC preparation. In certain embodiments a conjugate preparation may be substantially homogeneous with respect to its DAR distribution, meaning that within the ADC preparation is a predominant species of site-specific ADC with a particular drug loading (e.g., a drug loading of 2 or 4) that is also uniform with respect to the site of loading (i.e., on the free cysteines). In other certain embodiments of the invention it is possible to achieve the desired homogeneity through the use of site-specific antibodies and/or selective reduction and conjugation. In other embodiments the desired homogeneity may be achieved through the use of site-specific constructs in combination with selective reduction. In yet other embodiments compatible preparations may be purified using analytical or preparative chromatography techniques to provide the desired homogeneity. In each of these embodiments the homogeneity of the ADC sample can be analyzed using various techniques known in the art including but not limited to mass spectrometry, HPLC (e.g. size exclusion HPLC, RP-HPLC, HIC-HPLC etc.) or capillary electrophoresis.

With regard to the purification of ADC preparations it will be appreciated that standard pharmaceutical preparative methods may be employed to obtain the desired purity. As discussed herein liquid chromatography methods such as reverse phase (RP) and hydrophobic interaction chromatography (HIC) may separate compounds in the mixture by drug loading value. In some cases, ion-exchange (IEC) or mixed-mode chromatography (MMC) may also be used to isolate species with a specific drug load.

In any event the disclosed ADCs and preparations thereof may comprise drug and antibody moieties in various stoichiometric molar ratios depending on the configuration of the antibody and, at least in part, on the method used to effect conjugation. In certain embodiments the drug loading per ADC may comprise from 1-20 warheads (i.e., n is 1-20). Other selected embodiments may comprise ADCs with a drug loading of from 1 to 15 warheads. In still other embodiments the ADCs may comprise from 1-12 warheads or, more preferably, from 1-10 warheads. In some embodiments the ADCs will comprise from 1 to 8 warheads. While theoretical drug loading may be relatively high, practical limitations such as free cysteine cross reactivity and warhead hydrophobicity tend to limit the generation of homogeneous preparations comprising such DAR due to aggregates and other contaminants. That is, higher drug loading, e.g. >8 or 10, may cause aggregation, insolubility, toxicity, or loss of cellular permeability of certain antibody-drug conjugates depending on the payload. In view of such concerns drug loading provided by the instant invention preferably ranges from 1 to 8 drugs per conjugate, i.e. where 1, 2, 3, 4, 5, 6, 7, or 8 drugs are covalently attached to each antibody (e.g., for IgG1, other antibodies may have different loading capacity depending the number of disulfide bonds). Preferably the DAR of compositions of the instant invention will be approximately 2, 4 or 6 and in some embodiments the DAR will comprise approximately 2.

Despite the relatively high level of homogeneity provided by the instant invention the disclosed compositions actually comprise a mixture of conjugates with a range of drug compounds (potentially from 1 to 8 in the case of an IgG1). As such, the disclosed ADC compositions include mixtures of conjugates where most of the constituent antibodies are covalently linked to one or more drug moieties and (despite the relative conjugate specificity provided by engineered constructs and selective reduction) where the drug moieties may be attached to the antibody by various thiol groups. That is, following conjugation, compositions of the invention will comprise a mixture of ADCs with different drug loads (e.g., from 1 to 8 drugs per IgG1 antibody) at various concentrations (along with certain reaction contaminants primarily caused by free cysteine cross reactivity). However using selective reduction and post-fabrication purification the conjugate compositions may be driven to the point where they largely contain a single predominant desired ADC species (e.g., with a drug loading of 2) with relatively low levels of other ADC species (e.g., with a drug loading of 1, 4, 6, etc.). The average DAR value represents the weighted average of drug loading for the composition as a whole (i.e., all the ADC species taken together). Due to inherent uncertainty in the quantification methodology employed and the difficulty in completely removing the non-predominant ADC species in a commercial setting, acceptable DAR values or specifications are often presented as an average, a range or distribution (i.e., an average DAR of 2+/−0.5). Preferably compositions comprising a measured average DAR within the range (i.e., 1.5 to 2.5) would be used in a pharmaceutical setting.

Thus, in some embodiments the present invention will comprise compositions having an average DAR of 1, 2, 3, 4, 5, 6, 7 or 8 each +/−0.5. In other embodiments the present invention will comprise an average DAR of 2, 4, 6 or 8+/−0.5. Finally, in selected embodiments the present invention will comprise an average DAR of 2+/−0.5 or 4+/−0.5. It will be appreciated that the range or deviation may be less than 0.4 in some embodiments. Thus, in other embodiments the compositions will comprise an average DAR of 1, 2, 3, 4, 5, 6, 7 or 8 each +/−0.3, an average DAR of 2, 4, 6 or 8+/−0.3, even more preferably an average DAR of 2 or 4+/−0.3 or even an average DAR of 2+/−0.3. In other embodiments IgG1 conjugate compositions will preferably comprise a composition with an average DAR of 1, 2, 3, 4, 5, 6, 7 or 8 each +/−0.4 and relatively low levels (i.e., less than 30%) of non-predominant ADC species. In other embodiments the ADC composition will comprise an average DAR of 2, 4, 6 or 8 each +/−0.4 with relatively low levels (≤30%) of non-predominant ADC species. In some embodiments the ADC composition will comprise an average DAR of 2+/−0.4 with relatively low levels (≤30%) of non-predominant ADC species. In yet other embodiments the predominant ADC species (e.g., with a drug loading of 2 or drug loading of 4) will be present at a concentration of greater than 50%, at a concentration of greater than 55%, at a concentration of greater than 60%, at a concentration of greater than 65%, at a concentration of greater than 70%, at a concentration of greater than 75%, at a concentration of greater that 80%, at a concentration of greater than 85%, at a concentration of greater than 90%, at a concentration of greater than 93%, at a concentration of greater than 95% or even at a concentration of greater than 97% when measured against all other DAR species present in the composition.

As detailed in the Examples below the distribution of drugs per antibody in preparations of ADC from conjugation reactions may be characterized by conventional means such as UV-Vis spectrophotometry, reverse phase HPLC, HIC, mass spectroscopy, ELISA, and electrophoresis. The quantitative distribution of ADC in terms of drugs per antibody may also be determined. By ELISA, the averaged value of the drugs per antibody in a particular preparation of ADC may be determined. However, the distribution of drug per antibody values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues.

### VII. Diagnostics and Screening

A. Diagnostics

The invention provides in vitro and in vivo methods for detecting, diagnosing or monitoring proliferative disorders and methods of screening cells from a patient to identify tumor cells including tumorigenic cells. Such methods include identifying an individual having cancer for treatment or monitoring progression of a cancer, comprising contacting the patient or a sample obtained from a patient (either in vivo or in vitro) with a detection agent (e.g., an antibody or nucleic acid probe) capable of specifically recognizing and associating with DLL3 or ASCL1 and detecting the presence or absence, or level of association of the detection agent in the sample. In selected embodiments the detection agent will comprise an antibody associated with a detectable label or reporter molecule as described herein. In yet other embodiments (e.g., In situ hybridization or ISH) a nucleic acid probe that reacts with a genomic DLL3 or ASCL1 determinant will be used in the detection, diagnosis or monitoring of the proliferative disorder.

More generally the presence and/or levels of DLL3 or ASCL1 determinants may be measured using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), Polymerase Chain Reaction (PCR, RT-PCR; RT-qPCR) technology, branched oligonucleotide technology, Northern blot technology, oligonucleotide hybridization technology and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

In some embodiments, the association of the detection agent with particular cells or cellular components in the sample indicates that the sample may contain tumorigenic cells, thereby denoting that the individual having cancer may be effectively treated with an antibody or ADC as described herein.

In certain preferred embodiments the assays may comprise immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.) or In situ hybridization (ISH) or variants thereof (e.g., chromogenic in situ hybridization (CISH) or fluorescence in situ hybridization (DNA-FISH or RNA-FISH]))

In this regard certain aspects of the instant invention comprise the use of labeled DLL3 or labeled ASCL1 for immunohistochemistry (IHC). More particularly DLL3 IHC or ASCL1 IHC may be used as a diagnostic tool to aid in the diagnosis of various proliferative disorders and to monitor the potential response to treatments including DLL3 antibody therapy. As discussed herein and shown in the Examples below compatible diagnostic assays may be performed on tissues that have been chemically fixed (compatible techniques include, but are not limited to: formaldehyde, glutaraldehyde, osmium tetroxide, potassium dichromate, acetic acid, alcohols, zinc salts, mercuric chloride, chromium tetroxide and picric acid) and embedded (compatible methods include but are not limited to: glycol methacrylate, paraffin and resins) or preserved via freezing. Such assays can be used to guide treatment decisions and determine dosing regimens and timing.

Immunohistochemistry techniques may be used to derive an ASLC1 H-score as known in the art. Such H-scores may be used to indicate which patients may be amenable to treatment with the compositions of the instant invention. H-scores of approximately 90, approximately 100, approximately 110, approximately 120, approximately 130, approximately 140, approximately 150, approximately 160, approximately 170, approximately 180, approximately 190 or approximately 200 or above on a 300 point scale may be used in selected embodiments to indicate which patients may respond favorably to the treatment methods of the instant invention. Accordingly in one embodiment a patient to be treated with the DLL3 ADCs of the instant invention will have an H-score of at least 90 (i.e., the tumor is ASCL1⁺⁾ on a 300 point scale. In other embodiments a patient to be treated with the DLL3 ADCs of the instant invention will have an ASLC1 H-score of at least 120. In yet other embodiments a patient to be treated with the DLL3 ADCs of the instant invention will have an ASLC1 H-score of at least 180. For the purposes of the instant disclosure any tumor exhibiting an ASLC1 H-score of 90 or above on a 300 point scale will be considered ASCL1⁺ tumor and subject to treatment with the disclosed antibodies or ADCs.

In still other embodiments patient selection may be predicated on the percent of marker (e.g., ASCL1) positive cells staining with a certain intensity. By way of example, a tumor with >20% of the cells exhibiting 2+ intensity or greater will be a candidate for treatment with a DLL3 ADC. In other embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 1+ intensity or greater when stained with a marker antibody (e.g., an anti-ASCL1 antibody) and examined in accordance with standard IHC protocols as disclosed herein. In other certain embodiments a patient will be a candidate for treatment with a DLL3 ADC or other chemotherapeutic agent if ≥20%, ≥30%, ≥40%, ≥50%, ≥60%, ≥70% or ≥80% of the tumor cells exhibit 2+ intensity or greater when stained with a marker antibody and examined in accordance with standard IHC protocols as disclosed herein. In yet other selected embodiments a patient will be a candidate for treatment with a DLL3 ADC or other chemotherapeutic agent if ≥10%, ≥20%, ≥30%, ≥40% or ≥50% of the tumor cells exhibit 1+ intensity or greater when stained with a marker antibody and examined in accordance with standard IHC protocols as disclosed herein. In still other embodiments a patient will be a candidate for treatment with a DLL3 ADC or other chemotherapeutic agent if ≥10%, ≥20%, ≥30%, ≥40% or ≥50% of the tumor cells exhibit 2+ intensity or greater when stained with a marker antibody and examined in accordance with standard IHC protocols as disclosed herein. Yet another embodiment comprises a method of treating a subject having a tumor comprising tumor cells wherein ≥10% of the tumor cells exhibit 1+ intensity or greater when stained with a marker antibody and examined in accordance with standard IHC protocols comprising the step of administering an anti-DLL3 ADC. With regard to each of the aforementioned embodiments it will be appreciated that the intensity of staining with a marker antibody may be readily determined using standard pathology techniques and methodology familiar to those of skill in the art.

As discussed above certain marker levels and DLL3 expression will be decreased or reduced as compared to a reference expression level in a control sample. More specifically, tumors at risk of transitioning to a neuroendocrine phenotype may express lower levels of one markers selected from the group consisting of Retinoblastoma 1 (RB1), Repressor Element-1 Silencing Transcription Factor (REST), SAM Pointed Domain-containing Ets Transcription Factor (SPDEF), Prostaglandin E2 Receptor 4 (PTGER4), and ETS-Related Gene (ERG). In addition such tumors may express relatively low levels of DLL3 protein and may be classified as ASCL1⁺, DLL3^{-/low} wherein DLL3⁻ is indicative of non-detectable or barely detectable levels of expression and DLL3^{-/low} is indicative of relatively depressed levels of DLL3 found in certain tumors (e.g., adenocarcinoma). In this regard DLL3^{low} will be held to mean any tumor comprising a DLL3 expression level that is reduced by 5%, 10%, 15%, 20%, 25%, 30%, 40%, 50%, 60%, 70%, 80%, 85%, 90%, 95%, 96%, 97%, 98%, 99% or more as compared to a reference expression level in a control sample (e.g., a DLL3+ or hi tumor). In certain embodiments DLL3 expression will be reduced by 50%, 60%, 70%, 80%, 85%, 90%, 95%, 96%, 97%, 98%, 99% or more as compared to a reference expression level in a control sample. In still other embodiments DLL3 expression will be reduced by 80%, 85%, 90%, 95%, 96%, 97%, 98%, 99% or more as compared to a reference expression level in a control sample while in other embodiments DLL3 expression will be reduced by 90%, 95%, 96%, 97%, 98%, 99% or more as compared to a reference expression level in a control sample. In selected embodiments DLL3 expression will be reduced by at least 90%, by at least 95%, by at least 97% or by at least 99% when compared to a sample obtained from a DLL3⁺ tumor.

In other embodiments the tumor sample may compared to control tumor samples known not to express DLL3 (negative control). When such comparisons are made the tumor sample obtained from the subject may be classified as DLL3⁻ if it exhibits substantially the same level of DLL3 as the negative control.

In yet other embodiments DLL3^{-/low} tumors may readily be identified by trained pathologists using IHC in view of the instant disclosure. More specifically tumor samples may be obtained, preferably fixed and stained with anti-DLL3 antibodies as disclosed herein and read using art-recognized techniques. In certain embodiments the expression of DLL3 may be visually determined by the pathologist using appropriate positive and negative controls. In other embodiments the scoring could be based on a derived H-score may comprise the measurement of percent of positively stained cells in a tumor sample. With respect to the latter a tumor may be found to be DLL3^{-/low} if less than about 20%, 15%, 10%, 8%, 5%, 4%, 3%, 2% or 1% of the cells stain positive using standard IHC techniques. In other embodiments a tumor may be found to be DLL3^{-/low} if less than about 0.8%, 0.6%, 0.4%, 0.2% or 0.1% of the cells stain positive when interrogated with a DLL3 antibody as described herein. In other embodiments the skilled artisan may make a qualitative judgement as to what constitutes a DLL3^{-/low} tumor upon review of the slides based on factors such as relative intensity, staining patterns, sample origin and preparation, antibody and reporter employed, etc. As previously alluded to any determination as to the level of DLL3 expression is made in the context of appropriate positive and negative controls and is relatively accurate. Accordingly such determinations are indicative as to which patients are susceptible to treatment with DLL3 ADCs as described herein.

Other particularly compatible aspects of the invention involve the use of in situ hybridization to detect or monitor DLL3 or ASCL1 determinants. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic DLL3 or ASCL1 determinants. Probes preferably hybridize to a nucleotide sequence that corresponds to such determinants. Hybridization conditions can be routinely optimized to minimize background signal by non-fully complementary hybridization though preferably the probes are preferably fully complementary to the selected DLL3 or ASCL1 determinant. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In certain embodiments the antibodies of the instant invention may be used to detect and quantify levels of a particular determinant (e.g., DLL3 protein or ASCL1 protein) in a patient sample (e.g., plasma or blood) which may, in turn, be used to detect, diagnose or monitor proliferative disorders that are associated with the relevant determinant. In related embodiments the antibodies of the instant invention may be used to detect, monitor and/or quantify circulating tumor cells either in vivo or in vitro (WO 2012/0128801). In still other embodiments the circulating tumor cells may comprise tumorigenic cells.

In certain embodiments of the invention, the tumorigenic cells in a subject or a sample from a subject may be assessed or characterized using the disclosed antibodies prior to therapy or regimen to establish a baseline. In other examples, the tumorigenic cells can be assessed from a sample that is derived from a subject that was treated.

In another embodiment, the invention provides a method of analyzing cancer progression and/or pathogenesis in vivo. In another embodiment, analysis of cancer progression and/or pathogenesis in vivo comprises determining the extent of tumor progression. In another embodiment, analysis comprises the identification of the tumor. In another embodiment, analysis of tumor progression is performed on the primary tumor. In another embodiment, analysis is performed over time depending on the type of cancer as known to one skilled in the art. In another embodiment, further analysis of secondary tumors originating from metastasizing cells of the primary tumor is conducted in vivo. In another embodiment, the size and shape of secondary tumors are analyzed. In some embodiments, further ex vivo analysis is performed.

In another embodiment, the invention provides a method of analyzing cancer progression and/or pathogenesis in vivo including determining cell metastasis or detecting and quantifying the level of circulating tumor cells. In yet another embodiment, analysis of cell metastasis comprises determination of progressive growth of cells at a site that is discontinuous from the primary tumor. In some embodiments, procedures may be undertaken to monitor tumor cells that disperse via blood vasculature, lymphatics, within body cavities or combinations thereof. In another embodiment, cell metastasis analysis is performed in view of cell migration, dissemination, extravasation, proliferation or combinations thereof.

In certain examples, the tumorigenic cells in a subject or a sample from a subject may be assessed or characterized using the disclosed antibodies prior to therapy to establish a baseline. In other examples the sample is derived from a subject that was treated. In some examples the sample is taken from the subject at least about 1, 2, 4, 6, 7, 8, 10, 12, 14, 15, 16, 18, 20, 30, 60, 90 days, 6 months, 9 months, 12 months, or >12 months after the subject begins or terminates treatment. In certain examples, the tumorigenic cells are assessed or characterized after a certain number of doses (e.g., after 2, 5, 10, 20, 30 or more doses of a therapy). In other examples, the tumorigenic cells are characterized or assessed after 1 week, 2 weeks, 1 month, 2 months, 1 year, 2 years, 3 years, 4 years or more after receiving one or more therapies.

B. Screening

In certain embodiments, antibodies of the instant invention can be used to screen samples in order to identify compounds or agents (e.g., antibodies or ADCs) that alter a function or activity of tumor cells by interacting with a determinant. In one embodiment, tumor cells are put in contact with an antibody or ADC and the antibody or ADC can be used to screen the tumor for cells expressing a certain target (e.g. DLL3 or ASCL1) in order to identify such cells for purposes, including but not limited to, diagnostic purposes, to monitor such cells to determine treatment efficacy or to enrich a cell population for such target-expressing cells.

In yet another embodiment, a method includes contacting, directly or indirectly, tumor cells with a test agent or compound and determining if the test agent or compound modulates an activity or function of the determinant-associated tumor cells for example, changes in cell morphology or viability, expression of a marker, differentiation or de-differentiation, cell respiration, mitochondrial activity, membrane integrity, maturation, proliferation, viability, apoptosis or cell death. One example of a direct interaction is physical interaction, while an indirect interaction includes, for example, the action of a composition upon an intermediary molecule that, in turn, acts upon the referenced entity (e.g., cell or cell culture).

Screening methods include high throughput screening, which can include arrays of cells (e.g., microarrays) positioned or placed, optionally at pre-determined locations, for example, on a culture dish, tube, flask, roller bottle or plate. High-throughput robotic or manual handling methods can probe chemical interactions and determine levels of expression of many genes in a short period of time. Techniques have been developed that utilize molecular signals, for example via fluorophores or microarrays (Mocellin and Rossi, 2007, PMID: 17265713) and automated analyses that process information at a very rapid rate (see, e.g., Pinhasov et al., 2004, PMID: 15032660). Libraries that can be screened include, for example, small molecule libraries, phage display libraries, fully human antibody yeast display libraries (Adimab), siRNA libraries, and adenoviral transfection vectors.

### VIII. Pharmaceutical Preparations and Therapeutic Uses

Anti-DLL3 ADC therapy for DLL3⁺ tumors is well-described. See e.g, PCT Publication Nos. WO 2013126746 and WO 2014130879; and Pietanza et al., EMSO Abstract 2015. In particular, DLL3 expression correlates with tumors that transition to a neuroendocrine phenotype and thereafter are highly untreatable using standard of care. The present invention provides methods and compositions for identifying tumors at risk for neuroendocrine transition, such that patients having the disclosed risk factors are identifiable as candidates for treatment of tumors that are DLL3^{-/low} with an anti-DLL3 antibody drug conjugate. As detailed herein, risk factors include (1) various proteins are expressed in tumors during the transition to a neuroendocrine phenotype and prior to substantial DLL3 expression, and (2) prior treatment with a targeted therapy.

The early treatment of tumor at risk of transitioning to a neuroendocrine phenotype is beneficial for reducing or inhibiting tumor recurrence. In particular anti-DLL3 antibody drug conjugates may be effective in treating DLL3^{-/low} tumors that show one or more risk factors, as disclosed herein, for a neuroendocrine transition, to thereby reduce the incidence of recurrence. In the case of relapsed or recurrent cancer, the tumors at risk include those previously treated with a targeted cancer therapy.

In one aspect of the invention, an anti-DLL3 ADC is administered to the patient before the adenocarcinoma fully transitions to a neuroendocrine phenotype, wherein the tumor is typically DLL3^{-/low}. In a related aspect, the anti-DLL3 ADC is administered before treatment with a targeted cancer therapy. In another aspect, the anti-DLL3 ADC is administered as a combination therapy with a targeted cancer therapy, wherein the administering occurs concurrently or consecutively with the anti-DLL3 ADC. In still another aspect the anti-DLL3 antibody is administered to a subject having a DLL3^{-/low} tumor before, after or concurrently with a chemotherapeutic regimen. In yet another aspect, the anti-DLL3 ADC is administered to the patient after the adenocarcinoma has developed resistance, or otherwise shows a risk of developing resistance, to a targeted cancer therapy.

By reducing or inhibiting recurrence is meant any significant decrease in disease recurrence as compared to a control. For example, a significant reduction of at least 5%, 10%, 15%, 20%, 25%, 30%, 40%, 50%, 60%, 70%, 80%, 85%, 90%, 95%, 96%, 97%, 98%, 99% or more as compared to a control. Alternatively, reducing or inhibiting recurrence can be any fold decrease of at least 1.5-fold, 2-fold, 3-fold, 4-fold, 5-fold, 6-fold, 7-fold, 8-fold, 9-fold, 10-fold, 12-fold, 14-fold, 16-fold, 20-fold or more as compared to a control. Representative controls include, for example, the rate of recurrence in a patient population receiving a given targeted therapy in the absence of anti-DLL3 ADC therapy.

A. Formulations and Routes of Administration

Depending on the form of the antibody drug conjugate, the mode of intended delivery, the disease being treated or monitored and numerous other variables, compositions of the invention may be formulated as desired using art-recognized techniques. In some embodiments, the therapeutic compositions of the invention may be administered neat or with a minimum of additional components while others may optionally be formulated to contain suitable pharmaceutically acceptable carriers comprising excipients and auxiliaries that are well known in the art (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents and the like, are also available. Certain non-limiting exemplary carriers include saline, buffered saline, dextrose, water, glycerol, ethanol, and combinations thereof.

More particularly it will be appreciated that, in some embodiments, the therapeutic compositions of the invention may be administered neat or with a minimum of additional components. Conversely the DLL3 antibody drug conjugates of the present invention may optionally be formulated to contain suitable pharmaceutically acceptable carriers comprising excipients and auxiliaries that are well known in the art and are relatively inert substances that facilitate administration of the antibody drug conjugate or which aid processing of the active compounds into preparations that are pharmaceutically optimized for delivery to the site of action. For example, an excipient can give form or consistency or act as a diluent to improve the pharmacokinetics or stability of the antibody drug conjugate. Suitable excipients or additives include, but are not limited to, stabilizing agents, wetting and emulsifying agents, salts for varying osmolarity, encapsulating agents, buffers, and skin penetration enhancers. In certain preferred embodiments the pharmaceutical compositions may be provided in a lyophilized form and reconstituted in, for example, buffered saline prior to administration.

Disclosed antibody drug conjugates for systemic administration may be formulated for enteral, parenteral or topical administration. Indeed, all three types of formulation may be used simultaneously to achieve systemic administration of the active ingredient. Excipients as well as formulations for parenteral and nonparenteral drug delivery are set forth in Remington, The Science and Practice of Pharmacy 20th Ed. Mack Publishing (2000). Suitable formulations for parenteral administration include aqueous solutions of the active compounds in water-soluble form, for example, water-soluble salts. In addition, suspensions of the active compounds as appropriate for oily injection suspensions may be administered. Suitable lipophilic solvents or vehicles include fatty oils, for example, hexylsubstituted poly(lactide), sesame oil, or synthetic fatty acid esters, for example, ethyl oleate or triglycerides. Aqueous injection suspensions may contain substances that increase the viscosity of the suspension and include, for example, sodium carboxymethyl cellulose, sorbitol, and/or dextran. Optionally, the suspension may also contain stabilizers. Liposomes can also be used to encapsulate the agent for delivery into the cell.

Suitable formulations for enteral administration include hard or soft gelatin capsules, pills, tablets, including coated tablets, elixirs, suspensions, syrups or inhalations and controlled release forms thereof.

In general the compounds and compositions of the invention, comprising DLL3 antibody drug conjugates may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

B. Dosages

Similarly, the particular dosage regimen, i.e., dose, timing and repetition, will depend on the particular individual and that individual's medical history, as well as empirical considerations such as pharmacokinetics (e.g., half-life, clearance rate, etc.). Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In other embodiments the dosage administered may be adjusted or attenuated to manage potential side effects and/or toxicity. Alternatively, sustained continuous release formulations of a subject therapeutic composition may be appropriate.

In general, the antibody drug conjugates may be administered in various ranges. These include about 10 μg/kg body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In selected embodiments the antibody drug conjugates will be administered at approximately 10, 20, 30, 40, 50, 60, 70, 80, 90 or 100 μg/kg body weight per dose. Other embodiments will comprise the administration of antibody drug conjugates at 200, 300, 400, 500, 600, 700, 800 or 900 μg/kg body weight per dose. In other preferred embodiments the antibody drug conjugates will be administered at 1, 2, 3, 4, 5, 6, 7, 8, 9 or 10 mg/kg. In still other embodiments the antibody drug conjugates may be administered at 12, 14, 16, 18 or 20 mg/kg body weight per dose. In yet other embodiments the antibody drug conjugates may be administered at 25, 30, 35, 40, 45, 50, 55, 60, 65, 70, 75, 80, 90 or 100 mg/kg body weight per dose. In accordance with the teachings herein one of skill in the art could readily determine appropriate dosages for various antibody drug conjugates based on preclinical animal studies, clinical observations and standard medical and biochemical techniques and measurements. In particularly preferred embodiments such antibody drug conjugate dosages will be administered intravenously over a period of time. Moreover, such dosages may be administered multiple times over a defined course of treatment.

Other dosing regimens may be predicated on Body Surface Area (BSA) calculations as disclosed in U.S. Pat. No. 7,744,877. As is well known, the BSA is calculated using the patient's height and weight and provides a measure of a subject's size as represented by the surface area of his or her body. In certain embodiments, the antibody drug conjugates may be administered in dosages from 10 mg/m² to 800 mg/m², from 50 mg/m² to 500 mg/m² and at dosages of 100 mg/m², 150 mg/m², 200 mg/m², 250 mg/m², 300 mg/m², 350 mg/m², 400 mg/m² or 450 mg/m². It will also be appreciated that art recognized and empirical techniques may be used to determine appropriate dosage.

In any event, DLL3 antibody drug conjugates are preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like. Generally, an effective dose of the DLL3 antibody drug conjugate is administered to a subject one or more times. More particularly, an effective dose of the antibody drug conjugate is administered to the subject once a month, more than once a month, or less than once a month. In certain embodiments, the effective dose of the DLL3 antibody drug conjugate may be administered multiple times, including for periods of at least a month, at least six months, at least a year, at least two years or a period of several years. In yet other embodiments, several days (2, 3, 4, 5, 6 or 7), several weeks (1, 2, 3, 4, 5, 6, 7 or 8) or several months (1, 2, 3, 4, 5, 6, 7 or 8) or even a year or several years may lapse between administration of the antibody drug conjugates.

In certain preferred embodiments the course of treatment involving antibody drug conjugates will comprise multiple doses of the selected drug product over a period of weeks or months. More specifically, antibody drug conjugates may administered once every day, every two days, every four days, every week, every ten days, every two weeks, every three weeks, every month, every six weeks, every two months, every ten weeks or every three months. In this regard it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments the dosage may be gradually increased or reduced or attenuated based respectively on empirically determined or observed side effects or toxicity. To assess efficacy of the selected composition, a marker of the specific disease, disorder or condition can be followed as described previously. In embodiments where the individual has cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or an antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

C. Combination Therapies

As described herein, adenocarcinoma tumors are often heterogeneous and may comprise rare neuroendocrine cells that are resistant to targeted anti-cancer therapies. Accordingly, an effective therapeutic strategy comprises administering anti-DLL3 antibody drug conjugates as a combination therapy with anti-cancer agents. In one embodiment, the anti-DLL3 antibody drug conjugate is administered as a combination therapy with a targeted anti-cancer therapy (e.g. a targeted anti-cancer agent). In one aspect, the anti-DLL3 antibody drug conjugate may be administered simultaneously with a targeted anti-cancer therapy. In other aspects the administration of the anti-DLL3 antibody drug conjugate occurs after a tumor has become resistant to a targeted anti-cancer therapy. In yet other aspects, the administration of the anti-DLL3 antibody drug conjugate occurs before administration of a targeted anti-cancer therapy.

Combination therapies may be particularly useful in decreasing or inhibiting unwanted neoplastic cell proliferation, decreasing the occurrence of cancer, decreasing or preventing the recurrence of cancer, or decreasing or preventing the spread or metastasis of cancer. In such cases the antibody drug conjugates may function as sensitizing or chemosensitizing agents by removing the cancer cells that would otherwise prop up and perpetuate the tumor mass and thereby allow for more effective use of current standard of care debulking or anti-cancer agents. That is, the antibody drug conjugates may, in certain embodiments provide an enhanced effect (e.g., additive or synergistic in nature) that potentiates the mode of action of another administered therapeutic agent. In the context of the instant invention “combination therapy” shall be interpreted broadly and merely refers to the administration of an antibody drug conjugate and one or more anti-cancer agents that include, but are not limited to, cytotoxic agents, cytostatic agents, anti-angiogenic agents, debulking agents, chemotherapeutic agents, radiotherapy and radiotherapeutic agents, targeted anti-cancer therapies or agents (including both monoclonal antibodies and small molecule entities), BRMs, therapeutic antibodies, cancer vaccines, cytokines, hormone therapies, radiation therapy and anti-metastatic agents and immunotherapeutic agents, including both specific and non-specific approaches.

There is no requirement for the combined results to be additive of the effects observed when each treatment (e.g., antibody drug conjugate and anti-cancer agent) is conducted separately. Although at least additive effects are generally desirable, any increased anti-tumor effect above one of the single therapies is beneficial. Furthermore, the invention does not require the combined treatment to exhibit synergistic effects. However, those skilled in the art will appreciate that with certain selected combinations that comprise preferred embodiments, synergism may be observed.

In practicing combination therapy, the antibody drug conjugate and anti-cancer agent (e.g. a targeted anti-cancer therapy) may be administered to the subject simultaneously, either in a single composition, or as two or more distinct compositions using the same or different administration routes. Alternatively, the antibody drug conjugate may precede, or follow, the anti-cancer agent treatment by, e.g., intervals ranging from minutes to weeks. The time period between each delivery is such that the anti-cancer agent and antibody drug conjugate are able to exert a combined effect on the tumor. In at least one embodiment, both the anti-cancer agent and the antibody drug conjugate are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the antibody drug conjugate and the anti-cancer agent.

The combination therapy may be administered once, twice or at least for a period of time until the condition is treated, palliated or cured. In some embodiments, the combination therapy is administered multiple times, for example, from three times daily to once every six months. The administering may be on a schedule such as three times daily, twice daily, once daily, once every two days, once every three days, once weekly, once every two weeks, once every month, once every two months, once every three months, once every six months or may be administered continuously via a minipump. The combination therapy may be administered via any route, as noted previously. The combination therapy may be administered at a site distant from the site of the tumor.

In one embodiment an antibody drug conjugate is administered in combination with one or more anti-cancer agents (e.g. a targeted anti-cancer therapy) for a short treatment cycle to a subject in need thereof. The invention also contemplates discontinuous administration or daily doses divided into several partial administrations. The antibody drug conjugate and anti-cancer agent may be administered interchangeably, on alternate days or weeks; or a sequence of antibody drug conjugate treatments may be given, followed by one or more treatments of anti-cancer agent therapy. In any event, as will be understood by those of ordinary skill in the art, the appropriate doses of chemotherapeutic agents will be generally around those already employed in clinical therapies wherein the chemotherapeutics are administered alone or in combination with other chemotherapeutics.

In another preferred embodiment the anti-DLL3 antibody drug conjugates may be used in maintenance therapy to reduce or eliminate the chance of tumor recurrence following the initial presentation of the disease. Preferably the disorder will have been treated and the initial tumor mass eliminated, reduced or otherwise ameliorated so the patient is asymptomatic or in remission. At such time the subject may be administered pharmaceutically effective amounts of the disclosed antibody drug conjugates one or more times even though there is little or no indication of disease using standard diagnostic procedures. In some embodiments, the antibody drug conjugates will be administered on a regular schedule over a period of time, such as weekly, every two weeks, monthly, every six weeks, every two months, every three months every six months or annually. Given the teachings herein, one skilled in the art could readily determine favorable dosages and dosing regimens to reduce the potential of disease recurrence. Moreover such treatments could be continued for a period of weeks, months, years or even indefinitely depending on the patient response and clinical and diagnostic parameters.

In yet another preferred embodiment the antibody drug conjugates may be used to prophylactically or as an adjuvant therapy to prevent or reduce the possibility of tumor metastasis following a debulking procedure. As used in the instant disclosure a “debulking procedure” is defined broadly and shall mean any procedure, technique or method that eliminates, reduces, treats or ameliorates a tumor or tumor proliferation. Exemplary debulking procedures include, but are not limited to, surgery, radiation treatments (i.e., beam radiation), chemotherapy, immunotherapy or ablation. At appropriate times readily determined by one skilled in the art in view of the instant disclosure the disclosed antibody drug conjugates may be administered as suggested by clinical, diagnostic or theragnostic procedures to reduce tumor metastasis. The antibody drug conjugates may be administered one or more times at pharmaceutically effective dosages as determined using standard techniques. Preferably the dosing regimen will be accompanied by appropriate diagnostic or monitoring techniques that allow it to be modified.

D. Anti-Cancer Agents

The antibody drug conjugates may be used in combination with anti-cancer agents. The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents.

As used herein the term “cytotoxic agent” means a substance that is toxic to the cells and decreases or inhibits the function of cells and/or causes destruction of cells. Typically, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, saponaria officinalis inhibitor, gelonin, mitegellin, restrictocin, phenomycin, neomycin, and the tricothecenes) or animals, (e.g., cytotoxic RNases, such as extracellular pancreatic RNases; DNase I, including fragments and/or variants thereof).

For the purposes of the instant invention a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the antibody drug conjugates include, but are not limited to, alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, erlotinib, vemurafenib, crizotinib, ceritinib, sorafenib, ibrutinib, enzalutamide, folic acid analogues, purine analogs, androgens, anti-adrenals, folic acid replenisher such as frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane; rhizoxin; sizofiran; spirogermanium; tenuazonic acid; triaziquone; 2,2′,2″-trichlorotriethyl amine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine; methotrexate; platinum analogs, vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11), topoisomerase inhibitor RFS 2000; difluorometlhylornithine; retinoids; capecitabine; combretastatin; leucovorin; oxaliplatin; inhibitors of PKC-alpha, Raf, H-Ras, EGFR and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

Particularly preferred anti-cancer agents comprise commercially or clinically available compounds such as erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®). Additional commercially or clinically available anti-cancer agents comprise oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); vinorelbine (NAVELBINE®); capecitabine (XELODA®, Roche), tamoxifen (including NOLVADEX®; tamoxifen citrate, FARESTON® (toremifine citrate) MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca).

In other embodiments the antibody drug conjugates may be used in combination with any one of a number of antibodies (or immunotherapeutic agents) presently in clinical trials or commercially available. To this end the antibody drug conjugates may be used in combination with an antibody selected from the group consisting of abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, 3F8 and combinations thereof.

Still other particularly preferred embodiments will comprise the use of antibodies approved for cancer therapy including, but not limited to, rituximab, trastuzumab, gemtuzumab ozogamcin, alemtuzumab, ibritumomab tiuxetan, tositumomab, bevacizumab, cetuximab, panitumumab, ofatumumab, ipilimumab and brentuximab vedotin. Those skilled in the art will be able to readily identify additional anti-cancer agents that are compatible with the teachings herein.

E. Radiotherapy

The present invention also provides for the combination of antibody drug conjugates with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### IX. Indications

It will be appreciated that the anti-DLL3 antibody drug conjugates may be used to treat, prevent, manage, or inhibit the occurrence or recurrence of any DLL3⁺ cancer, as well as those cancers at risk for becoming DLL3⁺, for examples, those DLL3^{(-/low)} tumors at risk of neuroendocrine transition as described herein.

Accordingly, whether administered alone or in combination with an anti-cancer agent or radiotherapy, the antibody drug conjugates are particularly useful for generally treating neoplastic conditions in patients or subjects which may include benign or malignant tumors (e.g., adrenal, liver, kidney, bladder, breast, gastric, ovarian, colorectal, prostate, pancreatic, lung, thyroid, hepatic, cervical, endometrial, esophageal and uterine carcinomas; sarcomas; glioblastomas; and various head and neck tumors); leukemias and lymphoid malignancies; other disorders such as neuronal, glial, astrocytal, hypothalamic and other glandular, macrophagal, epithelial, stromal and blastocoelic disorders; and inflammatory, angiogenic, immunologic disorders and disorders caused by pathogens. Particularly, key targets for treatment are neoplastic conditions comprising solid tumors, although hematologic malignancies are within the scope of the invention. Preferably the “subject” or “patient” to be treated will be human although, as used herein, the terms are expressly held to comprise any mammalian species.

More specifically, neoplastic conditions subject to treatment in accordance with the instant invention may be selected from the group including, but not limited to, adrenal gland tumors, AIDS-associated cancers, alveolar soft part sarcoma, astrocytic tumors, bladder cancer (squamous cell carcinoma and transitional cell carcinoma), bone cancer (adamantinoma, aneurismal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's Sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancers (small cell carcinoma, adenocarcinoma, squamous cell carcinoma, large cell carcinoma etc.), medulloblastoma, melanoma, meningiomas, multiple endocrine neoplasia, multiple myeloma, myelodysplastic syndrome, neuroblastoma, neuroendocrine tumors, ovarian cancer, pancreatic cancers, papillary thyroid carcinomas, parathyroid tumors, pediatric cancers, peripheral nerve sheath tumors, phaeochromocytoma, pituitary tumors, prostate cancer, posterious unveal melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma).

In certain preferred embodiments the proliferative disorder will comprise a solid tumor including, but not limited to, adrenal, liver, kidney, bladder, breast, gastric, ovarian, cervical, uterine, esophageal, colorectal, prostate, pancreatic, lung (both small cell and non-small cell), thyroid, carcinomas, sarcomas, glioblastomas and various head and neck tumors. In other preferred embodiments, the antibody drug conjugates are especially effective at treating small cell lung cancer (SCLC) and non-small cell lung cancer (NSCLC) (e.g., squamous cell non-small cell lung cancer or squamous cell small cell lung cancer). In one embodiment, the lung cancer is refractory, relapsed or resistant to a platinum based agent (e.g., carboplatin, cisplatin, oxaliplatin, topotecan) and/or a taxane (e.g., docetaxel, paclitaxel, larotaxel or cabazitaxel). With regard to small cell lung cancer particularly preferred embodiments will comprise the administration of antibody drug conjugates. In selected embodiments the antibody drug conjugates will be administered to patients exhibiting limited stage disease. In other embodiments the antibody drug conjugates will be administered to patients exhibiting extensive stage disease. In other preferred embodiments the antibody drug conjugates will be administered to refractory patients (i.e., those who recur during or shortly after completing a course of initial therapy). Still other embodiments comprise the administration of the antibody drug conjugates to sensitive patients (i.e, those whose relapse is longer than 2-3 months after primary therapy).

As discussed above the anti-DLL3 antibody drug conjugates may be used to prevent, treat or diagnose tumors with neuroendocrine features or phenotypes including neuroendocrine tumors. The anti-DLL3 antibody drug conjugates may also be used to treat tumors that are at risk of transitioning to a neuroendocrine phenotype, which is identifiable by the risk factors disclosed herein. Accordingly, the anti-DLL3 antibody drug conjugates may be used to treat tumors of any of the foregoing cancer types, which are characterized at risk for neuroendocrine transition by the changes in biomarker expression and/or having previously received a targeted therapy. In particular aspects of the invention, tumors at risk of neuroendocrine transition include adenocarcinoma arising in the lung, prostate, bladder, kidney, genitourinary tract, including bladder, prostate, ovary, cervix, and endometrium; gastrointestinal tract, including colon, and stomach; thyroid, including medullary thyroid cancer; and lung, including small cell lung carcinoma and large cell neuroendocrine carcinoma.

### X. Articles of Manufacture

The invention includes pharmaceutical packs and kits comprising one or more containers or receptacles, wherein a container can comprise one or more doses of an antibody or ADC of the invention. Such kits or packs may be diagnostic or therapeutic in nature. In certain embodiments, the pack or kit contains a unit dosage, meaning a predetermined amount of a composition comprising, for example, an antibody or ADC of the invention, with or without one or more additional agents and optionally, one or more anti-cancer agents. In certain other embodiments, the pack or kit contains a detectable amount of an anti-DLL3 antibody, an anti-ASCL1 antibody, or an anti-DLL3 ADC, with or without an associated reporter molecule and optionally one or more additional agents for the detection, quantitation and/or visualization of cancerous cells. In yet other embodiments kits compatible with the instant invention may comprise one or more agents useful for detecting a marker selected from the group consisting of Achaete-scute Homolog 1 (ASCL1), Paternally Expressed 10 (PEG10), or Serine/Arginine Repetitive Matrix 4 (SRRM4), Retinoblastoma 1 (RB1), Repressor Element-1 Silencing Transcription Factor (REST), SAM Pointed Domain-containing Ets Transcription Factor (SPDEF), Prostaglandin E2 Receptor 4 (PTGER4) and ETS-Related Gene (ERG).

In any event kits of the invention will generally comprise an antibody or ADC of the invention in a suitable container or receptacle a pharmaceutically acceptable formulation and, optionally, one or more anti-cancer agents in the same or different containers. The kits may also contain other pharmaceutically acceptable formulations or devices, either for diagnosis or combination therapy. Examples of diagnostic devices or instruments include those that can be used to detect, monitor, quantify or profile cells or markers associated with proliferative disorders (for a full list of such markers, see above). In some embodiments the devices may be used to detect, monitor and/or quantify circulating tumor cells either in vivo or in vitro (see, for example, WO 2012/0128801). In still other embodiments the circulating tumor cells may comprise tumorigenic cells. The kits contemplated by the invention can also contain appropriate reagents to combine the antibody or ADC of the invention with an anti-cancer agent or diagnostic agent (e.g., see U.S. Pat. No. 7,422,739).

When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises the antibody or ADC of the invention in combination with additional therapeutics or agents, the solution may be pre-mixed, either in a molar equivalent combination, or with one component in excess of the other. Alternatively, the antibody or ADC of the invention and any optional anti-cancer agent or other agent can be maintained separately within distinct containers prior to administration to a patient.

The kit can comprise one or multiple containers or receptacles and a label or package insert in, on or associated with the container(s), indicating that the enclosed composition is used for diagnosing or treating the disease condition of choice (e.g., cancer). Suitable containers include, for example, bottles, vials, syringes, infusion bags (i.v. bags), etc. The containers can be formed from a variety of materials such as glass or pharmaceutically compatible plastics. In certain embodiments the container(s) can comprise a sterile access port, for example, the container may be an intravenous solution bag or a vial having a stopper that can be pierced by a hypodermic injection needle.

In some embodiments the kit can contain a means by which to administer the antibody and any optional components to a patient, e.g., one or more needles or syringes (pre-filled or empty), an eye dropper, pipette, or other such like apparatus, from which the formulation may be injected or introduced into the subject or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

### XI. Miscellaneous

Unless otherwise defined herein, scientific and technical terms used in connection with the invention shall have the meanings that are commonly understood by those of ordinary skill in the art. Further, unless otherwise required by context, singular terms shall include pluralities and plural terms shall include the singular. In addition, ranges provided in the specification and appended claims include both end points and all points between the end points. Therefore, a range of 2.0 to 3.0 includes 2.0, 3.0, and all points between 2.0 and 3.0.

Generally, techniques of cell and tissue culture, molecular biology, immunology, microbiology, genetics and chemistry described herein are those well known and commonly used in the art. The nomenclature used herein, in association with such techniques, is also commonly used in the art. The methods and techniques of the invention are generally performed according to conventional methods well known in the art and as described in various references that are cited throughout the present specification unless otherwise indicated.

### XII. References

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding only. No unnecessary limitations are to be understood therefrom. The invention is not limited to the exact details shown and described. Variations obvious to one skilled in the art are included in the invention defined by the claims. Any section headings used herein are for organizational purposes only and are not to be construed as limiting the subject matter described method.

### XIII. Sequences

Appended to the instant application are figures and a sequence listing comprising a number of nucleic acid and amino acid sequences. The following Table 3 provides a summary of the included sequences.

TABLE 3

| SEQ ID NO. | Description |
|---|---|
| 1 | DLL3 isoform 1 protein |
| 2 | DLL3 isoform 2 protein |
| 3 | Epitope SC16.23 protein |
| 4 | Epitope SC16.34 & SC 16.56 protein |
| 5 | Kappa constant region protein |
| 6 | IgGI constant region protein |
| 7-19 | reserved |
| 20 | SC16.3 VL DNA (aligned with encoded protein) |
| 21 | SC16.3 VL protein |
| 22 | SC16.3 VH DNA (aligned with encoded protein) |
| 23 | SC16.3 VH protein |
| 24-387 | Additional murine clones as in SEQ ID NOs: 20-23 |
| 388-407 | Humanized clones as in SEQ ID NOs: 20-23 |
| 408, 409, 410 | hSC16.13 CDRL1, CDRL2, CDRL3 |
| 411, 412, 413 | hSC16.13 CDRH1, CDRH2, CDRH3 |
| 414, 415, 416 | hSC16.15 CDRL1, CDRL2, CDRL3 |
| 417, 418, 419 | hSC16.15 CDRH1, CDRH2, CDRH3 |
| 420, 421, 422 | hSC16.25 CDRL1, CDRL2, CDRL3 |
| 423, 424, 425 | hSC16.25 CDRH1, CDRH2, CDRH3 |
| 426, 427, 428 | hSC16.34 CDRL1, CDRL2, CDRL3 |
| 429, 430, 431 | hSC16.34 CDRH1, CDRH2, CDRH3 |
| 432, 433, 434 | hSC16.56 CDRL1, CDRL2, CDRL3 |
| 435, 436, 437 | hSC16.56 CDRH1, CDRH2, CDRH3 |
| 438-519 | reserved |
| 520 | SC72.2 VL DNA |
| 521 | SC72.2 VL protein |
| 522 | SC72.2 VH DNA |
| 523 | SC72.2 VH protein |
| 524-571 | Additional ASCL1 murine clones as in SEQ ID NOs: |
| | 520-523 |
| 572 | SC72.93 VH DNA |
| 573 | SC72.93 VH protein |

As discussed in Example 2 below, Table 3 above may further be used to designate SEQ ID NOS for exemplary Kabat CDRs delineated in FIGS. 1A and 1B. More particularly FIGS. 1A and 1B denote the three Kabat CDRs of each heavy (CDRH) and light (CDRL) chain variable region sequence and Table 3 above provides for assignment of a SEQ ID designation that may be applied to each CDRL1, CDRL2 and CDRL3 of the light chain and each CDRH1, CDRH2 and CDRH3 of the heavy chain. Using this methodology each unique CDR set forth in FIGS. 1A and 1B (and also in FIGS. 3A and 3B) may be assigned a sequential SEQ ID NO and can be used to provide the derived antibodies of the instant invention.

### XIV. Tumor Listing

PDX tumor cell types are denoted by an abbreviation followed by a number, which indicates the particular tumor cell line. The passage number of the tested sample is indicated by p0-p# appended to the sample designation where p0 is indicative of an unpassaged sample obtained directly from a patient tumor and p# is indicative of the number of times the tumor has been passaged through a mouse prior to testing. As used herein, the abbreviations of the tumor types and subtypes are shown in Table 4 as follows:

TABLE 4

| Tumor Type | Abbreviation | Tumor subtype | Abbreviation |
|---|---|---|---|
| Breast | BR | estrogen receptor positive | BR-ERPR |
| | | and/or progesterone | |
| | | receptor positive | |
| | | ERBB2/Neu positive | BR- ERBB2/ |
| | | | Neu |
| | | HER2 positive | BR-HER2 |
| | | triple-negative | TNBC |
| | | claudin subtype of triple- | TNBC-CLDN |
| | | negative | |
| colorectal | CR | | |
| endometrial | EN | | |
| gastric | GA | diffuse adenocarcinoma | GA-Ad-Dif/ |
| | | | Muc |
| | | intestinal adenocarcinoma | GA-Ad-Int |
| | | stromal tumors | GA-GIST |
| glioblastoma | GB | | |
| head and | HN | | |
| neck | | | |
| kidney | KDY | clear renal cell carcinoma | KDY-CC |
| | | papillary renal cell | KDY-PAP |
| | | carcinoma | |
| | | transitional cell or | KDY-URO |
| | | urothelial carcinoma | |
| | | unknown | KDY-UNK |
| liver | LIV | hepatocellular carcinoma | LIV-HCC |
| | | cholangiocarcinoma | LIV-CHOL |
| lymphoma | LN | | |
| lung | LU | adenocarcinoma | LU-Ad |
| | | carcinoid | LU-CAR |
| | | large cell neuroendocrine | LU-LCC |
| | | non-small cell | NSCLC |
| | | squamous cell | LU-SCC |
| | | small cell | SCLC |
| | | spindle cell | LU-SPC |
| melanoma | MEL | | |
| ovarian | OV | clear cell | OV-CC |
| | | endometroid | OV-END |
| | | mixed subtype | OV-MIX |
| | | malignant mixed | OV-MMMT |
| | | mesodermal | |
| | | mucinous | OV-MUC |
| | | neuroendocrine | OV-NET |
| | | papillary serous | OV-PS |
| | | serous | OV-S |
| | | small cell | OV-SC |
| | | transitional cell carcinoma | OV-TCC |
| pancreatic | PA | acinar cell carcinoma | PA-ACC |
| | | duodenal carcinoma | PA-DC |
| | | mucinous adenocarcinoma | PA-MAD |
| | | neuroendocrine | PA-NET |
| | | adenocarcinoma | PA-PAC |
| | | adenocarcinoma exocrine | PA-PACe |
| | | type | |
| | | ductal adenocarcinoma | PA-PDAC |
| | | ampullary adenocarcinoma | PA-AAC |
| prostate | PR | | |
| skin | SK | melanoma | MEL |
| | | squamous cell carcinomas | SK-SCC |

### EXAMPLES

The invention, thus generally described above, will be understood more readily by reference to the following examples, which are provided by way of illustration and are not intended to be limiting of the instant invention. The examples are not intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1

### Generation of Murine Anti-DLL3 Antibodies

Anti-DLL3 murine antibodies were produced as follows. In a first immunization campaign, three mice (one from each of the following strains: Balb/c, CD-1, FVB) were inoculated with human DLL3-fc protein (hDLL3-Fc) emulsified with an equal volume of TiterMax® or alum adjuvant. The hDLL3-Fc fusion construct was purchased from Adipogen International (Catalog No. AG-40A-0113). An initial immunization was performed with an emulsion of 10 μg hDLL3-Fc per mouse in TiterMax. Mice were then boosted biweekly with 5 μg hDLL3-Fc per mouse in alum adjuvant. The final injection prior to fusion was with 5 μg hDLL3-Fc per mouse in PBS.

In a second immunization campaign six mice (two each of the following strains: Balb/c, CD-1, FVB), were inoculated with human DLL3-His protein (hDLL3-His), emulsified with an equal volume of TiterMax® or alum adjuvant. Recombinant hDLL3-His protein was purified from the supernatants of CHO—S cells engineered to overexpress hDLL3-His. The initial immunization was with an emulsion of 10 μg hDLL3-His per mouse in TiterMax. Mice were then boosted biweekly with 5 μg hDLL3-His per mouse in alum adjuvant. The final injection was with 2×10⁵ HEK-293T cells engineered to overexpress hDLL3.

Solid-phase ELISA assays were used to screen mouse sera for mouse IgG antibodies specific for human DLL3. A positive signal above background was indicative of antibodies specific for DLL3. Briefly, 96 well plates (VWR International, Cat. #610744) were coated with recombinant DLL3-His at 0.5 μg/ml in ELISA coating buffer overnight. After washing with PBS containing 0.02% (v/v) Tween 20, the wells were blocked with 3% (w/v) BSA in PBS, 200 μL/well for 1 hour at room temperature (RT). Mouse serum was titrated (1:100, 1:200, 1:400, and 1:800) and added to the DLL3 coated plates at 50 μL/well and incubated at RT for 1 hour. The plates are washed and then incubated with 50 μL/well HRP-labeled goat anti-mouse IgG diluted 1:10,000 in 3% BSA-PBS or 2% FCS in PBS for 1 hour at RT. Again the plates were washed and 40 μL/well of a TMB substrate solution (Thermo Scientific 34028) was added for 15 minutes at RT. After developing, an equal volume of 2N H₂SO₄ was added to stop substrate development and the plates were analyzed by spectrophotometer at OD 450.

Sera-positive immunized mice were sacrificed and draining lymph nodes (popliteal, inguinal, and medial iliac) were dissected and used as a source for antibody producing cells. Cell suspensions of B cells (approximately 229×10⁶ cells from the hDLL3-Fc immunized mice, and 510×10⁶ cells from the hDLL3-His immunized mice) were fused with non-secreting P3×63Ag8.653 myeloma cells at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum, 10% BM Condimed (Roche Applied Sciences), 1 mM nonessential amino acids, 1 mM HEPES, 100 IU penicillin-streptomycin, and 50 μM 2-mercaptoethanol, and were cultured in four T225 flasks in 100 mL selection medium per flask. The flasks were placed in a humidified 37° C. incubator containing 5% CO₂ and 95% air for six to seven days.

On day six or seven after the fusions the hybridoma library cells were collected from the flasks and plated at one cell per well (using the FACSAria I cell sorter) in 200 μL of supplemented hybridoma selection medium (as described above) into 64 Falcon 96-well plates, and 48 96-well plates for the hDLL3-His immunization campaign. The rest of the library was stored in liquid nitrogen.

The hybridomas were cultured for 10 days and the supernatants were screened for antibodies specific to hDLL3 using flow cytometry performed as follows. 1×10⁵ per well of HEK-293T cells engineered to overexpress human DLL3, mouse DLL3 (pre-stained with dye), or cynomolgus DLL3 (pre-stained with Dylight800) were incubated for 30 minutes with 25 μL hybridoma supernatant. Cells were washed with PBS/2% FCS and then incubated with 25 μL per sample DyeLight 649 labeled goat-anti-mouse IgG, Fc fragment specific secondary diluted 1:300 in PBS/2% FCS. After a 15 minute incubation cells were washed twice with PBS/2% FCS and re-suspended in PBS/2% FCS with DAPI and analyzed by flow cytometry for fluorescence exceeding that of cells stained with an isotype control antibody. Remaining unused hybridoma library cells were frozen in liquid nitrogen for future library testing and screening.

The hDLL3-His immunization campaign yielded approximately 50 murine anti-hDLL3 antibodies and the hDLL3-Fc immunization campaign yielded approximately 90 murine anti-hDLL3 antibodies.

### Example 2

### Sequencing of Anti-DLL3 Antibodies

Based on the foregoing, a number of exemplary distinct monoclonal antibodies that bind immobilized human DLL3 or h293-hDLL3 cells with apparently high affinity were selected for sequencing and further analysis. Sequence analysis of the light chain variable regions and heavy chain variable regions from selected monoclonal antibodies generated in Example 1 confirmed that many had novel complementarity determining regions and often displayed novel VDJ arrangements.

Initially selected hybridoma cells expressing the desired antibodies were lysed in Trizol® reagent (Trizol® Plus RNA Purification System, Life Technologies) to prepare the RNA encoding the antibodies. Between 10⁴ and 10⁵ cells were re-suspended in 1 mL Trizol and shaken vigorously after addition of 200 μL chloroform. Samples were then centrifuged at 4° C. for 10 minutes and the aqueous phase was transferred to a fresh microfuge tube and an equal volume of 70% ethanol was added. The sample was loaded on an RNeasy Mini spin column, placed in a 2 mL collection tube and processed according to the manufacturer's instructions. Total RNA was extracted by elution, directly to the spin column membrane with 100 μL RNase-free water. The quality of the RNA preparations was determined by fractionating 3 μL in a 1% agarose gel before being stored at −80° C. until used.

The variable region of the Ig heavy chain of each hybridoma was amplified using a 5′ primer mix comprising 32 mouse specific leader sequence primers designed to target the complete mouse VH repertoire in combination with a 3′ mouse Cγ primer specific for all mouse Ig isotypes. Similarly, a primer mix containing thirty two 5′ Vκ leader sequences designed to amplify each of the Vκ mouse families was used in combination with a single reverse primer specific to the mouse kappa constant region in order to amplify and sequence the kappa light chain. For antibodies containing a lambda light chain, amplification was performed using three 5′ VL leader sequences in combination with one reverse primer specific to the mouse lambda constant region. The VH and VL transcripts were amplified from 100 ng total RNA using the Qiagen One Step RT-PCR kit as follows. A total of eight RT-PCR reactions were run for each hybridoma, four for the Vκ light chain and four for the Vγ heavy chain. PCR reaction mixtures included 3 μL of RNA, 0.5 μL of 100 μM of either heavy chain or kappa light chain primers (custom synthesized by Integrated Data Technologies), 5 μL of 5×RT-PCR buffer, 1 μL dNTPs, 1 μL of enzyme mix containing reverse transcriptase and DNA polymerase, and 0.4 μL of ribonuclease inhibitor RNasin (1 unit). The thermal cycler program was RT step 50° C. for 30 minutes, 95° C. for 15 minutes followed by 30 cycles of (95° C. for 30 seconds, 48° C. for 30 seconds, 72° C. for 1 minute). There was then a final incubation at 72° C. for 10 minutes.

The extracted PCR products were sequenced using the same specific variable region primers as described above for the amplification of the variable regions. To prepare the PCR products for direct DNA sequencing, they were purified using the QIAquick™ PCR Purification Kit (Qiagen) according to the manufacturer's protocol. The DNA was eluted from the spin column using 50 μL of sterile water and then sequenced directly from both strands (MCLAB).

Selected nucleotide sequences were analyzed using the IMGT sequence analysis tool (http://www.imgt.org/IMGTmedical/sequence_analysis.html) to identify germline V, D and J gene members with the highest sequence homology. These derived sequences were compared to known germline DNA sequences of the Ig V- and J-regions by alignment of VH and VL genes to the mouse germline database using a proprietary antibody sequence database.

FIG. 1A depicts the contiguous amino acid sequences of numerous novel murine light chain variable regions from anti-DLL3 antibodies and exemplary humanized light chain variable regions derived from the variable light chains of representative murine anti-DLL3 antibodies (as per Example 3 below). FIG. 1B depicts the contiguous amino acid sequences of novel murine heavy chain variable regions from the same anti-DLL3 antibodies and humanized heavy chain variable regions derived from the same murine antibodies providing the humanized light chains (as per Example 3 below). Murine light and heavy chain variable region amino acid sequences are provided in SEQ ID NOS: 21-387, odd numbers while humanized light and heavy chain variable region amino acid sequences are provided in SEQ ID NOS: 389-407, odd numbers.

Thus, taken together FIGS. 1A and 1B provide the annotated sequences of numerous murine anti-DLL3 binding or targeting domains, termed SC16.3, SC16.4, SC16.5, SC16.7, SC16.8, SC16.10, SC16.11, SC16.13, SC16.15, SC16.18, SC16.19, SC16.20, SC16.21, SC16.22, SC16.23, SC16.25, SC16.26, SC16.29, SC16.30, SC16.31, SC16.34, SC16.35, SC16.36, SC16.38, SC16.41, SC16.42, SC16.45, SC16.47, SC16.49, SC16.50, SC16.52, SC16.55, SC16.56, SC16.57, SC16.58, SC16.61, SC16.62, SC16.63, SC16.65, SC16.67, SC16.68, SC16.72, SC16.73, SC16.78, SC16.79, SC16.80, SC16.81, SC16.84, SC16.88, SC16.101, SC16.103, SC16.104, SC16.105, SC16.106, SC16.107, SC16.108, SC16.109, SC16.110, SC16.111, SC16.113, SC16.114, SC16.115, SC16.116, SC16.117, SC16.118, SC16.120, SC16.121, SC16.122, SC16.123, SC16.124, SC16.125, SC16.126, SC16.129, SC16.130, SC16.131, SC16.132, SC16.133, SC16.134, SC16.135, SC16.136, SC16.137, SC16.138, SC16.139, SC16.140, SC16.141, SC16.142, SC16.143, SC16.144, SC16.147, SC16.148, SC16.149 and SC16.150 and humanized antibodies, termed hSC16.13, hSC16.15, hSC16.25, hSC16.34 and hSC16.56.

In particular aspects of the invention the ADC binding domain binds specifically to hDLL3 and comprises or competes for binding with an antibody comprising: a light chain variable region (VL) of SEQ ID NO: 21 and a heavy chain variable region (VH) of SEQ ID NO: 23; or a VL of SEQ ID NO: 25 and a VH of SEQ ID NO: 27; or a VL of SEQ ID NO: 29 and a VH of SEQ ID NO: 31; or a VL of SEQ ID NO: 33 and a VH of SEQ ID NO: 35; or a VL of SEQ ID NO: 37 and a VH of SEQ ID NO: 39; or a VL of SEQ ID NO: 41 and a VH of SEQ ID NO: 43; or a VL of SEQ ID NO: 45 and a VH of SEQ ID NO: 47; or a VL of SEQ ID NO: 49 and a VH of SEQ ID NO: 51; or a VL of SEQ ID NO: 53 and a VH of SEQ ID NO: 55; or a VL of SEQ ID NO: 57 and a VH of SEQ ID NO: 59; or a VL of SEQ ID NO: 61 and a VH of SEQ ID NO: 63; or a VL of SEQ ID NO: 65 and a VH of SEQ ID NO: 67; or a VL of SEQ ID NO: 69 and a VH of SEQ ID NO: 71; or a VL of SEQ ID NO: 73 and a VH of SEQ ID NO: 75; or a VL of SEQ ID NO: 77 and a VH of SEQ ID NO: 79; or a VL of SEQ ID NO: 81 and a VH of SEQ ID NO: 83; or a VL of SEQ ID NO: 85 and a VH of SEQ ID NO: 87; or a VL of SEQ ID NO: 89 and a VH of SEQ ID NO: 91; or a VL of SEQ ID NO: 93 and a VH of SEQ ID NO: 95; or a VL of SEQ ID NO: 97 and a VH of SEQ ID NO: 99; or a VL of SEQ ID NO: 101 and a VH of SEQ ID NO: 103; or a VL of SEQ ID NO: 105 and a VH of SEQ ID NO: 107; or a VL of SEQ ID NO: 109 and a VH of SEQ ID NO: 111; or a VL of SEQ ID NO: 113 and a VH of SEQ ID NO: 115; or a VL of SEQ ID NO: 117 and a VH of SEQ ID NO: 119; or a VL of SEQ ID NO: 121 and a VH of SEQ ID NO: 123; or a VL of SEQ ID NO: 125 and a VH of SEQ ID NO: 127; or a VL of SEQ ID NO: 129 and a VH of SEQ ID NO: 131; or a VL of SEQ ID NO: 133 and a VH of SEQ ID NO: 135; or a VL of SEQ ID NO: 137 and a VH of SEQ ID NO: 139; or a VL of SEQ ID NO: 141 and a VH of SEQ ID NO: 143; or a VL of SEQ ID NO: 145 and a VH of SEQ ID NO: 147; or a VL of SEQ ID NO: 149 and a VH of SEQ ID NO: 151; or a VL of SEQ ID NO: 153 and a VH of SEQ ID NO: 155; or a VL of SEQ ID NO: 157 and a VH of SEQ ID NO: 159; or a VL of SEQ ID NO: 161 and a VH of SEQ ID NO: 163; or a VL of SEQ ID NO: 165 and a VH of SEQ ID NO: 167; or a VL of SEQ ID NO: 169 and a VH of SEQ ID NO: 171; or a VL of SEQ ID NO: 173 and a VH of SEQ ID NO: 175; or a VL of SEQ ID NO: 177 and a VH of SEQ ID NO: 179; or a VL of SEQ ID NO: 181 and a VH of SEQ ID NO: 183; or a VL of SEQ ID NO: 185 and a VH of SEQ ID NO: 187; or a VL of SEQ ID NO: 189 and a VH of SEQ ID NO: 191; or a VL of SEQ ID NO: 193 and a VH of SEQ ID NO: 195; or a VL of SEQ ID NO: 197 and a VH of SEQ ID NO: 199; or a VL of SEQ ID NO: 201 and a VH of SEQ ID NO: 203; or a VL of SEQ ID NO: 205 and a VH of SEQ ID NO: 207; or a VL of SEQ ID NO: 209 and a VH of SEQ ID NO: 211; or a VL of SEQ ID NO: 213 and a VH of SEQ ID NO: 215; or a VL of SEQ ID NO: 217 and a VH of SEQ ID NO: 219; or a VL of SEQ ID NO: 221 and a VH of SEQ ID NO: 223; or a VL of SEQ ID NO: 225 and a VH of SEQ ID NO: 227; or a VL of SEQ ID NO: 229 and a VH of SEQ ID NO: 231; or a VL of SEQ ID NO: 233 and a VH of SEQ ID NO: 235; or a VL of SEQ ID NO: 237 and a VH of SEQ ID NO: 239; or a VL of SEQ ID NO: 241 and a VH of SEQ ID NO: 243; or a VL of SEQ ID NO: 245 and a VH of SEQ ID NO: 247; or a VL of SEQ ID NO: 249 and a VH of SEQ ID NO: 251; or a VL of SEQ ID NO: 253 and a VH of SEQ ID NO: 255; or a VL of SEQ ID NO: 257 and a VH of SEQ ID NO: 259; or a VL of SEQ ID NO: 261 and a VH of SEQ ID NO: 263; or a VL of SEQ ID NO: 265 and a VH of SEQ ID NO: 267; or a VL of SEQ ID NO: 269 and a VH of SEQ ID NO: 271; or a VL of SEQ ID NO: 273 and a VH of SEQ ID NO: 275; or a VL of SEQ ID NO: 277 and a VH of SEQ ID NO: 279; or a VL of SEQ ID NO: 281 and a VH of SEQ ID NO: 283; or a VL of SEQ ID NO: 285 and a VH of SEQ ID NO: 287; or a VL of SEQ ID NO: 289 and a VH of SEQ ID NO: 291; or a VL of SEQ ID NO: 293 and a VH of SEQ ID NO: 295; or a VL of SEQ ID NO: 297 and a VH of SEQ ID NO: 299; or a VL of SEQ ID NO: 301 and a VH of SEQ ID NO: 303; or a VL of SEQ ID NO: 305 and a VH of SEQ ID NO: 307; or a VL of SEQ ID NO: 309 and a VH of SEQ ID NO: 311; or a VL of SEQ ID NO: 313 and a VH of SEQ ID NO: 315; or a VL of SEQ ID NO: 317 and a VH of SEQ ID NO: 319; or a VL of SEQ ID NO: 321 and a VH of SEQ ID NO: 323; or a VL of SEQ ID NO: 325 and a VH of SEQ ID NO: 327; or a VL of SEQ ID NO: 329 and a VH of SEQ ID NO: 331; or a VL of SEQ ID NO: 333 and a VH of SEQ ID NO: 335; or a VL of SEQ ID NO: 337 and a VH of SEQ ID NO: 339; or a VL of SEQ ID NO: 341 and a VH of SEQ ID NO: 343; or a VL of SEQ ID NO: 345 and a VH of SEQ ID NO: 347; or a VL of SEQ ID NO: 349 and a VH of SEQ ID NO: 351; or a VL of SEQ ID NO: 353 and a VH of SEQ ID NO: 355; or a VL of SEQ ID NO: 357 and a VH of SEQ ID NO: 359; or a VL of SEQ ID NO: 361 and a VH of SEQ ID NO: 363; or a VL of SEQ ID NO: 365 and a VH of SEQ ID NO: 367; or a VL of SEQ ID NO: 369 and a VH of SEQ ID NO: 371; or a VL of SEQ ID NO: 373 and a VH of SEQ ID NO: 375; or a VL of SEQ ID NO: 377 and a VH of SEQ ID NO: 379; or a VL of SEQ ID NO: 381 and a VH of SEQ ID NO: 383; or a VL of SEQ ID NO: 385 and a VH of SEQ ID NO: 387; or a VL of SEQ ID NO: 389 and a VH of SEQ ID NO: 391; or a VL of SEQ ID NO: 393 and a VH of SEQ ID NO: 395; or a VL of SEQ ID NO: 397 and a VH of SEQ ID NO: 399; or a VL of SEQ ID NO: 401 and a VH of SEQ ID NO: 403; or a VL of SEQ ID NO: 405 and a VH of SEQ ID NO: 407.

For the purposes of the instant application the SEQ ID NOS of each particular antibody are sequential odd numbers. Thus the monoclonal anti-DLL3 antibody, SC16.3, comprises amino acid SEQ ID NOS: 21 and 23 for the light and heavy chain variable regions respectively; SC16.4 comprises SEQ ID NOS: 25 and 27; SC16.5 comprises SEQ ID NOS: 29 and 31, and so on. The corresponding nucleic acid sequence for each antibody amino acid sequence is included in the appended sequence listing and has the SEQ ID NO immediately preceding the corresponding amino acid SEQ ID NO. Thus, for example, the SEQ ID NOS of the VL and VH of the SC16.3 antibody are 21 and 23, respectively, and the SEQ ID NOS of the nucleic acid sequences encoding the VL and VH of the SC16.3 antibody are SEQ ID NOS: 20 and 22, respectively. The CDRs are defined as per Kabat et al. (supra) using a proprietary version of the Abysis database.

### Example 3

### Generation of Chimeric and Humanized Anti-DLL3 Antibodies

To provide a benchmark for humanized binding domains compatible with the instant invention chimeric anti-DLL3 antibodies were generated using art-recognized techniques as follows. Total RNA was extracted from the hybridomas and amplified as set forth in Example 1. Data regarding V, D and J gene segments of the VH and VL chains of the murine antibodies were obtained from the derived nucleic acid sequences. Primer sets specific to the leader sequence of the VH and VL chain of the antibody were designed using the following restriction sites: AgeI and XhoI for the VH fragments, and XmaI and DraIII for the VL fragments. PCR products were purified with a QIAquick PCR purification kit (Qiagen), followed by digestion with restriction enzymes AgeI and XhoI for the V_{H} fragments and XmaI and DraIII for the VL fragments. The VL and VH digested PCR products were purified and ligated into kappa CL (SEQ ID NO: 5) human light chain constant region expression vector or IgG1 (SEQ ID NO: 6) human heavy chain constant region expression vector, respectively.

Ligation reactions were performed in a total volume of 10 μL with 200U T4-DNA Ligase (New England Biolabs), 7.5 μL of digested and purified gene-specific PCR product and 25 ng linearized vector DNA. Competent E. coli DH10B bacteria (Life Technologies) were transformed via heat shock at 42° C. with 3 μL ligation product and plated onto plates with ampicillin at a concentration of 100 μg/mL. Following purification and digestion of the amplified ligation products, the V_{H} fragment was cloned into the AgeI-XhoI restriction sites of the pEE6.4HuIgG1 expression vector (Lonza) and the VL fragment was cloned into the XmaI-DraIII restriction sites of the pEE12.4Hu-Kappa expression vector (Lonza).

Chimeric antibodies were expressed by co-transfection of HEK-293T cells with pEE6.4HuIgG1 and pEE12.4Hu-Kappa expression vectors. Prior to transfection the HEK-293T cells were cultured in 150 mm plates under standard conditions in Dulbecco's Modified Eagle's Medium (DMEM) supplemented with 10% heat inactivated FCS, 100 μg/mL streptomycin and 100 U/mL penicillin G. For transient transfections cells were grown to 80% confluency. 12.5 μg each of pEE6.4HuIgG1 and pEE12.4Hu-Kappa vector DNA were added to 50 μL HEK-293T transfection reagent in 1.5 mL Opti-MEM. The mix was incubated for 30 minutes at room temperature and plated. Supernatants were harvested three to six days after transfection. Culture supernatants containing recombinant chimeric antibodies were cleared from cell debris by centrifugation at 800×g for 10 minutes and stored at 4° C. Recombinant chimeric antibodies were purified by Protein A affinity chromatography.

The same murine anti-DLL3 antibodies (e.g. SC16.13, SC16.15, SC16.25, SC16.34 and SC16.56) were also used to derive CDR-grafted or humanized binding domains. The murine antibodies were humanized using a proprietary computer-aided CDR-grafting method (Abysis Database, UCL Business) and standard molecular engineering techniques as follows. Human framework regions of the variable regions were designed based on the highest homology between the framework sequences and CDR canonical structures of human germline antibody sequences and the framework sequences and CDRs of the relevant mouse antibodies. For the purpose of the analysis the assignment of amino acids to each of the CDR domains was done in accordance with Kabat et al. Once the variable regions were selected, they were generated from synthetic gene segments (Integrated DNA Technologies). Humanized antibodies were cloned and expressed using the molecular methods described above for chimeric antibodies.

The genetic composition for the selected human acceptor variable regions are shown in TABLE 5 immediately below for each of the humanized antibodies. The sequences depicted in TABLE 5 correspond to the contiguous variable region sequences set forth in SEQ ID NOS: 389 and 391 (hSC16.13), SEQ ID NOS: 393 and 395 (hSC16.15), SEQ ID NOS: 397 and 399 (hSC16.25), SEQ ID NOS: 401 and 403 (hSC16.34) and SEQ ID NOS: 405 and 407 (hSC16.56). TABLE 5 shows that no framework changes or back mutations were necessary to maintain the favorable binding properties of the selected antibodies.

TABLE 5

| mAb | human VH | human DH | human JH | FW changes | human VK | human JK | FW changes |
|---|---|---|---|---|---|---|---|
| hSC16.13 | IGHV2-5*01 | IGHD1-1 | JH6 | None | IGKV1-39*01 | JK1 | None |
| hSC16.15 | IGHV1-46*01 | IGHD2-2 | JH4 | None | IGKV1-13*02 | JK4 | None |
| hSC16.25 | IGHV2-5*01 | IGHD3-16 | JH6 | None | IGKV6-21*01 | JK2 | None |
| hSC16.34 | IGHV1-3*02 | IGHD3-22 | JH4 | None | IGKV1-27*01 | JK1 | None |
| hSC16.56 | IGHV1-18*01 | IGHD2-21 | JH4 | None | IGKV3-15*01 | JK2 | None |

Although no residues were altered in the framework regions, in one of the humanized clones (hSC16.13) mutations were introduced into heavy chain CDR2 to address stability concerns. The binding affinity of the antibody with the modified CDR was checked to ensure that it was equivalent to either the corresponding chimeric or murine antibody.

Following humanization the resulting VL and VH chain amino acid sequences were analyzed to determine their homology with regard to the murine donor and human acceptor light and heavy chain variable regions. The results shown in TABLE 6, immediately below, reveal that the humanized constructs consistently exhibited a higher homology with respect to the human acceptor sequences than with the murine donor sequences. The murine heavy and light chain variable regions show a similar overall percentage homology to a closest match of human germline genes (85%-93%) compared with the homology of the humanized antibodies and the donor hybridoma protein sequences (74%-83%).

TABLE 6

| | Homology to Human | Homology to Murine Parent |
|---|---|---|
| mAb | (CDR acceptor) | (CDR donor) |
| hSC16.13 HC | 93% | 81% |
| hSC16.13 LC | 87% | 77% |
| hSC16.15 HC | 85% | 83% |
| hSC16.15 LC | 85% | 83% |
| hSC16.25 HC | 91% | 83% |
| hSC16.25 LC | 85% | 79% |
| hSC16.34 HC | 87% | 79% |
| hSC16.34 LC | 85% | 81% |
| hSC16.56 HC | 87% | 74% |
| hSC16.56 LC | 87% | 76% |

As with the chimeric antibodies the humanized VL and VH digested PCR products were purified and ligated into kappa CL (SEQ ID NO: 5) human light chain constant region expression vector or IgG1 (SEQ ID NO: 6) human heavy chain constant region expression vector, respectively. Following expression each of the derived humanized constructs were analyzed using surface plasmon resonance, to determine if the CDR grafting process had appreciably altered their apparent affinity for DLL3 protein. The humanized constructs were compared with chimeric antibodies comprising the murine parent (or donor) heavy and light chain variable domains and a human constant region substantially equivalent to that used in the humanized constructs. The humanized anti-DLL3 antibodies exhibited binding characteristics roughly comparable to those shown by the chimeric parent antibodies (data not shown).

### Example 4

### Generation of Site-Specific Antibodies

An engineered human IgG1/kappa anti-DLL3 site-specific antibody was constructed comprising a native light chain (LC) constant region and heavy chain (HC) constant region, wherein cysteine 220 (C220) in the upper hinge region of the HC, which forms an interchain disulfide bond with cysteine 214 (C214) in the LC, was substituted with serine (C220S). When assembled the HCs and LCs form an antibody comprising two free cysteines that are suitable for conjugation to a therapeutic agent. Unless otherwise noted, all numbering of constant region residues is in accordance with the EU numbering scheme as set forth in Kabat et al.

The engineered antibodies were generated as follows. An expression vector encoding the full-length humanized anti-DLL3 antibody hSC16.56 was used as a template for PCR amplification and site directed mutagenesis. Site directed mutagenesis was performed using the Quick-Change® system (Agilent Technologies) according to the manufacturer's instructions.

The vector encoding the mutant C220S heavy chain of hSC16.56 was co-transfected with the native full-length kappa light chain in CHO—S cells and expressed using a mammalian transient expression system. The engineered anti-DLL3 site-specific antibody containing the C220S mutant was termed hSC16.56ss1. Once expressed the engineered anti-DLL3 antibodies were characterized by SDS-PAGE to confirm that the correct mutants had been generated. SDS-PAGE was conducted on a pre-cast 10% Tris-Glycine mini gel from life technologies in the presence and absence of a reducing agent such as DTT (dithiothreitol). Following electrophoresis, the gels were stained with a colloidal Coomassie solution. Under reducing conditions, two bands corresponding to the free LCs and free HCs, were observed (data not shown). This pattern is typical of IgG molecules in reducing conditions. Under non-reducing conditions, the band patterns were different from native IgG molecules, indicative of the absence of a disulfide bond between the HC and LC. A band around 98 kD corresponding to the HC—HC dimer was observed. In addition, a faint band corresponding to the free LC and a predominant band around 48 kD that corresponded to a LC-LC dimer was observed. The formation of some amount of LC-LC species is expected due to the free cysteines on the C-terminus of each LC.

### Example 5

### Domain and Epitope Mapping of Anti-DLL3 Antibodies

In order to characterize and position the epitopes that the disclosed anti-DLL3 antibodies bind to, domain-level epitope mapping was performed using a modification of the protocol described by Cochran et al., 2004 (supra). Individual domains of DLL3 comprising specific amino acid sequences were expressed on the surface of yeast, and binding by each anti-DLL3 antibody was determined through flow cytometry.

Yeast display plasmid constructs were created for the expression of the following constructs: DLL3 extracellular domain (amino acids 27-466); DLL1-DLL3 chimera, which consists of the N-terminal region and DSL domain of DLL1 (amino acids 22-225) fused to EGF-like domains 1 through 6 of DLL3 (amino acids 220-466); DLL3-DLL1 chimera, which consists of the N-terminal region and DSL domain of DLL3 (amino acids 27-214) fused to EGF-like domains 1 through 8 of DLL1 (amino acids 222-518); EGF1 (amino acids 215-249); EGF2 (amino acids 274-310); EGF1 and EGF2 (amino acids 215-310); EGF3 (amino acids 312-351); EGF4 (amino acids 353-389); EGF5 (amino acids 391-427); and EGF6 (amino acids 429-465). For domain information see generally UniProtKB/Swiss-Prot database entry Q9NYJ7. Note that the amino acid numbering references an unprocessed DLL3 protein with a leader sequence included in the sequence set forth in SEQ ID NO. 1.) For analysis of the N-terminal region or the EGF domains as a whole, chimeras with the family member DLL1 (DLL1-DLL3 and DLL3-DLL1) were used as opposed to fragments to minimize potential problems with protein folding. Domain-mapped antibodies had previously been shown not to cross-react with DLL1 indicating that any binding to these constructs was occurring through association with the DLL3 portion of the construct. These plasmids were transformed into yeast, which were then grown and induced as described in Cochran et al.

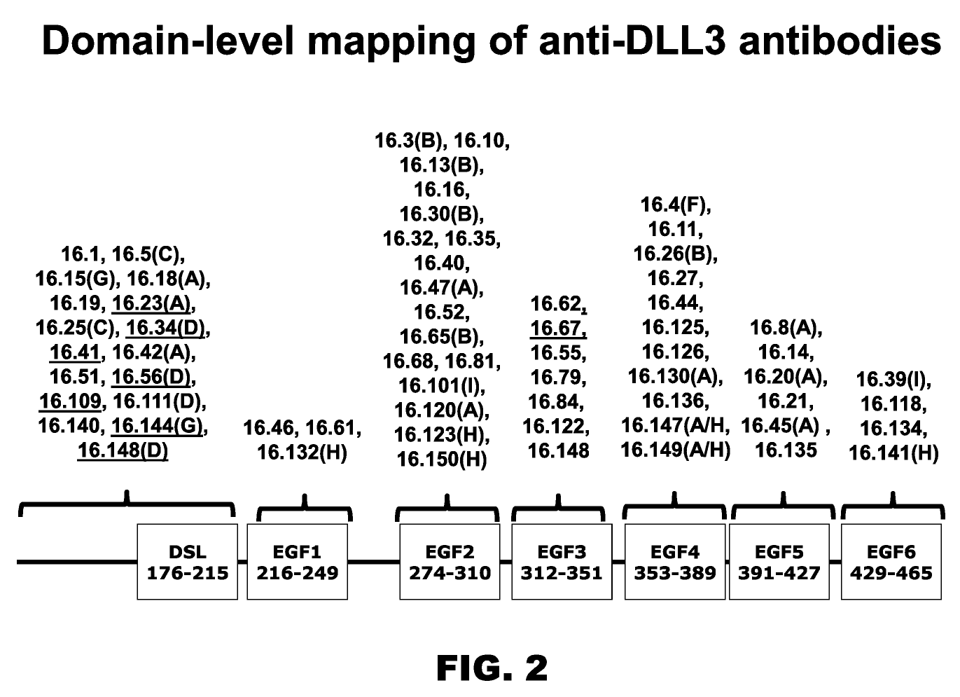

To test for binding to a particular construct, 200,000 induced yeast cells expressing the desired construct were washed twice in PBS+1 mg/mL BSA (PBSA), and incubated in 50 μL of PBSA with biotinylated anti-HA clone 3F10 (Roche Diagnostics) at 0.1 μg/mL and either 50 nM purified antibody or 1:2 dilution of unpurified supernatant from hybridomas cultured for 7 days. Cells were incubated for 90 minutes on ice, followed by two washes in PBSA. Cells were then incubated in 50 μL PBSA with the appropriate secondary antibodies: for murine antibodies, Alexa 488 conjugated streptavidin, and Alexa 647 conjugated goat anti mouse (Life Technologies) were added at 1 μg/mL each; and for humanized or chimeric antibodies, Alexa 647 conjugated streptavidin (Life Technologies) and R-phycoerythrin conjugated goat anti human (Jackson Immunoresearch) were added at 1 μg/mL each. After a twenty minute incubation on ice, cells were washed twice with PBSA and analyzed on a FACS Canto II. Antibodies that bound to DLL3-DLL1 chimera were designated as binding to the N-terminal region+DSL. Antibodies that bound specifically to an epitope present on a particular EGF-like domain were designated as binding to its respective domain (FIG. 2.)

In order to classify an epitope as conformational (e.g., discontinuous) or linear, yeast displaying the DLL3 ECD was heat treated for 30 minutes at 80° C. to denature the DLL3 ECD, and then washed twice in ice-cold PBSA. The ability of anti-DLL3 antibodies to bind the denatured yeast was tested by FACS using the same staining protocol as described above. Antibodies that bound to both the denatured and native yeast were classified as binding to a linear epitope, whereas antibodies that bound native yeast but not denatured yeast were classified as conformationally specific.

A schematic summary of the domain-level epitope mapping data of the antibodies tested is presented in FIG. 2, with antibodies binding a linear epitope underlined and, where determined, the corresponding bin noted in parenthesis. A review of FIG. 2 shows that the majority of anti-DLL3 antibodies tended to map to epitopes found either in the N-terminal/DSL region of DLL3 or EGF2. FIG. 2 presents similar data in a tabular form on bin determination and domain mapping for various anti-DLL3 antibodies.

Fine epitope mapping was further performed on selected antibodies using one of two methods. The first method employed the Ph.D.-12 phage display peptide library kit (New England Biolabs) which was used in accordance with the manufacturer's instructions. The antibody for epitope mapping was coated overnight at 50 μg/mL in 3 mL 0.1 M sodium bicarbonate solution, pH 8, onto a Nunc MaxiSorp tube (Nunc). The tube was blocked with 3% BSA solution in bicarbonate solution. Then, 101 input phage in PBS+0.1% Tween-20 was allowed to bind, followed by ten consecutive washes with 0.1% Tween-20 to wash away non-binding phage. Remaining phage were eluted with 1 mL 0.2 M glycine for 10 minutes at room temperature with gentle agitation, followed by neutralization with 150 μL 1M Tris-HCl pH 9. Eluted phage were amplified and panned again with 10¹¹ input phage, using 0.5% Tween-20 during the wash steps to increase selection stringency. DNA from 24 plaques of the eluted phage from the second round was isolated using the Qiaprep M13 Spin kit (Qiagen) and sequenced. Binding of clonal phage was confirmed using an ELISA assay, where the mapped antibody or a control antibody was coated onto an ELISA plate, blocked, and exposed to each phage clone. Phage binding was detected using horseradish peroxidase conjugated anti-M13 antibody (GE Healthcare), and the 1-Step Turbo TMB ELISA solution (Pierce). Phage peptide sequences from specifically binding phage were aligned using Vector NTI (Life Technologies) against the antigen ECD peptide sequence to determine the epitope of binding.

Alternatively, a yeast display method (Chao et al., 2007, PMID: 17406305) was used to map the epitopes of selected antibodies. Libraries of DLL3 ECD mutants were generated with error prone PCR using nucleotide analogues 8-oxo-2′deoxyguanosine-5′-triphosphate and 2′-deoxy-p-nucleoside-5′triphosphate (TriLink Bio) for a target mutagenesis rate of one amino acid mutation per clone. These were transformed into a yeast display format. Using the technique described above for domain-level mapping, the library was stained for HA and antibody binding at 50 nM. Using a FACS Aria (BD), clones that exhibited a loss of binding compared to wild type DLL3 ECD were sorted. These clones were re-grown, and subjected to another round of FACS sorting for loss of binding to the target antibody. Using the Zymoprep Yeast Plasmid Miniprep kit (Zymo Research), individual ECD clones were isolated and sequenced. Where necessary, mutations were reformatted as single-mutant ECD clones using the Quikchange site directed mutagenesis kit (Agilent).

Individual ECD clones were next screened to determine whether loss of binding was due to a mutation in the epitope, or a mutation that caused misfolding. Mutations that involved cysteine, proline, and stop codons were automatically discarded due to the high likelihood of a misfolding mutation. Remaining ECD clones were then screened for binding to a non-competing, conformationally specific antibody. ECD clones that lost binding to non-competing, conformationally specific antibodies were concluded to contain misfolding mutations, whereas ECD clones that retained equivalent binding to wild type DLL3 ECD were concluded to be properly folded. Mutations in the ECD clones in the latter group were concluded to be in the epitope.

A summary of selected antibodies with their derived epitopes comprising amino acid residues that are involved in antibody binding are listed in TABLE 7 below. Antibodies SC16.34 and SC16.56 interact with common amino acid residues which is consistent with the binning information and domain mapping results shown in FIG. 2. Moreover, SC16.23 was found to interact with a distinct contiguous epitope and was found not to bin with SC16.34 or SC16.56. Note that for the purposes of the appended sequence listing SEQ ID NO: 4 comprises a placeholder amino acid at position 204.

TABLE 7

| Antibody Clone | Epitope | SEQ ID NO: |
|---|---|---|
| SC16.23 | Q93, P94, G95, A96, P97 | 3 |
| SC16.34 | G203, R205, P206 | 4 |
| SC16.56 | G203, R205, P206 | 4 |

### Example 6

### Conjugation of Anti-DLL3 Antibodies to Pyrrolobenzodiazepines (PBDs)

A humanized anti-DLL3 antibody (hSC16.56) and a humanized site-specific anti-DLL3 antibody (hSC16.56ss1) were conjugated to a pyrrolobenzodiazepine (DL1, PBD1) via a terminal maleimido moiety with a free sulfhydryl group to create the ADCs termed hSC16.56PBD1 and hSC16.56ss1PBD1. hSC16.56PBD1 (i.e., SC16LD6.5) and hSC16.56ss1PBD1 were made under GMP conditions as it was intended for use in clinical trials. The humanized DLL3 (hSC16.56) antibody drug conjugates (ADCs) were prepared in two distinct stages; a reduction step and a conjugation step to conjugate PBD1 to hSC16.56. The ADCs were then processed through Cation Exchange (CEX) Chromatography, followed by diafiltration and formulation steps to produce the drug substance. The process is described in detail below.

The antibodies were adjusted to pH 7.5 with the addition of 200 mM Tris Base, 32 mM EDTA pH 8.5. Cysteine bonds of the pH adjusted DLL3 antibodies were then partially reduced with a pre-determined molar addition of mol tris(2-carboxyethyl)-phosphine (TCEP) per mol antibody for 90 min. at 20° C. The resulting partially reduced preparations were then conjugated to PBD1 (as set forth above) via a maleimide linker for a minimum of 30 minutes at 20° C. The reaction was then quenched with the addition of excess N-acetyl cysteine (NAC) compared to linker-drug using a 10 mM stock solution prepared in water. After a minimum quench time of 20 minutes the pH was adjusted to 5.5 with the addition of 0.5 M acetic acid. Preparations of the ADCs were then processed through Cation Exchange (CEX) Chromatography in a bind and elute mode with a step elution to remove aggregates formed during the conjugation step. CEX purified ADCs were then buffer exchanged into diafiltration buffer by diafiltration using a 30 kDa membrane. The dialfiltered anti-DLL3 ADC was then formulated with sucrose and polysorbate-20 to the target final concentration to produce drug substance.

The site specific humanized anti-DLL3 (hSC16.56ss1) ADCs were conjugated using a modified partial reduction process. In this respect the desired product is an ADC that is maximally conjugated on the unpaired cysteine (C214) on each LC constant region and that minimizes ADCs having a drug to antibody ratio (DAR) which is greater than 2 (DAR>2) while maximizing ADCs having a DAR of 2 (DAR=2). In order to further improve the specificity of the conjugation, the antibodies were selectively reduced using a process comprising a stabilizing agent (e.g. L-arginine) and a mild reducing agent (e.g. glutathione) prior to conjugation with the linker-drug. The ADCs were then processed through preparative Hydrophobic Interaction Chromatography (HIC), followed by a diafiltration and formulation step to produce the drug substance. The process is described in detail below.

The site-specific antibody constructs were partially reduced in a buffer containing 1M L-arginine/5 mM EDTA with a pre-determined concentration of reduced glutathione (GSH), pH 8.0 for a minimum of two hours at room temperature. All preparations were then buffer exchanged into a 20 mM Tris/3.2 mM EDTA, pH 7.0 buffer using a 30 kDa membrane to remove the reducing buffer. The resulting partially reduced preparations were then conjugated to PBD1 (as set forth above) via a maleimide linker for a minimum of 30 mins. at 20° C. The reaction was then quenched with the addition of excess NAC compared to linker-drug using a 10 mM stock solution prepared in water. After a minimum quench time of 20 minutes the pH was adjusted to 6.0 with the addition of 0.5 M acetic acid. The pH adjusted ADCs were then processed through preparative Hydropobic Interaction Chromatography (HIC) (Butyl Sepharose FF) in a bind and elute mode with a step elution to further purify the DAR 2 species. The purified ADCs were then buffer exchanged into diafiltration buffer by diafiltration using a 30 kDa membrane. The dialfiltered anti-DLL3 ADC was then formulated with sucrose and polysorbate-20 to the target final concentration to produce the site-specific drug substance.

### Example 7

### Cloning and Expression of Recombinant ASCL1 Fusion Proteins and Engineering of Cell Lines Overexpressing ASCL1 Protein

DNA Fragments Encoding Human ASCL1 and ASCL2 Protein.

To generate all cellular materials required in the present invention pertaining to the human ASCL1 (hASCL1) protein (GenBank accession NP_004307), a synthetic DNA fragment that encoded the open reading frame of the ASCL1 mRNA (GenBank accession NM_004316, nts 572-1282) was subcloned into a CMV driven expression vector in-frame and downstream of an IgK signal peptide sequence and upstream of either a 9-Histidine tag or a human IgG2 Fc cDNA, using standard molecular techniques. These CMV-driven expression vectors permit high level transient expression in HEK-293T and/or CHO—S cells. Suspension or adherent cultures of HEK-293T cells, or suspension CHO—S cells were transfected with these expression constructs, using polyethylenimine polymer as the transfecting reagent. Three to five days after transfection, the recombinant His-tagged or Fc-tagged proteins were purified from clarified cell-supernatants using an AKTA explorer and either Nickel-EDTA (Qiagen) or MabSelect SuRe™ Protein A (GE Healthcare Life Sciences) columns, respectively. Recombinant ASCL2 protein was created similarly, using a synthetic DNA fragment that encoded the open reading frame of the ASCL2 mRNA (GenBank accession NM_005170, nts 621-1202).

To create a lentiviral vector plasmid encoding the hASCL1 protein, the synthetic DNA encoding the hASCL1 open reading frame was subcloned in-frame into the multiple cloning site (MCS) of a lentiviral expression vector pCDH-EF1-MCS-T2A-GFP (System Biosciences, Mountain View Calif.), which had been previously modified to introduce nucleotide sequences encoding a DDDK epitope tag upstream of the multiple cloning site (MCS). The T2A sequence downstream of the MCS promotes ribosomal skipping of a peptide bond condensation, resulting in expression of two independent proteins: high level expression of DDDK-tagged proteins encoded upstream of the T2A peptide, with co-expression of the GFP marker protein encoded downstream of the T2A peptide. This cloning step yielded the lentiviral vector plasmid pLMEGPA-hASCL1-NFlag.

Cell Line Engineering

Engineered cell lines overexpressing hASCL1 protein were constructed using the pLMEGPA-hASCL1-NFlag lentiviral vector, described above, to transduce HEK-293T cell lines using standard lentiviral transduction techniques well known to those skilled in the art. hASCL1-positive cells were selected using fluorescent activated cell sorting (FACS) of high-expressing HEK-293T subclones (e.g., cells that were strongly positive for GFP, which serves as a surrogate for high intracellular expression of ASCL1 in cells).

### Example 8

### Generation of Murine Anti-ASCL1 Antibodies

Anti-ASCL1 murine antibodies were produced in two immunization campaigns as follows. Mice from the strains Balb/c, CD-1, and FVB were inoculated with 10 μg hASCL1-Fc emulsified with an equal volume of TiterMax® adjuvant. Following the initial inoculation the mice were injected twice weekly for 4 weeks with 10 μg hASCL1 protein emulsified with an equal volume of alum adjuvant plus CpG.

Mice were sacrificed and draining lymph nodes (popliteal, inguinal, and medial iliac) were dissected and used as a source for antibody producing cells. A single-cell suspension of B cells was produced and (122.5×10⁶ cells) were fused with non-secreting SP2/0-Ag14 myeloma cells (ATCC # CRL-1581) at a ratio of 1:1 by electro cell fusion using a model BTX Hybrimmune System (BTX Harvard Apparatus). Cells were re-suspended in hybridoma selection medium consisting of DMEM medium supplemented with azaserine, 15% fetal clone I serum (Thermo #SH30080-03), 10% BM condimed (Roche #10663573001), 1 mM nonessential amino acids (Corning #25-025-CI) 1 mM HEPES Corning #25-060-CI), 100 IU penicillin-streptomycin (Corning #30-002-CI), 100 IU L-glutamine (Corning #25-005-CI) and were cultured in three T225 flasks containing 100 mL selection medium. The flasks were placed in a humidified 37° C. incubator containing 7% CO2 and 95% air for 6 days.

On day 6 after the fusion the hybridoma library cells were frozen-down temporarily. The cells were thawed in hybridoma selection medium and allowed to rest in a humidified 37° C. incubator for 1 day. The cells were sorted from the flask and plated at one cell per well (using a BD FACSAria I cell sorter) in 90 μL of supplemented hybridoma selection medium (as described above) into 12 Falcon 384-well plates. Remaining unused hybridoma library cells were frozen in liquid nitrogen for future library testing and screening.

The hybridomas were cultured for 10 days and the supernatants from 12×384 clones were screened by ELISA for antibodies specific to hASCL1 yet not cross-reactive with the family member hASCL2, using the following method. Plates were coated with purified hASCL1-Fc or hASCL2-Fc at 0.5 μg/mL in PBS buffer and incubated at 4° C. overnight. Plates were then washed with PBST and blocked with PBS with 5% FBS for 30 min. at 37° C. The blocking solution was removed and 15 μl PBST was added to the wells. 25 μl of hybridoma supernatant was added and incubated overnight at 4° C. After washing with PBST, 30 μL/well HRP-labeled goat anti-mouse IgG diluted 1:10,000 in PBSA was added for 30 min. at room temperature. The plates were washed and developed by the addition of 25 μL/well of the TMB substrate solution (Thermo Scientific) for approximately 5 min. at room temperature. An equal volume of 0.2 M H₂SO₄ was added to stop substrate development. The samples were then analyzed by spectrophotometer at OD 450. Supernatants that had an OD 450 greater than 3 times the background on the ASCL1 plate were considered to be positively reactive, while any clones that also had an OD 450 greater than 3 times the background on the ASCL2 plate were rejected as cross-reactive. Screening of these 12×384 clones from the hASCL1-Fc immunization campaigns yielded numerous antibodies that bound specifically to hASCL1, but not the related family member ASCL2, eighty of which were carried forward for further characterization.

A second immunization campaign was conducted similar to the first with the following modifications: 1) 299×10⁶ cells) were fused with non-secreting SP2/0-Ag14 myeloma cells (ATCC # CRL-1581) at a ratio of 1:1 by electro cell fusion using a model BTX Hybrimmune System (BTX Harvard Apparatus). On day 6, after the fusion, the hybridoma cells were not frozen and were sorted on that day. After the 10 day culture, 6×384 clones were screened by ELISA selecting for antibodies positive to hASCL1-GST (glutathione S-transferase) and counter screened against an irrelevant protein tagged with GST. As part of this, ALK phosphatase-labeled goat anti-mouse IgG diluted 1:5000 in PBSA was used instead of HRP-labeled goat anti-mouse IgG diluted 1:10:000. ELISA samples were analyzed at OD405 instead of OD450.

### Example 9

### Sequencing of Anti-ASCL1 Antibodies

Based on the foregoing, a number of exemplary distinct monoclonal antibodies that bind immobilized human ASCL1 or h293-hASCL1 cells with apparently high affinity were selected for sequencing and further analysis. Sequence analysis of the light chain variable regions and heavy chain variable regions from selected monoclonal antibodies generated in Example 8 confirmed that many had novel complementarity determining regions and often displayed novel VDJ arrangements.

The anti-ASCL1 mouse antibodies that were generated in Example 2 were sequenced as described below. Total RNA was purified from selected hybridoma cells using the RNeasy Miniprep Kit (Qiagen) according to the manufacturer's instructions. Between 10⁴ and 10⁵ cells were used per sample. Isolated RNA samples were stored at −80° C. until used.

The variable region of the Ig heavy chain of each hybridoma was amplified using two 5′ primer mixes comprising eighty-six mouse specific leader sequence primers designed to target the complete mouse VH repertoire in combination with a 3′ mouse Cγ primer specific for all mouse Ig isotypes. Similarly, two primer mixes containing sixty-four 5′ Vκ leader sequences designed to amplify each of the Vκ mouse families was used in combination with a single reverse primer specific to the mouse kappa constant region in order to amplify and sequence the kappa light chain. The VH and VL transcripts were amplified from 100 ng total RNA using the Qiagen One Step RT-PCR kit as follows. A total of four RT-PCR reactions were run for each hybridoma, two for the Vκ light chain and two for the VH heavy chain. PCR reaction mixtures included 1.5 μL of RNA, 0.4 μL of 100 μM of either heavy chain or kappa light chain primers (custom synthesized by Integrated DNA Technologies), 5 μL of 5×RT-PCR buffer, 1 μL dNTPs, and 0.6 μL of enzyme mix containing reverse transcriptase and DNA polymerase. The thermal cycler program was RT step 50° C. for 60 min., 95° C. for 15 min. followed by 35 cycles of (94.5° C. for 30 seconds, 57° C. for 30 seconds, 72° C. for 1 min.). There was then a final incubation at 72° C. for 10 min.

The extracted PCR products were sequenced using the same specific variable region primers as described above for the amplification of the variable regions. PCR products were sent to an external sequencing vendor (MCLAB) for PCR purification and sequencing services. Nucleotide sequences were analyzed using the IMGT sequence analysis tool available online at the website identified as http://www.imgt.org/IMGTmedical/sequence_analysis.html to identify germline V, D and J gene members with the highest sequence homology. The derived sequences were compared to known germline DNA sequences of the Ig V- and J-regions by alignment of VH and VL genes to the mouse germline database using a proprietary antibody sequence database.

FIG. 3A depicts the contiguous amino acid sequences of novel murine light chain variable regions from anti-ASCL1 antibodies while FIG. 3B depicts the contiguous amino acid sequences of novel murine heavy chain variable regions from the same anti-ASCL1 antibodies. Taken together murine light and heavy chain variable region amino acid sequences are provided in SEQ ID NOS: 521-573 odd numbers.

More particularly FIGS. 3A and 3B provide the annotated sequences of murine anti-ASCL1 antibodies comprising: (1) a light chain variable region (VL) of SEQ ID NO: 521 and a heavy chain variable region (VH) of SEQ ID NO: 523; or (2) a VL of SEQ ID NO: 525 and a VH of SEQ ID NO: 527; or (3) a VL of SEQ ID NO: 529 and a VH of SEQ ID NO: 531; or (4) a VL of SEQ ID NO: 533 and a VH of SEQ ID NO: 535; or (5) a VL of SEQ ID NO: 537 and a VH of SEQ ID NO: 539; or (6) a VL of SEQ ID NO: 541 and a VH of SEQ ID NO: 543; or (7) a VL of SEQ ID NO: 545 and a VH of SEQ ID NO: 547; or (8) a VL of SEQ ID NO: 549 and a VH of SEQ ID NO: 551; or (9) a VL of SEQ ID NO: 553 and a VH of SEQ ID NO: 555; or (10) a VL of SEQ ID NO: 557 and a VH of SEQ ID NO: 559; or (11) a VL of SEQ ID NO: 561 and a VH of SEQ ID NO: 563; or (12) a VL of SEQ ID NO: 565 and a VH of SEQ ID NO: 567; or (13) a VL of SEQ ID NO: 569 and a VH of SEQ ID NO: 571; or (14) a VL of SEQ ID NO: 521 and a VH of SEQ ID NO: 573.

A summary of the disclosed antibodies (or clones producing them), with their respective designation (e.g., SC72.2, SC72.28, etc.) and variable region nucleic acid or amino acid SEQ ID NOS (see FIGS. 3A-3C) are shown immediately below in Table 8.

TABLE 8

| | VL | VH |
|---|---|---|
| | SEQ ID NO: | SEQ ID NO: |
| Clone | NA/AA | NA/AA |
| SC72.2 | 520/521 | 522/523 |
| SC72.28 | 524/525 | 526/527 |
| SC72.52 | 528/529 | 530/531 |
| SC72.63 | 532/533 | 534/535 |
| SC72.76 | 536/537 | 538/539 |
| SC72.91 | 540/541 | 542/543 |
| SC72.94 | 544/545 | 546/547 |
| SC72.96 | 548/549 | 550/551 |
| SC72.132 | 552/553 | 554/555 |
| SC72.165 | 556/557 | 558/559 |
| SC72.181 | 560/561 | 562/563 |
| SC72.201 | 564/565 | 566/567 |
| SC72.216 | 568/569 | 570/571 |
| SC72.93 | 520/521 | 572/573 |

The VL and VH amino acid sequences in FIGS. 3A and 3B are annotated to identify the framework regions (i.e. FR1-FR4) and the complementarity determining regions (i.e., CDR-L1-CDR-L3 in FIG. 3A or CDR-H1-CDR-H3 in FIG. 3B), defined as per Kabat et al. The variable region sequences were analyzed using a proprietary version of the Abysis database to provide the CDR and FR designations. Though the CDRs are defined as per Kabat et al., those skilled in the art will appreciate that the CDR and FR designations can also be defined according to Chothia, McCallum or any other accepted nomenclature system. In addition, FIG. 3C provides the nucleic acid sequences (SEQ ID NOS: 520-572, even numbers) encoding the amino acid sequences set forth in FIGS. 3A and 3B.

As seen in FIGS. 3A and 3B and Table 8, the SEQ ID NOS. of the heavy and light chain variable region amino acid sequences for each particular murine antibody are generally sequential odd numbers. Thus, the monoclonal anti-ASCL1 antibody SC72.2 comprises amino acid SEQ ID NOS: 521 and 523 for the light and heavy chain variable regions respectively; SC72.28 comprises SEQ ID NOS: 525 and 527; SC72.52 comprises SEQ ID NOS: 529 and 531, and so on. The single exception to the sequential numbering scheme is SC72.93 which comprises the same light chain variable region amino acid sequence as clone SC72.2 (SEQ ID NO: 521) along with a unique heavy chain variable region amino acid sequence (SEQ ID NO: 573). In any event the corresponding nucleic acid sequence encoding the murine antibody amino acid sequence (set forth in FIG. 3C) has a SEQ ID NO. immediately preceding the corresponding amino acid SEQ ID NO. Thus, for example, the SEQ ID NOS. of the nucleic acid sequences of the VL and VH of the SC72.2 antibody are SEQ ID NOS: 520 and 522, respectively.

### Example 10

### DLL3 Expression in Tumors that have Undergone Targeted Therapy

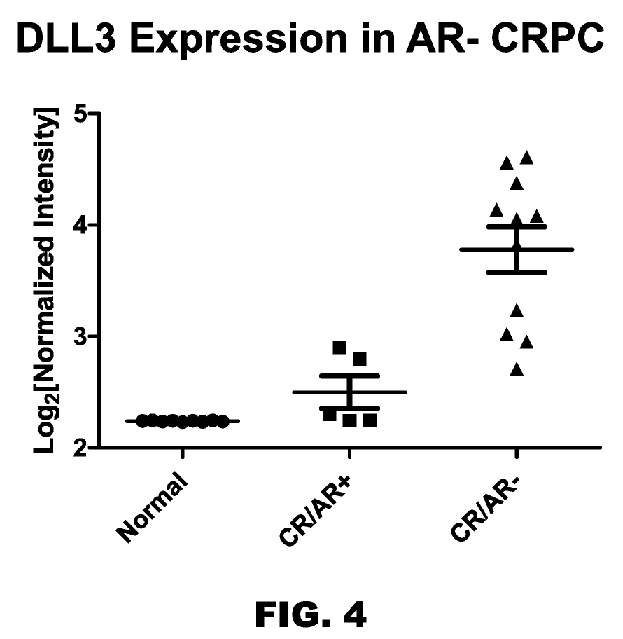

DLL3 is a target of the transcription factor ASCL1, and is expressed in SCLC and other neuroendocrine tumors. Publicly available data sets were analyzed for DLL3 expression and the resulting data is depicted in FIGS. 4-10. The results of this analysis suggest that DLL3 expression is upregulated upon transformation of various adenocarcinomas to a neuroendocrine phenotype, particularly those tumors that have previously undergone a targeted therapy.

FIG. 4 shows an analysis of data described in Tzelepi, 2012 (PMID: 22156612). This analysis revealed that DLL3 expression is limited to patient derived xenograft (PDX) samples of CRPC that lost AR expression (CR/AR-), and express neuroendocrine markers like CHGA and SYP, in contrast to normal prostate samples or CRPC that maintains AR expression (CR/AR+).

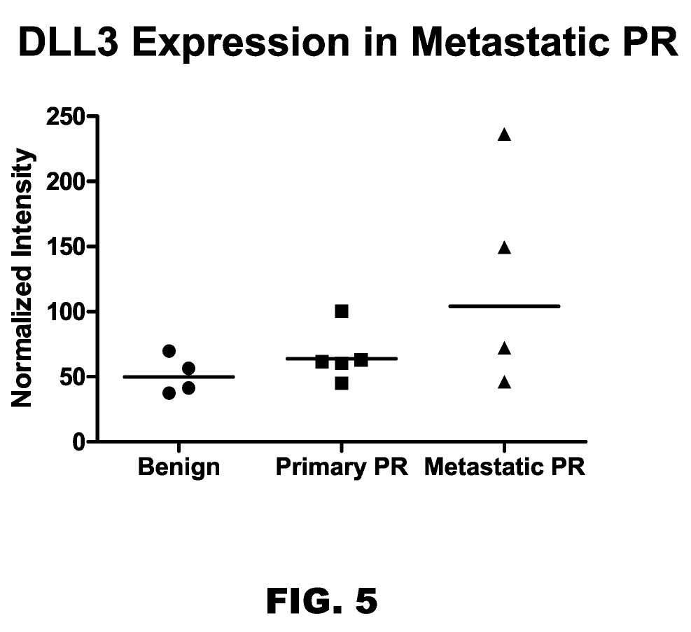

FIG. 5 shows analysis of data described in Varambally, 2005 (database GSE3325; PMID: 16286247) where the analysis revealed DLL3 expression in 2 of 4 metastatic prostate cancer samples, with no DLL3 expression in benign prostate samples or clinically localized prostate adenocarcinoma.

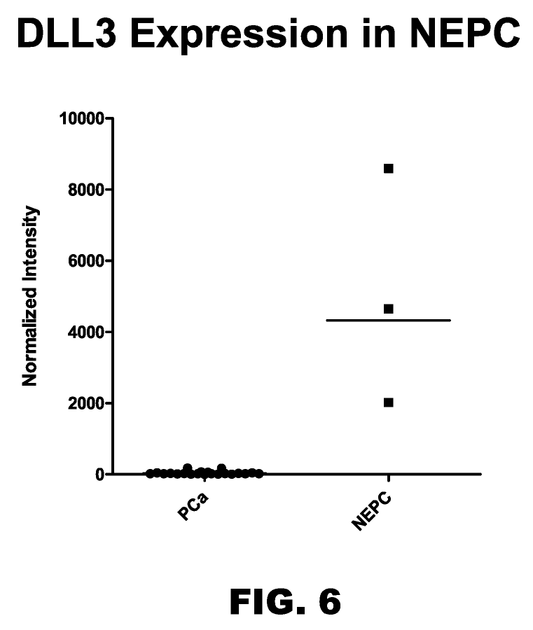

FIG. 6 shows analysis of data described in Lin, 2013 (PMID: 24356420) wherein the analysis revealed DLL3 expression in neuroendocrine transformed CRPC (NEPC) and not in prostate adenocarcinoma (PCa).

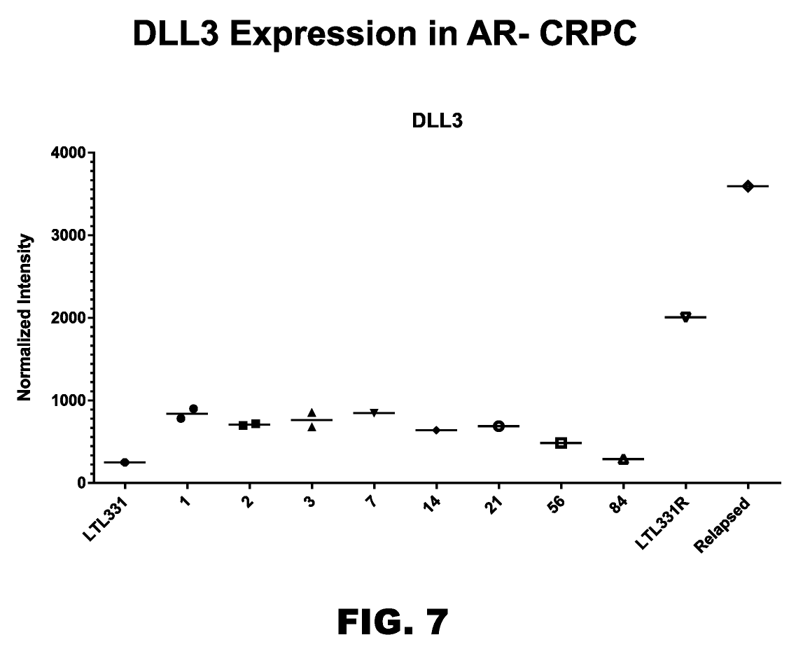

FIG. 7 shows analysis of data described in Akamatsu, 2015 (PMID: 26235627) wherein the data revealed that LTL331, which is a biopsy from a hormone naïve prostate adenocarcinoma that is AR+ and PSA+, is DLL3 negative. A subsequent biopsy from the patient after relapse and development of CRPC (relapsed) shows high expression of DLL3. LTL331 was established as a PDX in a mouse host that was then castrated through treatment with bicalutamide. Timepoints (days post castration) show no DLL3 expression until a CRPC develops in the castrated mouse (LTL331R). Neuroendocrine markers including ASCL1, ENO2, NCAM1 and SYP are also expressed exclusively in LTL331R and the patient biopsy upon relapse. CHGA expression turns on at day 84, just before CRPC develops.

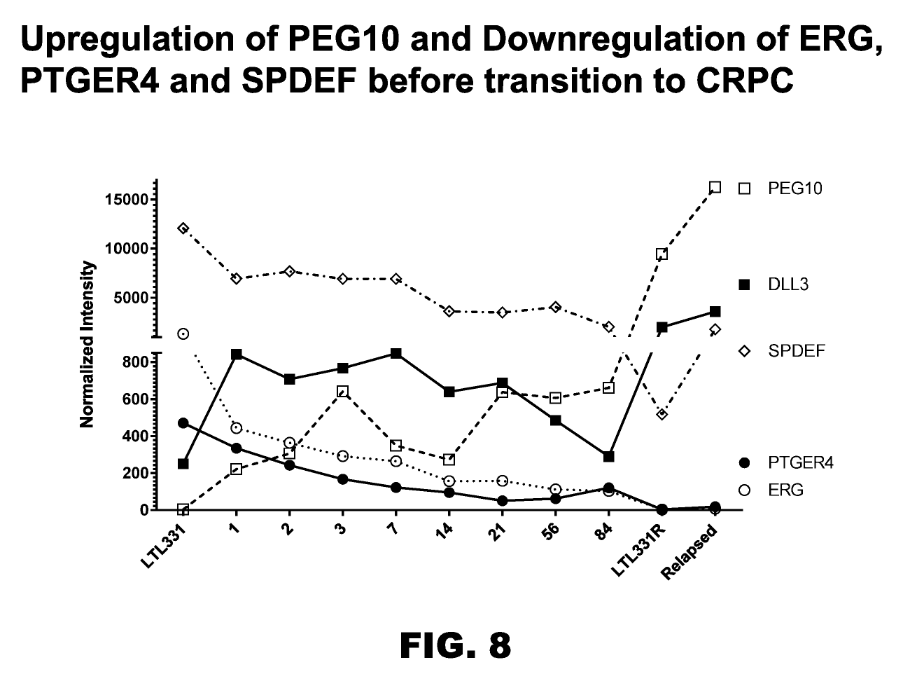

FIG. 8 shows further analysis of data described in Akamatsu, 2015 (PMID: 26235627) wherein the analysis revealed that the expression of certain markers, including PEG10, changed before relapse in this patient. More specifically PEG10 expression is elevated 3 weeks post castration and continues to rise during CRPC development. The analysis also reveals a gradual reduction and/or loss of SPDEF, PTGER4 and ERG expression starting early during mouse host castration, suggesting that PEG10, SPDEF, PTGER4 and ERG may be useful as markers of adenocarcinomas at risk for transitioning to a neuroendocrine phenotype.

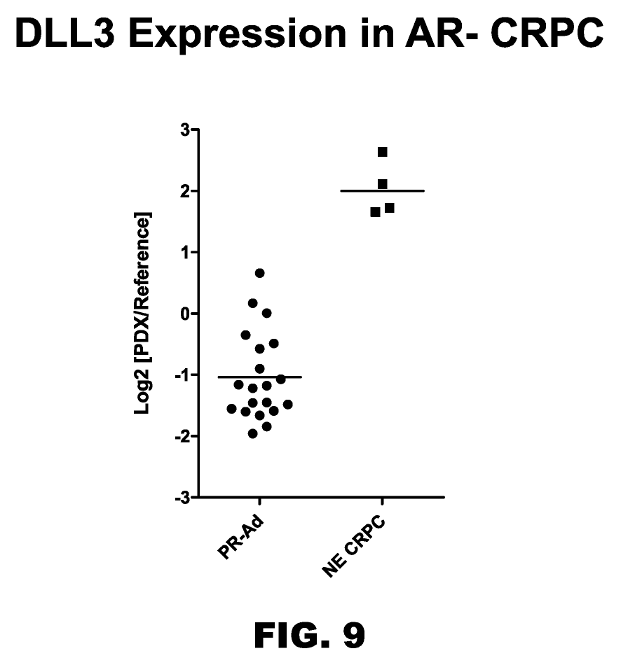

FIG. 9 shows analysis of data described in Zhang, 2015 (PMID: 26071481) where the analysis revealed that 20 prostate adenocarcinoma (PR-Ad) PDX have low or no DLL3 expression, while 4 neuroendocrine transformed CRPC PDX express high levels of DLL3.

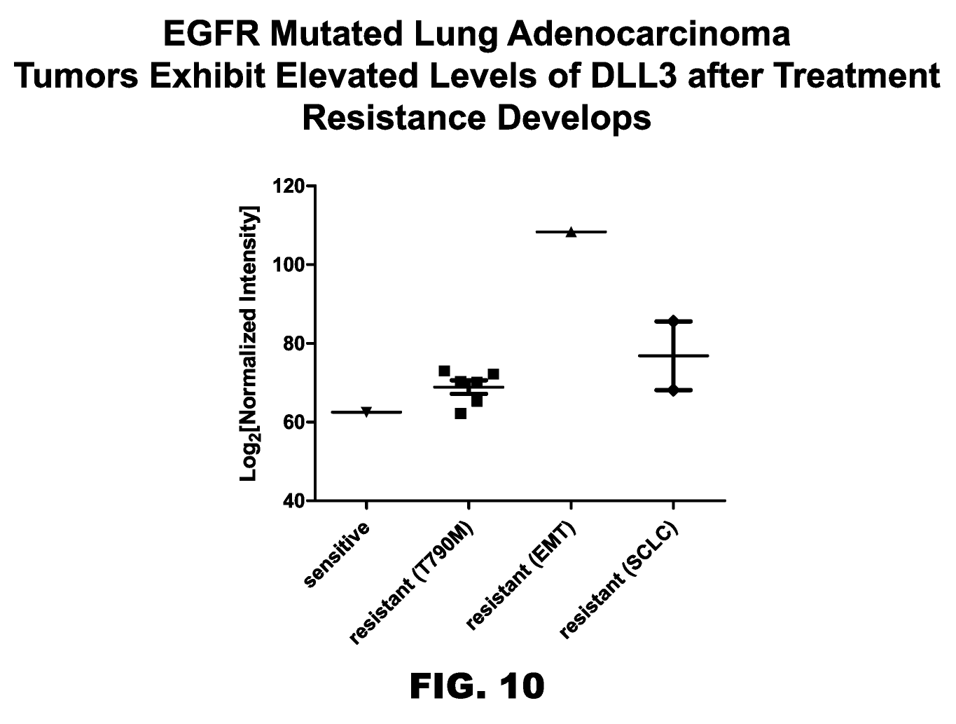

FIG. 10 shows analysis of data described in Niederst, 2015 (PMID 25758528) wherein the analysis revealed a lack of DLL3 expression in an EGFR mutant lung adenocarcinoma biopsy that is sensitive to an EGFR TKI (sensitive), as well as a lack of DLL3 expression in 6 biopsy samples from lung adenocarcinoma patients that harbor a T790M resistance mutation (resistant—T790M). In contrast, elevated DLL3 expression is seen in lung adenocarcinoma that has undergone physiological transformation and become resistant to EGFR TKIs, either due to epithelial to mesenchymal transition (EMT) (resistant—EMT) or due to SCLC transformation (resistant—SCLC; 2 biopsies from same patient). Collectively this analysis showed elevated DLL3 RNA expression in CRPC and lung adenocarcinoma that have transformed to a neuroendocrine phenotype.

In the aggregate these data show that DLL3 is not extensively expressed in either prostate adenocarcinoma or lung adenocarcinoma with EGFR activating mutations. However, following targeted therapy, androgen deprivation therapy for prostate adenocarcinoma, or treatment with a tyrosine kinase inhibitor for EGFR mutated lung adenocarcinoma, treatment resistant tumors arise that apparently have a neuroendocrine component. The treatment resistant tumors now express DLL3.

### Example 11

### Anti-DLL3 IHC Demonstrates that DLL3 is Expressed on CRPC Tumors

To confirm that DLL3 protein expression is seen in CRPC, 6 bone metastasis biopsy samples from CRPC patients were stained by immunohistochemistry with an anti-DLL3 antibody.

IHC was performed essentially as follows. Planar sections of formalin fixed and paraffin embedded (FFPE) tissues were cut and mounted on glass microscope slides. After xylene de-paraffinization sections were pre-treated with Antigen Retrieval Solution (Dako) for 20 mins. at 99° C., cooled to room temperature for 20 minutes and then treated with 0.3% hydrogen peroxide in PBS followed by Avidin/Biotin Blocking Solution (Vector Laboratories). FFPE sections were then blocked with 10% horse or donkey serum in 3% BSA in PBS buffer for 30 minutes at room temperature. Anti-DLL3 antibody (clone SC16.65 was diluted to 10 μg/ml in 3% BSA/PBS and Chromagranin A (clone SP12, Spring Biosciences) antibody was diluted at 1:200 in BSA/PBS. Primary antibodies were applied to sections for a 60 minute incubation at room temperature. The FFPE sections were incubated with biotin-conjugated horse anti-mouse (Vector Labs) or donkey anti-rabbit (Jackson Immunoresearch) secondary antibody diluted to 2.5 μg/ml in 3% BSA/PBS, for 30 mins. at room temperature followed by incubation in streptavidin-HRP (ABC Elite Kit; Vector Laboratories). For DLL3 staining an amplification step was used. FFPE sections were incubated in biotinyl tyramide for 5 minutes at room temperature followed by incubation in streptavidin HRP (Perkin Elmer) for 30 minutes. Chromogenic detection was developed with 3,3′-diaminobenzidine (Thermo Scientific) for 5 mins. at room temperature and tissues were counterstained with Meyer's hematoxylin (IHC World), washed with alcohol and immersed in xylene. Sections were then viewed by brightfield microscopy and DLL3 and CHGA expression was scored.

Immunohistochemistry found DLL3 protein expression in 1/6 CRPC bone metastasis biopsy sample further indicating that the disclosed DLL3 ADCs may be used to treat CRPC.

### Example 12

### IHC Demonstrates that DLL3 is Expressed on Selected Bladder Tumors

IHC Data was generated as set forth herein using a commercially available tumor array to determine if certain markers are indicative DLL3 positive bladder tumors.

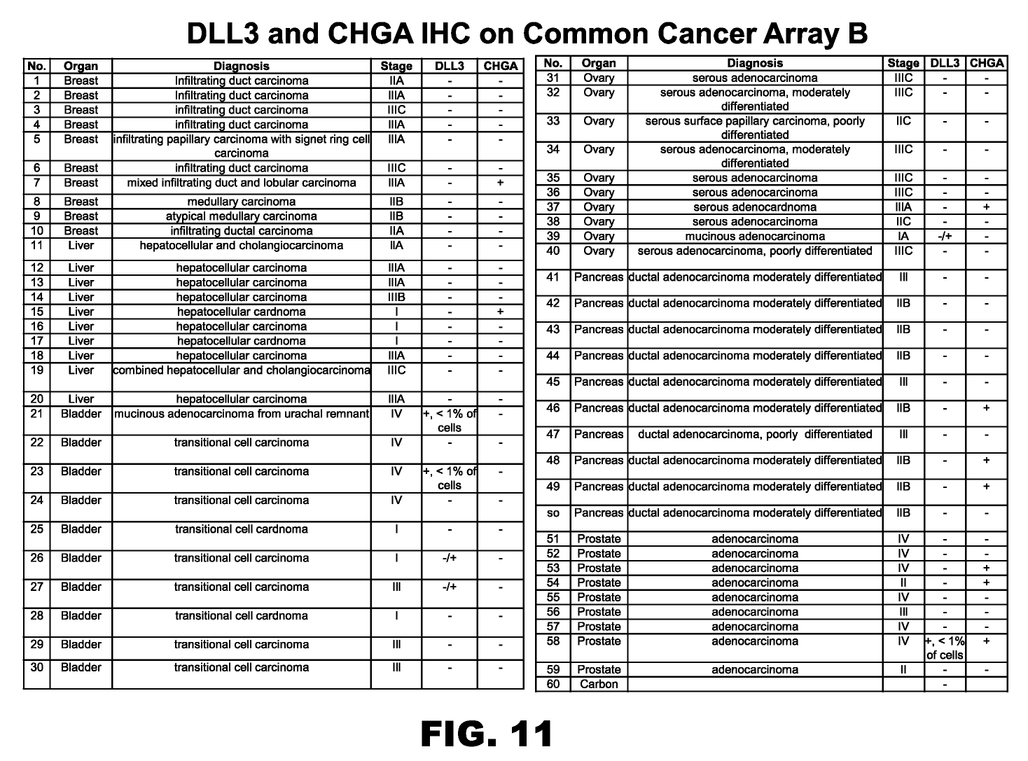

In this regard, FIG. 11 is a tabular summary of DLL3 and CHGA expression following immunohistochemical staining of a common cancer array (Imgenex; IMH-327) with multiple types of tumors. Further analysis of the data revealed that 3 of 9 prostate adenocarcinomas showed some expression of CHGA, and one of those CHGA positive tumors had ≤1% of tumor cells positive for DLL3 expression.

The results of this analysis suggested that tumor heterogeneity might allow for a rare neuroendocrine transformed cell to grow out after treatment with androgen deprivation therapy kills the majority of the AR dependent tumor cells. In addition to prostate, rare DLL3 positive cells were seen in 3/9 bladder transitional cell carcinoma (TCC) biopsy samples, and one bladder mucinous adenocarcinoma, although these were CHGA negative (FIG. 11).

Small cell neuroendocrine tumors do arise in the bladder and are very aggressive, but perhaps due to the lack of targeted therapies, bladder TCC treated with standard chemotherapy has not been reported to transform into small cell neuroendocrine tumors. Concurrent adenocarcinoma with a neuroendocrine carcinoma has been described (Jiang Y 2014; PMID: 25582251), and therefore, based upon the analysis presented herein, bladder carcinoma may also be identified as at risk for transitioning to a neuroendocrine phenotype due to tumor heterogeneity.

### Example 13

### Expression of ASCL1 mRNA in PDX Tumors

To characterize the cellular heterogeneity of solid tumors as they exist in cancer patients and to elucidate molecular subtypes within given tumor indications, a PDX tumor bank was developed and maintained using art recognized techniques. The PDX tumor bank, comprising a large number of discrete tumor cell lines, was propagated in immunocompromised mice through multiple passages of tumor cells originally obtained from cancer patients afflicted by a variety of solid tumor malignancies. Low passage PDX tumors are representative of tumors in their native environments, providing clinically relevant insight into underlying mechanisms driving tumor growth and resistance to current therapies. Selected PDX cell lines of pancreatic, colorectal and lung tumors were then interrogated as set forth immediately below.

In order to perform ASCL1 microarray analysis, PDX tumors were resected from mice after they reached 800-2,000 mm³. Resected PDX tumors were dissociated into single cell suspensions using art-recognized enzymatic digestion techniques (see, for example, U.S.P.N. 2007/0292414). Dissociated bulk tumor cells were incubated with 4′,6-diamidino-2-phenylindole (DAPI) to detect dead cells, anti-mouse CD45 and H-2K^{d} antibodies to identify mouse cells and anti-human EPCAM antibody to identify human cells. RNA was extracted from tumor cells by lysing the cells in RLTplus RNA lysis buffer (Qiagen) supplemented with 1% 2-mercaptoethanol, freezing the lysates at −80° C. and then thawing the lysates for RNA extraction using an RNeasy isolation kit (Qiagen). RNA was quantified using a Nanodrop spectrophotometer (Thermo Scientific) and/or a Bioanalyzer 2100 (Agilent Technologies). Normal tissue RNA was purchased from various sources (Life Technology, Agilent, ScienCell, BioChain, and Clontech). The resulting total RNA preparations were assessed by microarray analysis (Agilent Technologies).

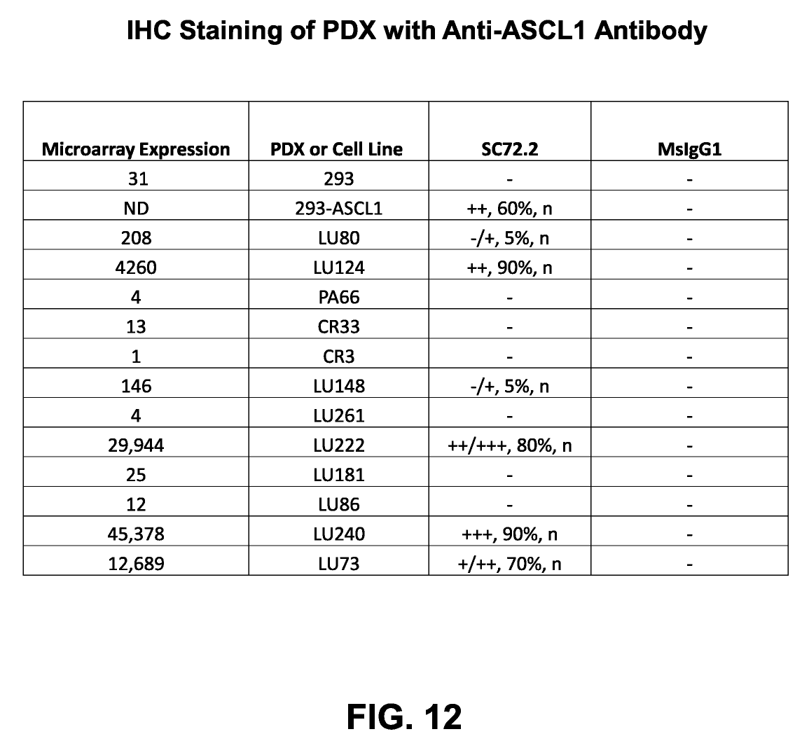

Microarray experiments on various PDX lines (and engineered 293 cell controls) were conducted and data was analyzed as follows: 1-2 μg of whole tumor total RNA was extracted, substantially as described above, from PDX lines. The RNA samples were analyzed using the Agilent SurePrint GE Human 8×60 v2 microarray platform, which contains 50,599 biological probes designed against 27,958 genes and 7,419 lncRNAs in the human genome. Standard industry practices were used to normalize and transform the intensity values to quantify gene expression for each sample. The normalized intensity of ASCL1 expression is set forth in column 1 of FIG. 12 entitled “Microarray Expression.”

As shown in FIG. 12 the microarray analysis demonstrates that ASCL1 is expressed in a number of lung cancer cell lines indicating that it may provide a marker for detecting, diagnosing or monitoring certain neoplastic disorders and a potential marker of tumors at risk of transition to a neuroendocrine phenotype.

### Example 14

### Expression of ASCL1 by IHC

Microarray analysis and immunohistochemistry (IHC) was performed on PDX tumor sections to assess the expression of ASCL1 in tumor cells.

IHC was performed essentially as follows. Planar sections of formalin fixed and paraffin embedded (FFPE) tissues were cut and mounted on glass microscope slides. After xylene de-paraffinization 5 μm sections were pre-treated with Antigen Retrieval Solution (Dako) for 20 mins. at 99° C., cooled to 75° C. and then treated with 0.3% hydrogen peroxide in PBS followed by Avidin/Biotin Blocking Solution (Vector Laboratories). FFPE slides were then blocked with 10% horse serum in 3% BSA in PBS buffer and incubated with a primary anti-ASCL1 antibody (clone SC72.2), diluted to 10 μg/ml in 3% BSA/PBS, for 30 mins. at room temperature. The FFPE slides were incubated with biotin-conjugated horse anti-mouse antibody (Vector Laboratories), diluted to 2.5 μg/ml in 3% BSA/PBS, for 30 mins. at room temperature followed by incubation in streptavidin-HRP (ABC Elite Kit; Vector Laboratories). Chromogenic detection was developed with 3,3′-diaminobenzidine (Thermo Scientific) for 5 mins. at room temperature and tissues were counterstained with Meyer's hematoxylin (IHC World), washed with alcohol and immersed in xylene. Sections were then viewed by brightfield microscopy and ASCL1 expression was noted. Results of the studies are shown in FIG. 12.

A review of FIG. 12 shows that ASCL1 expression was detected in the nucleus (n) of 293 cells engineered to express ASCL1 (293-ASCL1), but not in naïve 293 cells. PDX tumors were stained by IHC (FIG. 12) and ASCL1 protein expression by IHC correlates with the expected ASCL1 mRNA expression based on microarray results.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

We claim:

1. A method of treating an adenocarcinoma at risk of transitioning to a neuroendocrine phenotype in a subject, the method comprising administering to the subject a therapeutically effective amount of an anti-DLL3 antibody drug conjugate (ADC), or a pharmaceutically acceptable salt thereof, wherein the antibody drug conjugate (ADC) comprises the formula M-[L-D]n wherein:
M comprises an anti-DLL3 antibody;
L comprises an optional linker;
D comprises a cytotoxic agent; and
n is an integer from 1 to 20.

2. A method of reducing or inhibiting recurrence of an adenocarcinoma at risk of transitioning to a neuroendocrine phenotype in a subject, the method comprising administering to the subject a therapeutically effective amount of an anti-DLL3 antibody drug conjugate (ADC), or a pharmaceutically acceptable salt thereof, wherein the antibody drug conjugate (ADC) comprises the formula M-[L-D]n wherein:
M comprises an anti-DLL3 antibody;
L comprises an optional linker;
D comprises a cytotoxic agent; and
n is an integer from 1 to 20.

3. The method of claim 1 or 2, wherein the adenocarcinoma comprises ASCL1⁺ cells.

4. The method of any one of claims 1-3, wherein the adenocarcinoma shows reduced expression of one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG), as compared to a control sample.

5. The method of any one of claims 1-4 wherein the adenocarcinoma shows increased expression of paternally expressed 10 (PEG10) as compared to a control sample.

6. The method of any one of claims 1-5, wherein the adenocarcinoma is recurrent, refractory, relapsed or resistant.

7. The method of any one of claims 1-6, wherein the subject has undergone a targeted therapy.

8. The method of any one of claims 1-7, wherein the subject has previously undergone a debulking procedure.

9. The method of any one of claims 1-8, wherein the adenocarcinoma occurs in lung, prostate, genitourinary tract, gastrointestinal tract, thyroid, or kidney.

10. The method of claim 9, wherein the adenocarcinoma comprises prostate cancer.

11. The method of claim 10, wherein the prostate cancer comprises castration resistant prostate cancer.

12. The method of claim 10 or 11, wherein the adenocarcinoma is resistant to androgen deprivation therapy.

13. The method of claim 9, wherein the adenocarcinoma comprises lung cancer.

14. The method of claim 13, wherein the lung cancer comprises non-small cell lung cancer.

15. The method of claim 13 or 14, wherein the adenocarcinoma is characterized as having an activating EGFR mutation.

16. The method of any one of claims 13-15, wherein the adenocarcinoma is resistant to EGFR inhibitor therapy.

17. The method of any one of claims 1-16 wherein the adenocarcinoma is DLL3^{-/low}.

18. The method of any one of claims 1-17, wherein the anti-DLL3 antibody is selected from the group consisting of a monoclonal antibody, primatized antibody, multispecific antibody, bispecific antibody, monovalent antibody, multivalent antibody, anti-idiotypic antibody, diabody, Fab fragment, F(ab′)₂ fragment, Fv fragment, and ScFv fragment; or an immunoreactive fragment thereof.

19. The method of claim 18, wherein the anti-DLL3 antibody is selected from the group consisting of a chimeric antibody, a CDR-grafted antibody, and a humanized antibody.

20. The method of any one of claims 1-19, wherein the anti-DLL3 antibody specifically binds to an epitope within the DSL domain of a DLL3 protein set forth as SEQ ID NO: 1 or 2.

21. The method of any one of claims 1-20, wherein the anti-DLL3 antibody comprises or competes for binding to human DLL3 protein with an antibody comprising a light chain variable region set forth as SEQ ID NO: 149 and a heavy chain variable region set forth as SEQ ID NO: 151.

22. The method of any one of claims 1-21, wherein the anti-DLL3 antibody comprises three complementarity determining regions of a light chain variable region set forth as SEQ ID NO: 149, and three complementarity determining regions of a heavy chain variable region set forth as SEQ ID NO: 151.

23. The method of claim 22, wherein the anti-DLL3 antibody comprises residues 24-34 of SEQ ID NO: 149 for CDR-L1, residues 50-56 of SEQ ID NO: 149 for CDR-L2, residues 89-97 of SEQ ID NO: 149 for CDR-L3, residues 31-35 of SEQ ID NO: 151 for CDR-H1, residues 50-65 of SEQ ID NO: 151 for CDR-H2 and residues 95-102 of SEQ ID NO: 151 for CDR-H3, wherein the residues are numbered according to Kabat.

24. The method of claim 22, wherein the anti-DLL3 antibody comprises a light chain variable region comprising an amino acid sequence set forth as SEQ ID NO: 405 and a heavy chain variable region comprising an amino acid sequence set forth as SEQ ID NO: 407.

25. The method of any one of claims 1-24, wherein the cytotoxic agent is a pyrrolobenzodiazepine (PBD), an auristatin, a maytansinoid, a calicheamicin, or a radioisotope.

26. The method of claim 25, wherein the cytotoxic agent is a pyrrolobenzodiazepine (PBD).

27. The method of claim 26, wherein the PBD is covalently linked to the anti-DLL3 antibody via a linker.

28. The method of claim 27, wherein the cytotoxic agent is a pyrrolobenzodiazepine (PBD) comprising formula AC: 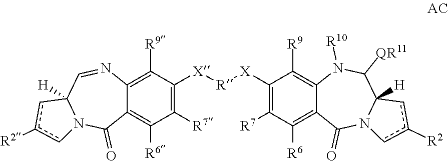
wherein:
the dotted lines indicate the optional presence of a double bond, and wherein only one of the dotted lines in a given ring can be a double bond;
R² is selected from H, OH, ═O, ═CH₂, CN, R, OR, ═CH—R^{D}, ═C(R^{D})₂, O SO₂ R, CO₂R, COR, and halo, where R^{D} is selected from R, CO₂R, COR, CHO, CO₂H, and halo;
R⁶ and R⁹ are each independently selected from H, R, OH, OR, SH, SR, NH₂, NHR, NRR′, NO₂, Me₃Sn and halo;
R⁷ is selected from H, R, OH, OR, SH, SR, NH₂, NHR, NRR′, NO₂, Me₃Sn and halo;
R¹⁰ is the linker L connected to the anti-DLL3 antibody;
Q is selected from O, S and NH;
R¹¹ is either H, or R or, where Q is O, SO₃M, where M is a metal cation;
R and R′ are each independently selected from optionally substituted C₁₋₁₂ alkyl, C₃₋₂₀ heterocyclyl and C₅₋₂₀ aryl groups, and optionally in relation to the group NRR′, R and R′ together with the nitrogen atom to which they are attached form an optionally substituted 4-, 5-, 6- or 7-membered heterocyclic ring;
X is selected from O, S, and N(H);
R^{2″}, R^{6″}, R^{7″}, R^{9″}, and X″ are as defined according to R², R⁶, R⁷, R⁹, and X, respectively; and
R″ is a C₃₋₁₂ alkylene group, which comprises a chain optionally interrupted by one or more heteroatoms, one or more rings, or both one or more heteroatoms and one or more rings, wherein the optional one or more rings are optionally substituted.

29. The method of claim 28, wherein:
R² is R, wherein R is a C₅₋₂₀ aryl group;
R⁶ and R⁹ are H;
R⁷ is OR, and wherein R is a C₁ alkyl;
Q is O, and wherein R¹¹ is H; and/or
X and X″ are O.

30. The method of claim 27, wherein the PBD comprises:

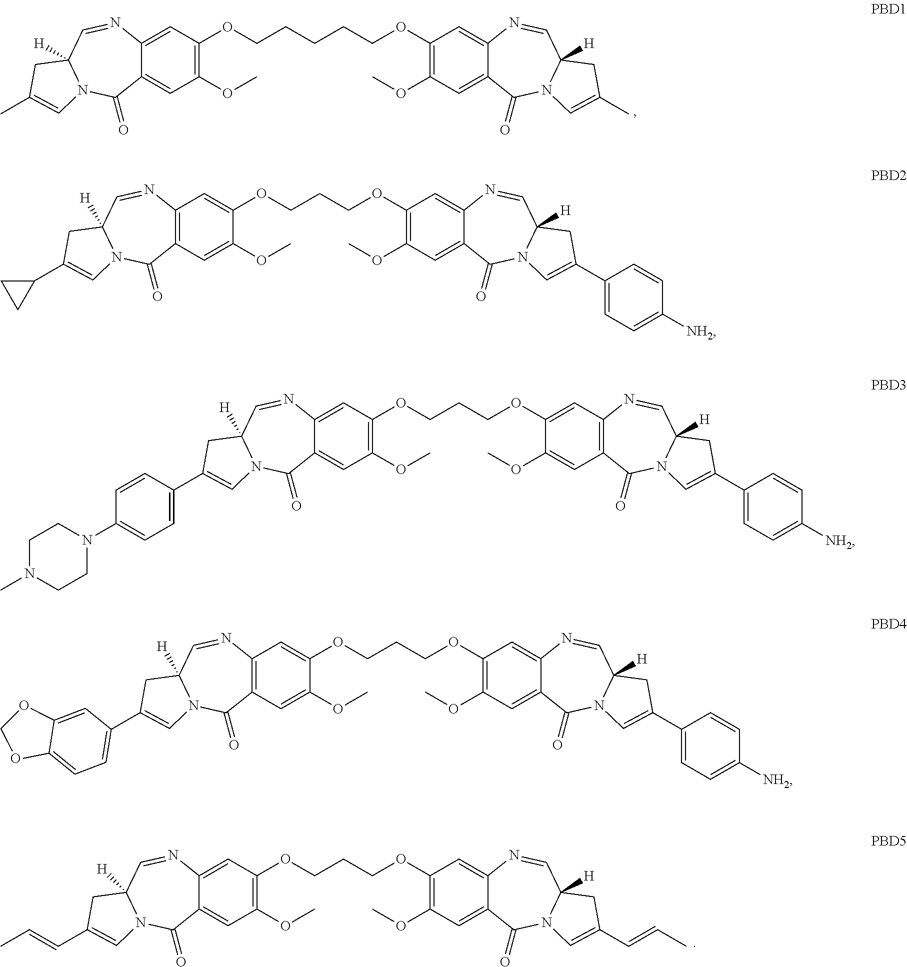

31. The method of any one of claims 1-30, wherein the linker comprises a cleavable linker.

32. The method of claim 31, wherein the cleavable linker comprises a dipeptide.

33. The method of claim 32, wherein the dipeptide is Phe-Lys, Val-Ala, Val-Lys, Ala-Lys, Val-Cit, Phe-Cit, Leu-Cit, Ile-Cit, Phe-Arg, or Trp-Cit.

34. The method of claim 33, wherein the dipeptide is Val-Ala.

35. The method of any one of claims 1-34, wherein the antibody drug conjugate comprises the structure: 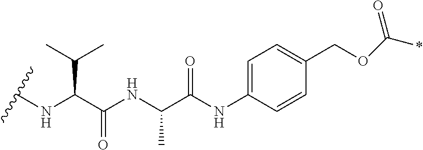 [SMILES: *N[C@H](C(=O)N[C@@H](C)C(=O)Nc1ccc(COC(*)=O)cc1)C(C)C]
wherein the asterisk indicates the point of attachment of the linker to the cytotoxic agent, and wherein the wavy line indicates the point of attachment to the remaining portion of the linker.

36. The method of any one of claims 30-35, wherein the linker further comprises a maleimide group.

37. The method of claim 27 wherein the PBD covalently linked to the anti-DLL3 antibody via a linker comprises an ADC of a structure selected from the group consisting of: 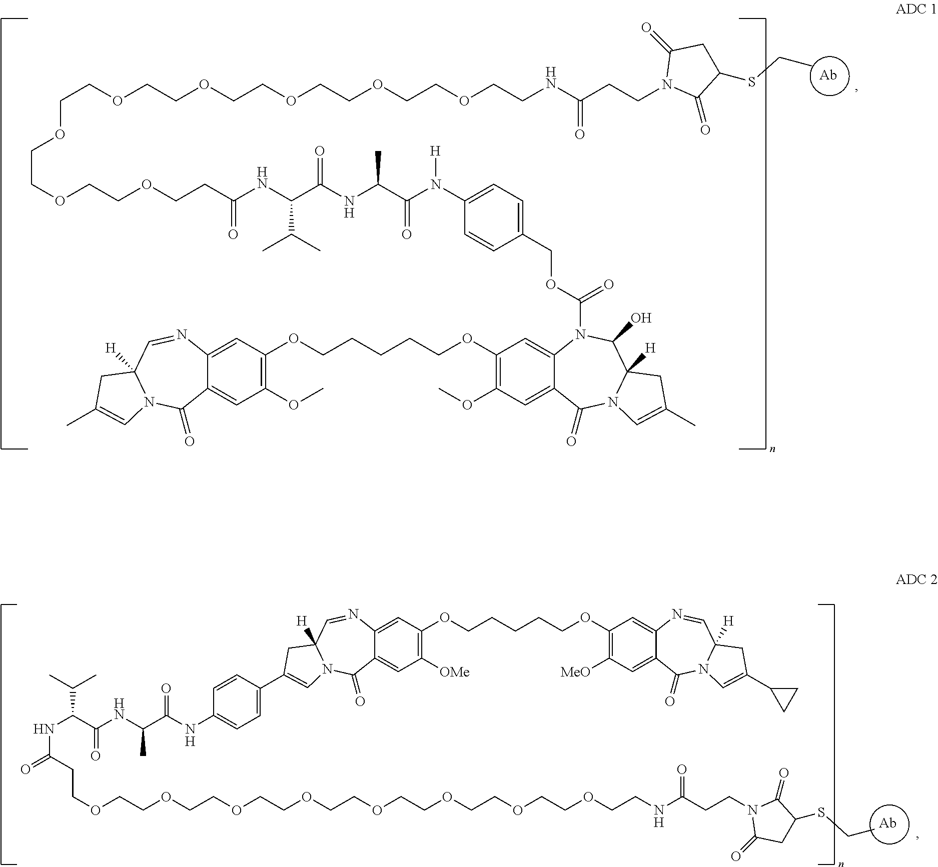 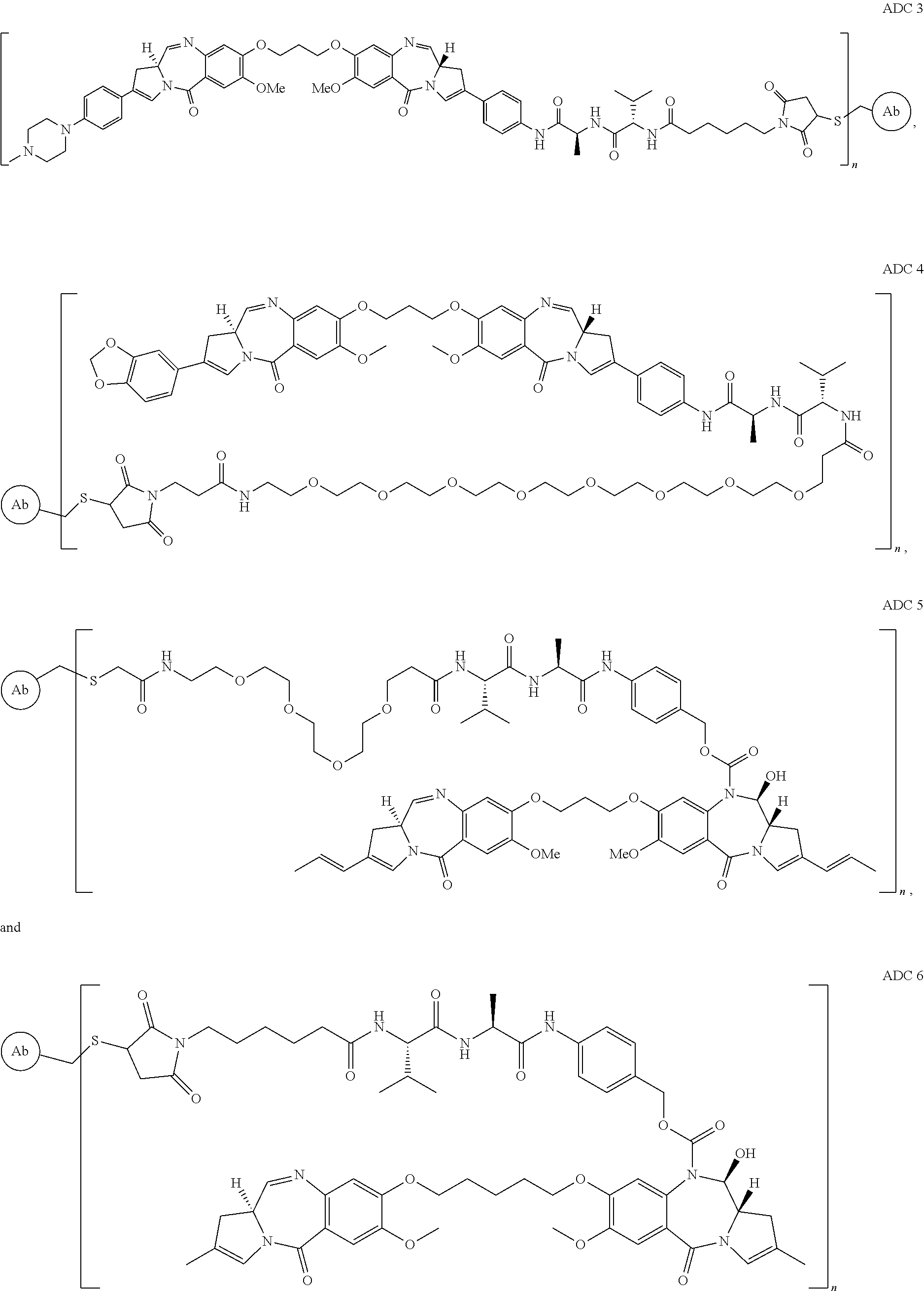
wherein Ab comprises an anti-DLL3 antibody or immunoreactive fragment thereof and n is an integer from about 1 to about 20.

38. A method of treating a subject suffering from a tumor at risk of transitioning to a neuroendocrine phenotype comprising the steps of:
(a) contacting a tumor sample obtained from the subject with an ASCL1 antibody;
(b) detecting the ASCL1 antibody bound to the tumor sample;
(c) selecting a subject having an ASCL1 tumor phenotype; and
(d) treating the subject selected in step (c) with an anti-DLL3 antibody drug conjugate (DLL3 ADC) wherein the tumor sample comprises a DLL3^{-/low} phenotype.

39. The method of claim 38 further comprising the step of contacting the tumor sample with a DLL3 antibody.

40. The method of claims 38 and 39 wherein detecting the ASCL1 antibody is performed using immunohistochemistry.

41. The method of claims 38 to 40, wherein the tumor sample is chemically fixed.

42. The method of claim 41, wherein the tumor sample is chemically fixed using formalin.

43. The method of any one of claims 38-42, wherein the tumor sample is paraffin embedded.

44. The method of claim 38, further comprising the steps:
contacting the tumor sample with an agent that detects one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG);
detecting the agent in the tumor sample; and
observing a reduced expression of one or more of Retinoblastoma 1 (RB1), Repressor Element-1 Silencing Transcription Factor (REST), SAM pointed domain-containing Ets transcription factor (SPDEF), Prostaglandin E2 Receptor 4 (PTGER4), and ETS-related gene (ERG), as compared to a control sample.

45. The method of claim 38 to 44, further comprising the steps:
contacting the tumor sample with a PEG10 agent that detects paternally expressed 10 (PEG10);
detecting the PEG10 agent of in the tumor sample; and
observing an increase in expression of paternally expressed 10 (PEG10) as compared to a control sample.

46. The method of any one of claims 38-45, wherein the tumor is recurrent, refractory, relapsed or resistant.

47. The method of any one of claims 38-46, wherein the subject has undergone a targeted therapy.

48. The method of any one of claims 38-47, wherein the subject has previously undergone a debulking procedure.

49. The method of any one of claims 38-48, wherein the tumor comprises an adenocarcinoma.

50. The method of claim 49 wherein the adenocarcinoma occurs in lung, prostate, genitourinary tract, gastrointestinal tract, thyroid, or kidney.

51. The method of claim 50, wherein the adenocarcinoma comprises prostate cancer.

52. The method of claim 51, wherein the prostate cancer comprises castration resistant prostate cancer.

53. The method of claim 51 or 52, wherein the prostate cancer is resistant to androgen deprivation therapy.

54. The method of claim 50, wherein the adenocarcinoma comprises lung cancer.

55. The method of claim 54, wherein the lung cancer comprises small cell lung cancer.

56. The method of claim 55, wherein the lung cancer comprises non-small cell lung cancer.

57. The method of claim 55 or 56, wherein the adenocarcinoma is characterized as having an activating EGFR mutation.

58. The method of any one of claims 55-57, wherein the adenocarcinoma is resistant to EGFR inhibitor therapy.

59. The method of any one of claims 38-58, wherein the ASCL1 antibody is conjugated or otherwise associated with a detectable label.

60. The method of any one of claims 38-58, wherein the ASCL1 antibody is unlabeled.

61. The method of claim 60, wherein detecting the ASCL1 antibody further comprises contacting the adenocarcinoma sample of (b) with an antibody that specifically binds to the ASCL1 antibody; and detecting the antibody that specifically binds to the ASCL1 antibody.

62. The method of claims 38-61 wherein the percentage of cells in the DLL3^{-/low} tumor that stain positive when interrogated with a DLL3 antibody is less than about 10%.

63. The method of claims 38-62 wherein the percentage of cells in the DLL3^{-/low} tumor that stain positive when interrogated with a DLL3 antibody is less than about 5%.

64. The method of claims 38-63 wherein the percentage of cells in the DLL3^{-/low} tumor that stain positive when interrogated with a DLL3 antibody is less than about 2%.

65. The method of claims 38-64 wherein the percentage of cells in the DLL3^{-/low} tumor that stain positive when interrogated with a DLL3 antibody is less than about 1%.
